SEQUENCE LISTING (339 sequences)

SEQ ID NO: 1 - protein, 42 aa, Homo sapiens
YAEGTFISDYSIAMDKIHQQDFVNWLLAQKGKKNDWKHNITQ

SEQ ID NO: 2 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence 2OEGgE-C18DA
YXEGTFISDYSIALDRXHQXNFVNWLLAQXPSSGAPPPS

SEQ ID NO: 3 - protein, 39 aa, Artificial Sequence
  source: /note=“Description of Artificial Sequence Synthetic polypeptide”
  MOD_RES (2)..(2): Aib
  MOD_RES (20)..(20): Aib
  MOD_RES (30)..(30): Lys, wherein the side chain of Lys is connected to the sequence 2OEGgE-C18DA
YXEGTFISDYSIALDRIHQXNFVNWLLAQXPSSGAPPPS

SEQ ID NO: 4 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C18DA
YXEGTFISDYSIAXDRIHQQNFVNWLLAQRPSSGAPPPS

SEQ ID NO: 5 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C18DA
YXEGTFISDYSIALDRXHQQNFVNWLLAQRPSSGAPPPS

SEQ ID NO: 6 - protein, 30 aa, Artificial Sequence
  source: /note=“Description of Artificial Sequence Synthetic polypeptide”
  MOD_RES (2)..(2): Aib
  MOD_RES (30)..(30): Lys, wherein the side chain of Lys is connected to the sequence 2OEGgE-C18DA
YXEGTFISDYSIALDRIHQQNFVNWLLAQX

SEQ ID NO: 7 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C18DA
  MOD_RES (17)..(17): Aib
  MOD_RES (20)..(20): Aib
YXEGTFISDYSIAXDRXAQXNFVNWLLAQK

SEQ ID NO: 8 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgEgE-C17DA
  MOD_RES (17)..(17): Aib
  MOD_RES (20)..(20): Aib
YXEGTFISDYSIAXDRXAQXNFVNWLLAQR

SEQ ID NO: 9 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C17DA
YXEGTFISDYSIALDRXAQXXFVNWLLAQR

SEQ ID NO: 10 - protein, 30 aa, Artificial Sequence; identical to SEQ ID NO: 9
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C17DA

SEQ ID NO: 11 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C17DA
  MOD_RES (17)..(17): Aib
  MOD_RES (20)..(20): Aib
YXEGTFISDYSIAXDRXAQXNFVNWLLAQS

SEQ ID NO: 12 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C17DA
  MOD_RES (20)..(20): Aib
YXEGTFISDYSIALDRXXQXNFVNWLLAQSPSSGAPPPS

SEQ ID NO: 13 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence gEgEgE-C17DA
  MOD_RES (17)..(17): Aib
  MOD_RES (20)..(20): Aib
YXEGTFISDYSIAXDRXAQXNFVNWLLAQSPSSGAPPPS

SEQ ID NO: 14 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C17DA
YXEGTFISDYSIXLDRXAQXXFVNWLLAQRPSSGAPPPS

SEQ ID NO: 15 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgEgE-C17DA
  MOD_RES (17)..(17): Aib
  MOD_RES (20)..(20): Aib
YXEGTFISDYSIXXDRXAQXNFVNWLLAQRPSSGAPPPS

SEQ ID NO: 16 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgEgE-C17DA
  MOD_RES (20)..(20): Aib
YXEGTFISDYSIXLDRXAQXNFVNWLLAQRPSSGAPPPS

SEQ ID NO: 17 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgEgE-C17DA
YXEGTFISDYSIXLDRXAQXNFVNWLLAQXPSSGAPPPS

SEQ ID NO: 18 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C18DA
  MOD_RES (20)..(20): Aib
YXEGTFISDYSIALDRXXQXNFVNWLLAQR

SEQ ID NO: 19 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIALDRXHQXXFVNWLLAQR

SEQ ID NO: 20 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgEgE-C18DA
YXEGTFISDYSIALDRXHQXNFVNWLLAXR

SEQ ID NO: 21 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G5gE-C17DA
  MOD_RES (20)..(20): Aib
YXEGTFISDYSIALDRXHQXNFVNWLLAQRPSSGAPPPS

SEQ ID NO: 22 - protein, 35 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIALDRXAQXXFVNWLLAQRGGGGS

SEQ ID NO: 23 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5gE-C17DA
  MOD_RES (17)..(17): Aib
  MOD_RES (20)..(20): Aib
YXEGTFISDYSIAXDRXAQXDFVNWLLAQRPSSGAPPPS

SEQ ID NO: 24 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C18DA
  MOD_RES (20)..(20): Aib
YXEGTFISDYSIALDRXAQXDFVNWLLAQRPSSGAPPPS

SEQ ID NO: 25 - protein, 39 aa, Artificial Sequence; identical to SEQ ID NO: 23
  source: /note=“Description of Artificial Sequence Synthetic polypeptide”
  MOD_RES (2)..(2): Aib
  MOD_RES (14)..(14): Lys, wherein the side chain of Lys is connected to the sequence G5gE-C18DA
  MOD_RES (17)..(17): Aib
  MOD_RES (20)..(20): Aib

SEQ ID NO: 26 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgE-C17DA
  MOD_RES (20)..(20): Aib
YXEGTFISDYSIALDRXXQXDFVNWLLAQRPSSGAPPPS

SEQ ID NO: 27 - protein, 39 aa, Artificial Sequence; identical to SEQ ID NO: 16
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgEgE-C18DA
  MOD_RES (20)..(20): Aib

SEQ ID NO: 28 - protein, 39 aa, Artificial Sequence; identical to SEQ ID NO: 17
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgEgE-C18DA

SEQ ID NO: 29 - protein, 39 aa, Artificial Sequence; identical to SEQ ID NO: 15
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgEgE-C18DA
  MOD_RES (17)..(17): Aib
  MOD_RES (20)..(20): Aib

SEQ ID NO: 30 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgEgE-C17DA
  MOD_RES (17)..(17): Aib
  MOD_RES (20)..(20): Aib
YXEGTFISDYSIXXDRXAQXNFVNWLLAQSPSSGAPPPS

SEQ ID NO: 31 - protein, 35 aa, Artificial Sequence; identical to SEQ ID NO: 22
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C17DA

SEQ ID NO: 32 - protein, 35 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C17DA
  MOD_RES (20)..(20): Aib
YXEGTFISDYSIALDRXXQXNFVNWLLAQSGGGGS

SEQ ID NO: 33 - protein, 35 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgEgE-C17DA
  MOD_RES (17)..(17): Aib
  MOD_RES (20)..(20): Aib
YXEGTFISDYSIXXDRXAQXNFVNWLLAQRGGGGS

SEQ ID NO: 34 - protein, 35 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgEgE-C17DA
YXEGTFISDYSIXLDRXAQXNFVNWLLAQXGGGGS

SEQ ID NO: 35 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5gEgE-C18DA
  MOD_RES (17)..(17): Aib
  MOD_RES (20)..(20): Aib
YXEGTFISDYSIAXDRXAQXNFVNWLLAQRPSSGAPPPS

SEQ ID NO: 36 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIALDRXAQXXFVNWLLAQRPSSGAPPPS

SEQ ID NO: 37 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C17DA
YXEGTFISDYSIALDRXAQXXFVNWLLAQRG

SEQ ID NO: 38 - protein, 31 aa, Artificial Sequence; identical to SEQ ID NO: 37
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA

SEQ ID NO: 39 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDRXAQXDFVNWLLAQSPSSGAPPPS

SEQ ID NO: 40 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C20DA
YXEGTFISDYSIALDRXAQXXFVNWLLAQKG

SEQ ID NO: 41 - protein, 39 aa, Artificial Sequence; identical to SEQ ID NO: 13
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C20DA
  MOD_RES (17)..(17): Aib
  MOD_RES (20)..(20): Aib

SEQ ID NO: 42 - protein, 31 aa, Artificial Sequence; identical to SEQ ID NO: 40
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA

SEQ ID NO: 43 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIALDRXAQXXFVNWLLAQSG

SEQ ID NO: 44 - protein, 39 aa, Artificial Sequence; identical to SEQ ID NO: 23
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 45 - protein, 39 aa, Artificial Sequence; identical to SEQ ID NO: 35
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 46 - protein, 39 aa, Artificial Sequence; identical to SEQ ID NO: 13
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 47 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C18DA
  MOD_RES (20)..(20): Aib
YXEGTFISDYSIALDRXXQXNFVNWLLAQSG

SEQ ID NO: 48 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIXXDRXAQXDFVNWLLAQSPSSGAPPPS

SEQ ID NO: 49 - protein, 39 aa, Artificial Sequence; identical to SEQ ID NO: 48
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 50 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C18DA
  MOD_RES (20)..(20): Aib
YXEGTFISDYSIALDRXXQXDFVNWLLAQRG

SEQ ID NO: 51 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C18DA
  MOD_RES (20)..(20): Aib
YXEGTFISDYSIALDRXAQXDFVNWLLAQSPSSGAPPPS

SEQ ID NO: 52 - protein, 39 aa, Artificial Sequence; identical to SEQ ID NO: 24
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C18DA
  MOD_RES (20)..(20): Aib

SEQ ID NO: 53 - protein, 39 aa, Artificial Sequence; identical to SEQ ID NO: 51
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C18DA
  MOD_RES (20)..(20): Aib

SEQ ID NO: 54 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIXXDRXAQXDFVNWLLAQSG

SEQ ID NO: 55 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIXXDRXAQXDFVNWLLAQRPSSGAPPPS

SEQ ID NO: 56 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgEgE-C18DA
YXEGTFISDYSIALDRXAQXNFVNWLLAXRG

SEQ ID NO: 57 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgEgE-C18DA
YXEGTFISDYSIALDRXAQXDFVNWLLAXRG

SEQ ID NO: 58 - protein, 35 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgEgE-C18DA
YXEGTFISDYSIALDRXAQXDFVNWLLAXRGGGGS

SEQ ID NO: 59 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgEgE-C18DA
YXEGTFISDYSIALDRXAQXDFVNWLLAXRPSSGAPPPS

SEQ ID NO: 60 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgEgE-C18DA
YXEGTFISDYSIALDRXAQXNFVNWLLAXSPSSGAPPPS

SEQ ID NO: 61 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDSXAQXDFVNWLLAQSG

SEQ ID NO: 62 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence 2OEGgEgE-C18DA
YXEGTFISDYSIALDRXAQXDFVNWLLAQXG

SEQ ID NO: 63 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (29)..(29): Lys, wherein the side chain of Lys is connected to the sequence 2OEGgEgE-C18DA
YXEGTFISDYSIXLDRXAQXDFVNWLLAXRG

SEQ ID NO: 64 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDRXAQXDFVNWLLAQRG

SEQ ID NO: 65 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence G6-C18DA
  MOD_RES (20)..(20): Aib
YXEGTFISDYSIXLDSXAQXDFVNWLLAQRG

SEQ ID NO: 66 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C20DA
YXEGTFISDYSIXLDRXAQXXFVNWLLAQKG

SEQ ID NO: 67 - protein, 31 aa, Artificial Sequence; identical to SEQ ID NO: 66
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA

SEQ ID NO: 68 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C20DA
  MOD_RES (17)..(17): Aib
  MOD_RES (20)..(20): Aib
YXEGTFISDYSIXXDRXAQXDFVNWLLAQRG

SEQ ID NO: 69 - protein, 39 aa, Artificial Sequence; identical to SEQ ID NO: 12
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C20DA
  MOD_RES (20)..(20): Aib

SEQ ID NO: 70 - protein, 39 aa, Artificial Sequence; identical to SEQ ID NO: 17
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgEgE-C20DA

SEQ ID NO: 71 - protein, 34 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDRXAQXDFVNWLLAQRPSSG

SEQ ID NO: 72 - protein, 34 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDRXAQXDFVNWLLAQSPSSG

SEQ ID NO: 73 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIXLDRXAQXXFVNWLLAQRG

SEQ ID NO: 74 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (18)..(18): Lys, wherein the side chain of Lys is connected to the sequence 2OEGgEgE-C18DA
  MOD_RES (20)..(20): Aib
YXEGTFISDYSIXLDRXXQXDFVNWLLAQRG

SEQ ID NO: 75 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (18)..(18): Lys, wherein the side chain of Lys is connected to the sequence 2OEGgEgE-C18DA
  MOD_RES (20)..(20): Aib
YXEGTFISDYSIXLDRXXQXNFVNWLLAQRG

SEQ ID NO: 76 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDSXAQXDFVNWLLAQKG

SEQ ID NO: 77 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDKXAQXDFVNWLLAQKG

SEQ ID NO: 78 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence G5-C18DA
  MOD_RES (20)..(20): Aib
YXEGTFISDYSIXLDSXAQXDFVNWLLAQKG

SEQ ID NO: 79 - protein, 31 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C18DA
  MOD_RES (20)..(20): Aib
YXEGTFISDYSIALDSXAQXDFVNWLLAQKG

SEQ ID NO: 80 - protein, 31 aa, Artificial Sequence; identical to SEQ ID NO: 76
  source: /note=“Description of Artificial Sequence Synthetic polypeptide”
  MOD_RES (2)..(2): Aib
  MOD_RES (14)..(14): Lys, wherein the side chain of Lys is connected to the sequence G4gE-C18DA
  MOD_RES (17)..(17): Aib
  MOD_RES (20)..(20): Aib

SEQ ID NO: 81 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G4gE-C18DA
  MOD_RES (17)..(17): Aib
  MOD_RES (20)..(20): Aib
YXEGTFISDYSIAXDRXAQXDFVNWLLAQKG

SEQ ID NO: 82 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
  MOD_RES (30)..(30): Lys(Ac)
YXEGTFISDYSIAXDRXAQXDFVNWLLAQXG

SEQ ID NO: 83 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDRXAQXDFVNWLLAQQG

SEQ ID NO: 84 - protein, 32 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIXXDRXAQXDFVNWLLAQSPG

SEQ ID NO: 85 - protein, 32 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDRXAQXDFVNWLLAQSPG

SEQ ID NO: 86 - protein, 32 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIXLDRXAQXXFVNWLLAQSPG

SEQ ID NO: 87 - protein, 32 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIALDRXAQXXFVNWLLAQSPG

SEQ ID NO: 88 - protein, 32 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (18)..(18): Lys, wherein the side chain of Lys is connected to the sequence 2OEGgEgE-C18DA
  MOD_RES (20)..(20): Aib
YXEGTFISDYSIXLDRXXQXDFVNWLLAQSPG

SEQ ID NO: 89 - protein, 32 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence G5-C18DA
  MOD_RES (20)..(20): Aib
YXEGTFISDYSIXLDRXAQXDFVNWLLAQSPG

SEQ ID NO: 90 - protein, 39 aa, Artificial Sequence; identical to SEQ ID NO: 14
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA

SEQ ID NO: 91 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIXLDRXAQXXFVNWLLAQSPSSGAPPPS

SEQ ID NO: 92 - protein, 39 aa, Artificial Sequence; identical to SEQ ID NO: 91
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgE-C18DA

SEQ ID NO: 93 - protein, 39 aa, Artificial Sequence; identical to SEQ ID NO: 91
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence OEGgEOEGgE-C18DA

SEQ ID NO: 94 - protein, 39 aa, Artificial Sequence; identical to SEQ ID NO: 91
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence OEGgEgE-C18DA

SEQ ID NO: 95 - protein, 39 aa, Artificial Sequence; identical to SEQ ID NO: 91
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C17DA

SEQ ID NO: 96 - protein, 39 aa, Artificial Sequence; identical to SEQ ID NO: 30
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C17DA
  MOD_RES (17)..(17): Aib
  MOD_RES (20)..(20): Aib

SEQ ID NO: 97 - protein, 39 aa, Artificial Sequence; identical to SEQ ID NO: 30
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 98 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIALDRXAQXXFVNWLLAQSPSSGAPPPS

SEQ ID NO: 99 - protein, 31 aa, Artificial Sequence; identical to SEQ ID NO: 68
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C17DA
  MOD_RES (17)..(17): Aib
  MOD_RES (20)..(20): Aib

SEQ ID NO: 100 - protein, 39 aa, Artificial Sequence; identical to SEQ ID NO: 12
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C20DA
  MOD_RES (20)..(20): Aib

SEQ ID NO: 101 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (18)..(18): Lys, wherein the side chain of Lys is connected to the sequence 2OEGgEgE-C18DA
  MOD_RES (20)..(20): Aib
YXEGTFISDYSIXLDRXXQXNFVNWLLAQSPSSGAPPPS

SEQ ID NO: 102 - protein, 39 aa, Artificial Sequence; identical to SEQ ID NO: 14
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C20DA

SEQ ID NO: 103 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence GGPAPAP-C18DA
  MOD_RES (20)..(20): Aib
YXEGTFISDYSIALDRXXQXDFVNWLLAQSPSSGAPPPS

SEQ ID NO: 104 - protein, 39 aa, Artificial Sequence; identical to SEQ ID NO: 98
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence GGPAPAP-C18DA

SEQ ID NO: 105 - protein, 39 aa, Artificial Sequence; identical to SEQ ID NO: 98
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence GGPAPAPgE-C18DA

SEQ ID NO: 106 - protein, 39 aa, Artificial Sequence; identical to SEQ ID NO: 39
  source: /note=“Description of Artificial Sequence Synthetic polypeptide”
  MOD_RES (2)..(2): Aib
  MOD_RES (14)..(14): Lys, wherein the side chain of Lys is connected to the sequence GGPAPAP-C18DA
  MOD_RES (17)..(17): Aib
  MOD_RES (20)..(20): Aib

SEQ ID NO: 107 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIXLDRXAAXXFVNWLLAQRPSSGAPPPS

SEQ ID NO: 108 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIXLDRXAAXXFVNWLLAQSPSSGAPPPS

SEQ ID NO: 109 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIXXDRXAQXDFVEWLLAQRG

SEQ ID NO: 110 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (18)..(18): Lys, wherein the side chain of Lys is connected to the sequence 2OEGgEgE-C18DA
  MOD_RES (20)..(20): Aib
YXEGTFISDYSIXLDRXXAXNFVNWLLAQSPSSGAPPPS

SEQ ID NO: 111 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIXLDRXAQXXFVEWLLAQKG

SEQ ID NO: 112 - protein, 32 aa, Artificial Sequence; identical to SEQ ID NO: 85
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C20DA
  MOD_RES (17)..(17): Aib
  MOD_RES (20)..(20): Aib

SEQ ID NO: 113 - protein, 39 aa, Artificial Sequence; identical to SEQ ID NO: 13
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C20DA
  MOD_RES (17)..(17): Aib
  MOD_RES (20)..(20): Aib

SEQ ID NO: 114 - protein, 32 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C18DA
  MOD_RES (20)..(20): Aib
YXEGTFISDYSIALDRXAQXDFVNWLLAQSPG

SEQ ID NO: 115 - protein, 32 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDRXAQXNFVNWLLAQSPG

SEQ ID NO: 116 - protein, 31 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C18DA
  MOD_RES (20)..(20): Aib
YXEGTFISDYSIALDRXAQXDFVEWLLAQRG

SEQ ID NO: 117 - protein, 34 aa, Artificial Sequence; identical to SEQ ID NO: 72
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 118 - protein, 32 aa, Artificial Sequence; identical to SEQ ID NO: 87
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence GGPAPAPgE-C18DA

SEQ ID NO: 119 - protein, 32 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDRXAQXDFVEWLLAQSPG

SEQ ID NO: 120 - protein, 32 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIALDRXAQXXFVEWLLAQSPG

SEQ ID NO: 121 - protein, 32 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
YXEGTFISDYSIAXDRXAQRDFVEWLLAQSPG

SEQ ID NO: 122 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
YXEGTFISDYSIAXDRXAQKDFVEWLLAQRG

SEQ ID NO: 123 - protein, 31 aa, Artificial Sequence
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgEgE-C18DA
YXEGTFISDYSIKLDREAQXXFVNWLLAQRG

SEQ ID NO: 124 - protein, 31 aa, Artificial Sequence
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgEgE-C18DA
YXEGTFISDYSIAKDRAEQXXFVNWLLAQRG

SEQ ID NO: 125 - protein, 32 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
YXEGTFISDYSIAXDRXAQKDFVEWLLAQSPG

SEQ ID NO: 126 - protein, 32 aa, Artificial Sequence; identical to SEQ ID NO: 87
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C17DA

SEQ ID NO: 127 - protein, 32 aa, Artificial Sequence; identical to SEQ ID NO: 86
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C17DA

SEQ ID NO: 128 - protein, 31 aa, Artificial Sequence; identical to SEQ ID NO: 73
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C17DA

SEQ ID NO: 129 - protein, 32 aa, Artificial Sequence; identical to SEQ ID NO: 85
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 130 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (21)..(21): Lys, wherein the side chain of Lys is connected to the sequence 2OEGgEgE-C17DA
YXEGTFISDYSIXLDKIAQQXFVEWLLAQRG

SEQ ID NO: 131 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (21)..(21): Lys, wherein the side chain of Lys is connected to the sequence 2OEGgEgE-C17DA
YXEGTFISDYSIALDKXAQQXFVEWLLAQRG

SEQ ID NO: 132 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (21)..(21): Lys, wherein the side chain of Lys is connected to the sequence 2OEGgEgE-C17DA
YXEGTFISDYSIALDKXAQAXFVEWLLAQRG

SEQ ID NO: 133 - protein, 31 aa, Artificial Sequence
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgEgE-C17DA
YXEGTFISDYSIALDKIAQXXFVEWLLAQRG

SEQ ID NO: 134 - protein, 31 aa, Artificial Sequence
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgEgE-C17DA
YXEGTFISDYSIALDKAAQXXFVEWLLAQRG

SEQ ID NO: 135 - protein, 39 aa, Artificial Sequence
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C17DA
YXEGTFISDYSIALDKIAQQXFVEWLLAQRPSSGAPPPS

SEQ ID NO: 136 - protein, 39 aa, Artificial Sequence
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C17DA
YXEGTFISDYSIALDKIAQRXFVEWLLAQRPSSGAPPPS

SEQ ID NO: 137 - protein, 32 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence GGPAPAPgE-C18DA
  MOD_RES (20)..(20): Aib
YXEGTFISDYSIALDRXXQXDFVNWLLAQSPG

SEQ ID NO: 138 - protein, 31 aa, Artificial Sequence; identical to SEQ ID NO: 68
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 139 - protein, 39 aa, Artificial Sequence; identical to SEQ ID NO: 13
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 140 - protein, 31 aa, Artificial Sequence; identical to SEQ ID NO: 109
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 141 - protein, 31 aa, Artificial Sequence; identical to SEQ ID NO: 37
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C17DA

SEQ ID NO: 142 - protein, 31 aa, Artificial Sequence; identical to SEQ ID NO: 73
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA

SEQ ID NO: 143 - protein, 31 aa, Artificial Sequence
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C17DA
YXEGTFISDYSIALDKIAQQXFVEWLLAQRG

SEQ ID NO: 144 - protein, 31 aa, Artificial Sequence
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C17DA
YXEGTFISDYSIALDKIAQRXFVEWLLAQRG

SEQ ID NO: 145 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDRXAQXDFVEWLLAQRG

SEQ ID NO: 146 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (20)..(20): Aib
YXEGTFISDYSIXXDRAAQXDFVEWLLAQRG

SEQ ID NO: 147 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
YXEGTFISDYSIXXDRXAQADFVEWLLAQRG

SEQ ID NO: 148 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
YXEGTFISDYSIXXDRXAQRDFVEWLLAQRG

SEQ ID NO: 149 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
YXEGTFISDYSIXXDRAAQADFVEWLLAQRG

SEQ ID NO: 150 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
YXEGTFISDYSIAXDRXAQADFVEWLLAQRG

SEQ ID NO: 151 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIXXDSXAQXDFVNWLLAQRG

SEQ ID NO: 152 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (20)..(20): Aib
YXEGTFISDYSIXXDSAAQXDFVNWLLAQRG

SEQ ID NO: 153 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
YXEGTFISDYSIXXDSXAQADFVNWLLAQRG

SEQ ID NO: 154 - protein, 31 aa, Artificial Sequence; identical to SEQ ID NO: 133
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgEgE-C18DA

SEQ ID NO: 155 - protein, 31 aa, Artificial Sequence; identical to SEQ ID NO: 134
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgEgE-C18DA

SEQ ID NO: 156 - protein, 31 aa, Artificial Sequence; identical to SEQ ID NO: 132
  source: /note=“Description of Artificial Sequence Synthetic polypeptide”
  MOD_RES (2)..(2): Aib
  MOD_RES (17)..(17): Aib
  MOD_RES (21)..(21): Lys, wherein the side chain of Lys is connected to the sequence 2OEGgEgE-C18DA

SEQ ID NO: 157 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (21)..(21): Lys, wherein the side chain of Lys is connected to the sequence 2OEGgEgE-C18DA
YXEGTFISDYSIALDKXAQRXFVEWLLAQRG

SEQ ID NO: 158 - protein, 31 aa, Artificial Sequence
  source: /note=“Description of Artificial Sequence Synthetic polypeptide”
  MOD_RES (2)..(2): Aib
  MOD_RES (14)..(14): Aib
  MOD_RES (21)..(21): Lys, wherein the side chain of Lys is connected to the sequence 2OEGgEgE-C18DA
YXEGTFISDYSIAXDKAAQQXFVEWLLAQRG

SEQ ID NO: 159 - protein, 31 aa, Artificial Sequence
  source: /note=“Description of Artificial Sequence Synthetic polypeptide”
  MOD_RES (2)..(2): Aib
  MOD_RES (14)..(14): Aib
  MOD_RES (21)..(21): Lys, wherein the side chain of Lys is connected to the sequence 2OEGgEgE-C18DA
YXEGTFISDYSIAXDKAAQAXFVEWLLAQRG

SEQ ID NO: 160 - protein, 31 aa, Artificial Sequence; identical to SEQ ID NO: 73
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C20DA

SEQ ID NO: 161 - protein, 31 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgE-C18DA
  MOD_RES (20)..(20): Aib
YXEGTFISDYSIALDKIXQXDFVEWLLAQRG

SEQ ID NO: 162 - protein, 31 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgE-C18DA
  MOD_RES (20)..(20): Aib
YXEGTFISDYSIALDKAXQXDFVEWLLAQRG

SEQ ID NO: 163 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgE-C18DA
YXEGTFISDYSIALDKXXQADFVEWLLAQRG

SEQ ID NO: 164 - protein, 31 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgE-C18DA
YXEGTFISDYSIALDKXXQANFVEWLLAQRG

SEQ ID NO: 165 - protein, 31 aa, Artificial Sequence
  source: /note=“Description of Artificial Sequence Synthetic polypeptide”
  MOD_RES (2)..(2): Aib
  MOD_RES (14)..(14): Aib
  MOD_RES (18)..(18): Lys, wherein the side chain of Lys is connected to the sequence 2OEGgE-C18DA
YXEGTFISDYSIAXDKAXQQDFVEWLLAQRG

SEQ ID NO: 166 - protein, 31 aa, Artificial Sequence
  source: /note=“Description of Artificial Sequence Synthetic polypeptide”
  MOD_RES (2)..(2): Aib
  MOD_RES (14)..(14): Aib
  MOD_RES (18)..(18): Lys, wherein the side chain of Lys is connected to the sequence 2OEGgE-C18DA
YXEGTFISDYSIAXDKAXQADFVEWLLAQRG

SEQ ID NO: 167 - protein, 31 aa, Artificial Sequence
  source: /note=“Description of Artificial Sequence Synthetic polypeptide”
  MOD_RES (2)..(2): Aib
  MOD_RES (14)..(14): Aib
  MOD_RES (18)..(18): Lys, wherein the side chain of Lys is connected to the sequence 2OEGgE-C18DA
YXEGTFISDYSIAXDKIXQADFVEWLLAQRG

SEQ ID NO: 168 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (21)..(21): Lys, wherein the side chain of Lys is connected to the sequence 2OEGgEgE-C18DA
YXEGTFISDYSIXLDKAAQQXFVEWLLAQRG

SEQ ID NO: 169 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (21)..(21): Lys, wherein the side chain of Lys is connected to the sequence 2OEGgEgE-C18DA
YXEGTFISDYSIXLDKAAQAXFVEWLLAQRG

SEQ ID NO: 170 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (18)..(18): Lys, wherein the side chain of Lys is connected to the sequence 2OEGgE-C18DA
YXEGTFISDYSIXLDKAXQQDFVEWLLAQRG

SEQ ID NO: 171 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (18)..(18): Lys, wherein the side chain of Lys is connected to the sequence 2OEGgE-C18DA
YXEGTFISDYSIXLDKAXQQDFVNWLLAQRG

SEQ ID NO: 172 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (18)..(18): Lys, wherein the side chain of Lys is connected to the sequence 2OEGgE-C18DA
YXEGTFISDYSIXLDKAXQADFVNWLLAQRG

SEQ ID NO: 173 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (18)..(18): Lys, wherein the side chain of Lys is connected to the sequence 2OEGgE-C18DA
YXEGTFISDYSIXLDKIXQADFVNWLLAQRG

SEQ ID NO: 174 - protein, 31 aa, Artificial Sequence; identical to SEQ ID NO: 133
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgEgE-C18DA

SEQ ID NO: 175 - protein, 31 aa, Artificial Sequence; identical to SEQ ID NO: 134
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgEgE-C18DA

SEQ ID NO: 176 - protein, 31 aa, Artificial Sequence
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgEgE-C18DA
YXEGTFISDYSIALDKIAQXXFVNWLLAQRG

SEQ ID NO: 177 - protein, 31 aa, Artificial Sequence
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgEgE-C18DA
YXEGTFISDYSIALDKAAQXXFVNWLLAQRG

SEQ ID NO: 178 - protein, 39 aa, Artificial Sequence
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C18DA
YXEGTFISDYSIALDRIAQQXFVNWLLAQSPSSGAPPPS

SEQ ID NO: 179 - protein, 32 aa, Artificial Sequence
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C18DA
YXEGTFISDYSIALDRAAQQXFVNWLLAQSPG

SEQ ID NO: 180 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
YXEGTFISDYSIAXDRIAQRNFVEWLLAQSPSSGAPPPS

SEQ ID NO: 181 - protein, 32 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
YXEGTFISDYSIAXDRIAQRNFVEWLLAQSPG

SEQ ID NO: 182 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (30)..(30): Lys, wherein the side chain of Lys is connected to the sequence 2OEGgE-C18DA
YXEGTFISDYSIALDRXHQQEFVKWLLAQXPSSGAPPPS

SEQ ID NO: 183 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (30)..(30): Lys, wherein the side chain of Lys is connected to the sequence 2OEGgE-C18DA
YXEGTFISDYSIALDRXHQENFKNWLLAQXPSSGAPPPS

SEQ ID NO: 184 - protein, 39 aa, Artificial Sequence
  source: /note=“Description of Artificial Sequence Synthetic polypeptide”
  MOD_RES (2)..(2): Aib
  MOD_RES (30)..(30): Lys, wherein the side chain of Lys is connected to the sequence 2OEGgE-C18DA
YXEGTFISDYSIALDREHQKNFVNWLLAQXPSSGAPPPS

SEQ ID NO: 185 - protein, 39 aa, Artificial Sequence
  source: /note=“Description of Artificial Sequence Synthetic polypeptide”
  MOD_RES (2)..(2): Aib
  MOD_RES (20)..(20): Aib
  MOD_RES (30)..(30): Lys, wherein the side chain of Lys is connected to the sequence 2OEGgE-C18DA
YXEGTFISDYSIAEDRKHQXNFVNWLLAQXPSSGAPPPS

SEQ ID NO: 186 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (30)..(30): Lys, wherein the side chain of Lys is connected to the sequence 2OEGgE-C18DA
YXEGTFISDYSIALDRXHQXNFVNELLKQXPSSGAPPPS

SEQ ID NO: 187 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (26)..(26): Lys, wherein the side chain of Lys is connected to the sequence 2OEGgE-C18DA
YXEGTFISDYSIALDRXHQXNFVNWXLAQR

SEQ ID NO: 188 - protein, 42 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C18DA
YXEGTFISDYSIAXDKIHQQDFVNWLLAQKGKKNDWKHNITQ

SEQ ID NO: 189 - protein, 42 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C18DA
YXEGTFISDYSIAMDKXHQQDFVNWLLAQKGKKNDWKHNITQ

SEQ ID NO: 190 - protein, 42 aa, Artificial Sequence
  source: /note=“Description of Artificial Sequence Synthetic polypeptide”
  MOD_RES (2)..(2): Aib
  MOD_RES (30)..(30): Lys, wherein the side chain of Lys is connected to the sequence 2OEGgE-C18DA
YXEGTFISDYSIAMDKIHQQDFVNWLLAQXGKKNDWKHNITQ

SEQ ID NO: 191 - protein, 39 aa, Artificial Sequence
  source: /note=“Description of Artificial Sequence Synthetic polypeptide”
  MOD_RES (2)..(2): Aib
  MOD_RES (30)..(30): Lys, wherein the side chain of Lys is connected to the sequence 2OEGgE-C18DA
YXEGTFISDYSIALDRIHQQNFVNWLLAQXPSSGAPPPS

SEQ ID NO: 192 - protein, 30 aa, Artificial Sequence
  source: /note=“Description of Artificial Sequence Synthetic polypeptide”
  MOD_RES (2)..(2): Aib
  MOD_RES (17)..(17): Aib
  MOD_RES (30)..(30): Lys, wherein the side chain of Lys is connected to the sequence 2OEGgE-C18DA
YXEGTFISDYSIALDRXHQQNFVNWLLAQX

SEQ ID NO: 193 - protein, 30 aa, Artificial Sequence; identical to SEQ ID NO: 11
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgEgE-C17DA
  MOD_RES (17)..(17): Aib
  MOD_RES (20)..(20): Aib

SEQ ID NO: 194 - protein, 30 aa, Artificial Sequence; identical to SEQ ID NO: 7
  source: /note=“Description of Artificial Sequence Synthetic polypeptide”
  MOD_RES (2)..(2): Aib
  MOD_RES (14)..(14): Lys, wherein the side chain of Lys is connected to the sequence gEgEgE-C17DA
  MOD_RES (17)..(17): Aib
  MOD_RES (20)..(20): Aib

SEQ ID NO: 195 - protein, 31 aa, Artificial Sequence; identical to SEQ ID NO: 37
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA

SEQ ID NO: 196 - protein, 39 aa, Artificial Sequence; identical to SEQ ID NO: 13
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 197 - protein, 32 aa, Artificial Sequence
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C18DA
YXEGTFISDYSIALDRIAQQXFVNWLLAQSPG

SEQ ID NO: 198 - protein, 32 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
YXEGTFISDYSIAXDRIAQQNFVNWLLAQSPG

SEQ ID NO: 199 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
YXEGTFISDYSIAXDRIAQQDFVNWLLAQSPSSGAPPPS

SEQ ID NO: 200 - protein, 32 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
YXEGTFISDYSIAXDRIAQQDFVNWLLAQSPG

SEQ ID NO: 201 - protein, 31 aa, Artificial Sequence; identical to SEQ ID NO: 40
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C17DA

SEQ ID NO: 202 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIXLDRXHQXXFVNWLLAQRG

SEQ ID NO: 203 - protein, 31 aa, Artificial Sequence; identical to SEQ ID NO: 73
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18 (stea)

SEQ ID NO: 204 - protein, 31 aa, Artificial Sequence; identical to SEQ ID NO: 40
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18 (stea)

SEQ ID NO: 205 - protein, 31 aa, Artificial Sequence; identical to SEQ ID NO: 73
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA

SEQ ID NO: 206 - protein, 31 aa, Artificial Sequence
  source: /note=“Description of Artificial Sequence Synthetic polypeptide”
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YGEGTFISDYSIXLDRXAQXXFVNWLLAQRG

SEQ ID NO: 207 - protein, 31 aa, Artificial Sequence
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgEgE-C18DA
YXEGTFISDYSIALDRAAQXXFVEWLLAQRG

SEQ ID NO: 208 - protein, 31 aa, Artificial Sequence; identical to SEQ ID NO: 73
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence OEGgEgE-C18DA

SEQ ID NO: 209 - protein, 31 aa, Artificial Sequence; identical to SEQ ID NO: 73
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgE-C18DA

SEQ ID NO: 210 - protein, 31 aa, Artificial Sequence; identical to SEQ ID NO: 73
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 3OEGgEgE-C18DA

SEQ ID NO: 211 - protein, 31 aa, Artificial Sequence; identical to SEQ ID NO: 73
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence G5gEgE-C18DA

SEQ ID NO: 212 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIXLDKXAQXXFVNWLLAQRG

SEQ ID NO: 213 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIXLDRXAAXXFVNWLLAQRG

SEQ ID NO: 214 - protein, 31 aa, Artificial Sequence; identical to SEQ ID NO: 37
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA

SEQ ID NO: 215 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (20)..(20): Aib
  MOD_RES (21)..(21): Lys, wherein the side chain of Lys is connected to the sequence 2OEGgEgE-C18DA
YXEGTFISDYSIXLDRAAQXXFVNWLLAQRG

SEQ ID NO: 216 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (21)..(21): Lys, wherein the side chain of Lys is connected to the sequence 2OEGgEgE-C18DA
YXEGTFISDYSIXLDRXAQAXFVNWLLAQRG

SEQ ID NO: 217 - protein, 31 aa, Artificial Sequence
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C18DA
YXEGTFISDYSIALDRAAQAXFVNWLLAQRG

SEQ ID NO: 218 - protein, 31 aa, Artificial Sequence
  source: /note=“Description of Artificial Sequence Synthetic polypeptide”
  MOD_RES (2)..(2): d-Ala
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YAEGTFISDYSIXLDRXAQXKFVNWLLAQRG

SEQ ID NO: 219 - protein, 31 aa, Artificial Sequence
  source: /note=“Description of Artificial Sequence Synthetic polypeptide”
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YAEGTFISDYSIXLDRXAQXXFVNWLLAQRG

SEQ ID NO: 220 - protein, 31 aa, Artificial Sequence; identical to SEQ ID NO: 73
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEG-C18DA

SEQ ID NO: 221 - protein, 31 aa, Artificial Sequence; identical to SEQ ID NO: 212
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA

SEQ ID NO: 222 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Lys, wherein the side chain of Lys is connected to the sequence 2OEGgEgE-C18DA
YXEGTFISDYSIXLDRXAQXDFVNWLLAQRG

SEQ ID NO: 223 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIXLDRXAQXXFVEWLLAQRG

SEQ ID NO: 224 - protein, 31 aa, Artificial Sequence; identical to SEQ ID NO: 222
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgEgE-C18DA
  MOD_RES (20)..(20): Aib

SEQ ID NO: 225 - protein, 31 aa, Artificial Sequence; identical to SEQ ID NO: 68
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgEgE-C18DA
  MOD_RES (17)..(17): Aib
  MOD_RES (20)..(20): Aib

SEQ ID NO: 226 - protein, 31 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgEgE-C18DA
YXEGTFISDYSIXLDRXAQXDFVNWLLAQXG

SEQ ID NO: 227 - protein, 31 aa, Artificial Sequence; identical to SEQ ID NO: 133
  source: /note=“Description of Artificial Sequence Synthetic polypeptide”
  MOD_RES (2)..(2): Aib
  MOD_RES (20)..(20): Aib
  MOD_RES (21)..(21): Lys, wherein the side chain of Lys is connected to the sequence 2OEGgEgE-C18DA

SEQ ID NO: 228 - protein, 39 aa, Artificial Sequence
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C18DA
YXEGTFISDYSIALDRXXQANFVNWLLAQSPSSGAPPPS

SEQ ID NO: 229 - protein, 39 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgEgE-C18DA
YXEGTFISDYSIALDRIXQANFVNWLLAQSPSSGAPPPS

SEQ ID NO: 230 - protein, 39 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgEgE-C18DA
  MOD_RES (20)..(20): Aib
YXEGTFISDYSIALDRIXQXNFVNWLLAQSPSSGAPPPS

SEQ ID NO: 231 - protein, 31 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgE-C18DA
YXEGTFISDYSIALDKIXQQDFVNWLLAQRG

SEQ ID NO: 232 - protein, 31 aa, Artificial Sequence; identical to SEQ ID NO: 231
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgE-C18DA

SEQ ID NO: 233 - protein, 39 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgEgE-C18DA
YXEGTFISDYSIALDRIXQADFVNWLLAQRPSSGAPPPS

SEQ ID NO: 234 - protein, 39 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgEgE-C18DA
YXEGTFISDYSIALDRIXQQDFVNWLLAQRPSSGAPPPS

SEQ ID NO: 235 - protein, 39 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgEgE-C18DA
YXEGTFISDYSIALDRIXQQNFVNWLLAQRPSSGAPPPS

SEQ ID NO: 236 - protein, 39 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgEgE-C18DA
YXEGTFISDYSIALDRIXQQNFVNWLLAQSPSSGAPPPS

SEQ ID NO: 237 - protein, 31 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgE-C18DA
YXEGTFISDYSIALDRIXQQDFVNWLLAQRG

SEQ ID NO: 238 - protein, 31 aa, Artificial Sequence
  source: /note=“Description of Artificial Sequence Synthetic polypeptide”
  MOD_RES (2)..(2): Aib
  MOD_RES (20)..(20): Lys, wherein the side chain of Lys is connected to the sequence 2OEGgE-C18DA
YXEGTFISDYSIALDKIAQXDFVNWLLAQRG

SEQ ID NO: 239 - protein, 39 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgEgE-C18DA
YXEGTFISDYSIALDRIXQQNFVEWLLAQRPSSGAPPPS

SEQ ID NO: 240 - protein, 39 aa, Artificial Sequence
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C18DA
YXEGTFISDYSIALDRIAQQXFVEWLLAQSPSSGAPPPS

SEQ ID NO: 241 - protein, 39 aa, Artificial Sequence
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C18DA
YXEGTFISDYSIALDRIAQRXFVEWLLAQSPSSGAPPPS

SEQ ID NO: 242 - protein, 31 aa, Artificial Sequence
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C18DA
YXEGTFISDYSIALDKAAQRXFVEWLLAQRG

SEQ ID NO: 243 - protein, 31 aa, Artificial Sequence; identical to SEQ ID NO: 144
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C18DA

SEQ ID NO: 244 - protein, 31 aa, Artificial Sequence
  source: /note=“Description of Artificial Sequence Synthetic polypeptide”
  MOD_RES (31)..(31): Lys, wherein the side chain of Lys is connected to the sequence 2OEGgE-C18DA
YDAEGTFISDYSIALDRIHQQNFVNWLLAQX

SEQ ID NO: 245 - protein, 30 aa, Artificial Sequence; identical to SEQ ID NO: 6
  source: /note=“Description of Artificial Sequence Synthetic polypeptide”
  MOD_RES (2)..(2): Aib
  MOD_RES (30)..(30): Lys, wherein the side chain of Lys is connected to the sequence 2OEGgE-C18DA

SEQ ID NO: 246 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C18DA
YXEGTFISDYSIAXDRIHQQNFVNWLLAQK

SEQ ID NO: 247 - protein, 30 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C18DA
YXEGTFISDYSIALDRXHQQNFVNWLLAQK

SEQ ID NO: 248 - protein, 30 aa, Artificial Sequence
  source: /note=“Description of Artificial Sequence Synthetic polypeptide”
  MOD_RES (2)..(2): Aib
  MOD_RES (20)..(20): Lys, wherein the side chain of Lys is connected to the sequence 2OEGgE-C18DA
YXEGTFISDYSIALDRIHQXNFVNWLLAQK

SEQ ID NO: 249 - protein, 30 aa, Artificial Sequence
  source: /note=“Description of Artificial Sequence Synthetic polypeptide”
  MOD_RES (2)..(2): Aib
  MOD_RES (20)..(20): Aib
  MOD_RES (30)..(30): Lys, wherein the side chain of Lys is connected to the sequence 2OEGgE-C18DA
YXEGTFISDYSIALDRIHQXNFVNWLLAQX

SEQ ID NO: 250 - protein, 30 aa, Artificial Sequence; identical to SEQ ID NO: 6
  source: /note=“Description of Artificial Sequence Synthetic polypeptide”
  MOD_RES (2)..(2): Aib
  MOD_RES (30)..(30): Lys, wherein the side chain of Lys is connected to the sequence 2OEGgE-C18DA

SEQ ID NO: 251 - protein, 30 aa, Artificial Sequence; identical to SEQ ID NO: 8
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 252 - protein, 30 aa, Artificial Sequence; identical to SEQ ID NO: 8
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 253 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C18DA
  MOD_RES (20)..(20): Aib
YXEGTFISDYSIAXDRIAQXNFVNWLLAQR

SEQ ID NO: 254 - protein, 30 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C18DA
  MOD_RES (17)..(17): Aib
YXEGTFISDYSIAXDRXAQQNFVNWLLAQR

SEQ ID NO: 255 - protein, 30 aa, Artificial Sequence; identical to SEQ ID NO: 8
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C18DA
  MOD_RES (17)..(17): Aib
  MOD_RES (20)..(20): Aib

SEQ ID NO: 256 - protein, 30 aa, Artificial Sequence; identical to SEQ ID NO: 7
  source: /note=“Description of Artificial Sequence Synthetic polypeptide”
  MOD_RES (2)..(2): Aib
  MOD_RES (14)..(14): Lys, wherein the side chain of Lys is connected to the sequence 2OEGgE-C17DA
  MOD_RES (17)..(17): Aib
  MOD_RES (20)..(20): Aib

SEQ ID NO: 257 - protein, 30 aa, Artificial Sequence; identical to SEQ ID NO: 18
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C17DA
  MOD_RES (20)..(20): Aib

SEQ ID NO: 258 - protein, 30 aa, Artificial Sequence; identical to SEQ ID NO: 19
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C17DA

SEQ ID NO: 259 - protein, 30 aa, Artificial Sequence; identical to SEQ ID NO: 20
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (29)..(29): Lys, wherein the side chain of Lys is connected to the sequence 2OEGgEgE-C17DA

SEQ ID NO: 260 - protein, 39 aa, Artificial Sequence; identical to SEQ ID NO: 21
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C17DA
  MOD_RES (20)..(20): Aib

SEQ ID NO: 261 - protein, 39 aa, Artificial Sequence; identical to SEQ ID NO: 35
  source: /note=“Description of Artificial Sequence Synthetic polypeptide”
  MOD_RES (2)..(2): Aib
  MOD_RES (14)..(14): Lys, wherein the side chain of Lys is connected to the sequence G5gE-C17DA
  MOD_RES (17)..(17): Aib
  MOD_RES (20)..(20): Aib

SEQ ID NO: 262 - protein, 39 aa, Artificial Sequence; identical to SEQ ID NO: 15
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5gE-C17DA
  MOD_RES (17)..(17): Aib
  MOD_RES (20)..(20): Aib

SEQ ID NO: 263 - protein, 39 aa, Artificial Sequence; identical to SEQ ID NO: 15
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5gE-C18DA
  MOD_RES (17)..(17): Aib
  MOD_RES (20)..(20): Aib

SEQ ID NO: 264 - protein, 39 aa, Artificial Sequence; identical to SEQ ID NO: 15
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgE-C17DA
  MOD_RES (17)..(17): Aib
  MOD_RES (20)..(20): Aib

SEQ ID NO: 265 - protein, 30 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5gEgE-C17DA
  MOD_RES (17)..(17): Aib
  MOD_RES (20)..(20): Aib
YXEGTFISDYSIXXDRXAQXNFVNWLLAQR

SEQ ID NO: 266 - protein, 30 aa, Artificial Sequence
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5gE-C17DA
  MOD_RES (17)..(17): Aib
  MOD_RES (20)..(20): Aib
YXEGTFISDYSIXXDRXAQXDFVNWLLAQR

SEQ ID NO: 267 - protein, 39 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence 2OEGgE-C17DA
  MOD_RES (20)..(20): Aib
YXEGTFISDYSIALDRXAQXNFVNWLLAQRPSSGAPPPS

SEQ ID NO: 268 - protein, 30 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA
YXEGTFISDYSIXLDRXAQXXFVNWLLAQR

SEQ ID NO: 269 - protein, 39 aa, Artificial Sequence; identical to SEQ ID NO: 12
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgE-C17DA
  MOD_RES (20)..(20): Aib

SEQ ID NO: 270 - protein, 30 aa, Artificial Sequence; identical to SEQ ID NO: 9
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18DA

SEQ ID NO: 271 - protein, 39 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgEgE-C17DA
  MOD_RES (20)..(20): Aib
YXEGTFISDYSIXLDRXAQXNFVNWLLAQSPSSGAPPPS

SEQ ID NO: 272 - protein, 35 aa, Artificial Sequence
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence 2OEGgEgE-C17DA
  MOD_RES (20)..(20): Aib
YXEGTFISDYSIXLDRXAQXNFVNWLLAQRGGGGS

SEQ ID NO: 273 - protein, 30 aa, Artificial Sequence; identical to SEQ ID NO: 9
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C20DA

SEQ ID NO: 274 - protein, 39 aa, Artificial Sequence; identical to SEQ ID NO: 13
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 275 - protein, 39 aa, Artificial Sequence; identical to SEQ ID NO: 23
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 276 - protein, 39 aa, Artificial Sequence; identical to SEQ ID NO: 30
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 277 - protein, 39 aa, Artificial Sequence; identical to SEQ ID NO: 55
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence G5-C18DA
  MOD_RES (17)..(17): Aib
  MOD_RES (20)..(20): Aib

SEQ ID NO: 278 - protein, 40 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G6-C18DA
  MOD_RES (20)..(20): Aib
YXEGTFISLYSIALDRXHQXNFVNWLLAQRPSSGAPPPSR

SEQ ID NO: 279 - protein, 40 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G6-C18DA
  MOD_RES (20)..(20): Aib
YXQGTFISDYSIALDRXHQXNFVNWLLAQRPSSGAPPPSR

SEQ ID NO: 280 - protein, 40 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G6-C18DA
  MOD_RES (20)..(20): Aib
YXEGTFISDYSIALSRXHQXNFVNWLLAQRPSSGAPPPSR

SEQ ID NO: 281 - protein, 40 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G6-C18DA
  MOD_RES (20)..(20): Aib
YXEGTFISDYSIALKRXHQXNFVNWLLAQRPSSGAPPPSR

SEQ ID NO: 282 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (17)..(17): Aib
  MOD_RES (20)..(20): Aib
YXEGTFISDYSIAXDKXHQXNFVNWLLAQKGKKNNWKHN

SEQ ID NO: 283 - protein, 40 aa, Artificial Sequence; identical to SEQ ID NO: 278
  source: /note=“Description of Artificial Sequence Synthetic polypeptide”
  MOD_RES (2)..(2): Aib
  MOD_RES (17)..(17): Lys, wherein the side chain of Lys is connected to the sequence G5K-C18DA
  MOD_RES (20)..(20): Aib

SEQ ID NO: 284 - protein, 40 aa, Artificial Sequence; identical to SEQ ID NO: 278
  source: /note=“Description of Artificial Sequence Synthetic polypeptide”
  MOD_RES (2)..(2): Aib
  MOD_RES (17)..(17): Lys, wherein the side chain of Lys is connected to the sequence G4KG-C18DA
  MOD_RES (20)..(20): Aib

SEQ ID NO: 285 - protein, 31 aa, Artificial Sequence; identical to SEQ ID NO: 222
  source: /note=“Description of Artificial Sequence Synthetic polypeptide”
  MOD_RES (2)..(2): Aib
  MOD_RES (13)..(13): Aib
  MOD_RES (17)..(17): Lys, wherein the side chain of Lys is connected to the sequence G5-C18DA
  MOD_RES (20)..(20): Aib

SEQ ID NO: 286 - protein, 34 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
YXEGTFISDYSIAXDRIAQQDFVNWLLAQRPSSG

SEQ ID NO: 287 - protein, 31 aa, Artificial Sequence
  source: /note=“Description of Artificial Sequence Synthetic polypeptide”
  MOD_RES (2)..(2): Aib
  MOD_RES (21)..(21): Lys, wherein the side chain of Lys is connected to the sequence 2OEGgEgE-C18DA
YXEGTFISDYSIALDRIAQQXFVNWLLAQRG

SEQ ID NO: 288 - protein, 34 aa, Artificial Sequence
  source: /note=“Description of Artificial Sequence Synthetic polypeptide”
  MOD_RES (2)..(2): Aib
  MOD_RES (18)..(18): Lys, wherein the side chain of Lys is connected to the sequence 2OEGgEgE-C18DA
YXEGTFISDYSIALDRIXQQDFVNWLLAQRPSSG

SEQ ID NO: 289 - protein, 34 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C18DA
YXEGTFISDYSIALDRXAQQDFVNWLLAQRPSSG

SEQ ID NO: 290 - protein, 31 aa, Artificial Sequence; identical to SEQ ID NO: 81
  source: /note=“Description of Artificial Sequence Synthetic polypeptide”
  MOD_RES (2)..(2): Aib
  MOD_RES (14)..(14): Lys, wherein the side chain of Lys is connected to the sequence GGPAPAP-C18DA
  MOD_RES (17)..(17): Aib
  MOD_RES (20)..(20): Aib

SEQ ID NO: 291 - protein, 32 aa, Artificial Sequence
  source: /note=“Description of Artificial Sequence Synthetic polypeptide”
  MOD_RES (2)..(2): Aib
  MOD_RES (17)..(17): Lys, wherein the side chain of Lys is connected to the sequence G5-C18DA
  MOD_RES (20)..(20): Aib
YXEGTFISDYSIALDRXAQXNFVNWLLAQSPG

SEQ ID NO: 292 - protein, 31 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
  MOD_RES (20)..(20): Aib
YXEGTFISDYSIAXDRAAQXDFVEWLLAQRG

SEQ ID NO: 293 - protein, 39 aa, Artificial Sequence
  source: /note=“Description of Artificial Sequence Synthetic polypeptide”
  MOD_RES (2)..(2): Aib
  MOD_RES (14)..(14): Lys, wherein the side chain of Lys is connected to the sequence G5-C18DA
YXEGTFISDYSIAXDRIAQQNFVNWLLAQSPSSGAPPPS

SEQ ID NO: 294 - protein, 39 aa, Artificial Sequence; identical to SEQ ID NO: 12
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C18DA
  MOD_RES (20)..(20): Aib

SEQ ID NO: 295 - protein, 31 aa, Artificial Sequence; identical to SEQ ID NO: 68
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence GGGGG-C18DA
  MOD_RES (17)..(17): Aib
  MOD_RES (20)..(20): Aib

SEQ ID NO: 296 - protein, 42 aa, Artificial Sequence
  source: /note=“Description of Artificial Sequence Synthetic polypeptide”
  VARIANT (2)..(2): /replace=“Aib” or “D-Ala” or “Gly” or “Pro”
  VARIANT (9)..(9): /replace=“Leu”
  VARIANT (13)..(13): /replace=“Aib”
  VARIANT (14)..(14): /replace=“Aib” or “Ile” or “Nle”
  VARIANT (16)..(16): /replace=“Ser” or “Lys”
  VARIANT (17)..(17): /replace=“Aib” or “Ile”
  VARIANT (18)..(18): /replace=“His” or “Lys”
  VARIANT (20)..(20): /replace=“Aib” or “Ala”
  VARIANT (21)..(21): /replace=“Asn” or “Lys”
  VARIANT (24)..(24): /replace=“Gln” or “Glu”
  VARIANT (30)..(30): /replace=“Ser” or “Gln” or “Lys”
  VARIANT (31)..(31): /replace=“Pro” or “ ”
  VARIANT (32)..(32): /replace=“Lys” or “Pro” or “Gly” or “ ”
  VARIANT (33)..(33): /replace=“Lys” or “Gly” or “ ”
  VARIANT (34)..(34): /replace=“Asn” or “ ”
  VARIANT (35)..(35): /replace=“Asp” or “Ser” or “Asn” or “ ”
  VARIANT (36)..(36): /replace=“Trp” or “ ”
  VARIANT (37)..(37): /replace=“Lys” or “ ”
  VARIANT (38)..(38): /replace=“His” or “ ”
  VARIANT (39)..(39): /replace=“Asn” or “ ”
  VARIANT (40)..(40): /replace=“ ”
  VARIANT (41)..(41): /replace=“ ”
  VARIANT (42)..(42): /replace=“ ”
  SITE (1)..(42): /note=“Variant residues given in the sequence have no preference with respect to those in the annotations for variant positions”
YAEGTFISDYSIALDRAAQQDFVNWLLAQRGSSGAPPPSITQ

SEQ ID NO: 297 - protein, 42 aa, Artificial Sequence
  source: /note=“Description of Artificial Sequence Synthetic polypeptide”
  VARIANT (2)..(2): /replace=“Aib” or “D-Ala”
  VARIANT (13)..(13): /replace=“Aib”
  VARIANT (14)..(14): /replace=“Aib” or “Ile” or “Nle” or “Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (16)..(16): /replace=“Ser” or “Lys”
  VARIANT (17)..(17): /replace=“Aib” or “Ile” or “Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (18)..(18): /replace=“His” or “Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (19)..(19): /replace=“Ala”
  VARIANT (20)..(20): /replace=“Aib” or “Arg” or “Ala”
  VARIANT (21)..(21): /replace=“Asn” or “Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (24)..(24): /replace=“Gln” or “Glu”
  VARIANT (29)..(29): /replace=“Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (30)..(30): /replace=“Lys” or “Ser” or “Gln” or “Lys, wherein the side chain of Lys is connected to the sequence 2OEGgEgE, OEGgEgE, 2OEGgE, 3OEGgEgE, G5gEgE, 2OEGgEgEgE, 2OEG or G5gEgE”
  VARIANT (31)..(31): /replace=“Pro” or “ ”
  VARIANT (32)..(32): /replace=“Lys” or “Pro” or “Gly” or “ ”
  VARIANT (33)..(33): /replace=“Lys” or “Gly” or “ ”
  VARIANT (34)..(34): /replace=“Asn” or “ ”
  VARIANT (35)..(35): /replace=“Asp” or “Ser” or “Asn” or “ ”
  VARIANT (36)..(36): /replace=“Trp” or “ ”
  VARIANT (37)..(37): /replace=“Lys” or “ ”
  VARIANT (38)..(38): /replace=“His” or “ ”
  VARIANT (39)..(39): /replace=“Asn” or “ ”
  VARIANT (40)..(40): /replace=“ ”
  VARIANT (41)..(41): /replace=“ ”
  VARIANT (42)..(42): /replace=“ ”
  SITE (1)..(42): /note=“Variant residues given in the sequence have no preference with respect to those in the annotations for variant positions”
YGEGTFISDYSIALDRAAQQDFVNWLLAQRGSSGAPPPSITQ

SEQ ID NO: 298 - protein, 7 aa, Artificial Sequence
  source: /note=“Description of Artificial Sequence Synthetic peptide”
  MOD_RES (6)..(6): gamma-Glu
  MOD_RES (7)..(7): gamma-Glu
GGGGGXX

SEQ ID NO: 299 - protein, 39 aa, Artificial Sequence
  source: /note=“Description of Artificial Sequence Synthetic polypeptide”
  MOD_RES (2)..(2): Aib
  VARIANT (13)..(13): /replace=“Aib” or “Lys”
  VARIANT (14)..(14): /replace=“Aib” or “Lys” or “Lys, wherein the side chain of Lys is connected to the sequence 2OEGgE, 2OEGgEgE, G4gE, GGGGG, G5gE, G5gEgE, G6, gEgEgE, OEGgEgE, OEGgEOEGgE, GGPAPAP or GGPAPAPgE”
  SITE (14)..(14): /note=“Each sequence connected to the side chain of Lys may be linked to one of the following lipids C17-C22 monoacid or C17-C22 diacid”
  VARIANT (16)..(16): /replace=“Ser” or “Lys”
  VARIANT (17)..(17): /replace=“Aib” or “Ile” or “Glu” or “Lys” or “Lys, wherein the side chain of Lys is connected to the sequence 2OEGgE, 2OEGgEgE, G4gE, GGGGG, G5gE, G5gEgE, G6, gEgEgE, OEGgEgE, OEGgEOEGgE, GGPAPAP or GGPAPAPgE”
  SITE (17)..(17): /note=“Each sequence connected to the side chain of Lys may be linked to one of the following lipids C17-C22 monoacid or C17-C22 diacid”
  VARIANT (18)..(18): /replace=“His” or “Glu” or “Lys” or “Lys, wherein the side chain of Lys is connected to the sequence 2OEGgE, 2OEGgEgE, G4gE, GGGGG, G5gE, G5gEgE, G6, gEgEgE, OEGgEgE, OEGgEOEGgE, GGPAPAP or GGPAPAPgE”
  SITE (18)..(18): /note=“Each sequence connected to the side chain of Lys may be linked to one of the following lipids C17-C22 monoacid or C17-C22 diacid”
  VARIANT (19)..(19): /replace=“Ala”
  VARIANT (20)..(20): /replace=“Aib” or “Gln” or “Arg” or “Lys”
  VARIANT (21)..(21): /replace=“Asn” or “Lys” or “Lys, wherein the side chain of Lys is connected to the sequence 2OEGgE, 2OEGgEgE, G4gE, GGGGG, G5gE, G5gEgE, G6, gEgEgE, OEGgEgE, OEGgEOEGgE, GGPAPAP or GGPAPAPgE”
  SITE (21)..(21): /note=“Each sequence connected to the side chain of Lys may be linked to one of the following lipids C17-C22 monoacid or C17-C22 diacid”
  VARIANT (24)..(24): /replace=“Glu”
  VARIANT (29)..(29): /replace=“Lys” or “Lys, wherein the side chain of Lys is connected to the sequence 2OEGgE, 2OEGgEgE, G4gE, GGGGG, G5gE, G5gEgE, G6, gEgEgE, OEGgEgE, OEGgEOEGgE, GGPAPAP or GGPAPAPgE”
  SITE (29)..(29): /note=“Each sequence connected to the side chain of Lys may be linked to one of the following lipids C17-C22 monoacid or C17-C22 diacid”
  VARIANT (30)..(30): /replace=“Ser” or “Gln” or “Lys” or “Lys(Ac)” or “Lys, wherein the side chain of Lys is connected to the sequence 2OEGgE, 2OEGgEgE, G4gE, GGGGG, G5gE, G5gEgE, G6, gEgEgE, OEGgEgE, OEGgEOEGgE, GGPAPAP, or GGPAPAPgE”
  SITE (30)..(30): /note=“Each sequence connected to the side chain of Lys may be linked to one of the following lipids C17-C22 monoacid or C17-C22 diacid”
  VARIANT (31)..(31): /replace=“Pro” or “ ”
  VARIANT (32)..(32): /replace=“Gly” or “ ”
  VARIANT (33)..(33): /replace=“Gly” or “ ”
  VARIANT (34)..(34): /replace=“ ”
  VARIANT (35)..(35): /replace=“Ser” or “ ”
  VARIANT (36)..(36): /replace=“ ”
  VARIANT (37)..(37): /replace=“ ”
  VARIANT (38)..(38): /replace=“ ”
  VARIANT (39)..(39): /replace=“ ”
  SITE (1)..(39): /note=“Variant residues given in the sequence have no preference with respect to those in the annotations for variant positions”
YXEGTFISDYSIALDRAAQADFVNWLLAQRGSSGAPPPS

SEQ ID NO: 300 - protein, 5 aa, Artificial Sequence
  source: /note=“Description of Artificial Sequence Synthetic peptide”
  MOD_RES (5)..(5): gamma-Glu
GGGGX

SEQ ID NO: 301 - protein, 5 aa, Artificial Sequence
  source: /note=“Description of Artificial Sequence Synthetic peptide”
GGGGG

SEQ ID NO: 302 - protein, 6 aa, Artificial Sequence
  source: /note=“Description of Artificial Sequence Synthetic peptide”
  MOD_RES (6)..(6): gamma-Glu
GGGGGX

SEQ ID NO: 303 - protein, 6 aa, Artificial Sequence
  source: /note=“Description of Artificial Sequence Synthetic peptide”
GGGGGG

SEQ ID NO: 304 - protein, 7 aa, Artificial Sequence
  source: /note=“Description of Artificial Sequence Synthetic peptide”
GGPAPAP

SEQ ID NO: 305 - protein, 8 aa, Artificial Sequence
  source: /note=“Description of Artificial Sequence Synthetic peptide”
  MOD_RES (8)..(8): gamma-Glu
GGPAPAPX

SEQ ID NO: 306 - protein, 10 aa, Artificial Sequence
  source: /note=“Description of Artificial Sequence Synthetic peptide”
  SITE (1)..(10): /note=“This sequence may encompass 2-10 residues”
GGGGGGGGGG

SEQ ID NO: 307 - protein, 6 aa, Artificial Sequence
  source: /note=“Description of Artificial Sequence Synthetic peptide”
  SITE (1)..(6): /note=“This sequence may encompass 2-6 residues”
GGGGGG

SEQ ID NO: 308 - protein, 39 aa, Artificial Sequence
  source: /note=“Description of Artificial Sequence Synthetic polypeptide”
  MOD_RES (2)..(2): Aib
  VARIANT (13)..(13): /replace=“Aib”
  VARIANT (14)..(14): /replace=“Lys” or “Lys, wherein the side chain of Lys is connected to the sequence 2OEGgEgE or GGGGG”
  SITE (14)..(14): /note=“Each sequence connected to the side chain of Lys may be linked to the lipid C18 diacid”
  VARIANT (17)..(17): /replace=“Aib” or “Lys, wherein the side chain of Lys is connected to the sequence 2OEGgEgE or GGGGG”
  SITE (17)..(17): /note=“Each sequence connected to the side chain of Lys may be linked to the lipid C18 diacid”
  MOD_RES (20)..(20): Aib
  VARIANT (21)..(21): /replace=“Asn” or “Lys” or “Lys, wherein the side chain of Lys is connected to the sequence 2OEGgEgE or GGGGG”
  SITE (21)..(21): /note=“Each sequence connected to the side chain of Lys may be linked to the lipid C18 diacid”
  VARIANT (30)..(30): /replace=“Ser” or “Lys” or “Lys, wherein the side chain of Lys is connected to the sequence 2OEGgEgE or GGGGG”
  SITE (30)..(30): /note=“Each sequence connected to the side chain of Lys may be linked to the lipid C18 diacid”
  VARIANT (31)..(31): /replace=“Pro”
  VARIANT (32)..(32): /replace=“Gly” or “ ”
  VARIANT (33)..(33): /replace=“ ”
  VARIANT (34)..(34): /replace=“ ”
  VARIANT (35)..(35): /replace=“ ”
  VARIANT (36)..(36): /replace=“ ”
  VARIANT (37)..(37): /replace=“ ”
  VARIANT (38)..(38): /replace=“ ”
  VARIANT (39)..(39): /replace=“ ”
  SITE (1)..(39): /note=“Variant residues given in the sequence have no preference with respect to those in the annotations for variant positions”
YXEGTFISDYSIALDRKAQXDFVNWLLAQRGSSGAPPPS

SEQ ID NO: 309 - protein, 32 aa, Artificial Sequence; identical to SEQ ID NO: 85
  source: /note=“Description of Artificial Sequence Synthetic polypeptide”
  MOD_RES (2)..(2): Aib
  MOD_RES (14)..(14): Lys, wherein the side chain of Lys is connected to the sequence GGGGG-C18 diacid
  MOD_RES (17)..(17): Aib
  MOD_RES (20)..(20): Aib

SEQ ID NO: 310 - protein, 32 aa, Artificial Sequence; identical to SEQ ID NO: 85
  source: /note=“Description of Artificial Sequence Synthetic polypeptide”
  MOD_RES (2)..(2): Aib
  MOD_RES (14)..(14): Lys, wherein the side chain of Lys is connected to the sequence GGGGG-C18 diacid
  MOD_RES (17)..(17): Aib
  MOD_RES (20)..(20): Aib

SEQ ID NO: 311 - protein, 39 aa, Artificial Sequence; identical to SEQ ID NO: 13
  source: /note=“Description of Artificial Sequence Synthetic polypeptide”
  MOD_RES (2)..(2): Aib
  MOD_RES (14)..(14): Lys, wherein the side chain of Lys is connected to the sequence GGGGG-C18 diacid
  MOD_RES (17)..(17): Aib
  MOD_RES (20)..(20): Aib

SEQ ID NO: 312 - protein, 39 aa, Artificial Sequence; identical to SEQ ID NO: 13
  source: /note=“Description of Artificial Sequence Synthetic polypeptide”
  MOD_RES (2)..(2): Aib
  MOD_RES (14)..(14): Lys, wherein the side chain of Lys is connected to the sequence GGGGG-C18 diacid
  MOD_RES (17)..(17): Aib
  MOD_RES (20)..(20): Aib

SEQ ID NO: 313 - protein, 39 aa, Artificial Sequence; identical to SEQ ID NO: 51
  source: /note=“Description of Artificial Sequence Synthetic polypeptide”
  MOD_RES (2)..(2): Aib
  MOD_RES (17)..(17): Lys, wherein the side chain of Lys is connected to the sequence GGGGG-C18 diacid
  MOD_RES (20)..(20): Aib

SEQ ID NO: 314 - protein, 39 aa, Artificial Sequence; identical to SEQ ID NO: 51
  source: /note=“Description of Artificial Sequence Synthetic polypeptide”
  MOD_RES (2)..(2): Aib
  MOD_RES (17)..(17): Lys, wherein the side chain of Lys is connected to the sequence GGGGG-C18 diacid
  MOD_RES (20)..(20): Aib

SEQ ID NO: 315 - protein, 31 aa, Artificial Sequence; identical to SEQ ID NO: 40
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18 diacid

SEQ ID NO: 316 - protein, 31 aa, Artificial Sequence; identical to SEQ ID NO: 40
  source: /note=“Description of Artificial Sequence Synthetic polypeptide”
  MOD_RES (2)..(2): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18 diacid

SEQ ID NO: 317 - protein, 31 aa, Artificial Sequence; identical to SEQ ID NO: 73
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18 diacid

SEQ ID NO: 318 - protein, 31 aa, Artificial Sequence; identical to SEQ ID NO: 73
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (21)..(21): Lys, wherein the side chain of Lys is connected to the sequence 2OEGgEgE-C18 diacid

SEQ ID NO: 319 - protein, 39 aa, Artificial Sequence; identical to SEQ ID NO: 17
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgEgE-C18 diacid

SEQ ID NO: 320 - protein, 39 aa, Artificial Sequence; identical to SEQ ID NO: 17
  source: /note=“Description of Artificial Sequence Synthetic polypeptide”
  MOD_RES (2)..(2): Aib
  MOD_RES (13)..(13): Aib
  MOD_RES (17)..(17): Aib
  MOD_RES (20)..(20): Aib
  MOD_RES (30)..(30): Lys, wherein the side chain of Lys is connected to the sequence 2OEGgEgE-C18 diacid

SEQ ID NO: 321 - protein, 39 aa, Artificial Sequence; identical to SEQ ID NO: 12
  source: /note=“Description of Artificial Sequence Synthetic polypeptide”
  MOD_RES (2)..(2): Aib
  MOD_RES (17)..(17): Aib
  MOD_RES (18)..(18): Lys, wherein the side chain of Lys is connected to the sequence 2OEGgEgE-C18 diacid
  MOD_RES (20)..(20): Aib

SEQ ID NO: 322 - protein, 31 aa, Artificial Sequence; identical to SEQ ID NO: 68
  source: /note=“Description of Artificial Sequence Synthetic polypeptide”
  MOD_RES (2)..(2): Aib
  MOD_RES (13)..(13): Aib
  MOD_RES (14)..(14): Lys, wherein the side chain of Lys is connected to the sequence 2OEGgEgE-C18 diacid
  MOD_RES (17)..(17): Aib
  MOD_RES (20)..(20): Aib

SEQ ID NO: 323 - protein, 39 aa, Artificial Sequence
  source: /note=“Description of Artificial Sequence Synthetic polypeptide”
  VARIANT (2)..(2): /replace=“Aib” or “D-Ala”
  VARIANT (13)..(13): /replace=“Aib”
  VARIANT (14)..(14): /replace=“Aib” or “Lys, wherein the side chain of Lys is connected to the gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE
  VARIANT (16)..(16): /replace=”Ser“ or ”Lys“
  VARIANT (17)..(17): /replace=”Aib“ or ”Ala“ or ”Lys, wherein the side chain of Lys is connected to the gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE“
  VARIANT (18)..(18): /replace=”Ala“ or ”Lys, wherein the side chain of Lys is connected to the gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE“
  VARIANT (19)..(19): /replace=”Ala“
  VARIANT (20)..(20): /replace=”Aib“ or ”Arg“ or ”Ala“
  VARIANT (21)..(21): /replace=”Asn“ or ”Lys, wherein the side chain of Lys is connected to the gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE“
  VARIANT (24)..(24): /replace=”Glu“
  VARIANT (29)..(29): /replace=”Lys, wherein the side chain of Lys is connected to the gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE“
  VARIANT (30)..(30): /replace=”Lys“ or ”Ser“ or ”Gln“ or ”Lys, wherein the side chain of Lys is connected to the gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG or G5gEgE“
  VARIANT (31)..(31): /replace=”Pro“ or ” “
  VARIANT (32)..(32): /replace=”Gly“ or ”Lys“ or ” “
  VARIANT (33)..(33): /replace=”Lys“ or ”Gly“ or ” “
  VARIANT (34)..(34): /replace=”Asn“ or ” “
  VARIANT (35)..(35): /replace=”Asp“ or ”Ser“ or ”Asn“ or ” “
  VARIANT (36)..(36): /replace=”Trp“ or ” “
  VARIANT (37)..(37): /replace=”Lys“ or ” “
  VARIANT (38)..(38): /replace=”His“ or ” “
  VARIANT (39)..(39): /replace=”Asn“ or ” “
  SITE (1)..(39): /note=”Variant residues given in the sequence have no preference with respect to those in the annotations for variant positions“
YGEGTFISDYSIALDRIHQQDFVNWLLAQRGSSGAPPPS

SEQ ID NO: 324 - protein, 8 aa, Artificial Sequence
  source: /note=”Description of Artificial Sequence Synthetic peptide“
  SITE (1)..(8): /note=”This sequence may encompass 2-8 residues“
GGGGGGGG

SEQ ID NO: 325 - protein, 5 aa, Artificial Sequence
  source: /note=”Description of Artificial Sequence Synthetic peptide“
GGEEE

SEQ ID NO: 326 - protein, 5 aa, Artificial Sequence
  source: /note=”Description of Artificial Sequence Synthetic peptide“
  MOD_RES (4)..(4): gamma-Glu
  MOD_RES (5)..(5): gamma-Glu
GGGXX

SEQ ID NO: 327 - protein, 7 aa, Artificial Sequence
  source: /note=”Description of Artificial Sequence Synthetic peptide“
  MOD_RES (7)..(7): gamma-Glu
GGGGGGX

SEQ ID NO: 328 - protein, 6 aa, Artificial Sequence
  source: /note=”Description of Artificial Sequence Synthetic peptide“
  SITE (1)..(6): /note=”This sequence may encompass 5-6 residues“
GGGGGG

SEQ ID NO: 329 - protein, 9 aa, Artificial Sequence
  source: /note=”Description of Artificial Sequence Synthetic peptide“
  SITE (1)..(9): /note=”This sequence may encompass 1-9 residues“
GGGGGGGGG

SEQ ID NO: 330 - protein, 5 aa, Artificial Sequence
  source: /note=”Description of Artificial Sequence Synthetic peptide“
  MOD_RES (1)..(5): gamma-Glu
  SITE (1)..(5): /note=”This sequence may encompass 1-5 residues“
XXXXX

SEQ ID NO: 331 - protein, 10 aa, Artificial Sequence
  source: /note=”Description of Artificial Sequence Synthetic peptide“
  SITE (1)..(10): /note=”This sequence may encompass 1-10 residues“
GGGGGGGGGG

SEQ ID NO: 332 - protein, 4 aa, Artificial Sequence
  source: /note=”Description of Artificial Sequence Synthetic peptide“
GGGG

SEQ ID NO: 333 - protein, 9 aa, Artificial Sequence
  source: /note=”Description of Artificial Sequence Synthetic peptide“
GGGGGGGGG

SEQ ID NO: 334 - protein, 6 aa, Artificial Sequence
  source: /note=”Description of Artificial Sequence Synthetic peptide“
  SITE (1)..(6): /note=”This sequence may encompass 3-6 residues“
GGGGGG

SEQ ID NO: 335 - protein, 6 aa, Artificial Sequence
  source: /note=”Description of Artificial Sequence Synthetic peptide“
  MOD_RES (6)..(6): gamma-Glu
GGGGGX

SEQ ID NO: 336 - protein, 39 aa, Artificial Sequence; identical to SEQ ID NO: 12
  source: /note=”Description of Artificial Sequence Synthetic polypeptide“
  MOD_RES (2)..(2): Aib
  MOD_RES (17)..(17): Aib
  MOD_RES (18)..(18): Lys, wherein the side chain of Lys is connected to the sequence 2OEGgEgE-C18DA
  MOD_RES (20)..(20): Aib

SEQ ID NO: 337 - protein, 31 aa, Artificial Sequence; identical to SEQ ID NO: 68
  source: /note=”Description of Artificial Sequence Synthetic polypeptide“
  MOD_RES (2)..(2): Aib
  MOD_RES (13)..(13): Aib
  MOD_RES (14)..(14): Lys, wherein the side chain of Lys is connected to the sequence GGGGG-C18DA
  MOD_RES (17)..(17): Aib
  MOD_RES (20)..(20): Aib

SEQ ID NO: 338 - protein, 4 aa, Artificial Sequence
  source: /note=”Description of Artificial Sequence Synthetic peptide“
  MOD_RES (1)..(1): gamma-Glu
XGGG

SEQ ID NO: 339 - protein, 4 aa, Artificial Sequence
  source: /note=”Description of Artificial Sequence Synthetic peptide“
  MOD_RES (1)..(1): epsilon-Lys
XGGG

(19) United States
(12) Patent Application Publication
(10) Pub. No.: US 2023/0143604 A1
(43) Pub. Date: May 11, 2023

(54) QW DOSING OF GIP RECEPTOR AGONIST PEPTIDE COMPOUNDS AND USES THEREOF

(71) Applicant: TAKEDA PHARMACEUTICAL COMPANY LIMITED, Osaka-shi, Osaka (JP)

(72) Inventors: Antoine Charles Olivier HENNINOT, San Diego, CA (US); Derek Cecil COLE, San Diego, CA (US); Nicholas SCORAH, San Diego, CA (US)

(21) Appl. No.: 17/914,013

(22) PCT Filed: Mar. 25, 2021
(86) PCT No.: PCT/JP2021/014422

§ 371 Date: Sep. 23, 2022

Related U.S. Application Data
(60) Provisional application No. 62/994,721, filed on Mar. 25, 2020.

(51) Int. Cl. A61K 38/26; C07K 14/605

(52) U.S. Cl. CPC A61K 38/26; C07K 14/605

(57) ABSTRACT

The present disclosure provides GIP receptor agonist peptide compounds suitable for once per week dosing (QW), said peptide compounds having an activating action on GIP receptors and use of the GIP receptor agonist peptide as a medicament for the treatment and/or prevention of emesis, or a symptom or condition associated with emesis. Specifically, a GIP receptor agonist peptide containing a sequence represented by any of the formulas (I)-(V) or a salt thereof, and a medicament comprising the same are provided.

### CROSS-REFERENCE TO RELATED APPLICATIONS

This application claims priority to U.S. Provisional Application Ser. No. 62/994,721, the entire contents of which are incorporated by reference herein.

### TECHNICAL FIELD

The present disclosure relates to a novel peptide compound having an activating action on GIP receptors and use of the peptide compound as a medicament which may be dosed in a once weekly dosing regimen.

### BACKGROUND

The statements in this section merely provide background information related to the present disclosure and may not constitute prior art.

Both glucagon-like peptide-1 (GLP-1) and glucose-dependent insulinotropic polypeptide (GIP) are peptides called incretin. GLP-1 and GIP are secreted from small intestinal L cells and K cells, respectively.

GLP-1 acts via GLP-1 receptors and is known to have a glucose-dependent insulinotropic action and a feeding suppressive action. On the other hand, GIP is known to have a glucose-dependent insulinotropic action via the GIP receptors (GIPr), though an influence of GIP only on feeding is not clear.

Attempts have been made to search for peptides having GLP-1 receptor/GIP receptor coagonist or glucagon receptor/GLP-1 receptor/GIP receptor triagonist activity and modifications thereof and develop these peptides as anti-obesity drugs, therapeutic drugs for diabetes, or therapeutic drugs for neurodegenerative disorders based on the structure of natural glucagon, GIP, or GLP-1. However, the peptide compound and the compound having a selective activating action on GIP receptors of the present disclosure for the use in treating emesis and similar symptoms associated with nausea and vomiting have not been disclosed.

Patients who experience nausea and vomiting are often unwilling or unable to take their medication regularly; several studies have shown that a less frequent dosing results in higher degree of compliance and thus eventually better treatment of the patients. Therefore, there is an unmet need for long acting preparations of antiemetic medicine. In particular there is a need for long acting preparations of antiemetic GIP receptor agonist peptides that represent an alternative to twice per day (BID) dosing formulations in order to make a change in dosing regimen, frequency of medication or type of medication, more flexible. Extending the duration of action will also provide benefit in diseases where the duration of emetic episodes is longer.

All publications, patents, and patent applications cited herein are incorporated herein by reference in their entirety.

### SUMMARY

It is an object of the present invention to provide a GIP receptor agonist peptide compound which has a GIP receptor activation action and is useful as a preventive/therapeutic agent for diabetes, obesity, and/or an antiemetic agent to prevent/treat diseases accompanied by vomiting or nausea.

The present disclosure provides GIPr agonist peptide compounds comprising a sequence represented by formulae (I)-(V) that are useful as therapeutic agents for the prevention or treatment of emesis as described herein. Surprisingly, the compounds of formulae (I)-(V) exhibit excellent GIP receptor activation action, a longer %₂ life of elimination and improved solubility. Unexpectedly, in some instances, the peptides of formulae (I)-(V) relative to other known GIPr agonist peptides in the art possess improved properties in one or more of: (1) stability in serum, (2) half-life of elimination and (3) solubility. In certain embodiments of this disclosure, the peptides of formulae (I)-(V) relative to other known GIPr agonist peptides that are dosed once per week to treat emesis, or which may be useful as preventative agents of nausea and/or vomiting and other symptoms of emesis, possess improved properties in one or more of: (1) stability in serum, (2) half-life of elimination and (3) solubility.

More specifically, the present disclosure includes the following embodiments:

Embodiment (1). A GIP receptor agonist peptide represented by formula (I):

P¹-Tyr-A2-Glu-Gly-Thr-Phe-Ile-Ser-A9-Tyr-Ser-Ile-A13-A14-Asp-A16-A17-A18-Gln-A20-A21-Phe-Val-A24-Trp-Leu-Leu-Ala-Gln-A30-A31-A32-A33-A34-A35-A36-A37-A38-A39-A40-A41-A42-P², or a pharmaceutically acceptable salt thereof;
wherein
P¹ represents a group represented by formula

### —R^{A1},

### —CO—R^{A1},

### —CO—OR^{A1},

### —CO—COR^{A1},

### —SO—R^{A1},

—SO₂—R^{A1},
—SO₂—OR^{A1},
—CO—NR^{A2}R^{A3}
—SO₂—NR^{A2}R^{A3},
—C(═NR^{A1})—NR^{A2}R^{A3}, or
is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, D-Ala, Ala, Gly, or Pro;
A9: represents Asp or Leu;
A13: represents Aib, or Ala;
A14: represents Leu, Aib, Ile, or Nle;
A16: represents Arg, Ser, or Lys;
A17: represents Aib, Ala, or Ile;
A18: represents Ala, His, or Lys;
A19: represents Gln, or Ala;
A20: represents Aib, Gln, or Ala;
A21: represents Asp, Asn, or Lys;
A24: represents Asn, Gln, or Glu;
A30: represents Arg, Ser, Gln, or Lys;
A31: represents Gly, Pro, or a deletion;
A32: represents Ser, Lys, Pro, Gly, or a deletion;
A33: represents Ser, Lys, Gly, or a deletion;
A34: represents Gly, Asn, or a deletion;
A35: represents Ala, Asp, Ser, Asn, or a deletion;
A36: represents Pro, Trp, or a deletion;
A37: represents Pro, Lys, or a deletion;
A38: represents Pro, His, or a deletion;
A39: represents Ser, Asn, or a deletion;
A40: represents Ile, or a deletion;
A41: represents Thr, or a deletion; and
A42: represents Gln, or a deletion.

Embodiment (2). A GIP receptor agonist peptide represented by formula (II):

P¹-Tyr-A2-Glu-Gly-Thr-Phe-Ile-Ser-Asp-Tyr-Ser-Ile-A13-A14-Asp-A16-A17-A18-A19-A20-A21-Phe-Val-A24-Trp-Leu-Leu-Ala-A29-A30-A31-A32-A33-A34-A35-A36-A37-A38-A39-A40-A41-A42-P², or a pharmaceutically acceptable salt thereof, wherein:
P¹ represents a group represented by formula

### —R^{A1},

### —CO—R^{A1},

### —CO—OR^{A1},

### —CO—COR^{A1},

### —SO—R^{A1},

—SO₂—R^{A1},
—SO₂—OR^{A1},
—CO—NR^{A2}R^{A3},
—SO₂—NR^{A2}R^{A3}, or
—C(═NR^{A1})—NR^{A2}R^{A3}
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, D-Ala, or Gly;
A13: represents Aib, or Ala;
A14: represents Leu, Aib, Ile, Nle, or Lys(R);
A16: represents Arg, Ser, or Lys;
A17: represents Aib, Ala, Ile, or Lys(R);
A19: represents Gin or Ala;
A18: represents Ala, His, or Lys(R);
A20: represents Aib, Gin, Arg, or Ala;
A21: represents Asp, Asn, or Lys(R);
A24: represents Asn, Gin, or Glu;
A29: represents Gin, or Lys(R)
A30: represents Arg, Lys, Ser, Gin, or Lys(R);
A31: represents Gly, Pro, or a deletion;
A32: represents Ser, Lys, Pro, Gly, or a deletion;
A33: represents Ser, Lys, Gly, or a deletion;
A34: represents Gly, Asn, or a deletion;
A35: represents Ala, Asp, Ser, Asn, or a deletion;
A36: represents Pro, Trp, or a deletion;
A37: represents Pro, Lys, or a deletion;
A38: represents Pro, His, or a deletion;
A39: represents Ser, Asn, or a deletion;
A40: represents Ile, or a deletion;
A41: represents Thr, or a deletion;
A42: represents Gln, or a deletion.
wherein in the residue Lys(R), the (R) portion represents X-L-, wherein L represents a linker, and is selected from the following group consisting of 2OEGgEgE, OEGgEgE, 2OEGgE, 3OEGgEgE, G5gEgE, 2OEGgEgEgE, 2OEG and G5gEgE; and X represents a lipid.

Embodiment (3). A GIP receptor agonist peptide represented by formula (IV):

P¹-Tyr-Aib-Glu-Gly-Thr-Phe-Ile-Ser-Asp-Tyr-Ser-Ile-A13-A14-Asp-A16-A17-A18-A19-A20-A21-Phe-Val-A24-Trp-Leu-Leu-Ala-A29-A30-A31-A32-A33-A34-A35-A36-A37-A38-A39-P², or a pharmaceutically acceptable salt thereof, wherein:
P¹ represents H or C₁₋₆ alkyl;
P² represents —NH₂ or —OH;
A13: represents Aib, Ala, or Lys;
A14: represents Leu, Aib, Lys, or Lys(R);
A16: represents Arg, Ser, or Lys;
A17: represents Aib, Ala, Ile, Glu, Lys, or Lys(R);
A18: represents Ala, His, Glu, Lys, or Lys(R);
A19: represents Gln or Ala;
A20: represents Aib, Ala, Gln, Arg, or Lys;
A21: represents Asp, Asn, Lys, or Lys(R);
A24: represents Asn or Glu;
A29: represents Gln, Lys, or Lys(R);
A30: represents Arg, Ser, Gln, Lys, Lys(Ac), or Lys(R);
A31: represents Gly, Pro, or a deletion;
A32: represents Ser, Gly, or a deletion;
A33: represents Ser, Gly, or a deletion;
A34: represents Gly or a deletion;
A35: represents Ala, Ser, or a deletion;
A36: represents Pro or a deletion;
A37: represents Pro or a deletion;
A38: represents Pro or a deletion; and
A39: represents Ser or a deletion;
wherein in the residue Lys(R), the (R) portion represents X-L-, wherein L represents a linker and is selected from the group consisting of 2OEGgE, 2OEGgEgE, G4gE, GGGGG, G5gE, G5gEgE, G6, gEgEgE, OEGgEgE, OEGgEOEGgE, GGPAPAP, and GGPAPAPgE; and X represents C₁₇-C₂₂ monoacid or C₁₇-C₂₂ diacid.

Embodiment (4). The GIP receptor agonist peptide according to embodiment (3) or a pharmaceutically acceptable salt thereof, wherein:

A17: represents Aib, Ala, Ile, Glu, or Lys(R);
A18: represents Ala, His, Glu, or Lys(R);
A21: represents Asp, Asn, or Lys(R); and
A29: represents Gln or Lys(R).

Embodiment (5). The GIP receptor agonist peptide or the pharmaceutically acceptable salt thereof according to embodiment (4) has a solubility of at least 15 mg/mL in phosphate buffer at pH 7.4.

Embodiment (6). The GIP receptor agonist peptide or the pharmaceutically acceptable salt thereof according to embodiment (4) has a solubility of at least 30 mg/mL in phosphate buffer at pH 7.4.

Embodiment (7). The GIP receptor agonist peptide according to embodiment (3) or a pharmaceutically acceptable salt thereof, wherein:

A13: represents Aib or Ala;
A14: represents Leu, Lys, or Lys(R);
A16: represents Arg;
A17: represents Aib, Lys, or Lys(R);
A18: represents Ala, Lys, or Lys(R);
A20: represents Aib;
A29: represents Gln;
A30: represents Arg, Ser, or Lys;
A31: represents Gly or Pro;
A33: represents Ser or a deletion; and
A35: represents Ala or a deletion;
wherein L is selected from the group consisting of 2OEGgE, 2OEGgEgE, OEGgEgE, OEGgEOEGgE, G5, GGPAPAP, and GGPAPAPgE.

Embodiment (8). The GIP receptor agonist peptide according to embodiment (7) or a pharmaceutically acceptable salt thereof, wherein:

A14: represents Leu or Lys(R);
A17: represents Aib or Lys(R);
A18: represents Ala or Lys(R); and
A21: represents Asp, Asn, or Lys(R).

Embodiment (9). The GIP receptor agonist peptide or the pharmaceutically acceptable salt thereof according to embodiment (8) has a solubility of at least 60 mg/mL in phosphate buffer at pH 7.4.

Embodiment (10). The GIP receptor agonist peptide according to embodiment (1) or (2) or the pharmaceutically acceptable salt thereof, wherein A31 is Gly, A32-A41 are deletion; or A32 is Gly and 33-A41 are deletion.

Embodiment (11). The GIP receptor agonist peptide according to any one of embodiments (1)-(10) or the pharmaceutically acceptable salt thereof, wherein P² is —OH.

Embodiment (12). The GIP receptor agonist peptide according to any one of embodiments (2)-(12) or the pharmaceutically acceptable salt thereof, wherein Lys(R) is a Lys residue, and wherein the side chain of said Lys residue is substituted with (R).

Embodiment (13). The GIP receptor agonist peptide according to embodiment (12) or the pharmaceutically acceptable salt thereof, wherein Lys(R) or Km (used interchangeably herein), is a Lys residue substituted with (R), and (R) is represented by X-L-, wherein L is selected from 2OEGgE, 2OEGgEgE, G4gE, GGGGG, G5gE, G5gEgE, G6, gEgEgE, OEGgEgE, OEGgEOEGgE, GGPAPAP, and GGPAPAPgE; and X is C₁₇-C₂₂ diacid.

Embodiment (14). The GIP receptor agonist peptide according to any one of embodiments (2)-(13) or the pharmaceutically acceptable salt thereof, wherein L is 2OEGgEgE, OEGgEgE, 2OEGgE, GGGGG, or G5gEgE; and X is a C₁₈ diacid.

Embodiment (15). The GIP receptor agonist peptide according to embodiment (14) or the pharmaceutically acceptable salt thereof, wherein the linker (L) is 2OEGgEgE or GGGGG, and (R) is 2OEGgEgE-C₁₈ diacid or (R) is GGGGG-C₁₈ diacid.

Embodiment (16). The GIPR agonist peptide according to any one of embodiments (2)-(4), represented by formula (V):

Me-Tyr-Aib-Glu-Gly-Thr-Phe-Ile-Ser-Asp-Tyr-Ser-Ile-A13-A14-Asp-Arg-A17-Ala-Gln-Aib-A21-Phe-Val-Asn-Trp-Leu-Leu-Ala-Gln-A30-A31-A32-A33-A34-A35-A36-A37-A38-A39-P², or a pharmaceutically acceptable salt thereof, wherein
P² represents —NH₂ or —OH;
A13: represents Aib or Ala;
A14: represents Leu, Lys, or Lys(R);
A17: represents Aib, Lys, or Lys(R);
A21: represents Asp, Asn, Lys, or Lys(R);
A30: represents Arg, Ser, Lys, or Lys(R);
A31: represents Gly or Pro;
A32: represents Ser, Gly, or a deletion;
A33: represents Ser or a deletion;
A34: represents Gly or a deletion;
A35: represents Ala or a deletion;
A36: represents Pro or a deletion;
A37: represents Pro or a deletion;
A38: represents Pro or a deletion; and
A39: represents Ser or a deletion,
wherein L is 2OEGgEgE or GGGGG; and X represents C₁₈ diacid.

Embodiment (17). The GIPR agonist peptide according to embodiment (16) or a pharmaceutically acceptable salt thereof, wherein:

A14: represents Leu or Lys(R);
A17: represents Aib or Lys(R);
A21: represents Asp, Asn, or Lys(R); and
A30: represents Arg, Ser, Lys, or Lys(R).

Embodiment (18). The GIP receptor agonist peptide according to embodiment (16) or (17) or the pharmaceutically acceptable salt thereof, wherein the amino acid sequence comprises: P¹-Y-Aib-E-G-T-F-I-S-D-Y-S-I-A-Km-D-R-Aib-A-Q-Aib-D-F-V-N-W-L-L-A-Q-S-P-G-P²; wherein Km is Lys-GGGGG-C₁₈ diacid.

Embodiment (19). The GIPR agonist peptide according to embodiment (18) or the pharmaceutically acceptable salt thereof, wherein the amino acid sequence comprises: Me-Y-Aib-E-G-T-F-I-S-D-Y-S-I-A-Km-D-R-Aib-A-Q-Aib-D-F-V-N-W-L-L-A-Q-S-P-G-OH; wherein Km is Lys-GGGGG-Cis diacid.

Embodiment (20). The GIP receptor agonist peptide according to embodiment (16) or (17) or the pharmaceutically acceptable salt thereof, wherein the amino acid sequence comprises: P¹-Y-Aib-E-G-T-F-I-S-D-Y-S-I-A-Km-D-R-Aib-A-Q-Aib-N-F-V-N-W-L-L-A-Q-S-P-S-S-G-A-P-P-P-S-P²; wherein Km is Lys-GGGGG-Cis diacid.

Embodiment (21). The GIPR agonist peptide according to embodiment (20) or the pharmaceutically acceptable salt thereof, wherein the amino acid sequence comprises: Me-Y-Aib-E-G-T-F-I-S-D-Y-S-I-A-Km-D-R-Aib-A-Q-Aib-N-F-V-N-W-L-L-A-Q-S-P-S-S-G-A-P-P-P-S-OH; wherein Km is Lys-GGGGG-C₁₈ diacid.

Embodiment (22). The GIP receptor agonist peptide according to embodiment (16) or (17) or the pharmaceutically acceptable salt thereof, wherein the amino acid sequence comprises: P¹-Y-Aib-E-G-T-F-I-S-D-Y-S-I-A-L-D-R-Km-A-Q-Aib-D-F-V-N-W-L-L-A-Q-S-P-S-S-G-A-P-P-P-S-P²; wherein Km is Lys-GGGGG-C₁₈ diacid.

Embodiment (23). The GIPR agonist peptide of embodiment (22) or the pharmaceutically acceptable salt thereof, wherein the amino acid sequence comprises: Me-Y-Aib-E-G-T-F-I-S-D-Y-S-I-A-L-D-R-Km-A-Q-Aib-D-F-V-N-W-L-L-A-Q-S-P-S-S-G-A-P-P-P-S-NH₂; wherein Km is Lys-GGGGG-C₁₈ diacid.

Embodiment (24). The GIP receptor agonist peptide according to embodiment (16) or (17) or the pharmaceutically acceptable salt thereof, wherein the amino acid sequence comprises: P1-Y-Aib-E-G-T-F-I-S-D-Y-S-I-A-L-D-R-Aib-A-Q-Aib-Km-F-V-N-W-L-L-A-Q-K-G-P2; wherein Km is Lys-2OEGgEgE-C₁₈ diacid.

Embodiment (25). The GIPR agonist peptide of embodiment (24) or the pharmaceutically acceptable salt thereof, wherein the amino acid sequence comprises: Me-Y-Aib-E-G-T-F-I-S-D-Y-S-I-A-L-D-R-Aib-A-Q-Aib-Km-F-V-N-W-L-L-A-Q-K-G-OH; wherein Km is Lys-2OEGgEgE-C₁₈ diacid.

Embodiment (26). The GIP receptor agonist peptide according to embodiment (16) or (17) or the pharmaceutically acceptable salt thereof, wherein the amino acid sequence comprises: P¹-Y-Aib-E-G-T-F-I-S-D-Y-S-I-Aib-L-D-R-Aib-A-Q-Aib-Km-F-V-N-W-L-L-A-Q-R-G-P²; wherein Km is Lys-2OEGgEgE-C₁₈ diacid.

Embodiment (27). The GIPR agonist peptide of embodiment (26) or the pharmaceutically acceptable salt thereof, wherein the amino acid sequence comprises: Me-Y-Aib-E-G-T-F-I-S-D-Y-S-I-Aib-L-D-R-Aib-A-Q-Aib-Km-F-V-N-W-L-L-A-Q-R-G-OH; wherein Km is Lys-2OEGgEgE-C₁₈ diacid.

Embodiment (28). The GIP receptor agonist peptide according to embodiment (16) or (17) or the pharmaceutically acceptable salt thereof, wherein the amino acid sequence comprises: P¹-Y-Aib-E-G-T-F-I-S-D-Y-S-I-Aib-L-D-R-Aib-A-Q-Aib-N-F-V-N-W-L-L-A-Q-Km-P-S-S-G-A-P-P-P-S-P²; wherein Km is Lys-2OEGgEgE-C₁₈ diacid.

Embodiment (29). The GIPR agonist peptide of embodiment (28) or the pharmaceutically acceptable salt thereof, wherein the amino acid sequence comprises: Me-Y-Aib-E-G-T-F-I-S-D-Y-S-I-Aib-L-D-R-Aib-A-Q-Aib-N-F-V-N-W-L-L-A-Q-Km-P-S-S-G-A-P-P-P-S-NH₂; wherein Km is Lys-2OEGgEgE-C₁₈ diacid.

Embodiment (30). The GIP receptor agonist peptide according to embodiment (16) or (17) or the pharmaceutically acceptable salt thereof, wherein the amino acid sequence comprises: P¹-Y-Aib-E-G-T-F-I-S-D-Y-S-I-A-L-D-R-Aib-Km-Q-Aib-N-F-V-N-W-L-L-A-Q-S—P-S-S-G-A-P-P-P-S-P²; wherein Km is Lys-2OEGgEgE-C₁₈ diacid.

Embodiment (31). The GIP receptor agonist peptide according to embodiment (16) or (17) or the pharmaceutically acceptable salt thereof, wherein the amino acid sequence comprises: P¹-Y-Aib-E-G-T-F-I-S-D-Y-S-I-Aib-Km-D-R-Aib-A-Q-Aib-D-F-V-N-W-L-L-A-Q-R-G-P²; wherein Km is Lys-GGGGG-C₁₈ diacid.

Embodiment (32). The GIP receptor agonist peptide according to any one of embodiments (1)-(31) or the pharmaceutically acceptable salt thereof, wherein P¹ is Methyl-(Me) and P² is —OH or NH₂.

Embodiment (33). The GIP receptor agonist peptide according to any one of embodiments (1)-(32) or the pharmaceutically acceptable salt thereof, wherein the GIP receptor agonist peptide has a selectivity ratio, expressed as a ratio of (GLP1R EC₅₀/GIPR EC₅₀) of greater than 10, or greater than 100, or greater than 1,000, or greater than 100,000.

Embodiment (34). A medicament comprising the GIP receptor agonist peptide according to any one of embodiments (1)-(33) or a pharmaceutically acceptable salt thereof.

Embodiment (35). A pharmaceutical composition comprising the GIP receptor agonist peptide according to any one of embodiments (1)-(33) or a pharmaceutically acceptable salt thereof.

Embodiment (36). The GIP receptor agonist peptide according to any one of embodiments (1)-(33) or the pharmaceutically acceptable salt thereof, or the medicament according to embodiment (34), or the pharmaceutical composition according to embodiment (35), which is administered once per week (QW) to treat emesis as a monotherapy or as an adjunct therapy.

Embodiment (37). Use of the GIP receptor agonist peptide according to any one of embodiments (1)-(33), or a salt thereof, or the medicament according to embodiment (34), or the pharmaceutical composition according to embodiment (35), for the manufacture of a suppressant for vomiting or nausea.

Embodiment (38). The peptide of according to any one of embodiments (1)-(33), or a salt thereof, or the medicament according to embodiment (34), or the pharmaceutical composition according to embodiment (35), for use in suppressing vomiting or nausea.

Embodiment (39). A method for preventing or treating emesis in a subject, comprising administering an effective amount of the peptide of any one of embodiments (1)-(33), or a salt thereof, or the medicament according to embodiment (34), or the pharmaceutical composition according to embodiment (35), to the subject.

Embodiment (40). The medicament according to embodiment (34), the use according to embodiment (37), the peptide, medicament, or pharmaceutical composition according to embodiment (38), the method according to embodiment (39), where the emesis, vomiting or the nausea is caused by one or more conditions or causes selected from the following (1) to (10):

(1) Diseases accompanied by vomiting or nausea such as gastroparesis, gastrointestinal hypomotility, peritonitis, abdominal tumor, constipation, gastrointestinal obstruction, chronic intestinal pseudo-obstruction, functional dyspepsia, cyclic vomiting syndrome, chemotherapy-induced nausea and vomiting (CINV), nausea and/or vomiting associated with gastroparesis, chronic unexplained nausea and vomiting, acute pancreatitis, chronic pancreatitis, hepatitis, hyperkalemia, cerebral edema, intracranial lesion, metabolic disorder, gastritis caused by an infection, postoperative disease, myocardial infarction, migraine, intracranial hypertension, and intracranial hypotension (e.g., altitude sickness);
(2) Vomiting and/or nausea induced by chemotherapeutic drugs such as (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome;
(3) Vomiting or nausea caused by radiation sickness or radiation therapy for the chest, the abdomen, or the like used to treat cancers;
(4) Vomiting or nausea caused by a poisonous substance or a toxin;
(5) Vomiting and nausea caused by pregnancy including hyperemesis gravidarium; and
(6) Vomiting and nausea caused by a vestibular disorder such as motion sickness or dizziness
(7) Opioid withdrawal;
(8) Vomiting and nausea caused by chronic unexplained nausea and vomiting;
(9) A vestibular disorder such as motion sickness or dizziness; and
(10) A physical injury causing local, systemic, acute or chronic pain.

Embodiment (41). The method according to embodiment (39), wherein emesis is treated in a subject not taking a medicament to control a metabolic syndrome disorder.

Embodiment (42). A GIP receptor agonist peptide of any one of embodiments (1)-(33) or the salt thereof, wherein the peptide selectively activates the GIP receptor and demonstrates an antiemetic action in vivo, and wherein the antiemetic action is achieved by dosing the peptide to a subject in need thereof, once per week, or once per 5-7 days, or four to six times per month.

It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims. Other features and advantages of the disclosure will be apparent from the following Detailed Description, the drawings, and the claims.

### BRIEF DESCRIPTION OF THE FIGURES

FIG. 1. Exemplary GIP receptor agonist peptides of the present disclosure which are represented by any one of formulas (I)-(V).

### DETAILED DESCRIPTION

The definition of each substituent used in the present specification is described in detail in the following. Unless otherwise specified, each substituent has the following definition.

In the present specification, examples of the “halogen atom” include fluorine, chlorine, bromine and iodine.

In the present specification, examples of the “C₁₋₆ alkyl group” include methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl and 2-ethylbutyl.

In the present specification, examples of the “optionally halogenated C₁₋₆ alkyl group” include a C₁₋₆ alkyl group optionally having 1 to 7, or 1 to 5, halogen atoms. Specific examples thereof include methyl, chloromethyl, difluoromethyl, trichloromethyl, trifluoromethyl, ethyl, 2-bromoethyl, 2,2,2-trifluoroethyl, tetrafluoroethyl, pentafluoroethyl, propyl, 2,2-difluoropropyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl and 6,6,6-trifluorohexyl.

In the present specification, examples of the “C₂₋₆ alkenyl group” include ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl and 5-hexenyl.

In the present specification, examples of the “C₂₋₆ alkynyl group” include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and 4-methyl-2-pentynyl.

In the present specification, examples of the “C₃₋₁₀ cycloalkyl group” include cyclopropyl, cyclobutyl, cyclopentyl, cyclohexyl, cycloheptyl, cyclooctyl, bicyclo[2.2.1]heptyl, bicyclo[2.2.2]octyl, bicyclo[3.2.1]octyl and adamantyl.

In the present specification, examples of the “optionally halogenated C₃₋₁₀ cycloalkyl group” include a C₃₋₁₀ cycloalkyl group optionally having 1 to 7, or 1 to 5, halogen atoms. Specific examples thereof include cyclopropyl, 2,2-difluorocyclopropyl, 2,3-difluorocyclopropyl, cyclobutyl, difluorocyclobutyl, cyclopentyl, cyclohexyl, cycloheptyl and cyclooctyl.

In the present specification, examples of the “C₃₋₁₀ cycloalkenyl group” include cyclopropenyl, cyclobutenyl, cyclopentenyl, cyclohexenyl, cycloheptenyl and cyclooctenyl.

In the present specification, examples of the “C₆₋₁₄ aryl group” include phenyl, 1-naphthyl, 2-naphthyl, 1-anthryl, 2-anthryl and 9-anthryl.

In the present specification, examples of the “C₇₋₁₆ aralkyl group” include benzyl, phenethyl, naphthylmethyl and phenylpropyl.

In the present specification, examples of the “C₁₋₆ alkoxy group” include methoxy, ethoxy, propoxy, isopropoxy, butoxy, isobutoxy, sec-butoxy, tert-butoxy, pentyloxy and hexyloxy.

In the present specification, examples of the “optionally halogenated C₁₋₆ alkoxy group” include a C₁₋₆ alkoxy group optionally having 1 to 7, or 1 to 5, halogen atoms. Specific examples thereof include methoxy, difluoromethoxy, trifluoromethoxy, ethoxy, 2,2,2-trifluoroethoxy, propoxy, isopropoxy, butoxy, 4,4,4-trifluorobutoxy, isobutoxy, sec-butoxy, pentyloxy and hexyloxy.

In the present specification, examples of the “C₃₋₁₀ cycloalkyloxy group” include cyclopropyloxy, cyclobutyloxy, cyclopentyloxy, cyclohexyloxy, cycloheptyloxy and cyclooctyloxy.

In the present specification, examples of the “C₁₋₆ alkylthio group” include methylthio, ethylthio, propylthio, isopropylthio, butylthio, sec-butylthio, tert-butylthio, pentylthio and hexylthio.

In the present specification, examples of the “optionally halogenated C₁₋₆ alkylthio group” include a C₁₋₆ alkylthio group optionally having 1 to 7, or 1 to 5, halogen atoms. Specific examples thereof include methylthio, difluoromethylthio, trifluoromethylthio, ethylthio, propylthio, isopropylthio, butylthio, 4,4,4-trifluorobutylthio, pentylthio and hexylthio.

In the present specification, examples of the “C₁₋₆ alkyl-carbonyl group” include acetyl, propanoyl, butanoyl, 2-methylpropanoyl, pentanoyl, 3-methylbutanoyl, 2-methylbutanoyl, 2,2-dimethylpropanoyl, hexanoyl and heptanoyl.

In the present specification, examples of the “optionally halogenated C₁₋₆ alkyl-carbonyl group” include a C₁₋₆ alkyl-carbonyl group optionally having 1 to 7, or 1 to 5, halogen atoms. Specific examples thereof include acetyl, chloroacetyl, trifluoroacetyl, trichloroacetyl, propanoyl, butanoyl, pentanoyl and hexanoyl.

In the present specification, examples of the “C₁₋₆ alkoxy-carbonyl group” include methoxycarbonyl, ethoxycarbonyl, propoxycarbonyl, isopropoxycarbonyl, butoxycarbonyl, isobutoxycarbonyl, sec-butoxycarbonyl, tert-butoxycarbonyl, pentyloxycarbonyl and hexyloxycarbonyl.

In the present specification, examples of the “C₆₋₁₄ aryl-carbonyl group” include benzoyl, 1-naphthoyl and 2-naphthoyl.

In the present specification, examples of the “C₇₋₁₆ aralkyl-carbonyl group” include phenylacetyl and phenylpropionyl.

In the present specification, examples of the “5- to 14-membered aromatic heterocyclylcarbonyl group” include nicotinoyl, isonicotinoyl, thenoyl and furoyl.

In the present specification, examples of the “3- to 14-membered non-aromatic heterocyclylcarbonyl group” include morpholinylcarbonyl, piperidinylcarbonyl and pyrrolidinylcarbonyl.

In the present specification, examples of the “mono- or di-C₁₋₆ alkyl-carbamoyl group” include methylcarbamoyl, ethylcarbamoyl, dimethylcarbamoyl, diethylcarbamoyl and N-ethyl-N-methylcarbamoyl.

In the present specification, examples of the “mono- or di-C₇₋₁₆ aralkyl-carbamoyl group” include benzylcarbamoyl and phenethylcarbamoyl.

In the present specification, examples of the “C₁₋₆ alkylsulfonyl group” include methylsulfonyl, ethylsulfonyl, propylsulfonyl, isopropylsulfonyl, butylsulfonyl, sec-butylsulfonyl and tert-butylsulfonyl.

In the present specification, examples of the “optionally halogenated C₁₋₆ alkylsulfonyl group” include a C₁₋₆ alkylsulfonyl group optionally having 1 to 7, or 1 to 5, halogen atoms. Specific examples thereof include methylsulfonyl, difluoromethylsulfonyl, trifluoromethylsulfonyl, ethylsulfonyl, propylsulfonyl, isopropylsulfonyl, butylsulfonyl, 4,4,4-trifluorobutylsulfonyl, pentylsulfonyl and hexylsulfonyl.

In the present specification, examples of the “C₆₋₁₄ arylsulfonyl group” include phenylsulfonyl, 1-naphthylsulfonyl and 2-naphthylsulfonyl.

In the present specification, examples of the “substituent” include a halogen atom, a cyano group, a nitro group, an optionally substituted hydrocarbon group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group and an optionally substituted silyl group.

In the present specification, examples of the “hydrocarbon group” (including “hydrocarbon group” of “optionally substituted hydrocarbon group”) include a C₁₋₆ alkyl group, a C₂₋₆ alkenyl group, a C₂₋₆ alkynyl group, a C₃₋₁₀ cycloalkyl group, a C₃₋₁₀ cycloalkenyl group, a C₆₋₁₄ aryl group and a C₇₋₁₆ aralkyl group.

In the present specification, examples of the “optionally substituted hydrocarbon group” include a hydrocarbon group optionally having substituent(s) selected from the following substituent group A.

### [Substituent Group A]

(1) a halogen atom,
(2) a nitro group,
(3) a cyano group,
(4) an oxo group,
(5) a hydroxy group,
(6) an optionally halogenated C₁₋₆ alkoxy group,
(7) a C₆₋₁₄ aryloxy group (e.g., phenoxy, naphthoxy),
(8) a C₇₋₁₆ aralkyloxy group (e.g., benzyloxy),
(9) a 5- to 14-membered aromatic heterocyclyloxy group (e.g., pyridyloxy),
(10) a 3- to 14-membered non-aromatic heterocyclyloxy group (e.g., morpholinyloxy, piperidinyloxy),
(11) a C₁₋₆ alkyl-carbonyloxy group (e.g., acetoxy, propanoyloxy),
(12) a C₆₋₁₄ aryl-carbonyloxy group (e.g., benzoyloxy, 1-naphthoyloxy, 2-naphthoyloxy),
(13) a C₁₋₆ alkoxy-carbonyloxy group (e.g., methoxycarbonyloxy, ethoxycarbonyloxy, propoxycarbonyloxy, butoxycarbonyloxy),
(14) a mono- or di-C₁₋₆ alkyl-carbamoyloxy group (e.g., methylcarbamoyloxy, ethylcarbamoyloxy, dimethylcarbamoyloxy, diethylcarbamoyloxy),
(15) a C₆₋₁₄ aryl-carbamoyloxy group (e.g., phenylcarbamoyloxy, naphthylcarbamoyloxy),
(16) a 5- to 14-membered aromatic heterocyclylcarbonyloxy group (e.g., nicotinoyloxy),
(17) a 3- to 14-membered non-aromatic heterocyclylcarbonyloxy group (e.g., morpholinylcarbonyloxy, piperidinylcarbonyloxy),
(18) an optionally halogenated C₁₋₆ alkylsulfonyloxy group (e.g., methylsulfonyloxy, trifluoromethylsulfonyloxy),
(19) a C₆₋₁₄ arylsulfonyloxy group optionally substituted by a C₁₋₆ alkyl group (e.g., phenylsulfonyloxy, toluenesulfonyloxy),
(20) an optionally halogenated C₁₋₆ alkylthio group,
(21) a 5- to 14-membered aromatic heterocyclic group,
(22) a 3- to 14-membered non-aromatic heterocyclic group,
(23) a formyl group,
(24) a carboxy group,
(25) an optionally halogenated C₁₋₆ alkyl-carbonyl group,
(26) a C₆₋₁₄ aryl-carbonyl group,
(27) a 5- to 14-membered aromatic heterocyclylcarbonyl group,
(28) a 3- to 14-membered non-aromatic heterocyclylcarbonyl group,
(29) a C₁₋₆ alkoxy-carbonyl group,
(30) a C₆₋₁₄ aryloxy-carbonyl group (e.g., phenyloxycarbonyl, 1-naphthyloxycarbonyl, 2-naphthyloxycarbonyl),
(31) a C₇₋₁₆ aralkyloxy-carbonyl group (e.g., benzyloxycarbonyl, phenethyloxycarbonyl),
(32) a carbamoyl group,
(33) a thiocarbamoyl group,
(34) a mono- or di-C₁₋₆ alkyl-carbamoyl group,
(35) a C₆₋₁₄ aryl-carbamoyl group (e.g., phenylcarbamoyl),
(36) a 5- to 14-membered aromatic heterocyclylcarbamoyl group (e.g., pyridylcarbamoyl, thienylcarbamoyl),
(37) a 3- to 14-membered non-aromatic heterocyclylcarbamoyl group (e.g., morpholinylcarbamoyl, piperidinylcarbamoyl),
(38) an optionally halogenated C₁₋₆ alkylsulfonyl group,
(39) a C₆₋₁₄ arylsulfonyl group,
(40) a 5- to 14-membered aromatic heterocyclylsulfonyl group (e.g., pyridylsulfonyl, thienylsulfonyl),
(41) an optionally halogenated C₁₋₆ alkylsulfinyl group,
(42) a C₆₋₁₄ arylsulfinyl group (e.g., phenylsulfinyl, 1-naphthylsulfinyl, 2-naphthylsulfinyl),
(43) a 5- to 14-membered aromatic heterocyclylsulfinyl group (e.g., pyridylsulfinyl, thienylsulfinyl),
(44) an amino group,
(45) a mono- or di-C₁₋₆ alkylamino group (e.g., methylamino, ethylamino, propylamino, isopropylamino, butylamino, dimethylamino, diethylamino, dipropylamino, dibutylamino, N-ethyl-N-methylamino),
(46) a mono- or di-C₆₋₁₄ arylamino group (e.g., phenylamino),
(47) a 5- to 14-membered aromatic heterocyclylamino group (e.g., pyridylamino),
(48) a C₇₋₁₆ aralkylamino group (e.g., benzylamino),
(49) a formylamino group,
(50) a C₁₋₆ alkyl-carbonylamino group (e.g., acetylamino, propanoylamino, butanoylamino),
(51) a (C₁₋₆ alkyl)(C₁₋₆ alkyl-carbonyl)amino group (e.g., N-acetyl-N-methylamino),
(52) a C₆₋₁₄ aryl-carbonylamino group (e.g., phenylcarbonylamino, naphthylcarbonylamino),
(53) a C₁₋₆ alkoxy-carbonylamino group (e.g., methoxycarbonylamino, ethoxycarbonylamino, propoxycarbonylamino, butoxycarbonylamino, tert-butoxycarbonylamino),
(54) a C₇₋₁₆ aralkyloxy-carbonylamino group (e.g., benzyloxycarbonylamino),
(55) a C₁₋₆ alkylsulfonylamino group (e.g., methylsulfonylamino, ethylsulfonylamino),
(56) a C₆₋₁₄ arylsulfonylamino group optionally substituted by a C₁₋₆ alkyl group (e.g., phenylsulfonylamino, toluenesulfonylamino),
(57) an optionally halogenated C₁₋₆ alkyl group,
(58) a C₂₋₆ alkenyl group,
(59) a C₂₋₆ alkynyl group,
(60) a C₃₋₁₀ cycloalkyl group,
(61) a C₃₋₁₀ cycloalkenyl group and
(62) a C₆₋₁₄ aryl group.

The number of the above-mentioned substituents in the “optionally substituted hydrocarbon group” is, for example, 1 to 5, or 1 to 3. When the number of the substituents is two or more, the respective substituents may be the same or different.

In the present specification, examples of the “heterocyclic group” (including “heterocyclic group” of “optionally substituted heterocyclic group”) include (i) an aromatic heterocyclic group, (ii) a non-aromatic heterocyclic group and (iii) a 7- to 10-membered bridged heterocyclic group, each containing, as a ring-constituting atom besides carbon atom, 1 to 4 hetero atoms selected from a nitrogen atom, a sulfur atom and an oxygen atom.

In the present specification, examples of the “aromatic heterocyclic group” (including “5- to 14-membered aromatic heterocyclic group”) include a 5- to 14-membered (e.g., 5- to 10-membered) aromatic heterocyclic group containing, as a ring-constituting atom besides carbon atom, 1 to 4 hetero atoms selected from a nitrogen atom, a sulfur atom and an oxygen atom.

Examples of the “aromatic heterocyclic group” include 5- or 6-membered monocyclic aromatic heterocyclic groups such as thienyl, furyl, pyrrolyl, imidazolyl, pyrazolyl, thiazolyl, isothiazolyl, oxazolyl, isoxazolyl, pyridyl, pyrazinyl, pyrimidinyl, pyridazinyl, 1,2,4-oxadiazolyl, 1,3,4-oxadiazolyl, 1,2,4-thiadiazolyl, 1,3,4-thiadiazolyl, triazolyl, tetrazolyl, triazinyl and the like; and 8- to 14-membered fused polycyclic (e.g., bi or tricyclic) aromatic heterocyclic groups such as benzothiophenyl, benzofuranyl, benzimidazolyl, benzoxazolyl, benzisoxazolyl, benzothiazolyl, benzisothiazolyl, benzotriazolyl, imidazopyridinyl, thienopyridinyl, furopyridinyl, pyrrolopyridinyl, pyrazolopyridinyl, oxazolopyridinyl, thiazolopyridinyl, imidazopyrazinyl, imidazopyrimidinyl, thienopyrimidinyl, furopyrimidinyl, pyrrolopyrimidinyl, pyrazolopyrimidinyl, oxazolopyrimidinyl, thiazolopyrimidinyl, pyrazolotriazinyl, naphtho[2,3-b]thienyl, phenoxathiinyl, indolyl, isoindolyl, 1H-indazolyl, purinyl, isoquinolyl, quinolyl, phthalazinyl, naphthyridinyl, quinoxalinyl, quinazolinyl, cinnolinyl, carbazolyl, O-carbolinyl, phenanthridinyl, acridinyl, phenazinyl, phenothiazinyl, phenoxazinyl and the like.

In the present specification, examples of the “non-aromatic heterocyclic group” (including “3- to 14-membered non-aromatic heterocyclic group”) include a 3- to 14-membered (e.g., 4- to 10-membered) non-aromatic heterocyclic group containing, as a ring-constituting atom besides carbon atom, 1 to 4 hetero atoms selected from a nitrogen atom, a sulfur atom and an oxygen atom.

Examples of the “non-aromatic heterocyclic group” include 3- to 8-membered monocyclic non-aromatic heterocyclic groups such as aziridinyl, oxiranyl, thiiranyl, azetidinyl, oxetanyl, thietanyl, tetrahydrothienyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydroisothiazolyl, tetrahydrooxazolyl, tetrahydroisooxazolyl, piperidinyl, piperazinyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, thiomorpholinyl, azepanyl, diazepanyl, azepinyl, oxepanyl, azocanyl, diazocanyl and the like; and 9- to 14-membered fused polycyclic (e.g., bi or tricyclic) non-aromatic heterocyclic groups such as dihydrobenzofuranyl, dihydrobenzimidazolyl, dihydrobenzoxazolyl, dihydrobenzothiazolyl, dihydrobenzisothiazolyl, dihydronaphtho[2,3-b]thienyl, tetrahydroisoquinolyl, tetrahydroquinolyl, 4H-quinolizinyl, indolinyl, isoindolinyl, tetrahydrothieno[2,3-c]pyridinyl, tetrahydrobenzazepinyl, tetrahydroquinoxalinyl, tetrahydrophenanthridinyl, hexahydrophenothiazinyl, hexahydrophenoxazinyl, tetrahydrophthalazinyl, tetrahydronaphthyridinyl, tetrahydroquinazolinyl, tetrahydrocinnolinyl, tetrahydrocarbazolyl, tetrahydro-β-carbolinyl, tetrahydroacrydinyl, tetrahydrophenazinyl, tetrahydrothioxanthenyl, octahydroisoquinolyl and the like.

In the present specification, examples of the “7- to 10-membered bridged heterocyclic group” include quinuclidinyl and 7-azabicyclo[2.2.1]heptanyl.

In the present specification, examples of the “nitrogen-containing heterocyclic group” include a “heterocyclic group” containing at least one nitrogen atom as a ring-constituting atom.

In the present specification, examples of the “optionally substituted heterocyclic group” include a heterocyclic group optionally having substituent(s) selected from the aforementioned substituent group A.

The number of the substituents in the “optionally substituted heterocyclic group” is, for example, 1 to 3. When the number of the substituents is two or more, the respective substituents may be the same or different.

In the present specification, examples of the “acyl group” include a formyl group, a carboxy group, a carbamoyl group, a thiocarbamoyl group, a sulfino group, a sulfo group, a sulfamoyl group and a phosphono group, each optionally having “1 or 2 substituents selected from a C₁₋₆ alkyl group, a C₂₋₆ alkenyl group, a C₃₋₁₀ cycloalkyl group, a C₃₋₁₀ cycloalkenyl group, a C₆₋₁₄ aryl group, a C₇₋₁₆ aralkyl group, a 5- to 14-membered aromatic heterocyclic group and a 3- to 14-membered non-aromatic heterocyclic group, each of which optionally has 1 to 3 substituents selected from a halogen atom, an optionally halogenated C₁₋₆ alkoxy group, a hydroxy group, a nitro group, a cyano group, an amino group and a carbamoyl group”.

Examples of the “acyl group” (also referred to as “Ac”) also include a hydrocarbon-sulfonyl group, a heterocyclylsulfonyl group, a hydrocarbon-sulfinyl group and a heterocyclylsulfinyl group.

In some embodiments, the hydrocarbon-sulfonyl group means a hydrocarbon group-bonded sulfonyl group, the heterocyclylsulfonyl group means a heterocyclic group-bonded sulfonyl group, the hydrocarbon-sulfinyl group means a hydrocarbon group-bonded sulfinyl group and the heterocyclylsulfinyl group means a heterocyclic group-bonded sulfinyl group.

Examples of the “acyl group” include a formyl group, a carboxy group, a C₁₋₆ alkyl-carbonyl group, a C₂₋₆ alkenyl-carbonyl group (e.g., crotonoyl), a C₃₋₁₀ cycloalkyl-carbonyl group (e.g., cyclobutanecarbonyl, cyclopentanecarbonyl, cyclohexanecarbonyl, cycloheptanecarbonyl), a C₃₋₁₀ cycloalkenyl-carbonyl group (e.g., 2-cyclohexenecarbonyl), a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a C₆₋₁₄ aryloxy-carbonyl group (e.g., phenyloxycarbonyl, naphthyloxycarbonyl), a C₇₋₁₆ aralkyloxy-carbonyl group (e.g., benzyloxycarbonyl, phenethyloxycarbonyl), a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl), a mono- or di-C₆₋₁₄ aryl-carbamoyl group (e.g., phenylcarbamoyl), a mono- or di-C₇₋₁₆ aralkyl-carbamoyl group, a 5- to 14-membered aromatic heterocyclylcarbamoyl group (e.g., pyridylcarbamoyl), a thiocarbamoyl group, a mono- or di-C₁₋₆ alkyl-thiocarbamoyl group (e.g., methylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a 5- to 14-membered aromatic heterocyclylthiocarbamoyl group (e.g., pyridylthiocarbamoyl), a sulfino group, a C₁₋₆ alkylsulfinyl group (e.g., methylsulfinyl, ethylsulfinyl), a sulfo group, a C₁₋₆ alkylsulfonyl group, a C₆₋₁₄ arylsulfonyl group, a phosphono group and a mono- or di-C₁₋₆ alkylphosphono group (e.g., dimethylphosphono, diethylphosphono, diisopropylphosphono, dibutylphosphono).

In the present specification, examples of the “optionally substituted amino group” include an amino group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from substituent group A”.

Examples of the optionally substituted amino group include an amino group, a mono- or di-(optionally halogenated C₁₋₆ alkyl)amino group (e.g., methylamino, trifluoromethylamino, dimethylamino, ethylamino, diethylamino, propylamino, dibutylamino), a mono- or di-C₂₋₆ alkenylamino group (e.g., diallylamino), a mono- or di-C₃₋₁₀ cycloalkylamino group (e.g., cyclopropylamino, cyclohexylamino), a mono- or di-C₆₋₁₄ arylamino group (e.g., phenylamino), a mono- or di-C₇₋₁₆ aralkylamino group (e.g., benzylamino, dibenzylamino), a mono- or di-(optionally halogenated C₁₋₆ alkyl)-carbonylamino group (e.g., acetylamino, propionylamino), a mono- or di-C₆₋₁₄ aryl-carbonylamino group (e.g., benzoylamino), a mono- or di-C₇₋₁₆ aralkyl-carbonylamino group (e.g., benzylcarbonylamino), a mono- or di-5- to 14-membered aromatic heterocyclylcarbonylamino group (e.g., nicotinoylamino, isonicotinoylamino), a mono- or di-3- to 14-membered non-aromatic heterocyclylcarbonylamino group (e.g., piperidinylcarbonylamino), a mono- or di-C₁₋₆ alkoxy-carbonylamino group (e.g., tert-butoxycarbonylamino), a 5- to 14-membered aromatic heterocyclylamino group (e.g., pyridylamino), a carbamoylamino group, a (mono- or di-C₁₋₆ alkyl-carbamoyl)amino group (e.g., methylcarbamoylamino), a (mono- or di-C₇₋₁₆ aralkyl-carbamoyl)amino group (e.g., benzylcarbamoylamino), a C₁₋₆ alkylsulfonylamino group (e.g., methylsulfonylamino, ethylsulfonylamino), a C₆₋₁₄ arylsulfonylamino group (e.g., phenylsulfonylamino), a (C₁₋₆ alkyl)(C₁₋₆ alkyl-carbonyl)amino group (e.g., N-acetyl-N-methylamino) and a (C₁₋₆ alkyl)(C₆₋₁₄ aryl-carbonyl)amino group (e.g., N-benzoyl-N-methylamino).

In the present specification, examples of the “optionally substituted carbamoyl group” include a carbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from substituent group A”.

Examples of the optionally substituted carbamoyl group include a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl, cyclohexylcarbamoyl), a mono- or di-C₆₋₁₄ aryl-carbamoyl group (e.g., phenylcarbamoyl), a mono- or di-C₇₋₁₆ aralkyl-carbamoyl group, a mono- or di-C₁₋₆ alkyl-carbonyl-carbamoyl group (e.g., acetylcarbamoyl, propionylcarbamoyl), a mono- or di-C₆₋₁₄ aryl-carbonyl-carbamoyl group (e.g., benzoylcarbamoyl) and a 5- to 14-membered aromatic heterocyclylcarbamoyl group (e.g., pyridylcarbamoyl).

In the present specification, examples of the “optionally substituted thiocarbamoyl group” include a thiocarbamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from substituent group A”.

Examples of the optionally substituted thiocarbamoyl group include a thiocarbamoyl group, a mono- or di-C₁₋₆ alkyl-thiocarbamoyl group (e.g., methylthiocarbamoyl, ethylthiocarbamoyl, dimethylthiocarbamoyl, diethylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a mono- or di-C₁₋₆ alkyl-carbonyl-thiocarbamoyl group (e.g., acetylthiocarbamoyl, propionylthiocarbamoyl), a mono- or di-C₆₋₁₄ aryl-carbonyl-thiocarbamoyl group (e.g., benzoylthiocarbamoyl) and a 5- to 14-membered aromatic heterocyclylthiocarbamoyl group (e.g., pyridylthiocarbamoyl).

In the present specification, examples of the “optionally substituted sulfamoyl group” include a sulfamoyl group optionally having “1 or 2 substituents selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group and a mono- or di-C₇₋₁₆ aralkyl-carbamoyl group, each of which optionally has 1 to 3 substituents selected from substituent group A”.

Examples of the optionally substituted sulfamoyl group include a sulfamoyl group, a mono- or di-C₁₋₆ alkyl-sulfamoyl group (e.g., methylsulfamoyl, ethylsulfamoyl, dimethylsulfamoyl, diethylsulfamoyl, N-ethyl-N-methylsulfamoyl), a mono- or di-C₂₋₆ alkenyl-sulfamoyl group (e.g., diallylsulfamoyl), a mono- or di-C₃₋₁₀ cycloalkyl-sulfamoyl group (e.g., cyclopropylsulfamoyl, cyclohexylsulfamoyl), a mono- or di-C₆₋₁₄ aryl-sulfamoyl group (e.g., phenylsulfamoyl), a mono- or di-C₇₋₁₆ aralkyl-sulfamoyl group (e.g., benzylsulfamoyl, phenethylsulfamoyl), a mono- or di-C₁₋₆ alkyl-carbonyl-sulfamoyl group (e.g., acetylsulfamoyl, propionylsulfamoyl), a mono- or di-C₆₋₁₄ aryl-carbonyl-sulfamoyl group (e.g., benzoylsulfamoyl) and a 5- to 14-membered aromatic heterocyclylsulfamoyl group (e.g., pyridylsulfamoyl).

In the present specification, examples of the “optionally substituted hydroxy group” include a hydroxyl group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a 5- to 14-membered aromatic heterocyclic group, a carbamoyl group, a mono- or di-C₁₋₆ alkyl-carbamoyl group, a mono- or di-C₇₋₁₆ aralkyl-carbamoyl group, a C₁₋₆ alkylsulfonyl group and a C₆₋₁₄ arylsulfonyl group, each of which optionally has 1 to 3 substituents selected from substituent group A”.

Examples of the optionally substituted hydroxy group include a hydroxy group, a C₁₋₆ alkoxy group, a C₂₋₆ alkenyloxy group (e.g., allyloxy, 2-butenyloxy, 2-pentenyloxy, 3-hexenyloxy), a C₃₋₁₀ cycloalkyloxy group (e.g., cyclohexyloxy), a C₆₋₁₄ aryloxy group (e.g., phenoxy, naphthyloxy), a C₇₋₁₆ aralkyloxy group (e.g., benzyloxy, phenethyloxy), a C₁₋₆ alkyl-carbonyloxy group (e.g., acetyloxy, propionyloxy, butyryloxy, isobutyryloxy, pivaloyloxy), a C₆₋₁₄ aryl-carbonyloxy group (e.g., benzoyloxy), a C₇₋₁₆ aralkyl-carbonyloxy group (e.g., benzylcarbonyloxy), a 5- to 14-membered aromatic heterocyclylcarbonyloxy group (e.g., nicotinoyloxy), a 3- to 14-membered non-aromatic heterocyclylcarbonyloxy group (e.g., piperidinylcarbonyloxy), a C₁₋₆ alkoxy-carbonyloxy group (e.g., tert-butoxycarbonyloxy), a 5- to 14-membered aromatic heterocyclyloxy group (e.g., pyridyloxy), a carbamoyloxy group, a C₁₋₆ alkyl-carbamoyloxy group (e.g., methylcarbamoyloxy), a C₇₋₁₆ aralkyl-carbamoyloxy group (e.g., benzylcarbamoyloxy), a C₁₋₆ alkylsulfonyloxy group (e.g., methylsulfonyloxy, ethylsulfonyloxy) and a C₆₋₁₄ arylsulfonyloxy group (e.g., phenylsulfonyloxy).

In the present specification, examples of the “optionally substituted sulfanyl group” include a sulfanyl group optionally having “a substituent selected from a C₁₋₆ alkyl group, a C₂₋₆ alkenyl group, a C₃₋₁₀ cycloalkyl group, a C₆₋₁₄ aryl group, a C₇₋₁₆ aralkyl group, a C₁₋₆ alkyl-carbonyl group, a C₆₋₁₄ aryl-carbonyl group and a 5- to 14-membered aromatic heterocyclic group, each of which optionally has 1 to 3 substituents selected from substituent group A” and a halogenated sulfanyl group.

Examples of the optionally substituted sulfanyl group include a sulfanyl (—SH) group, a C₁₋₆ alkylthio group, a C₂₋₆ alkenylthio group (e.g., allylthio, 2-butenylthio, 2-pentenylthio, 3-hexenylthio), a C₃₋₁₀ cycloalkylthio group (e.g., cyclohexylthio), a C₆₋₁₄ arylthio group (e.g., phenylthio, naphthylthio), a C₇₋₁₆ aralkylthio group (e.g., benzylthio, phenethylthio), a C₁₋₆ alkyl-carbonylthio group (e.g., acetylthio, propionylthio, butyrylthio, isobutyrylthio, pivaloylthio), a C₆₋₁₄ aryl-carbonylthio group (e.g., benzoylthio), a 5- to 14-membered aromatic heterocyclylthio group (e.g., pyridylthio) and a halogenated thio group (e.g., pentafluorothio).

In the present specification, examples of the “optionally substituted silyl group” include a silyl group optionally having “1 to 3 substituents selected from a C₁₋₆ alkyl group, a C₂₋₆ alkenyl group, a C₃₋₁₀ cycloalkyl group, a C₆₋₁₄ aryl group and a C₇₋₁₆ aralkyl group, each of which optionally has 1 to 3 substituents selected from substituent group A”.

Examples of the optionally substituted silyl group include a tri-C₁₋₆ alkylsilyl group (e.g., trimethylsilyl, tert-butyl(dimethyl)silyl).

For descriptions of amino acid residues, the following conventions may be exemplified: Asp=D=Aspartic Acid; Ala=A=Alanine; Arg=R=Arginine; Asn=N=Asparagine; Cys=C=Cysteine; Gly=G=Glycine; Glu=E=Glutamic Acid; Gln=Q=Glutamine; His=H=Histidine; Ile=I=Isoleucine; Leu=L=Leucine; Lys=K=Lysine; Met=M=Methionine; Phe=F=Phenylalanine; Pro=P=Proline; Ser=S=Serine; Thr=T=Threonine; Trp=W=Tryptophan; Tyr-Y=Tyrosine; and Val=V=Valine.

Also for convenience, and readily known to one skilled in the art, the following abbreviations or symbols are used to represent the moieties, reagents and the like used in present disclosure:

Aib is alpha-aminoisobutyric acid;

mono-halo Phe—mono-halo phenylalanine;

bis-halo Phe—bis-halo phenylalanine;

mono-halo Tyr—mono-halo tyrosine;

bis-halo Tyr—bis-halo Tyrosine;

(D)-Tyr—D-tyrosine;

(D)-Ala—D-Alanine

DesNH₂-Tyr—desaminotyrosine;

(D)-Phe—D-phenylalanine;

DesNH₂-Phe—desaminophenylalanine;

(D)-Trp—D-tryptophan;

(D)₃Pya—D-3-pyridylalanine;

2-Cl-(D)Phe—D-2-chlorophenylalanine;

3-Cl-(D)Phe—D-3-chlorophenylalanine;

4-Cl-(D)Phe—D-4-chlorophenylalanine;

2-F-(D)Phe—D-2-fluorophenylalanine;

3-F(D)Phe—D-3-fluorophenylalanine;

3,5-DiF-(D)Phe—D-3,5-difluorophenylalanine;

3,4,5-TriF-(D)Phe—D-3,4,5-trifluorophenylalanine;

D-Iva—D-Isovaline

SSA—succinimidyl succinamide;

PEG—polyethylene glycol;

PEGₘ—(methoxy)polyethylene glycol;

PEGₘ(12,000)—(methoxy)polyethylene glycol having a molecular weight of about 12 kD;

PEGₘ(20,000)—(methoxy)polyethylene glycol having a molecular weight of about 20 kD;

PEGₘ(30,000)—(methoxy)polyethylene glycol having a molecular weight of about 30 kD;

Fmoc—9-fluorenylmethyloxycarbonyl;

DMF—dimethylformamide;

DIPEA—N,N-diisopropylethylamine;

TFA—trifluoroacetic acid;

HOBT—N-hydroxybenzotriazole;

BOP—benzotriazol-1-yloxy-tris-(dimethylamino)phosphoniumhexafluorophosphate;

HBTU—2-(1H-benzotriazole-1-yl)-1,1,3,3-tetramethyluroniumhexafluorophosphate;

NMP—N-methyl-pyrrolidone;

FAB-MS fast atom bombardment mass spectrometry;

ES-MS—electro spray mass spectrometry.

Abu: α-aminobutyric acid;

Acc: 1-amino-1-cyclo(C₃-C₉)alkyl carboxylic acid;

A3c: 1-amino-1-cyclopropane carboxylic acid;

A4c: 1-amino-1-cyclobutanecarboxylic acid;

A5c: 1-amino-1-cyclopentanecarboxylic acid;

A6c: 1-amino-1-cyclohexanecarboxylic acid;

Act: 4-amino-4-carboxytetrahydropyran;

Ado: 12-aminododecanoic acid;

Aib: alpha-aminoisobutyric acid;

Aic: 2-aminoindan-2-carboxylic acid;

β-Ala: beta-alanine;

Amp: 4-amino-phenylalanine;

Apc: 4-amino-4-carboxypiperidine;

hArg: homoarginine;

Aun: 11-aminoundecanoic acid;

Ava: 5-aminovaleric acid;

Cha: β-cyclohexylalanine;

Dhp: 3,4-dehydroproline;

Dmt: 5,5-dimethylthiazolidine-4-carboxylic acid;

Gaba: γ-aminobutyric acid;

4Hppa: 3-(4-hydroxyphenyl)propionic acid;

Hyp: -hydroxyproline

3Hyp: 3-hydroxyproline;

4Hyp: 4-hydroxyproline;

hPro: homoproline;

4Ktp: 4-ketoproline;

Nle: norleucine;

NMe-Tyr: N-methyl-tyrosine;

1Nal or 1-Nal: β-(1-naphthyl)alanine;

2Nal or 2-Nal: β-(2-naphthyl)alanine;

Nva: norvaline;

Orn: ornithine;

2Pal or 2-Pal: β-(2-pyridinyl)alanine;

3Pal or 3-Pal: β-(3-pyridinyl)alanine;

4Pal or 4-Pal: β-(4-pyridinyl)alanine;

Pen: penicillamine;

(3,4,5F)Phe: 3,4,5-trifluorophenylalanine;

(2,3,4,5,6)Phe: 2,3,4,5,6-pentafluorophenylalanine;

Psu: N-propylsuccinimide;

Iva: Isovaline;

Sar: Sarcosine;

Taz: β-(4-thiazolyl)alanine;

3Thi: β-(3-thienyl)alanine;

Thz: thioproline;

Tic: tetrahydroisoquinoline-3-carboxylic acid;

Tle: tert-leucine;

Act: acetonitrile;

Boc: tert-butyloxycarbonyl;

BSA: bovine serum albumin;

DCM: dichloromethane;

DTT: dithiothrieitol;

ESI: electrospray ionization;

Fmoc: 9-fluorenylmethyloxycarbonyl;

HBTU: 2-(1H-benzotriazole-1-yl)-1,1,3,3-tetramethyluronium hexafluorophosphate;

HPLC: high performance liquid chromatography;

IBMX: isobutylmethylxanthine;

LC-MS: liquid chromatography-mass spectrometry;

Mtt: methyltrityl;

NMP: N-methylpyrrolidone;

5K PEG: polyethylene glycol, which may include other functional groups or moieties such as a linker, and which is either linear or branched as defined herein below, with a weight average molecular weight of about 5,000 Daltons.

10K PEG: polyethylene glycol, which may include other functional groups or moieties such as a linker, and which is either linear or branched as defined herein below, with a weight average molecular weight of about 10,000 Daltons.

20K PEG: polyethylene glycol, which may include other functional groups or moieties such as a linker, and which is either linear or branched as defined herein below, with a weight average molecular weight of about 20,000 Daltons.

30K PEG: polyethylene glycol, which may include other functional groups or moieties such as a linker, and which is either linear or branched as defined herein below, with a weight average molecular weight of about 30,000 Daltons.

40K PEG: polyethylene glycol, which may include other functional groups or moieties such as a linker, and which is either linear or branched as defined herein below, with a weight average molecular weight of about 40,000 Daltons.

50K PEG: polyethylene glycol, which may include other functional groups or moieties such as a linker, and which is either linear or branched as defined herein below, with a weight average molecular weight of about 50,000 Daltons.

60K PEG: polyethylene glycol, which may include other functional groups or moieties such as a linker, and which is either linear or branched as defined herein below, with a weight average molecular weight of about 60,000 Daltons.

PEG is available in a variety of molecular weights based on the number of repeating subunits of ethylene oxide (i.e. —OCH₂CH₂—) within the molecule. mPEG formulations are usually followed by a number that corresponds to their average molecular weight. For example, PEG-200 has a weight average molecular weight of 200 Daltons and may have a molecular weight range of 190-210 Daltons. Molecular weight in the context of a water-soluble polymer, such as PEG, can be expressed as either a number average molecular weight or a weight average molecular weight. Unless otherwise indicated, all references to molecular weight of mPEG herein refer to the weight average molecular weight. Both molecular weight determinations, number average and weight average, can be measured using gel permeation chromatography or other liquid chromatography techniques. Other methods for measuring molecular weight values can also be used, such as the use of end-group analysis or the measurement of colligative properties (e.g., freezing-point depression, boiling-point elevation, or osmotic pressure) to determine number average molecular weight or the use of light scattering techniques, ultracentrifugation or viscometry to determine weight average molecular weight.

tBu: tert-butyl

TIS: triisopropylsilane

Trt: trityl

Z: benzyloxycarbonyl

As used herein, “PEG moiety” refers to polyethylene glycol (PEG) or a derivative thereof, for example (methoxy)polyethylene glycol (PEGₘ).

As used herein, “PEGylated peptide” refers to a peptide wherein at least one amino acid residue, for example, Lys, or Cys has been conjugated with a PEG moiety. By “conjugated”, it is meant that the PEG moiety is either directly linked to said residue or is linked to the residue via a spacer moiety, for example a cross-linking agent. When said conjugation is at a lysine residue, that lysine residue is referred to herein as “PEGylated Lys”. A peptide that is conjugated to only one MPEG moiety is said to be “mono-PEGylated”.

As used herein, “Lys-PEG” and “Lys-PEGₘ” refer respectively to lysine residues which have been conjugated with PEG. “Lys(epsilon-SSA-PEGₘ)” refers to a lysine residue wherein the epsilon-amino group has been cross-linked with MPEG using a suitably functionalized SSA.

In the present specification, the term “human native GIP peptide” refers to the naturally occurring human GIP peptide. This human native GIP peptide (42 amino acids) has an amino acid sequence: YAEGTFISDYSIAMDKIHQQDFVNWLLAQKGKKNDWKHNITQ (SEQ ID NO: 1) and is the functionally active molecule derived from the parent precursor described in National Center for Biotechnology Information (NCBI) Reference Sequence: NP_004114.1; REFSEQ: accession NM_004123.2 This full length precursor is encoded from the mRNA sequence of human gastric inhibitory polypeptide (GIP), mRNA; ACCESSION: NM_004123; VERSION; NM_004123.2.

“Percent (%) amino acid sequence identity” with respect to a reference polypeptide sequence is defined as the percentage of amino acid residues in a candidate polypeptide sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, “treatment” (and variations such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of a condition, alleviation of symptoms, diminishment of any direct or indirect pathological consequences of the condition or treatment, preventing emesis, i.e., by preventing the occurrence of symptoms in whole or in part associated with a condition or side-effects known to accompany a specific treatment, decreasing the rate of progression, amelioration or palliation of the symptoms associated with emesis, such as nausea and/or vomiting, and remission or improved prognosis. In some embodiments, GIP receptor agonist peptides of the disclosure are used to inhibit or delay development of emesis, i.e. nausea or vomiting or to slow the progression of emesis or the symptoms associated with emesis, or to prevent, delay or inhibit the development of emesis, nausea and/or vomiting related to the treatment of a different disease being actively treated.

By “reduce” or “inhibit” is meant the ability to cause an overall decrease of 20%, 30%, 40%, 50%, 60%, 70%, 75%, 80%, 85%, 90%, 95%, or greater. In some embodiments, reduce or inhibit can refer to a relative reduction compared to a reference (e.g., reference level of biological activity (e.g., the number of episodes of nausea and/or vomiting after administration to a subject of a prescribed amount of chemotherapy, for example, a prescribed dose of a chemotherapeutic agent that is known to cause emesis). In some embodiments, reduce or inhibit can refer to the relative reduction of a side effect (i.e. nausea and/or vomiting) associated with a treatment for a condition or disease.

Optimal alignment of sequences for comparison can be conducted, for example, by the local homology algorithm of Smith and Waterman (Adv. Appl. Math. 2:482 (1981), which is incorporated by reference herein), by the homology alignment algorithm of Needleman and Wunsch (J. Mol. Biol. 48:443-53 (1970), which is incorporated by reference herein), by the search for similarity method of Pearson and Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, 4th ed., John Wiley and Sons, New York (1999)).

One illustrative example of an algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al., Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information internet web site. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff and Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, an amino acid sequence is considered similar to a reference amino acid sequence if the smallest sum probability in a comparison of the test amino acid to the reference amino acid is less than about 0.1, more typically less than about 0.01, and most typically less than about 0.001.

Variants can also be synthetic, recombinant, or chemically modified polynucleotides or polypeptides isolated or generated using methods well known in the art. Variants can include conservative or non-conservative amino acid changes, as described below. Polynucleotide changes can result in amino acid substitutions, additions, deletions, fusions and truncations in the polypeptide encoded by the reference sequence. Variants can also include insertions, deletions or substitutions of amino acids, including insertions and substitutions of amino acids and other molecules) that do not normally occur in the peptide sequence that is the basis of the variant, for example but not limited to insertion of ornithine which do not normally occur in human proteins. The term “conservative substitution,” when describing a polypeptide, refers to a change in the amino acid composition of the polypeptide that does not substantially alter the polypeptide's activity. For example, a conservative substitution refers to substituting an amino acid residue for a different amino acid residue that has similar chemical properties. Conservative amino acid substitutions include replacement of a leucine with an isoleucine or valine, an aspartate with a glutamate, or a threonine with a serine.

“Conservative amino acid substitutions” as referenced herein result from replacing one amino acid with another having similar structural and/or chemical properties, such as the replacement of a leucine with an isoleucine or valine, an aspartate with a glutamate, or a threonine with a serine. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the peptide, (i.e. the ability of the peptide to penetrate the blood brain barrier (BBB)). Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). (See also Creighton, Proteins, W. H. Freeman and Company (1984), incorporated by reference in its entirety.) In some embodiments, individual substitutions, deletions or additions that alter, add or delete a single amino acid or a small percentage of amino acids can also be considered “conservative substitutions” if the change does not reduce the activity of the peptide. Insertions or deletions are typically in the range of about 1 to 5 amino acids. The choice of conservative amino acids may be selected based on the location of the amino acid to be substituted in the peptide, for example if the amino acid is on the exterior of the peptide and expose to solvents, or on the interior and not exposed to solvents.

In alternative embodiments, one can also select conservative amino acid substitutions encompassed suitable for amino acids on the interior of a protein or peptide, for example one can use suitable conservative substitutions for amino acids is on the interior of a protein or peptide (i.e., the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants.

As used herein, the term “selectivity” of a molecule for a first receptor relative to a second receptor refers to the following ratio: EC₅₀ of the molecule at the second receptor divided by the EC₅₀ of the molecule at the first receptor. For example, a molecule that has an EC₅₀ of 1 nM at a first receptor and an EC₅₀ of 100 nM at a second receptor has 100-fold selectivity for the first receptor relative to the second receptor.

As is understood by one skilled in the art, reference to “about” a value or parameter herein includes (and describes) embodiments that are directed to that value or parameter per se or that have a variance plus or minus of that value ranging from less than 10%, or less than 9%, or less than 8%, or less 7%, or less than 6%, or less than 5%, or less than 4%, or less than 3%, or less than 2%, or less than 1%, or less than 0.1% than the stated value. For example, description referring to “about X” includes description of “X”.

It is understood that aspect and embodiments of the disclosure described herein include “consisting” and/or “consisting essentially” of aspects and embodiments. As used herein, the singular form “a”, “an”, and “the” includes plural references unless indicated otherwise.

### A. GIP Receptor Agonist Peptides

In various embodiments of the present disclosure, GIP receptor agonist peptides are provided. In addition, methods are provided for the prevention and/or treatment of diabetes mellitus (e.g., type-2 diabetes mellitus) obesity, a metabolic syndrome and emesis in a subject in need thereof. In various embodiments, the methods provide administration of a therapeutically effective amount of a GIP receptor agonist peptide once per week or QW (for example, Q1W, used interchangeably herein) to the subject.

As used herein, GIPr agonist peptides of the present disclosure refer to peptides that preferentially bind to GIP receptors compared to other receptors, such as GLP receptors. In some embodiments, an exemplary GIPr agonist peptide of the present disclosure are GIPr agonist peptides that have a selectivity ratio as defined as the ratio of (EC₅₀ GLP1R/EC₅₀ GIPR) greater than 10, or greater than 100, or greater than 1,000, or greater than 10,000, or greater than 100,000. An exemplary GIP receptor agonist peptide is a GIPr agonist peptide when the peptide has a selectivity ratio of (EC₅₀ GLP1R/EC₅₀ GIPR) of greater than 10, or 100, or 1,000, or 10,000, or from about 100-1,000,000 or more.

As used herein, “Lys(R)” is synonymous with “Km” and are used interchangeably.

In some embodiments, a GIP receptor agonist peptide, or a salt thereof is provided. The GIP receptor agonist peptide is represented by formula (I):

P¹-Tyr-A2-Glu-Gly-Thr-Phe-Ile-Ser-A9-Tyr-Ser-Ile-A13-A14-Asp-A16-A17-A18-Gln-A20-A21-Phe-Val-A24-Trp-Leu-Leu-Ala-Gln-A30-A31-A32-A33-A34-A35-A36-A37-A38-A39-A40-A41-A42-P², or a pharmaceutically acceptable salt thereof;
wherein
P¹ represents a group represented by formula

### —R^{A1},

### —CO—R^{A1},

### —CO—OR^{A1},

### —CO—COR^{A1},

### —SO—R^{A1},

—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or
is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, D-Ala, Ala, Gly, or Pro;
A9: represents Asp or Leu;
A13: represents Aib, or Ala;
A14: represents Leu, Aib, Ile, or Nle;
A16: represents Arg, Ser, or Lys;
A17: represents Aib, Ala, or Ile;
A18: represents Ala, His, or Lys;
A19: represents Gln, or Ala;
A20: represents Aib, Gln, or Ala;
A21: represents Asp, Asn, or Lys;
A24: represents Asn, Gln, or Glu;
A30: represents Arg, Ser, Gln, or Lys;
A31: represents Gly, Pro, or a deletion;
A32: represents Ser, Lys, Pro, Gly, or a deletion;
A33: represents Ser, Lys, Gly, or a deletion;
A34: represents Gly, Asn, or a deletion;
A35: represents Ala, Asp, Ser, Asn, or a deletion;
A36: represents Pro, Trp, or a deletion;
A37: represents Pro, Lys, or a deletion;
A38: represents Pro, His, or a deletion;
A39: represents Ser, Asn, or a deletion;
A40: represents Ile, or a deletion;
A41: represents Thr, or a deletion; and
A42: represents Gln, or a deletion.

In related embodiments, the GIP receptor agonist peptide according to Formula (I) has an amino acid sequence of Formula (I), wherein A31 is Gly, A32-A42 are deletion, or A32 is Gly and 33-A42 are deletion.

In various embodiments, the GIP receptor agonist peptide of Formula (I) comprises a peptide wherein P² is —OH.

On other embodiments, the GIP receptor agonist peptide of Formula (I) comprises a peptide wherein P¹ is methyl, (Me).

In various embodiments, the GIP receptor agonist peptide of Formula (I) comprises a peptide wherein P¹ is methyl, (Me), and P² is —OH.

In some embodiments, a GIP receptor agonist peptide, or a salt thereof is provided. The GIP receptor agonist peptide is represented by formula (II):

P¹-Tyr-A2-Glu-Gly-Thr-Phe-Ile-Ser-Asp-Tyr-Ser-Ile-A13-A14-Asp-A16-A17-A18-A19-A20-A21-Phe-Val-A24-Trp-Leu-Leu-Ala-A29-A30-A31-A32-A33-A34-A35-A36-A37-A38-A39-A40-A41-A42-P², or a pharmaceutically acceptable salt thereof, wherein:
P¹ represents a group represented by formula

### —R^{A1},

### —CO—R^{A1},

### —CO—OR^{A1},

### —CO—COR^{A1},

### —SO—R^{A1},

—SO₂—R^{A1},
—SO₂—OR^{A1},
—CO—NR^{A2}R^{A3},
—SO₂—NR^{A2}R^{A3}, or
—C(═NR^{A1})—NR^{A2}R^{A3}
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, D-Ala, or Gly;
A13: represents Aib, or Ala;
A14: represents Leu, Aib, Ile, Nle, or Lys(R);
A16: represents Arg, Ser, or Lys;
A17: represents Aib, Ala, Ile, or Lys(R);
A19: represents Gln or Ala;
A18: represents Ala, His, or Lys(R);
A20: represents Aib, Gln, Arg, or Ala;
A21: represents Asp, Asn, or Lys(R);
A24: represents Asn, Gln, or Glu;
A29: represents Gln, or Lys(R)
A30: represents Arg, Lys, Ser, Gln, or Lys(R);
A31: represents Gly, Pro, or a deletion;
A32: represents Ser, Lys, Pro, Gly, or a deletion;
A33: represents Ser, Lys, Gly, or a deletion;
A34: represents Gly, Asn, or a deletion;
A35: represents Ala, Asp, Ser, Asn, or a deletion;
A36: represents Pro, Trp, or a deletion;
A37: represents Pro, Lys, or a deletion;
A38: represents Pro, His, or a deletion;
A39: represents Ser, Asn, or a deletion;
A40: represents Ile, or a deletion;
A41: represents Thr, or a deletion;
A42: represents Gln, or a deletion.
wherein the residue Lys(R), the (R) portion represents X-L-, wherein L represents a linker, and is selected from the following group consisting of gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE; and X represents a lipid.

In some embodiments, a GIP receptor agonist peptide, or a pharmaceutically acceptable salt thereof is provided. The GIP receptor agonist peptide is represented by formula (III):

P¹-Tyr-A2-Glu-Gly-Thr-Phe-Ile-Ser-Asp-Tyr-Ser-Ile-A13-A14-Asp-A16-A17-A18-A19-A20-A21-Phe-Val-A24-Trp-Leu-Leu-Ala-A29-A30-A31-A32-A33-A34-A35-A36-A37-A38-A39-P², or a pharmaceutically acceptable salt thereof, wherein:
P¹ represents a group represented by formula

### —R^{A1},

### —CO—R^{A1},

### —CO—OR^{A1},

### —CO—COR^{A1},

### —SO—R^{A1},

—SO₂—R^{A1},
—SO₂—OR^{A1},
—CO—NR^{A2}R^{A3},
—SO₂—NR^{A2}R^{A3}, or
—C(═NR^{A1})—NR^{A2}R^{A3}
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, D-Ala, or Gly;
A13: represents Aib, or Ala;
A14: represents Leu, Aib, or Lys(R);
A16: represents Arg, Ser, or Lys;
A17: represents Aib, Ile, Ala, or Lys(R);
A18: represents Ala, His, or Lys(R);
A19: represents Gln or Ala;
A20: represents Aib, Gln, Arg, or Ala;
A21: represents Asp, Asn, or Lys(R);
A24: represents Asn, or Glu;
A29: represents Gln, or Lys(R)
A30: represents Arg, Lys, Ser, Gln, or Lys(R);
A31: represents Gly, Pro, or a deletion;
A32: represents Ser, Gly, Lys, or a deletion;
A33: represents Ser, Lys, Gly, or a deletion;
A34: represents Gly, Asn, or a deletion;
A35: represents Ala, Asp, Ser, Asn, or a deletion;
A36: represents Pro, Trp, or a deletion;
A37: represents Pro, Lys, or a deletion;
A38: represents Pro, His, or a deletion;
A39: represents Ser, Asn, or a deletion;
wherein the residue Lys(R), the (R) portion represents X-L-, wherein L represents a linker, and is selected from the following group consisting of gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE; and X represents a lipid.

In some embodiments, a GIP receptor agonist peptide, or a salt thereof is provided. The GIP receptor agonist peptide is represented by formula (IV):

P¹-Tyr-Aib-Glu-Gly-Thr-Phe-Ile-Ser-Asp-Tyr-Ser-Ile-A13-A14-Asp-A16-A17-A18-A19-A20-A21-Phe-Val-A24-Trp-Leu-Leu-Ala-A29-A30-A31-A32-A33-A34-A35-A36-A37-A38-A39-P², or a pharmaceutically acceptable salt thereof, wherein:
P¹ represents H or C₁₋₆ alkyl;
P² represents —NH₂ or —OH;
A13: represents Aib, Ala, or Lys;
A14: represents Leu, Aib, Lys, or Lys(R);
A16: represents Arg, Ser, or Lys;
A17: represents Aib, Ala, Ile, Glu, Lys, or Lys(R);
A18: represents Ala, His, Glu, Lys, or Lys(R);
A19: represents Gln or Ala;
A20: represents Aib, Ala, Gln, Arg, or Lys;
A21: represents Asp, Asn, Lys, or Lys(R);
A24: represents Asn or Glu;
A29: represents Gln, Lys, or Lys(R);
A30: represents Arg, Ser, Gln, Lys, Lys(Ac), or Lys(R);
A31: represents Gly, Pro, or a deletion;
A32: represents Ser, Gly, or a deletion;
A33: represents Ser, Gly, or a deletion;
A34: represents Gly or a deletion;
A35: represents Ala, Ser, or a deletion;
A36: represents Pro or a deletion;
A37: represents Pro or a deletion;
A38: represents Pro or a deletion; and
A39: represents Ser or a deletion;
wherein in the residue Lys(R), the (R) portion represents X-L-, wherein L represents a linker and is selected from the group consisting of 2OEGgE, 2OEGgEgE, G4gE, GGGGG, G5gE, G5gEgE, G6, gEgEgE, OEGgEgE, OEGgEOEGgE, GGPAPAP, and GGPAPAPgE; and X represents C₁₇-C₂₂ monoacid or C₁₇-C₂₂ diacid.

In some embodiments, A13 represents Aib or Ala.

In some embodiments, A16 represents Arg or Lys.

In some embodiments, A19 represents Gln.

In some embodiments, A20 represents Aib.

In some embodiments, A31 represents Gly or Pro, and A32-A30 are deletion.

In some embodiments of formula (IV), A14 represents Leu, Aib, or Lys(R).

In some embodiments of formula (IV), A14 represents Leu or Lys(R).

In some embodiments of formula (IV), A17 represents Aib, Ala, Ile, Glu, or Lys(R).

In some embodiments of formula (IV), A17 represents Aib or Lys(R).

In some embodiments of formula (IV), A18 represents Ala, His, Glu, or Lys(R).

In some embodiments of formula (IV), A18 represents Ala or Lys(R).

In some embodiments of formula (IV), A21 represents Asp, Asn, or Lys(R).

In some embodiments of formula (IV), A29 represents Gln or Lys(R).

In some embodiments of formula (IV), A30 represents Arg, Ser, Gln, Lys, Lys(Ac), or Lys(R).

In some embodiments of formula (IV), A30 represents Arg, Ser, Gln, Lys(Ac), or Lys(R).

In some embodiments of formula (IV),

A14: represents Leu, Aib, Lys, or Lys(R);
A17: represents Aib, Ala, Ile, Glu, or Lys(R);
A18: represents Ala, His, Glu, or Lys(R);
A21: represents Asp, Asn, or Lys(R);
A29: represents Gln or Lys(R); and
A30: represents Arg, Ser, Gln, Lys, Lys(Ac), or Lys(R).

In some embodiments of formula (IV),

A13: represents Aib or Ala;
A14: represents Leu, Lys, or Lys(R);
A16: represents Arg;
A17: represents Aib, Lys, or Lys(R);
A18: represents Ala, Lys, or Lys(R);
A20: represents Aib;
A29: represents Gln;
A30: represents Arg, Ser, or Lys;
A31: represents Gly or Pro;
A33: represents Ser or a deletion; and
A35: represents Ala or a deletion;
wherein L is selected from the group consisting of 2OEGgE, 2OEGgEgE, OEGgEgE, OEGgEOEGgE, G5, GGPAPAP, and GGPAPAPgE; and X represents C₁₇-C₂₂ monoacid or C₁₇-C₂₂ diacid.

In some embodiments of formula (IV),

A13: represents Aib or Ala;
A14: represents Leu or Lys(R);
A16: represents Arg;
A17: represents Aib or Lys(R);
A18: represents Ala or Lys(R);
A20: represents Aib;
A21: represents Asp, Asn, or Lys(R).
A29: represents Gln;
A30: represents Arg, Ser, or Lys;
A31: represents Gly or Pro;
A33: represents Ser or a deletion; and
A35: represents Ala or a deletion,

wherein L is selected from the group consisting of 2OEGgE, 2OEGgEgE, OEGgEgE, OEGgEOEGgE, G5, GGPAPAP, and GGPAPAPgE; and X represents C₁₇-C₂₂ monoacid or C₁₇-C₂₂ diacid. In some embodiments, the GIP receptor agonist peptide comprises a peptide wherein P² is —OH. In some embodiments, the GIP receptor agonist peptide comprises a peptide wherein P² is —NH₂.

In some embodiments, the GIP receptor agonist peptide comprises a peptide wherein P¹ is a C₁₋₆ alkyl group. In some embodiments, the GIP receptor agonist peptide comprises a peptide wherein P¹ is methyl, (Me).

In some embodiments, the GIP receptor agonist peptide comprises a peptide wherein P¹ is Me and P² is —OH.

In some embodiments, the GIP receptor agonist peptide comprises a peptide wherein L is 2OEGgEgE or GGGGG.

In some embodiments, the GIP receptor agonist peptide comprises a peptide wherein X is C₁₈ diacid.

In some embodiments, the GIP receptor agonist peptide is represented by formula (V):

Me-Tyr-Aib-Glu-Gly-Thr-Phe-Ile-Ser-Asp-Tyr-Ser-Ile-A13-A14-Asp-Arg-A17-Ala-Gln-Aib-A21-Phe-Val-Asn-Trp-Leu-Leu-Ala-Gln-A30-A31-A32-A33-A34-A35-A36-A37-A38-A39-P², or a pharmaceutically acceptable salt thereof, wherein
P² represents —NH₂ or —OH;
A13: represents Aib or Ala;
A14: represents Leu, Lys, or Lys(R);
A17: represents Aib, Lys, or Lys(R);
A21: represents Asp, Asn, Lys, or Lys(R);
A30: represents Arg, Ser, Lys, or Lys(R);
A31: represents Gly or Pro;
A32: represents Ser, Gly, or a deletion;
A33: represents Ser or a deletion;
A34: represents Gly or a deletion;
A35: represents Ala or a deletion;
A36: represents Pro or a deletion;
A37: represents Pro or a deletion;
A38: represents Pro or a deletion; and
A39: represents Ser or a deletion,
wherein L is 2OEGgEgE or GGGGG; and X represents C₁₈ diacid.

In some embodiments of formula (V), A14 represents Leu or Lys(R).

In some embodiments of formula (V), A17 represents Aib or Lys(R).

In some embodiments of formula (V), A21 represents Asp, Asn, or Lys(R).

In some embodiments of formula (V), A30 represents Arg, Ser, Lys, or Lys(R).

In some embodiments of formula (V),

A14: represents Leu or Lys(R);
A17: represents Aib or Lys(R);
A21: represents Asp, Asn, or Lys(R); and
A30: represents Arg, Ser, Lys, or Lys(R).

In various embodiments, an illustrative GIP receptor agonist peptide for use in the methods, compositions and medicaments exemplified herein, has at least 80%, or at least 85%, or at least 90%, or at least 95%, or at least 96%, or at least 97%, or at least 98%, or at least 99%, or 100% sequence identity to any GIP receptor agonist peptide as defined by formulas (I), (II), (III), (IV), or (V).

In various embodiments, an illustrative GIP receptor agonist peptide for use in the methods, compositions and medicaments exemplified herein, has 100% sequence identity to any GIP receptor agonist peptide as defined by formulas (I), (II), (III), (IV), or (V).

In various embodiments, the GIP receptor agonist peptide as defined by formulas (I), (II), (III), (IV), or (V), has a P² defined by a hydroxyl (—OH) group. In various embodiments, the GIP receptor agonist peptide as defined by formulas (I), (II), (III), (IV), or (V), has a P² defined by an amino (—NH₂) group. In various embodiments, the GIP receptor agonist peptide as defined by formulas (I), (II), (III), (IV), or (V), has a P¹ defined by a methyl (Me) group.

With reference to the above GIP receptor agonist peptides as defined by formulas (I), (II), (III), (IV), or (V), in various embodiments, a GIP receptor agonist peptide has at least one amino acid having a bivalent substituent, covalently coupled to a side chain of an amino acid. For example, in some embodiments, a GIP receptor agonist peptide has an amino acid sequence having a side chain of at least one amino acid, or modified amino acid for example, a Lys residue of the GIP receptor agonist peptide being covalently attached to a substituent group (R). In various embodiments, a Lys residue of the GIP receptor agonist peptide may be covalently attached to a substituent (R) as shown in the present disclosure as Lys(R).

For example, a selective GIP receptor agonist peptide of the present disclosure may have a Lys residue substituted by an (R) group at an amino acid position A14-A30, for example, at amino acid position: A14, or A17, A18, A21, A29, or A30. In various embodiments, the (R) group represents X-L-, wherein L represents a bivalent linker. In some embodiments, the bivalent linker can include a PEG, Abu-, (Gly)(2-8)-, gGlu(1-3)-, gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE, one to ten amino acids, for example, a glycine linker having two to ten glycine residues, two to six or from five to six glycines linked, or combinations of the foregoing linkers. In various embodiments, OEG=PEG3 and 2OEG=(PEG3)2. In these embodiments, X represents a substituent group, for example, a lipid. In various embodiments, X represents a monoacid or diacid lipid having C₁₇ to C₂₂ carbons in length, for example, a C₁₇, a C₁₈, a C₂₀ monoacid or diacid lipid. In some embodiments, X is a C₁₈ diacid.

In various embodiments, the GIP receptor agonist peptide may include one or two Lys residues is substituted with an X-L- substituent. In some embodiments, a Lys residue is substituted with an X-L- substituent, wherein L represents (PEG3)2-, Abu-, (Gly)(2-8)-, gGlu(1-3)-, or combinations thereof, for example, (PEG3)2-gGlu-, Abu-gGlu-, (Gly)₅-gGlu-, or (Gly)₆-gGlu-, GGGGG-, (PEG3)2-, PEG3)2-(Gly)5-6-, gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG, G5gEgE, or combinations thereof.

In some embodiments, the GIP receptor agonist peptide has one, or two Lys residues having a substituted side chain. For example, a selective GIPr agonist peptide may have a Lys residue substituted by X-L-, wherein L represents a bivalent linker, as discussed herein, for example, L may represent a bond or a bivalent substituent group, and wherein X represents an optionally substituted hydrocarbon group, for example a monoacid or diacid lipid, or a salt thereof. In some embodiments, the bivalent substituent group comprises: an alkylene group, a carbonyl group, an oxycarbonyl group, an imino group, an alkylimino group, a sulfonyl group, an oxy group, a sulfide group, an ester bond, an amide bond, a carbonate bond or combinations thereof.

In various embodiments, the GIP receptor agonist peptide may include one, or two Lys residues which may be substituted with an (R) group defined as an X-L- substituent. In some embodiments, Lys(R) is a Lys residue having a side chain substituted with X-L-. In related embodiments, the GIP receptor agonist peptide, the X moiety can be an optionally substituted hydrocarbon. In some embodiments, the X moiety in the X-L- substituent can include a C₁₇-C₂₂ monoacid, a C₁₇-C₂₂ diacid, an acetyl group, or combinations thereof. Some exemplary X moieties may include: (Hepda:C17 diacid), (Oda:C18 diacid), or (Eda:C20 diacid).

In various embodiments, a GIP receptor agonist peptide of formulas (I) to (V), the L moiety of the X-L- group can include, a bivalent linker. In some examples, the bivalent linker can include PEG, Abu-, (Gly)₍₂₋₈₎-, gGlu₍₁₋₃₎-, one to ten amino acids, or combinations thereof. In these examples of X-L, X may represents a substituent group.

In some embodiments, (R) represents X-L- wherein L represents (PEG3)2-, Abu-, (Gly)₍₂₋₈₎-, gGlu₍₁₋₃₎-, or combinations thereof. In some embodiments, L represents (PEG3)2-gGlu-, Abu-gGlu-, (Gly)₅-gGlu, (Gly)₆-gGlu-, GGGGG-, GGGGGG-, (PEG3)2-, or (PEG3)2-(Gly)₅₋₆-, gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE, or combinations thereof.

In some related embodiments, L represents a bond or a bivalent substituent group, and X represents an optionally substituted hydrocarbon group, or a salt thereof. For example, an illustrative GIP receptor agonist peptide has a Lys(R) residue, wherein the (R) portion of the Lys(R) residue is represented as X-L-, wherein X is a bivalent substituent group comprising an alkylene group, a carbonyl group, an oxycarbonyl group, an imino group, an alkylimino group, a sulfonyl group, an oxy group, a sulfide group, an ester bond, an amide bond, a carbonate bond or combinations thereof.

In some embodiments, an illustrative Lys(R) can include an (R) group defined as X-L- group, wherein the bivalent substituent X is a C₁₇-C₂₂ monoacid, a C₁₇-C₂₂ diacid or an acetyl group. Some exemplary X moieties may include: (Hepda:C17 diacid), (Oda:C18 diacid), (Eda:C20 diacid).

In some embodiments, an illustrative GIP receptor agonist peptide of formulas (I), (II), (III), (IV), or (V), can include a peptide having one, to two Lys(R) lipidated amino acids positioned in the amino acid sequence of the peptide ranging from residue A14 to A30, wherein the Lys(R) residue has a substituted side chain defined by X-L-. In exemplary embodiments, the X-L- group of the Lys(R) residue in the illustrative GIP receptor agonist peptide of formulas (I), (II), (III), (IV), or (V), may include: -(g-Glu)₂-Oda, -(g-Glu)₂-Eda, -(g-Glu)₂-Heda, -(PEG3)2-gGlu-Eda, -(PEG3)2-gGlu-Heda, -(PEG3)2-gGlu-Oda, -(PEG3)2-gGlu-Ida, -(PEG3)-gGlu-Eda, -(PEG3)-gGlu-Heda, -(PEG3)-gGlu-Oda, -Abu-gGlu-Oda, -(Gly)₅-gGlu-Eda, -(Gly)₅-gGlu-Heda, -(Gly)₅-gGlu-Oda, -(Gly)₅-Heda, -(Gly)₅-Oda, -(Gly)₅-Eda, -(PEG3)2-Heda, -(PEG3)2-Eda, -(PEG3)2-Oda, 2OEGgEgE-Hepda:C17 diacid, OEGgEgE-Hepda:C17 diacid, 2OEGgE-Hepda:C17 diacid, 3OEGgEgE-Hepda:C17 diacid, G5gEgE-Hepda:C17 diacid, 2OEGgEgEgE-Hepda:C17 diacid, 2OEG-Hepda:C17 diacid, G5gEgE-Hepda:C17 diacid, 2OEGgEgE-Oda:C18 diacid, OEGgEgE-Oda:C18 diacid, 2OEGgE-Oda:C18 diacid, 3OEGgEgE-Oda:C18 diacid, G5gEgE-Oda:C18 diacid, 2OEGgEgEgE-Oda:C18 diacid, 2OEG-Oda:C18 diacid, G5gEgE-Oda:C18 diacid, 2OEGgEgE-Eda:C20 diacid, OEGgEgE-Eda:C20 diacid, 2OEGgE-Eda:C20 diacid, 3OEGgEgE-Eda:C20 diacid, G5gEgE-Eda:C20 diacid, 2OEGgEgEgE-Eda:C20 diacid, 2OEG-Eda:C20 diacid, G5gEgE-Eda:C20 diacid, or combinations thereof.

In some illustrative examples, the (R) group may be covalently linked to a side chain of a Lys amino acid. In some examples, an exemplary (R) group represents X-L-, wherein L represents a bivalent linker comprising PEG and/or two or more amino acids, and X represents a substituent group, or a salt thereof. In various embodiments, the GIP receptor agonist peptide of formulas (I)-(V) or a salt thereof, has one or two Lys(R), residues located at a position between A14 to A30, wherein (R) represents a substituent group.

In some examples, R represents X-L-, wherein L is one or a combination of more than one selected from gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE, and X represents C₁₇-C₂₀ monoacid or diacid lipid, or an acetyl group. In some embodiments, X-L-, wherein L is one or a combination of more than one selected from gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE, and X represents C18 diacid.

Alternatively, in some embodiments, (R) represents X-L-, wherein L represents a linker selected from gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE, and X represents C₁₇-C₂₀ linear saturated dicarboxylic acid.

In various embodiments, in each of the examples of GIP receptor agonist peptides of formulas (I) to (V), at least one amino acid between A14 to A30, or from A14 to A21, or A14 or A21 is Lys(R), wherein (R) represents X-L-, wherein L represents a bivalent linker L, wherein L represents 2OEGgEgE, OEGgEgE, 2OEGgE, 3OEGgEgE, G5gEgE, 2OEGgEgEgE, 2OEG, or G5gEgE. In some related embodiments, (R) represents X-L-, wherein L represents a bond or a bivalent substituent group, and X represents an optionally substituted hydrocarbon group, or a salt thereof. In various embodiments related to the various L moiety exemplifications, (R) represents X-L, wherein L is discussed above and X is a C₁₇-C₂₂ monoacid, or a C₁₇-C₂₂ diacid or an acetyl group. For example, in some embodiments, X is (Hepda:C17 diacid), (Oda:C18 diacid), or (Eda:C20 diacid). In various embodiments, an exemplary GIP receptor agonist peptide of formulas (I) to (V), comprises a peptide having at least one Lys amino acid positioned between A14 to A30, or from A14 to A21, for example, at an amino acid position A14, or A17, A18, A20, A21, AA26, A29, or A30 of the peptide. The (R) substituent portion of the Lys(R) residue, represents X-L-, wherein L represents a bivalent linker L, for example, L represents gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE and X is a C₁₇-C₂₂ monoacid, or a C₁₇-C₂₂ diacid or an acetyl group, for example, a C₁₇ monoacid or a C₁₇ diacid or a C₁₈ monoacid or a C₁₈ diacid or a C₂₀ monoacid or a C₂₀ diacid. In various embodiments, an exemplary GIP receptor agonist peptide of formulas (I) to (V), comprises at least one Lys amino acid positioned between A14 to A30, or from A14 to A21, or A14 or A21, wherein (R) represents X-L-, wherein L represents a bivalent linker L, wherein L represents 2× γGlu-2×OEG (miniPEG), and X is a C₁₈ monoacid or C₁₈ diacid.

In some embodiments, (R) represents X-L-, wherein L represents a bivalent linker comprising PEG and/or amino acid or consisting of PEG and/or one or more amino acids, for example, a Gly₂₋₁₀-linker, and X represents a substituent group. A known PEG linker, an amino acid linker or combinations thereof may be used as illustrative examples of a bivalent linker, as long as it is able to link Lys to a substituent group. Alternatively, in some embodiments, R represents X-L-, wherein L represents a bond or a bivalent substituent group, and X represents an optionally substituted hydrocarbon group, or a salt thereof. A known bivalent substituent group may include, but is not limited to, an alkylene group, a carbonyl group, an oxycarbonyl group, an imino group, an alkylimino group, a sulfonyl group, an oxy group, a sulfide group, an ester bond, an amide bond, a carbonate bond or combinations thereof may be used.

In some embodiments, L represents (PEG3)2-, Abu-, (Gly)₍₂₋₁₀₎-, gGlu₍₁₋₃₎-, or combinations thereof. In some embodiments, L represents (PEG3)2-gGlu-. In some examples, L represents Abu-gGlu-. In other examples, L represents (Gly)₅-gGlu-, or (Gly)₆-gGlu-. In some embodiments, L represents a glycine peptide having from about two to about ten glycines linked, or from about two to about seven glycines linked. In some examples, L represents (Gly)₅₋₆-, or (Gly)₅-, GGGGG-, or GGGGG-gGlu-. In some examples, L represents gE, GGGGG, GGEEE, G2E3, G3gEgE, 2OEGgEgE, OEGgEgE, GGPAPAP, 2OEGgE, 3OEGgEgE, G4gE, G5gE, 2OEGgEgEgE, 2OEG and G5gEgE.

In some embodiments, L represents (PEG3)2-. In some embodiments, L represents (Gly)₂₋₁₀-, for example, (Gly)(5.6). In some further embodiments, L represents a combination of groups, such as one or more PEG molecules linked to a glycine peptide: Gly₂₋₁₀ for example, L may be (PEG3)2-(Gly)₅₋₆-, or (PEG3)2-(Gly)₅-.

In some embodiments, the (R) group attached to an amino acid, for example, a Lys residue represents X-L-, wherein L represents a bivalent linker comprising PEG and/or one or more amino acids or consisting of PEG and/or one or more amino acids, and X represents a substituent group. A known PEG linker, an amino acid linker or combinations thereof may be used as the bivalent linker as long as it is able to link, a Lys residue to a substituent group. Alternatively, R represents X-L-, wherein L represents a bond or a bivalent substituent group, and X represents an optionally substituted hydrocarbon group, or a salt thereof. A known bivalent substituent group including, but are not limited to, an alkylene group, a carbonyl group, an oxycarbonyl group, an imino group, an alkylimino group, a sulfonyl group, an oxy group, a sulfide group, an ester bond, an amide bond, a carbonate bond or combinations thereof may be used. In some examples, (R) represents X-L-, wherein L is one or a combination of more than one selected from:

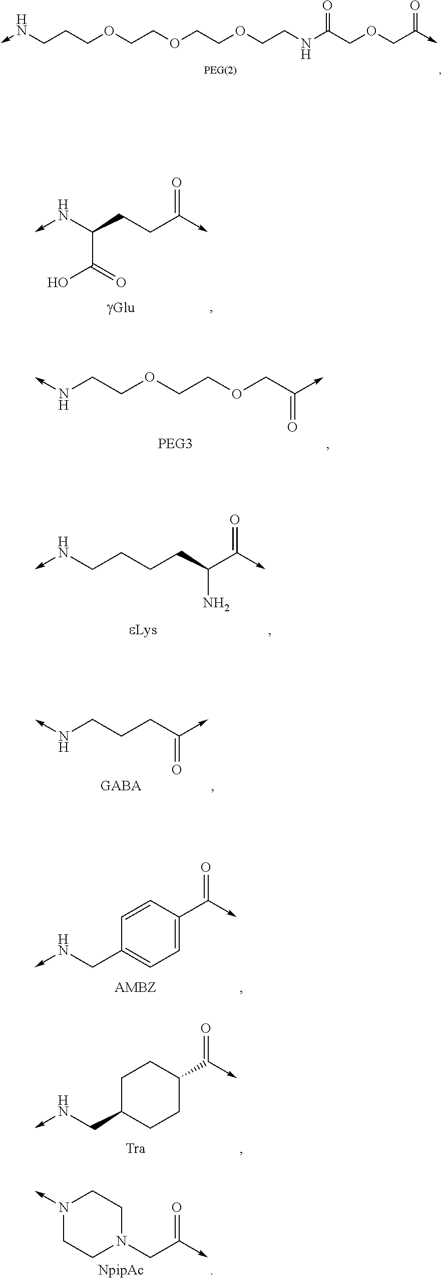

a glycine linker comprising one or two to nine-linked glycine(s) or a single bond, and X represents C₁₇-C₂₂ monoacid or diacid, or an acetyl group. In some embodiments, a linker L, can be coupled or linked covalently to a side chain of at least one amino acid, or modified amino acid for example, a Lys residue of the GIP receptor agonist peptide being covalently attached to a substituent group. In an embodiment, the selective GIP receptor agonist peptide is covalently attached to an (R) group, wherein the (R) group is a hydrophilic polymer, and the Lys(R) residue is positioned at an amino acid position ranging from A14 to A30. In an embodiment, the selective GIP receptor agonist peptide is covalently attached to a hydrophilic polymer, for example, the hydrophilic polymer is a polyethylene glycol (PEG) molecule or a variant thereof.

In some embodiments, the linker L is a PEG molecule, for example, PEG3(n), PEG(2)(n), or mPEG having a weight average molecular weight of about 5-30 kDa. In some embodiments, L can be any combination of PEG3(n), PEG(2)(n), gGlu(n), D-gGlu(n), AMBZ(n), GABA(n), G(x), NpipAc(n), Tra(n), eLya(n), where n=1-5 and x=1-10. Exemplary PEG linkers can be used as part of an (R) group in a substituted Lys residue, for example, located at one or more of A14-A30, for example, at an amino acid position: A14, A17, A18, A20, A21, AA26, A29, or A30, wherein the MPEG linker can include one or more of the following additional MPEG linkers:

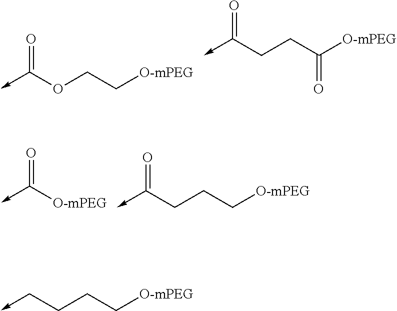

In some embodiments, exemplary MPEG linkers which may be used for coupling a substituent X to a Cys amino acid can include a MPEG molecule having an weight average molecular weight of about 5-30 kDa. In some embodiments, illustrative PEG linkers for attachment to a Cys side chain can include:

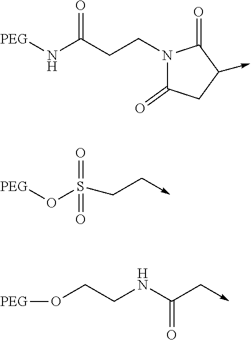

In various examples, R represents X-L-, wherein X-L- represents Hepda-GGGG-(Hepda:C17 diacid), Hepda-GGGGG-, Hepda-GGGGGG-, Oda-GGGG-(Oda:C18 diacid), Oda-GGGGG-, Oda-GGGGGG-, Eda-GGGG-(Eda:C20 diacid), Eda-GGGGG-, Eda-GGGGGG-, Eda-GGGGGGGGG-.

Alternatively, the (R) group represents X-L-, wherein L represents a glycine linker comprising five or six-linked glycines, and X represents C₁₇-C₂₀ linear saturated dicarboxylic acid.

Alternatively, the (R) group represents X-L-, wherein L represents a bond or a bivalent substituent group, and X represents an a C₁₇-C₂₂ fatty acid, or a C₁₇-C₂₂ acylated fatty acid or a C₁₇-C₂₂ dicarboxylic acid, or a salt thereof. In some embodiments, the X represents a palmitic fatty acid used to add a palmitoyl group to the epsilon amine side group of a Lys residue, for example, a Lys reside in the GIP receptor agonist peptide.

In other embodiments, the GIP receptor agonist peptide has one, or two modified lysine residues, i.e. Lys(R), wherein the (R) group represents X-L-, wherein L represents a glycine linker comprising three, four, five or six-linked glycines, and X represents C₁₇-C₂₂ linear saturated dicarboxylic acid. In an embodiment, the acyl group is a C₁₇ to C₂₂ fatty acyl group, for example a palmitoyl or myristoyl fatty acyl group.

In an embodiment, the GIP receptor agonist peptide is covalently attached to an (R) group, wherein the (R) group is a hydrophilic polymer at any amino acid position ranging from A14 to A30. In an embodiment, the GIP receptor agonist peptide is covalently attached to a hydrophilic polymer at amino acid position, A14, A17, A18, A20, A21, AA26, A29, or A30, or combinations thereof, for example, at positions A14-A30 or from A14 to A21. For example, the hydrophilic polymer may be attached to the side chain of a Lys residue of the GIP receptor agonist peptide. In an embodiment, the hydrophilic polymer is a polyethylene glycol (mPEG). The mPEG polymer may also be further conjugated to a glycine linker, i.e. (Gly)₍₂₋₈₎-, or to one or more gGlu-residues, for example, gGlu₍₁₋₃₎-. In some examples, the mPEG has a weight average molecular weight of about 1,000 Daltons to about 60,000 Daltons, such as about 5,000 Daltons to about 40,000 Daltons, or about 1,000 Daltons, or 5,000 Daltons, or 10,000 Daltons, or 12,000 Daltons, or 14,000 Daltons to about 20,000 Daltons.

In some embodiments, methods for conjugating a polyethylene glycol (mPEG) polymer to a reactive amine or sulfhydryl group is well known in the art. For example, mPEG can be conjugated to a lysine amine sidechain using an amine-reactive pegylated crosslinker. A Bis(succinimidyl)penta(ethylene glycol) spacer arm can be used as a homobifunctional, amine-to-amine crosslinker that contain N-hydroxy-succinimide (NHS) esters at both ends of a mPEG spacer arm. An amine-reactive crosslinker that contains a PEG spacer arm. A bis-succinimide ester-activated mPEG compound may be used for crosslinking between primary amines (—NH₂) in GIP receptor agonist peptides of the present disclosure. The N-hydroxysuccinimide ester (NHS) groups at either end of the mPEG spacer react specifically and efficiently with lysine and N-terminal amino groups at pH 7-9 to form stable amide bonds. Other homobifunctional, sulfhydryl-reactive crosslinkers that contain the maleimide group at either end of a PEG spacer may be used to couple PEG to a Cys amino acid of a GIP receptor agonist peptide. Heterofunctional crosslinking spacer arms may also be used when two different reactive groups are used as the linkage groups, e.g. an amine group and a sulfhydryl group. A sulfhydryl-reactive crosslinker that contains a PEG spacer arm, may be used to couple a PEG polymer to a GIP receptor agonist peptide. In some embodiments, a bismaleimide-activated PEG compound may be used for crosslinking between sulfhydryl (—SH) groups in proteins and other thiol molecules. The maleimide groups at either end of the PEG spacer may react specifically and efficiently with reduced sulfhydryls at pH 6.5-7.5 to form stable thioether bonds. In other embodiments, direct coupling of a PEG molecule to a GIP receptor agonist peptide may be accomplished using known methods in the art. For example, a well known technique whereby a peptide may be covalently modified with PEG groups requiring PEG compounds that contain a reactive or targetable functional group at one end. The simplest method to pegylate peptides, which are rich in surface primary amines, is to use a PEG compound that contains an NHS ester group at one end, for example, a methyl-(PEG)n-NHS ester. In a similar fashion, methyl-(PEG)n-maleimide (wherein n can be from 20-300) may be used to couple a PEG molecule to a Cys containing peptide of the present disclosure. Methods known in the art for conjugation of polyethylene glycol polymers of various lengths ranging from 1,000 Daltons to 20,000 Daltons or more are provided in 1. Hermanson, G. T. (2013). 3rd Edition. Bioconjugate Techniques, Academic Press, Veronese, F. and Harris, J. M. Eds. (2002). Peptide and protein PEGylation. Advanced Drug Delivery Review 54(4), 453-609, Zalipsky, S., et al., “Use of Functionalized Poly(Ethylene Glycols) for Modification of Polypeptides” in Polyethylene Glycol Chemistry: Biotechnical and Biomedical Applications, J. M. Harris, Plenus Press, New York (1992), and in Zalipsky (1995) Advanced Drug Reviews 16:157-182 the disclosures of all of these references are hereby incorporated by reference herein in their entireties.

In various embodiments, the GIP receptor agonist peptide disclosed herein with the lipidated Lys(R) residues positioned between amino acids A14 and A30, for example, at amino acid positions A14, A17, A18, A20, A21, AA26, A29, or A30, provide GIPR agonist peptides having enhanced ½ life of elimination, % remaining after 48 hours in serum, and solubility in various media, when compared to GIPR agonist peptides in the art. In some embodiments, the position of the lipidated lysine residue, the sequence of the GIPR peptide and the length of the lipid used in the (R) substituent on the Lys residue play a role in the improved half life and solubility of the GIPR peptide, that enables the GIPR agonist peptides to be dosed in a therapeutically effective way to a subject in need of antiemetic activity once per week (Q1W), for example, once per four to seven days, or once per four to five days, or once every four days, or once every five days, or once every six days, or once every seven days, or once every eight days, or once every nine days, or once every ten days. The enhanced % A life of elimination, % remaining after 48 hours in serum, and solubility in various media are illustrated in the Examples section of the present disclosure.

In various embodiments, GIP receptor agonist peptides disclosed herein which are suitable for Q1W, or once per week dosing, for example, dosing once every 4 to 6 days, or every 5 to 8 days, or every 6 to 7 days to treat emesis, including nausea and/or vomiting, have a human intravenous T1/2 life of elimination in human serum, ranging between 40-160 hours, or for example, ranging between 50-140 hours. In various embodiments, GIP receptor agonist peptides disclosed herein which are suitable for Q1W dosing, or once per week dosing to treat emesis, including nausea and/or vomiting, have a solubility of greater than 10 mg/mL, or greater than 15 mg/mL, or greater than 20 mg/mL, or greater than 30 mg/mL, or greater than 40 mg/mL, or greater than 50 mg/mL, or greater than 60 mg/mL, or greater than 75 mg/mL, or greater than 100 mg/mL, or greater than 125 mg/mL (for example, when tested in a dissolution test using phosphate buffer at pH 7.4 performed at 37° C.); and a human intravenous T1/2 life of elimination in human serum ranging between 40 to 160 hours, or for example, ranging between 50 to 150 hours, or from 90 to 145 hours. In various embodiments, GIP receptor agonist peptides disclosed herein which are suitable for Q1W dosing, or or once per week dosing, for example, dosing once every 4 to 6 days, or every 5 to 8 days, or every 6 to 7 days dosing to treat emesis, including nausea and/or vomiting, in a mammal, for example, a human, have a solubility of 15 mg/mL, or greater; and a human intravenous T1/2 life of elimination ranging between 50-160 hours, or for example, ranging between 60-160 hours. In various embodiments, the GIPR agonist peptides of the present disclosure have a T1/2 life of elimination in humans ranging from 60 hours to 160 hours as determined with the methods of the Examples below, and a solubility greater than 25 mg/mL, for example, greater than 30 mg/mL, or greater than 40 mg/mL, or greater than 45 mg/ml, or greater than 50 mg/mL or higher.

In various embodiments, GIP receptor agonist peptides disclosed herein which are suitable for Q1W dosing, or once per week dosing, for example, dosing once every 4 to 6 days, or every 5 to 8 days, or every 6 to 7 days dosing to treat emesis, including nausea and/or vomiting, in a mammal, for example, a human, have a solubility of 15-100 mg/mL, or greater; and a human intravenous T1/2 life of elimination ranging from 100 to 150 hours as determined with the methods of the Examples below, and a an amino acid sequence length of 30-32 or 39 amino acids, a substituted (Lys(R) Lysine residue positioned in the amino acid position of 14, 17, 21, or 30, a lipid characterized as a C18 diacid and a linker selected from 2OEGgEgE or GGGGG.

Solubility of the GIPR peptides may be determined by dissolution in a phosphate buffer followed by quantitation using liquid chromatography, for example, High Performance Liquid Chromatogry (HPLC). An illustrative method is provided. For determination of the solubility of the GIPr agonist peptides, 3 mg of peptides are weighted out in a small glass vial. 100 μL of 200 mM Phosphate buffer pH 7.4 are added and the vial is sonicated/votexed as necessary for a maximum of 1 min. A visual inspection is performed, If the sample is fully dissolved, the solubility is recorded as 30 mg/mL. If insoluble material is observed in the tube the addition of 100 μL of buffer and mixing is repeated until complete dissolution. If the peptide is not soluble in 500 μL of buffer, it is labeled as solubility <6 mg/mL. The solubility can be confirmed by RP-HPLC after filtration on 0.2 m filter on an Agilent 1200 system with a Kinetex column form Phenomenex® (2.6 μm EVO C18 100 Å, LC Column 50×3.0 mm) kept at 40° C., the eluent A is 0.05% TFA in Water, B is 0.035% TFA in Acetonitrile at a 0.6 ml/min flow rate. The gradient was from 20 to 70 over 5 min, the column is then washed for 1 min at 90% B. UV monitoring at 215 nm was used to monitor peptide concentration. Standards, may also be run on the same chromatographical experiment, to obtain standard measurements at 215 nm, from which a standard curve may be calculated and soluble peptide concentrations may be extrapolated from the standard curve.

In various embodiments, the GIP receptor agonist peptide disclosed herein, for example, as used in the preparation of a medicament, a composition, or for use in the prevention and/or treatment of a condition, or disorder, or in a method of prevention and/or treatment as disclosed herein, as represented by a GIP receptor agonist peptide has an amino acid sequence as provided in any one of formulas (I), (II), (III), (IV), and (V).

In various embodiments, suitable GIPR agonist peptides having the appropriate pharmacokinetics and pharmacodynamics required for therapeutically effective treatment of a subject with emesis or displaying one or more symptoms of emesis, or for use to prevent emesis by dosing Q1W, or once per week, once per four to seven days, or once per four to five days, or once every four days, or once every five days, or once every six days, or once every seven days, or once every eight days, or once every nine days, or once every ten days have the following amino acid sequence and lipid-linker characteristics:

TABLE 1
Exemplary GIPR agonist peptides of the present disclosure.

| Compound | Amino Acid Sequence | | |
|---|---|---|---|
| No. | (Single Amino Acid Letter) | Linker | Lipid |
| 84 | Me-Y-Aib-E-G-T-F-I-S-D-Y-S-I-A-Km-D-R-Aib-A-Q-Aib-D-F-V- | GGGGG | C18 |
| | N-W-L-L-A-Q-S-P-G-OH | | diacid |
| 45 | Me-Y-Aib-E-G-T-F-I-S-D-Y-S-I-A-Km-D-R-Aib-A-Q-Aib-N-F-V- | GGGGG | C18 |
| | N-W-L-L-A-Q-S-P-S-S-G-A-P-P-P-S-OH | | Diacid |
| 50 | Me-Y-Aib-E-G-T-F-I-S-D-Y-S-I-A-L-D-R-Km-A-Q-Aib-D-F-V-N | GGGGG | C18 |
| | W-L-L-A-Q-S-P-S-S-G-A-P-P-P-S-NH₂ | | Diacid |
| 41 | Me-Y-Aib-E-G-T-F-I-S-D-Y-S-I-A-L-D-R-Aib-A-Q-Aib-Km-F-V- | 2OEGgEgE | C18 |
| | N-W-L-L-A-Q-K-G-OH | | Diacid |
| 72 | Me-Y-Aib-E-G-T-F-I-S-D-Y-S-I-Aib-L-D-R-Aib-A-Q-Aib-Km-F- | 2OEGgEgE | C18 |
| | V-N-W-L-L-A-Q-R-G-OH | | Diacid |
| 27 | Me-Y-Aib-E-G-T-F-I-S-D-Y-S-I-Aib-L-D-R-Aib-A-Q-Aib-N-F-V- | 2OEGgEgE | C18 |
| | N-W-L-L-A-Q-Km-P-S-S-G-A-P-P-P-S-NH₂ | | Diacid |
| 293 | Me-Y-Aib-E-G-T-F-I-S-D-Y-S-I-A-L-D-R-Aib-Km-Q-Aib-N-F-V- | 2OEGgEgE | C18 |
| | N-W-L-L-A-Q-S-P-S-S-G-A-P-P-P-S-NH₂ | | Diacid |
| 294 | Me-Y-Aib-E-G-T-F-I-S-D-Y-S-I-Aib-Km-D-R-Aib-A-Q-Aib-D-F- | GGGGG | C18 |
| | V-N-W-L-L-A-Q-R-G-OH | | Diacid |

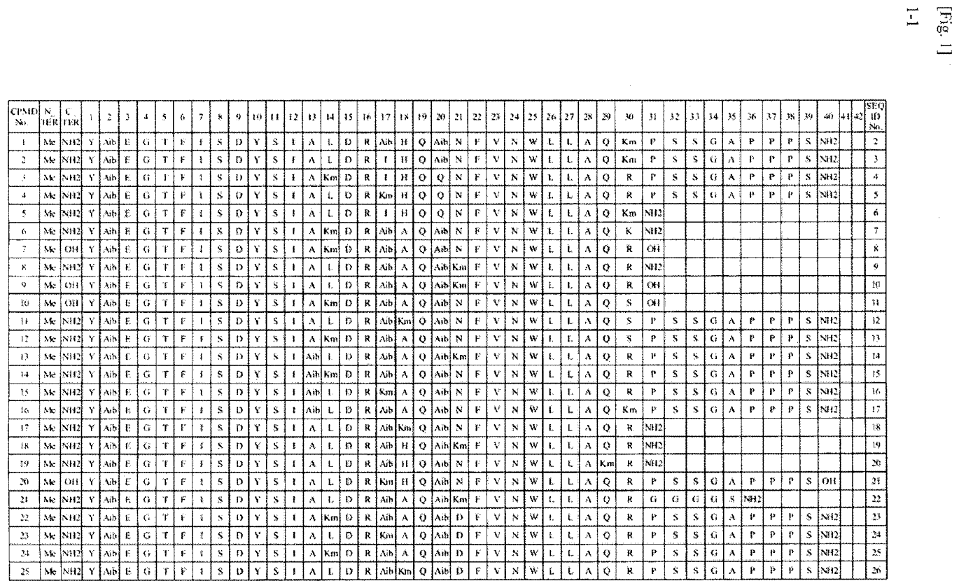

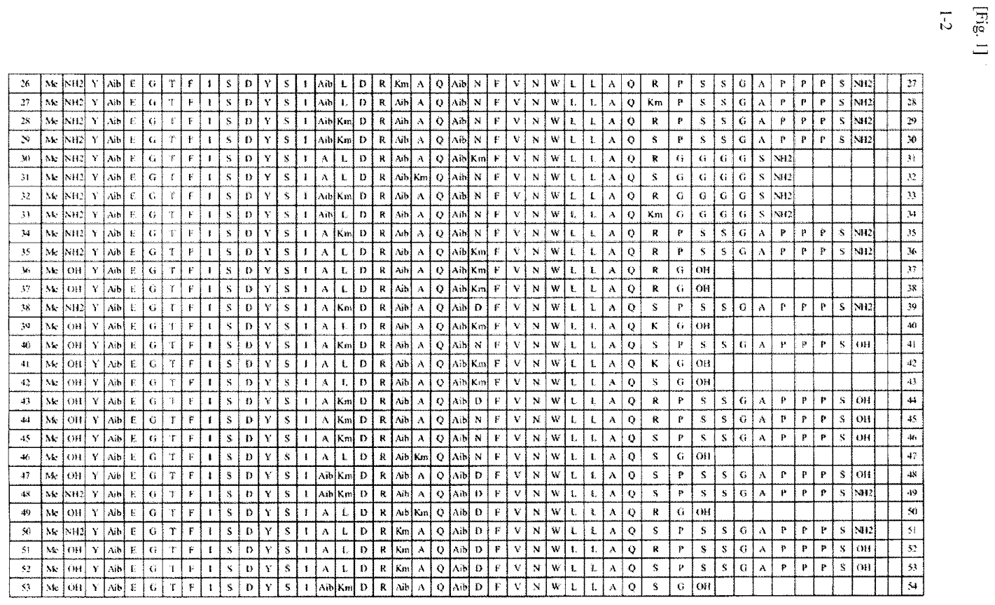

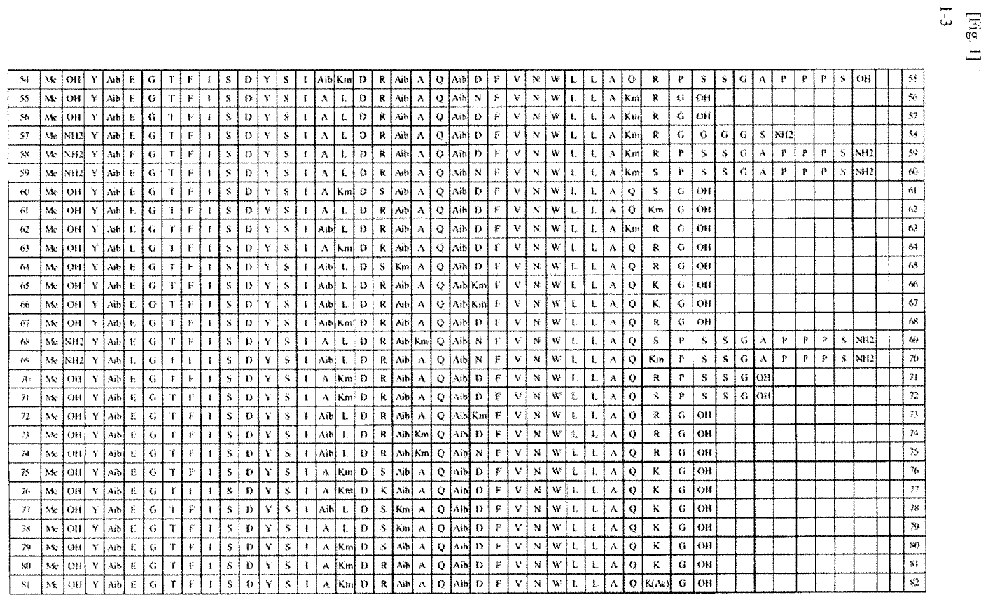

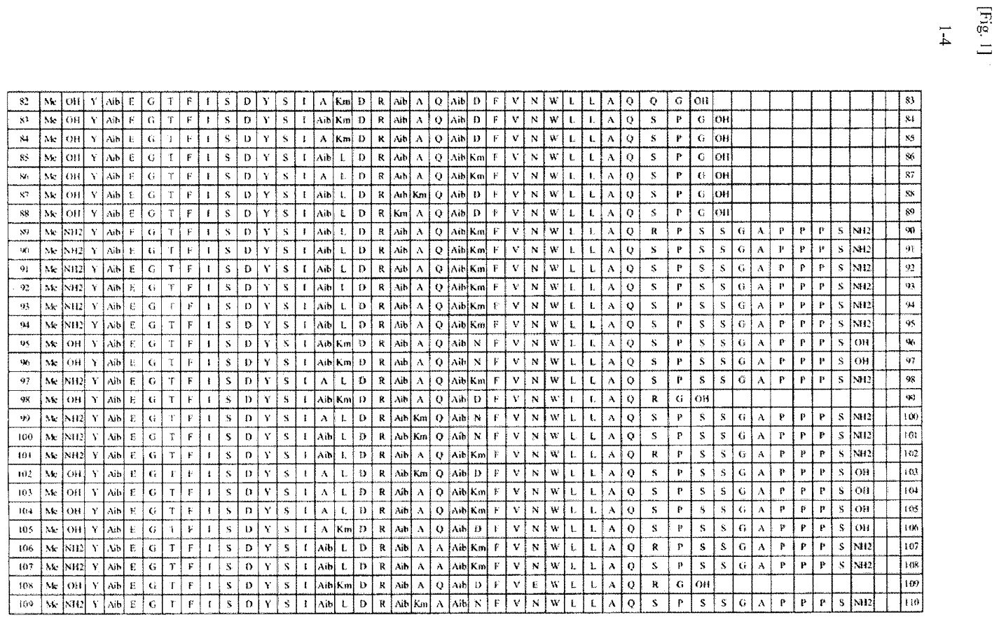

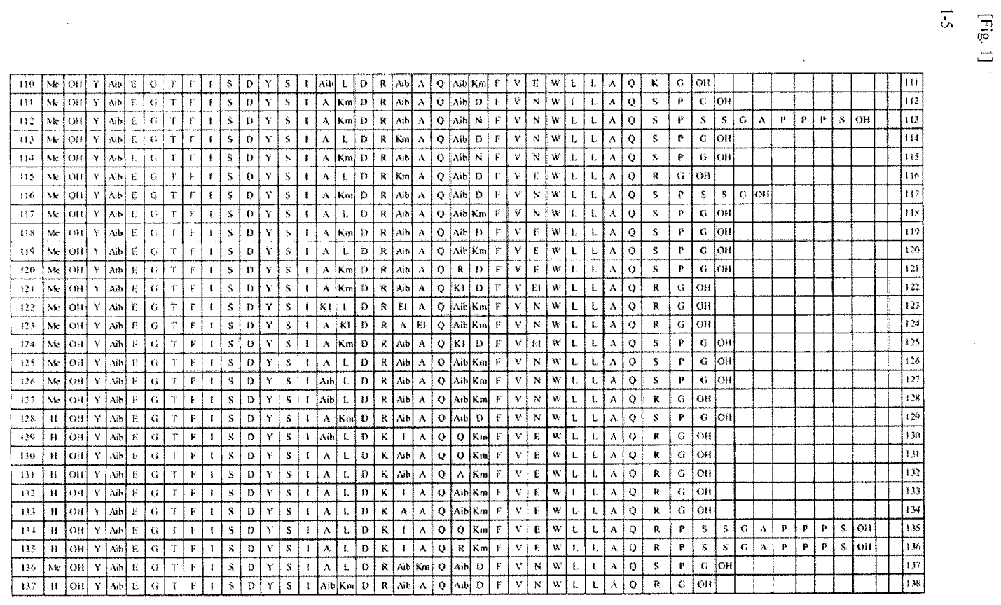

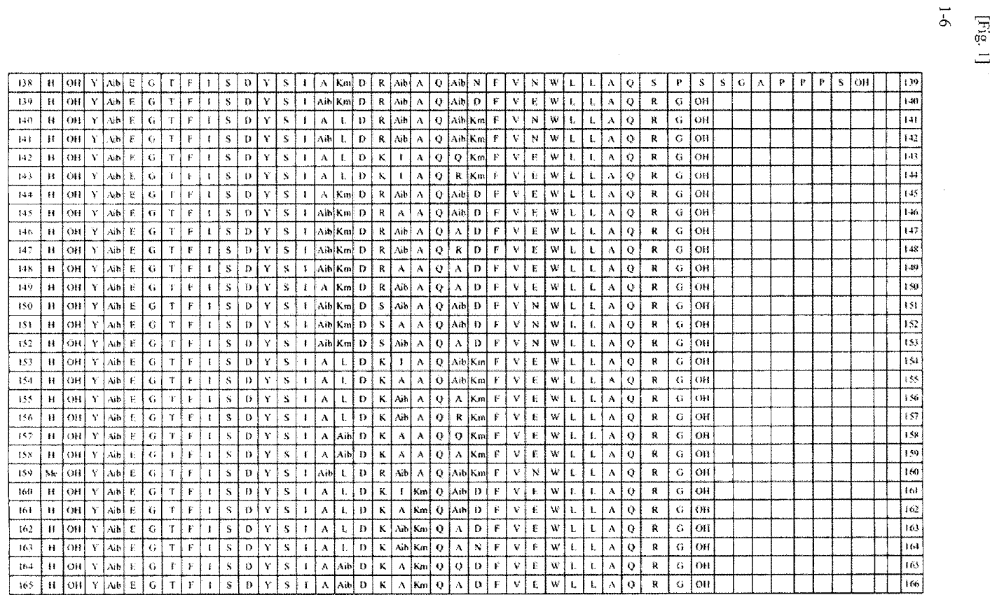

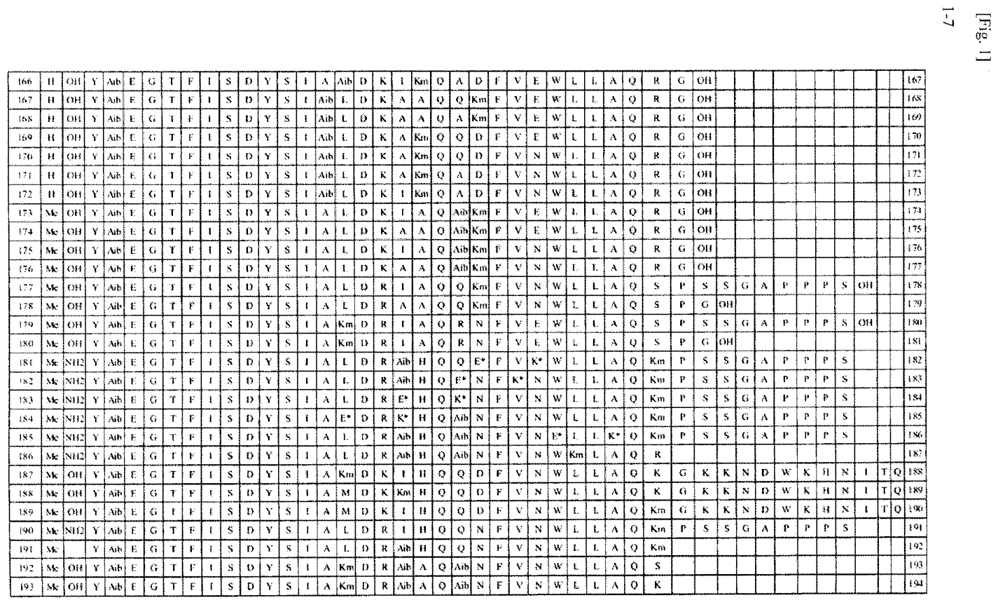

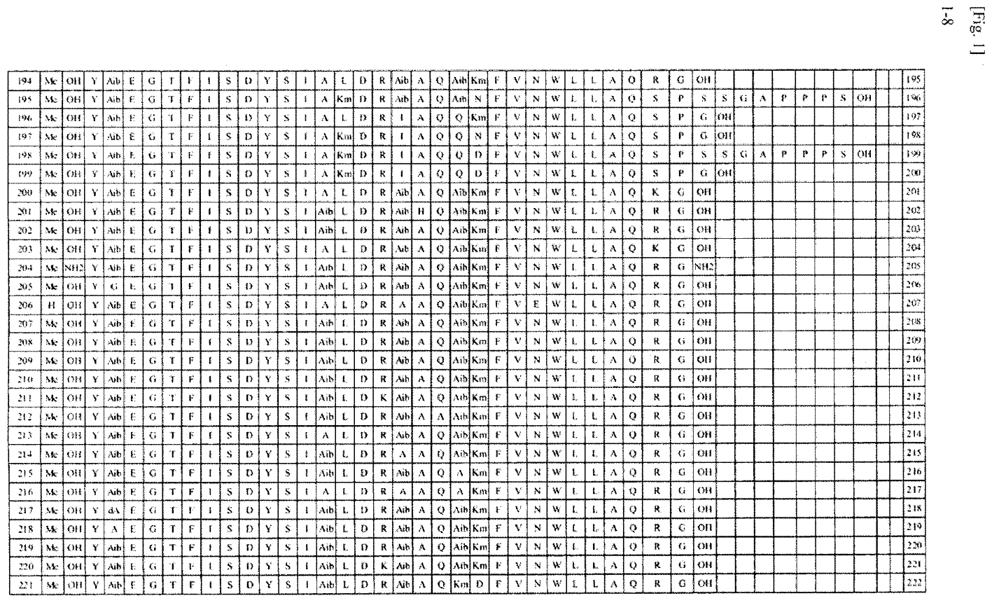

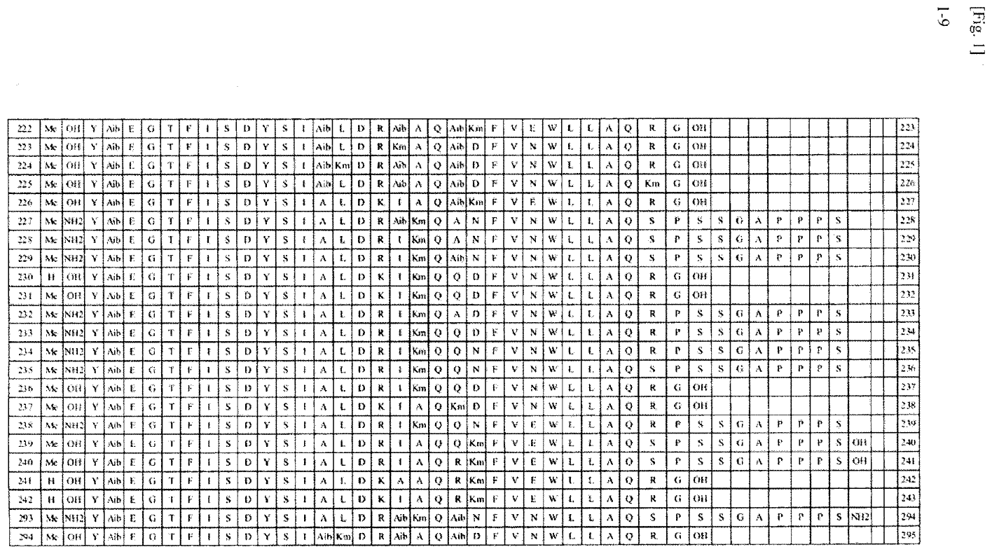

In various embodiments, exemplary GIP receptor agonist peptides having a structure as defined in any one of formulas (I), (II), (III), (IV), and (V), are provided in FIG. 1.

### B. Synthesis GIPR Agonist Peptides

The GIP receptor agonist peptide may be synthesized recombinantly or can be produced according to a peptide synthesis method known in the art. The peptide synthesis method may be any of, for example, a solid phase synthesis process and a liquid phase synthesis process. That is, the object GIP receptor agonist peptide can be produced by repeating condensation of a partial peptide or amino acid capable of constituting the GIP receptor agonist peptide, and the remaining portion (which may be constituted by two or more amino acids) according to a desired sequence. When a product having the desirable sequence has a protecting group, the object GIP receptor agonist peptide can be produced by eliminating a protecting group. Examples of the condensing method and eliminating method of a protecting group to be known include methods described in the following (1)-(5).

(1) M. Bodanszky and M. A. Ondetti: Peptide synthesis, Interscience Publishers, New York (1966)

### (2) Schroeder and Luebke: The Peptide, Academic Press, New York (1965)

(3) Nobuo Izumiya, et al.: Peptide Gosei-no-Kiso to Jikken (Basics and experiments of peptide synthesis), published by Maruzen Co. (1975)

### (4) Haruaki Yajima and Shunpei Sakakibara: Seikagaku Jikken Koza (Biochemical Experiment) 1, Tanpakushitsu no Kagaku (Chemistry of Proteins) IV, 205 (1977)

(5) Haruaki Yajima, ed.: Zoku Iyakuhin no Kaihatsu (A sequel to Development of Pharmaceuticals), Vol. 14, peptide synthesis, published by Hirokawa Shoten.

After the reaction, the GIP receptor agonist peptide can be purified and isolated using conventional methods of purification, such as solvent extraction, distillation, column chromatography, liquid chromatography, recrystallization, etc., in combination thereof. When the peptide obtained by the above-mentioned method is in a free form, it can be converted to a suitable salt by a known method; conversely, when the peptide is obtained in the form of a salt, the salt can be converted to a free form or other salt by a known method.

The starting compound may also be a salt. Examples of such salt include those exemplified as salts of the exemplified selective GIPr agonists mentioned bellow.

For condensation of protected amino acid or peptide, various activation reagents usable for peptide synthesis can be used, which include trisphosphonium salts, tetramethyluronium salts, carbodiimides and the like. Examples of the trisphosphonium salt include benzotriazol-1-yloxytris(pyrrolizino)phosphoniumhexafluorophosphate (PyBOP), bromotris(pyrrolizino)phosphoniumhexafluorophosphate (PyBroP), 7-azabenzotriazol-1-yloxytris(pyrrolizino)phosphoniumhexafluorophosphate (PyAOP), examples of the tetramethyluronium salt include 2-(1H-benzotriazol-1-yl)-1,1,3,3-tetramethyluroniumhexafluorophosphate (HBTU), 2-(7-azabenzotriazol-1-yl)-1,1,3,3-tetramethyluroniumhexafluorophosphate (HATU), 2-(1H-benzotriazol-1-yl)-1,1,3,3-tetramethyluroniumtetrafluoroborate (TBTU), 2-(5-norbornane-2,3-dicarboxyimide)-1,1,3,3-tetramethyluroniumtetrafluoroborate (TNTU), O-(N-succimidyl)-1,1,3,3-tetramethyluroniumtetrafluoroborate (TSTU), and examples of the carbodiimide include N,N′-Dicyclohexylcarbodiimide (DCC), N,N′-diisopropylcarbodiimide (DIPCDI), N-ethyl-N′-(3-dimethylaminopropyl)carbodiimide hydrochloride (EDCI.HCl) and the like. For condensation using these, addition of a racemization inhibitor [e.g., N-hydroxy-5-norbornene-2,3-dicarboxylic imide (HONB), 1-hydroxybenzotriazole (HOBt), 1-Hydroxy-7-azabenzotriazole (HOAt), 3,4-Dihydro-3-hydroxy-4-oxo-1,2,3-benzotriazine (HOOBt), ethyl 2-cyano-2-(hydroxyimino)acetate (Oxyma) etc.] is an example. A solvent to be used for the condensation can be appropriately selected from those known to be usable for peptide condensation reaction. For example, acid amides such as anhydrous or water-containing N,N-dimethylformamide, N,N-dimethylacetamide, N-methylpyrrolidone and the like, halogenated hydrocarbons such as methylene chloride, chloroform and the like, alcohols such as trifluoroethanol, phenol and the like, sulfoxides such as dimethylsulfoxide and the like, tertiary amines such as pyridine and the like, ethers such as dioxane, tetrahydrofuran and the like, nitriles such as acetonitrile, propionitrile and the like, esters such as methyl acetate, ethyl acetate and the like, an appropriate mixture of these and the like can be used. Reaction temperature is appropriately selected from the range known to be usable for peptide binding reactions, and is normally selected from the range of about −20° C. to 90° C. An activated amino acid derivative is normally used from 1.5 to 6 times in excess. In solid phase synthesis, when a test using the ninhydrin reaction reveals that the condensation is insufficient, sufficient condensation can be conducted by repeating the condensation reaction without elimination of protecting groups. If the condensation is yet insufficient even after repeating the reaction, unreacted amino acids can be acylated with acetic anhydride, acetylimidazole or the like so that an influence on the subsequent reactions can be avoided.

Examples of the protecting groups for the amino groups of the starting amino acid include benzyloxycarbonyl (Z), tert-butoxycarbonyl (Boc), tert-pentyloxycarbonyl, isobornyloxycarbonyl, 4-methoxybenzyloxycarbonyl, 2-chlorobenzyloxycarbonyl (Cl-Z), 2-bromobenzyloxycarbonyl (Br-Z), adamantyloxycarbonyl, trifluoroacetyl, phthaloyl, formyl, 2-nitrophenylsulphenyl, diphenylphosphinothioyl, 9-fluorenylmethyloxycarbonyl (Fmoc), trityl and the like.

Examples of the carboxyl-protecting group for the starting amino acid include aryl, 2-adamantyl, 4-nitrobenzyl, 4-methoxybenzyl, 4-chlorobenzyl, phenacyl and benzyloxycarbonylhydrazide, tert-butoxycarbonylhydrazide, tritylhydrazide and the like, in addition to the above-mentioned C₁₋₆ alkyl group, C₃₋₁₀ cycloalkyl group, C₇₋₁₄ aralkyl group.

The hydroxyl group of serine or threonine can be protected, for example, by esterification or etherification. Examples of the group suitable for the esterification include lower (C₂₋₄) alkanoyl groups such as an acetyl group and the like, aroyl groups such as a benzoyl group and the like, and the like, and a group derived from an organic acid and the like. In addition, examples of the group suitable for etherification include benzyl, tetrahydropyranyl, tert-butyl(Bu^{t}), trityl (Trt) and the like.

Examples of the protecting group for the phenolic hydroxyl group of tyrosine include Bzl, 2,6-dichlorobenzyl, 2-nitrobenzyl, Br-Z, tert-butyl and the like.

Examples of the protecting group for the imidazole of histidine include p-toluenesulfonyl (Tos), 4-methoxy-2,3,6-trimethylbenzenesulfonyl (Mtr), dinitrophenyl (DNP), benzyloxymethyl (Bom), tert-butoxymethyl (Bum), Boc, Trt, Fmoc and the like.

Examples of the protecting group for the guanidino group of arginine include Tos, Z, 4-methoxy-2,3,6-trimethylbenzenesulfonyl (Mtr), p-methoxybenzenesulfonyl (MBS), 2,2,5,7,8-pentamethylchromane-6-sulfonyl (Pmc), mesitylene-2-sulfonyl (Mts), 2,2,4,6,7-pentamethyldihydrobenzofuran-5-sulfonyl (Pbf), Boc, Z, NO₂ and the like.

Examples of the protecting group for a side chain amino group of lysine include Z, Cl-Z, trifluoroacetyl, Boc, Fmoc, Trt, Mtr, 4,4-dimethyl-2,6-dioxocyclohexylideneyl (Dde) and the like.

Examples of the protecting group for indolyl of tryptophan include formyl (For), Z, Boc, Mts, Mtr and the like.

Examples of the protecting group for asparagine and glutamine include Trt, xanthyl (Xan), 4,4′-dimethoxybenzhydryl (Mbh), 2,4,6-trimethoxybenzyl (Tmob) and the like.

Examples of activated carboxyl groups in the starting material include corresponding acid anhydride, azide, active esters [ester with alcohol (e.g., pentachlorophenol, 2,4,5-trichlorophenol, 2,4-dinitrophenol, cyanomethylalcohol, paranitrophenol, HONB, N-hydroxysuccimide, 1-hydroxybenzotriazole (HOBt), 1-hydroxy-7-azabenzotriazole (HOAt))] and the like. Examples of the activated amino group in the starting material include corresponding phosphorous amide.

Examples of the method for removing (eliminating) a protecting group include a catalytic reduction in a hydrogen stream in the presence of a catalyst such as Pd-black or Pd-carbon; an acid treatment using anhydrous hydrogen fluoride, methanesulfonic acid, trifluoromethanesulfonic acid, trifluoroacetic acid (TFA), trimethylsilyl bromide (TMSBr), trimethylsilyl trifluoromethanesulfonate, tetrafluoroboric acid, tris(trifluoro)boric acid, boron tribromide, or a mixture solution thereof; a base treatment using diisopropylethylamine, triethylamine, piperidine, piperazine or the like; and reduction with sodium in liquid ammonia, and the like. The elimination reaction by the above-described acid treatment is generally carried out at a temperature of −20° C. to 40° C.; the acid treatment is efficiently conducted by adding a cation scavenger such as anisole, phenol, thioanisole, metacresol and paracresol; dimethylsulfide, 1,4-butanedithiol, 1,2-ethanedithiol, triisopropylsilane and the like. Also, a 2,4-dinitrophenyl group used as a protecting group of the imidazole of histidine is removed by thiophenol treatment; a formyl group used as a protecting group of the indole of tryptophan is removed by deprotection by acid treatment in the presence of 1,2-ethanedithiol, 1,4-butanedithiol, or the like, as well as by alkali treatment with dilute sodium hydroxide, dilute ammonia, or the like.

Protection of a functional group that should not be involved in the reaction of a starting material and a protecting group, elimination of the protecting group, activation of a functional group involved in the reaction and the like can be appropriately selected from known protecting groups and known means.

In a method of preparing an amide of the peptide, it is formed by a solid phase synthesis using a resin for amide synthesis, or the α-carboxyl group of the carboxy terminal amino acid is amidated, and a peptide chain is elongated to a desired chain length toward the amino group side, thereafter a peptide wherein the protecting group for the N-terminal α-amino group of the peptide chain only removed and a peptide wherein the protecting group for the C-terminal carboxyl group only removed of the peptide chain are prepared, and the both peptides are condensed in a mixed solvent described above. For details about the condensation reaction, the same as above applies. After the protected peptide obtained by the condensation is purified, all protecting groups can be removed by the above-described method to yield a desired crude polypeptide. By purifying this crude peptide using various publicly known means of purification, and freeze-drying the main fraction, a desired amide of the peptide can be prepared.

When the GIP receptor agonist peptide is present as a configurational isomer such as enantiomer, diastereomer etc., a conformer or the like, they are also encompassed within the description of a GIP receptor agonist peptide and each can be isolated by a means known per se or the above separation and purification methods on demand. In addition, when the GIP receptor agonist peptide is in the form of a racemate, it can be separated into S- and R-forms by conventional optical resolution.

When a GIP receptor agonist peptide includes stereoisomers, both the isomers alone and mixtures of each isomers are also encompassed within the meaning of a GIP receptor agonist peptide. A GIP receptor agonist peptide can be chemically modified according to a method known per se and using substituent and polyethylene glycol. For example, a chemically modified GIP receptor agonist peptide can be produced by introducing substituent and/or conjugatedly binding polyethylene glycol to Cys residue, Asp residue, Glu residue, Lys residue and the like of a GIP receptor agonist peptide. Additionally, there may be a linker structure between the amino acid of the GIP receptor agonist peptide and substituent and polyethylene glycol.

A GIP receptor agonist peptide modified by a substituent and/or polyethylene glycol (PEG) produces for example, one or more effects related to promoting the biological activity, prolonging the blood circulation time, resistance to elimination, reducing the immunogenicity, enhancing the solubility, and enhancing the resistance to metabolism, of a therapeutically and diagnostically important peptide.

The molecular weight of PEG is not particularly limited and is normally about 1 K to about 1000 K daltons, or about 10 K to about 100 K daltons, or about 20 K to about 60 K Daltons.

Modifying a selective GIPr agonist of the present disclosure by adding an (R) substituent can be conducted by introducing the (R) substituent based on known oxidation reaction and reduction reaction.

A method well known in the art can be used as a method for modifying a GIP receptor agonist peptide by PEG, and, for example, in addition to the exemplary methods listed above, the methods described below can be used.

(1) A PEGylating reagent having an active ester (e.g., SUNBRIGHT MEGC-30TS (trade name), NOF Corp.) is bound to an amino group of the GIP receptor agonist peptide.
(2) A PEGylating reagent having an aldehyde (e.g., SUNBRIGHT ME-300AL (trade name), NOF Corp.) is bound to the amino group of the GIP receptor agonist peptide.
(3) A divalent cross-linking reagent (e.g., GMBS (Dojindo Laboratories), EMCS (Dojindo Laboratories), KMUS (Dojindo Laboratories), SMCC (Pierce)) is bound to an amino acid, (for example, a Lys and/or a Cys), of the GIP receptor agonist peptide, to which a PEGylating reagent having a thiol group (e.g., SUNBRIGHT ME-300-SH (trade name), NOF Corp.) is then bound.
(4) A thiol group is introduced to a GIP receptor agonist peptide through an SH-introducing agent (e.g., D-cysteine residue, L-cysteine residue, Traut's reagent), and this thiol group is reacted with a PEGylating reagent having a maleimide group (e.g., SUNBRIGHT ME-300MA (trade name), NOF Corp.).
(5) A thiol group is introduced to GIP receptor agonist peptide through an SH-introducing agent (e.g., D-cysteine residue, L-cysteine residue, Traut's reagent), and this thiol group is reacted with a PEGylating reagent having an iodoacetamide group (e.g., SUNBRIGHT ME-300IA (trade name), NOF Corp.).
(6) A ω-aminocarboxylic acid, an α-amino acid or the like is introduced as a linker to the N-terminal amino group of a GIP receptor agonist peptide, and an amino group derived from this linker is reacted with a PEGylating reagent having an active ester (e.g., SUNBRIGHT MEGC-30TS (trade name), NOF Corp.).
(7) A ω-aminocarboxylic acid, an α-amino acid or the like is introduced as a linker to the N-terminal amino group of a GIP receptor agonist peptide, and an amino group derived from this linker is reacted with a PEGylating reagent having an aldehyde group (e.g., SUNBRIGHT ME-300AL (trade name), NOF Corp.).

In addition, the GIP receptor agonist peptide may be a solvate (e.g., hydrate) or a non-solvate (e.g., non-hydrate).

The GIP receptor agonist peptide may be labeled with an isotope (e.g., ³H, ¹⁴C, ³⁵S, ¹²⁵I) or the like.

Furthermore, GIP receptor agonist peptide may be a deuterium conversion form wherein ¹H is converted to ²H(D).

In some embodiments, a GIP receptor agonist peptide labeled with or substituted with an isotope can be used as, for example, a tracer (PET tracer) for use in Positron Emission Tomography (PET), and is useful in the fields of medical diagnosis and the like.

For the GIP receptor agonist peptide mentioned herein, the left end is the N-terminal (amino terminal) and the right end is the C-terminal (carboxyl terminal) in accordance with the conventional peptide marking. The C-terminal of peptide may be any of an amide (—CONH₂), a carboxyl group (—COOH), a carboxylate (—COO⁻), an alkylamide (—CONHR^{a}), and an ester (—COOR^{a}). In some embodiments, C-terminal of peptide is an amide (—CONH₂).

A GIP receptor agonist peptide of the present disclosure may be in a salt form. Examples of such salt include metal salts, ammonium salts, salts with organic base, salts with inorganic acid, salts with organic acid, salts with basic or acidic amino acid, and the like.

Examples of the metal salt include alkali metal salts such as sodium salt, potassium salt and the like; alkaline earth metal salts such as calcium salt, magnesium salt, barium salt and the like; aluminum salt and the like.

Examples of the salt with organic base include salts with trimethylamine, triethylamine, pyridine, picoline, 2,6-lutidine, ethanolamine, diethanolamine, triethanolamine, cyclohexylamine, dicyclohexylamine, N,N-dibenzylethylenediamine and the like.

Examples of the salt with inorganic acid include salts with hydrochloric acid, hydrobromic acid, nitric acid, sulfuric acid, phosphoric acid and the like.

Examples of the salt with organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

Examples of the salt with basic amino acid include salts with arginine, lysine, ornithine and the like. Examples of the salt with acidic amino acid include salts with aspartic acid, glutamic acid and the like.

Among the above-mentioned salts, a pharmaceutically acceptable salt is of interest. For example, when a compound has an acidic functional group, an inorganic salt such as alkali metal salt (e.g., sodium salt, potassium salt etc.), alkaline earth metal salt (e.g., calcium salt, magnesium salt, barium salt etc.) and the like, ammonium salt etc., and when a compound has a basic functional group, for example, a salt with inorganic acid such as hydrochloric acid, hydrobromic acid, nitric acid, sulfuric acid, phosphoric acid and the like, or a salt with organic acid such as acetic acid, phthalic acid, fumaric acid, oxalic acid, tartaric acid, maleic acid, citric acid, succinic acid, methanesulfonic acid, p-toluenesulfonic acid and the like can be used.

In some embodiments, the GIP receptor agonist peptide may be synthesized and/or used in a prodrug form to treat or prevent a disease of the present disclosure, for example, diabetes, obesity and/or emesis. A prodrug means a compound which is converted to a GIP receptor agonist peptide with a reaction due to an enzyme, gastric acid, etc. under the physiological condition in the living body, that is, a compound which is converted to a GIP receptor agonist peptide with oxidation, reduction, hydrolysis, etc. according to an enzyme; a polypeptide which is converted to GIP receptor agonist peptide by hydrolysis etc. due to gastric acid, etc.

Examples of a prodrug of a GIP receptor agonist peptide may include a compound wherein an amino group of a GIP receptor agonist peptide is acylated, alkylated or phosphorylated (e.g., compound wherein amino group of a GIP receptor agonist peptide is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated, and the like); a compound wherein a hydroxy group of a GIP receptor agonist peptide is acylated, alkylated, phosphorylated or borated (e.g., a compound wherein a hydroxy group of a GIP receptor agonist peptide is acetylated, palmitoylated, propanoylated, pivaloylated, succinylated, fumarylated, alanylated or dimethylaminomethylcarbonylated); a compound wherein a carboxy group of a GIP receptor agonist peptide is esterified or amidated (e.g., a compound wherein a carboxy group of a GIP receptor agonist peptide is C₁₋₆ alkyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified or methylamidated) and the like. Among others, a compound wherein a carboxy group of a GIP receptor agonist peptide is esterified with C₁₋₆ alkyl such as methyl, ethyl, tert-butyl or the like can be used. These compounds, peptides and polypeptides can be produced from a GIP receptor agonist peptide by a method known per se.

A prodrug of a GIP receptor agonist peptide may also be one which is converted into a GIP receptor agonist peptide under a physiological condition, such as those described in IYAKUHIN no KAIHATSU (Development of Pharmaceuticals), Vol. 7, Design of Molecules, p. 163-198, Published by HIROKAWA SHOTEN (1990).

In the present specification, the prodrug may form a salt. Examples of such a salt include those exemplified as the salt of a GIP receptor agonist peptide.

In some embodiments, a GIP receptor agonist peptide of the present disclosure may be synthesized and/or used as a crystal. Crystals having a singular crystal form or a mixture of plural crystal forms are also encompassed by the examples of GIP receptor agonist peptides. Crystals can be produced by crystallizing a GIP receptor agonist peptide according to a crystallization method known per se.

In addition, a GIP receptor agonist peptide may be a pharmaceutically acceptable cocrystal or cocrystal salt. Here, the cocrystal or cocrystal salt means a crystalline substance consisting of two or more particular substances which are solids at room temperature, each having different physical properties (e.g., structure, melting point, heat of melting, hygroscopicity, solubility, stability etc.). The cocrystal and cocrystal salt can be produced by cocrystallization known per se.

The crystal of a GIP receptor agonist peptide of the present disclosure is superior in physicochemical properties (e.g., melting point, solubility, stability) and biological properties (e.g., pharmacokinetics (absorption, distribution, metabolism, excretion), efficacy expression), and thus it is extremely useful as a medicament.

In some embodiments, a GIP receptor agonist peptide and/or a prodrug thereof (hereinafter to be sometimes abbreviated as a GIP receptor agonist peptide of the present disclosure) have a GIP receptor activating action, and may have selectivity as agonists of the GIP receptor over other receptors such as the GLP1R. The compounds of the present disclosure have a high GIP receptor selective activation action in vivo.

### C. Methods of Prophylaxis and Treatment of GIP Mediated Conditions, Diseases, and Disorders

GIP is a gastrointestinal hormone called incretin and has a promoting action on insulin secretion from the pancreas. Incretin is closely related to glucose metabolism and thus the compound having a GIP receptor activation action is useful for preventing and treating symptoms related to abnormal glucose metabolism including diabetes and obesity. Additionally, the compounds of the present disclosure have a GIP receptor selective activation action and suppresses vomiting by activating GABAergic neurons in the area postrema.

More specifically, the GIP receptor agonist peptides of the present disclosure have a hypoglycemic action, an antiemetic action, and the like.

The GIP receptor agonist peptides of the present disclosure have a high chemical stability and excellent persistence of the effects in vivo.

The GIP receptor agonist peptides of the present disclosure may be used as a GIP receptor activator.

In the present disclosure, the GIP receptor activator (GIP receptor agonist) means an agent having a GIP receptor activation action. Additionally, the GIP receptor selective activator (GIP receptor peptide agonist) specifically means an agent having an EC₅₀ for the GIP receptor of 1/10 or less, or 1/100 or less, or 1/1000 or less, or 1/10000 or less, times the EC₅₀ for the GLP-1 receptor.

The GIP receptor agonist peptides of the present disclosure is low in its toxicity (e.g., acute toxicity, chronic toxicity, genetic toxicity, reproductive toxicity, cardiac toxicity, carcinogenicity), shows a few side effects, and can be safely administered to a mammal (e.g., human, bovine, horse, dog, cat, monkey, mouse, rat) as an agent for the prophylaxis or treatment of various diseases mentioned below and the like.

The GIP receptor agonist peptides of the present disclosure can be used as an agent for the treatment or prophylaxis of various diseases including diabetes and obesity, by virtue of the above-mentioned activating action on GIP receptors. The GIP receptor agonist peptides of the present disclosure can be used as an agent for the prophylaxis or treatment of, for example, symptomatic obesity, obesity based on simple obesity, disease state or disease associated with obesity, eating disorder, diabetes (e.g., type 1 diabetes, type 2 diabetes, gestational diabetes, obese diabetes), hyperlipidemia (e.g., hypertriglyceridemia, hypercholesterolemia, high LDL-cholesterolemia, low HDL-cholesterolemia, postprandial hyperlipemia), hypertension, cardiac failure, diabetic complications [e.g., neuropathy, nephropathy, retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, peripheral blood circulation disorder], metabolic syndrome (disease states having 3 or more selected from hypertriglyceridemia, (TG), low HDL cholesterol (HDL-C) emia, hypertension, abdominal obesity and impaired glucose tolerance), sarcopenia and the like.

Examples of the symptomatic obesity include endocrine obesity (e.g., Cushing syndrome, hypothyroidism, insulinoma, obese type II diabetes, pseudohypoparathyroidism, hypogonadism), central obesity (e.g., hypothalamic obesity, frontal lobe syndrome, Kleine-Levin syndrome), hereditary obesity (e.g., Prader-Willi syndrome, Laurence-Moon-Biedl syndrome), drug-induced obesity (e.g., steroid, phenothiazine, insulin, sulfonylurea (SU) agent, β-blocker-induced obesity) and the like.

Examples of the disease state or disease associated with obesity include glucose tolerance disorders, diabetes (particularly type 2 diabetes (T2DM), obese diabetes), lipid metabolism abnormality (synonymous with the above-mentioned hyperlipidemia), hypertension, cardiac failure, hyperuricemia.gout, fatty liver (including non-alchoholic steato-hepatitis), coronary heart disease (myocardial infarction, angina pectoris), cerebral infarction (brain thrombosis, transient cerebral ischemic attack), bone/articular disease (knee osteoarthritis, hip osteoarthritis, spondylitis deformans, lumbago), sleep apnea syndrome/Pickwick syndrome, menstrual disorder (abnormal menstrual cycle, abnormality of menstrual flow and cycle, amenorrhea, abnormal catamenial symptom), metabolic syndrome and the like.

New diagnostic criteria were reported by The Japan Diabetes Society in 1999 about the diagnostic criteria of diabetes.

According to this report, diabetes refers to a state that meets any of a fasting blood glucose level (glucose concentration in venous plasma) of 126 mg/dl or more, a 2-hr value (glucose concentration in venous plasma) of 200 mg/dl or more in the 75 g oral glucose tolerance test (75 g OGTT), and a casual blood glucose level (glucose concentration in venous plasma) of 200 mg/dl or more. Also, a state that does not apply to the above-mentioned diabetes, and is not a state exhibiting “a fasting blood glucose level (glucose concentration in venous plasma) less than 110 mg/dl or a 2-hr value (glucose concentration in venous plasma) less than 140 mg/dl in the 75 g oral glucose tolerance test (75 g OGTT)” (normal type) is called “borderline type”.

Moreover, new diagnostic criteria were reported by American Diabetes Association (ADA) in 1997 and by World Health Organization (WHO) in 1998 about the diagnostic criteria of diabetes.

According to these reports, diabetes refers to a state that meets a fasting blood glucose level (glucose concentration in venous plasma) of 126 mg/dl or more and a 2-hr value (glucose concentration in venous plasma) of 200 mg/dl or more in the 75 g oral glucose tolerance test.

According to the above-mentioned reports, impaired glucose tolerance refers to a state that meets a fasting blood glucose level (glucose concentration in venous plasma) less than 126 mg/dl and a 2-hr value (glucose concentration in venous plasma) of 140 mg/dl or more and less than 200 mg/dl in the 75 g oral glucose tolerance test. According to the report of ADA, a state exhibiting a fasting blood glucose level (glucose concentration in venous plasma) of 110 mg/dl or more and less than 126 mg/dl is called IFG (Impaired Fasting Glucose). On the other hand, according to the report of WHO, a state of the IFG (Impaired Fasting Glucose) exhibiting a 2-hr value (glucose concentration in venous plasma) less than 140 mg/dl in the 75 g oral glucose tolerance test is called IFG (Impaired Fasting Glycemia).

The GIP receptor agonist peptides of the present disclosure may also be used as an agent for the prophylaxis or treatment of diabetes determined according to the above-mentioned new diagnostic criteria, borderline type diabetes, impaired glucose tolerance, IFG (Impaired Fasting Glucose) and IFG (Impaired Fasting Glycemia). Moreover, the GIP receptor agonist peptides of the present disclosure can prevent progress of borderline type, impaired glucose tolerance, IFG (Impaired Fasting Glucose) or IFG (Impaired Fasting Glycemia) into diabetes.

The GIP receptor agonist peptides of the present disclosure are also useful as an agent for the prophylaxis or treatment of metabolic syndrome. The incidence of cardiovascular disease is significantly high in metabolic syndrome patients, compared with patients with a single lifestyle-related disease. Thus, the prophylaxis or treatment of metabolic syndrome is exceedingly important for preventing cardiovascular disease.

The diagnostic criteria of metabolic syndrome were announced by WHO in 1999 and by NCEP in 2001. According to the diagnostic criteria of WHO, an individual having hyperinsulinemia or abnormal glucose tolerance as a requirement and two or more of visceral obesity, dyslipidemia (high TG or low HDL) and hypertension is diagnosed as having metabolic syndrome (World Health Organization: Definition, Diagnosis and Classification of Diabetes Mellitus and Its Complications. Part I: Diagnosis and Classification of Diabetes Mellitus, World Health Organization, Geneva, 1999). According to the diagnostic criteria of the Adult Treatment Panel III of the National Cholesterol Education Program (guideline of ischemic heart disease) in USA, an individual having three or more of visceral obesity, hypertriglyceridemia, low HDL-cholesterolemia, hypertension and abnormal glucose tolerance is diagnosed as having metabolic syndrome (National Cholesterol Education Program: Executive Summary of the Third Report of National Cholesterol Education Program (NCEP) Expert Panel on Detection, Evaluation, and Treatment of High Blood Cholesterol in Adults (Adults Treatment Panel III). The Journal of the American Medical Association, Vol. 285, 2486-2497, 2001).

More specifically, the GIP receptor agonist peptides of the present disclosure have an antiemetic action, and may inhibit or reduce the number and severity of the occurrence of nausea, and/or vomiting when associated with various stimuli disclosed herein, for example, when a subject has cyclic vomiting syndrome or is administered a chemotherapeutic drug, for example, a chemotherapeutic drug with emetic potential, such as platinum based chemotherapeutics such as cisplatin, oxaliplatin, and carboplatin; irinotecan and other topo isomerase inhibitors used in the treatment of cancer. The GIP receptor agonist peptides of the present disclosure have a high chemical stability and excellent persistence of the effects in vivo.

The GIP receptor agonist peptides of the present disclosure may be used as a GIP receptor activator. In the present disclosure, the GIP receptor activator (GIP receptor agonist) means an agent having a GIP receptor activation action. Additionally, the GIP receptor selective activator (i.e. a GIP receptor agonist as used herein) specifically means an agent having an EC₅₀ for the GIP receptor of 1/1000 or less, or 1/10000 or less, times the EC₅₀ for the GLP-1 receptor, or in other words the ratio of EC₅₀ GLP1R/EC₅₀ GIPR is greater than 10, greater than 100, or greater than 1,000, or greater than 10,000, or from 100 to 1,000,000 or more.

The GIP receptor agonist peptides of the present disclosure have low toxicity (e.g., acute toxicity, chronic toxicity, genetic toxicity, reproductive toxicity, cardiac toxicity, carcinogenicity), shows a few side effects, and can be safely administered to a mammal (e.g., human, bovine, horse, dog, cat, monkey, mouse, rat) as an agent for the prophylaxis or treatment of emesis.

“Treatment,” in the context of treating emesis by administering at least one of the GIP receptor agonist peptides disclosed herein, includes both prophylactic treatment and the treatment of emesis after a subject experiences emesis. Prophylactic treatment includes administration of a GIP receptor agonist peptide before a subject experiences emesis, such as when the subject experiences nausea, as well as administration of the GIP receptor agonist peptide before the subject is exposed to a substance, agent, or event, or before the subject contracts a condition, which results in or is likely to result in the subject experiencing emesis. As used herein, “therapeutically effective amount” refers to an amount of the GIP receptor agonist peptide sufficient to elicit the desired biological response. In the present disclosure, the desired biological response is treating and/or preventing an abnormal glucose metabolism in a subject, for example, in a subject in need thereof, including diabetes and obesity, or the prevention and/or treatment of emesis in a subject in need thereof.

The compound of the present invention can also be used for secondary prevention or suppression of progression of the above-mentioned various diseases (e.g., cardiovascular events such as myocardial infarction and the like). In addition, the compound of the present invention is also useful as a feeding suppressant and a weight reducing agent. The compound of the present invention can also be used in combination with a diet therapy (e.g., diet therapy for diabetes), and an exercise therapy. The GIP receptor agonist peptides of the present disclosure can be used to treat or prevent diabetes and/or obesity, a pathophysiological condition related to diabetes and/or obesity, emesis, for example, when a subject experiences or is about to experience emesis, such as nausea and/or vomiting. In various embodiments, the subject, for example, a mammal, for example, humans, non-human primates, apes, monkeys, laboratory mammals for example, mice, rats, rabbits, guinea-pigs, ferrets, domesticated mammals, such as companion mammals, dogs, cats and horses, and farm mammals, such as cattle, pigs, sheep and goats purely as examples, but not intended to be an exhaustive list, may be treated with a GIP receptor agonist peptide of the present disclosure. In each of these cases, the methods of the present disclosure are provided to treat or prevent diabetes, obesity, or emesis in a subject in need thereof, to reduce or inhibit diabetes, obesity, or emesis, to reduce or inhibit a symptom associated with diabetes, obesity, or emesis, or to reduce or inhibit a pathological condition or symptom associated with diabetes, obesity, or emesis, for example, nausea and/or vomiting.

In order to prevent or treat emesis, an effective amount of one or more of the present compounds in a pharmaceutical composition is administered to a subject/patient (used interchangeably herein) in need thereof. A subject is determined to be in need of treatment with the present GIP receptor agonist peptide either through observation of vomiting by the subject, or through a subject's self-reporting of emesis (in the case of a human subject). A patient is determined to be in need of preventative therapy by assessing that the patient is at risk of experiencing emesis due to another medical condition or due to exposure to an agent known to be associated with emesis, such as an infection by a virus or bacteria or chemical agent or radiation.

The present GIP receptor agonist peptides are beneficial in the therapy of acute, delayed or anticipatory emesis, including emesis induced by chemotherapy, radiation, toxins, viral or bacterial infections, pregnancy, vestibular disorders (e.g. motion sickness, vertigo, dizziness and Meniere's disease), surgery, pain, opioid use and withdrawal, migraine, and variations in intracranial pressure. The uses of this invention are of particular benefit in the therapy of emesis induced by radiation, for example during the treatment of cancer, or radiation sickness, and in the treatment of post-operative nausea and vomiting. Most especially, use of the invention is beneficial in the therapy of emesis induced by antineoplastic (cytotoxic) agents including those routinely used in cancer chemotherapy, emesis induced by other pharmacological agents, for example, alpha-2 adrenoceptor antagonists, such as yohimbine, MK-912 and MK-467, and type IV cyclic nucleotide phosphodiesterase (PDE4) inhibitors, such as RS14203, CT-2450 and rolipram.

Particular examples of chemotherapeutic agents are described, for example, by D. J. Stewart in Nausea and Vomiting: Recent Research and Clinical Advances, ed. J. Kucharczyk et al., CRC Press Inc., Boca Raton, Fla., USA, 1991, pages 177-203, especially page 188. Commonly used chemotherapeutic agents include cisplatin, carboplatin, oxaliplatin, cyclophosphamide, dacarbazine (DTIC), dactinomycin, mechlorethamine (nitrogen mustard), streptozocin, cyclophosphamide, carmustine (BCNU), irinotecan, and other topoisomerase inhibitors, lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel and chlorambucil (R. J. Gralle et al. in Cancer Treatment Reports, 1984, 68, 163-172). Emesis due to other chemical agents, such as the toxins soman or sarin, or opioid drug usage and/or withdrawal, e.g. morphine, heroin, oxycodone, and the like can also be prevented and/or treated.

The present compounds are administered to a patient in a quantity sufficient to treat or prevent the symptoms and/or underlying etiology associated with emesis in the patient. In a preferred embodiment, the GIP receptor agonist peptides are administered prior to administration of an agent which is likely to cause emesis, such as one or more of the chemotherapeutic agents described above. The present GIP receptor agonist peptides can also be administered in combination with such agents, either in physical combination or in combined therapy through the administration of the present compounds and agents in succession (in any order). Although the present invention is useful in any mammal suffering from emesis, a preferred subject is a human.

In some embodiments, the selective GIPr agonists of the present disclosure may be administered to treat emesis when a subject is concomitantly being treated for diabetes and/or obesity. Several known anti-diabetic medicaments are known for causing emesis, for example, Metformin (Glucophage, Glumetza, others), sulfonylureas, meglitinides, thiazolidinediones, DPP-4 inhibitors, SGLT2 inhibitors, and GLP-1 receptor agonists. In some embodiments, methods for treating emesis in a subject, for example in a subject in need thereof, may include administering an effective amount of a GIP receptor agonist peptide to a subject that does not have type-2 diabetes mellitus or a subject that is not taking a medicament to treat type-2 diabetes mellitus while experiencing emesis.

Nausea is a subjective unpleasant feeling in the back of one's throat and stomach that may lead to vomiting. There are many words that describe nausea including, but not limited to: sick to my stomach, queasy, or upset stomach. Nausea can have other symptoms that happen at the same time, such as increased saliva (spit), dizziness, light-headedness, trouble swallowing, skin temperature changes, and a fast heart rate. Vomiting is also described as “throwing up.” When one vomits, one's stomach muscles contract (squeeze) and push the contents of one's stomach out through their mouth. One might or might not feel nauseated. Retching is when one tries to vomit without bringing anything up from one's stomach. Other words used to describe retching are gagging or dry heaves. Nausea and vomiting often happen at the same time, but they can be 2 different conditions that may be mutually exclusive or mutually associated. Some chemotherapy drugs are more likely to cause nausea and vomiting than others. Doctors classify chemotherapy drugs according to their emetogenic potential (how likely the drug will cause nausea or vomiting) as high, moderate, low, or minimal risk.

In various embodiments, the GIPR agonist peptide compounds may be dosed once per week (QW or Q1W which are used interchangeable herein) to provide treatment and prophylactic treatment against emesis and emesis related symptoms. In particular, the GIPR agonist peptide compounds of the present disclosure may be used to preferentially treat chemotherapy-induced nausea and vomiting (CINV), chronic unexplained nausea and/or vomiting, Cyclic vomiting syndrome (CVS), and nausea and/or vomiting associated with gastroparesis. Cyclic vomiting syndrome is a chronic functional gastrointestinal disorder that is being increasingly recognized in adults. It is characterized by episodic nausea and vomiting and is associated with significant morbidity.

An estimated 80% of patients with cancer will experience chemotherapy-induced nausea and vomiting (CINV). The term CINV includes emesis and nausea, which can involve a loss of appetite and result in decreased oral intake of fluids and calories. Five different types of CINV have been defined and include acute, delayed, breakthrough, anticipatory, and refractory CINV.

In an exemplary embodiment, the present disclosure provides for the prophylactic treatment or maintenance therapy for chemotherapy-induced nausea and vomiting (CINV), chronic unexplained nausea and/or vomiting, Cyclic vomiting syndrome (CVS), and nausea and/or vomiting associated with gastroparesis, comprising administering one or more GIPR agonist peptide compounds of the present disclosure, for example, a GIPR agonist peptide compound selected from compounds 84, 45, 50, 41, 72, and 27, in a therapeutically effective amount to a subject in need thereof.

The GIP receptor agonist peptides of the present disclosure may be used as a preventive/therapeutic agent, ie. prophylactic treatment or maintenance therapy for vomiting and/or nausea caused, for example, by clinical pathological conditions or causes described in the following (1) to (10). Additionally, the GIP receptor agonist peptide of the present disclosure may be used as a preventive/therapeutic agent for chronic unexplained nausea and vomiting. The vomiting or nausea also includes imminent unpleasant sensations of wanting to eject the contents of the stomach through the mouth such as feeling queasy and retching, and may also be accompanied by autonomic symptoms such as facial pallor, cold sweat, salivary secretion, tachycardia, and diarrhea. The vomiting also includes acute vomiting, protracted vomiting, and anticipatory vomiting.

(1) Diseases accompanied by vomiting or nausea such as gastroparesis, gastrointestinal hypomotility, peritonitis, abdominal tumor, constipation, gastrointestinal obstruction, chronic intestinal pseudo-obstruction, functional dyspepsia, cyclic vomiting syndrome, chemotherapy-induced nausea and vomiting (CINV), nausea and/or vomiting associated with gastroparesis, chronic unexplained nausea and vomiting, acute pancreatitis, chronic pancreatitis, hepatitis, hyperkalemia, cerebral edema, intracranial lesion, metabolic disorder, gastritis caused by an infection, postoperative disease, myocardial infarction, migraine, intracranial hypertension, and intracranial hypotension (e.g., altitude sickness);
(2) Vomiting and/or nausea induced by chemotherapeutic drugs such as (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome;
(3) Vomiting or nausea caused by radiation sickness or radiation therapy for the chest, the abdomen, or the like used to treat cancers;
(4) Vomiting or nausea caused by a poisonous substance or a toxin;
(5) Vomiting and nausea caused by pregnancy including hyperemesis gravidarium; and
(6) Vomiting and nausea caused by a vestibular disorder such as motion sickness or dizziness
(7) Opioid withdrawal;
(8) Vomiting and nausea caused by chronic unexplained nausea and vomiting;
(9) A vestibular disorder such as motion sickness or dizziness; and
(10) A physical injury causing local, systemic, acute or chronic pain.

These causes of emesis, or nausea, or vomiting are not meant to be exhaustive. Other conditions, activities, side effects may cause emesis, for example, nausea and/or vomiting. Nausea can be measured in ways known to the art, such as through the use of a visual analog scale (VAS).

### D. Formulations

A medicament containing a GIP receptor agonist peptide of the present disclosure shows low toxicity and is obtained using the compound of the present disclosure alone or in admixture with a pharmacologically acceptable carrier according to a method known per se (e.g., the method described in the Japanese Pharmacopoeia) generally used as production methods of pharmaceutical preparations, and safely administered orally or parenterally (e.g., topically, rectally, intravenously administered) as a pharmaceutical preparation, for example, tablets (inclusive of sugar-coated tablets, film-coated tablets, sublingual tablets, orally disintegrating tablets), powders, granules, capsules (inclusive of soft capsules, microcapsules), liquids, troches, syrups, emulsions, suspensions, injections (e.g., subcutaneous injections, intravenous injections, intramuscular injections, intraperitoneal injections etc.), external preparations (e.g., transnasal preparations, dermal preparations, ointments), suppository (e.g., rectal suppositories, vaginal suppositories), pellets, nasal preparations, pulmonary preparations (inhalants), transfusions and the like.

These preparations may be controlled release preparations such as a rapid release preparation, a sustained release preparation and the like (e.g., a sustained release microcapsule). The content of the compound of the present disclosure in a pharmaceutical preparation is about 0.01-about 100 wt % of the whole preparation.

The above-mentioned pharmaceutically acceptable carrier may be exemplified by various organic or inorganic carrier materials that are conventionally used as preparation materials, for example, excipient, lubricant, binding agent and disintegrant for solid preparations; or solvent, solubilizing agent, suspending agent, isotonic agent, buffering agent, soothing agent and the like for liquid preparations. Further, if necessary, general additives such as preservative, antioxidant, colorant, sweetening agent, adsorbing agent, wetting agent and the like can be also used appropriately in a suitable amount.

Examples of the excipient include lactose, sucrose, D-mannitol, starch, corn starch, crystalline cellulose, light anhydrous silicic acid and the like.

Examples of the lubricant include magnesium stearate, calcium stearate, talc, colloidal silica and the like.

Examples of the binding agent include crystalline cellulose, sucrose, D-mannitol, dextrin, hydroxypropylcellulose, hydroxypropylmethylcellulose, polyvinylpyrrolidone, starch, sucrose, gelatin, methylcellulose, carboxymethylcellulose sodium and the like.

Examples of the disintegrant include starch, carboxymethylcellulose, carboxymethylcellulose calcium, carboxymethylstarch sodium, L-hydroxypropylcellulose and the like.

Examples of the solvent include water for injection, alcohol, propylene glycol, Macrogol, sesame oil, corn oil, olive oil and the like.

Examples of the solubilizing agent include polyethylene glycol, propylene glycol, D-mannitol, benzyl benzoate, ethanol, trisaminomethane, cholesterol, triethanolamine, sodium carbonate, sodium citrate and the like.

Examples of the suspending agent include surfactants such as stearyl triethanolamine, sodium lauryl sulfate, laurylaminopropionic acid, lecithin, benzalkonium chloride, benzetonium chloride, glycerin monostearate and the like; hydrophilic polymers such as polyvinyl alcohol, polyvinylpyrrolidone, carboxymethylcellulose sodium, methylcellulose, hydroxymethylcellulose, hydroxyethylcellulose, hydroxypropylcellulose and the like; and the like.

Examples of the isotonic agent include glucose, D-sorbitol, sodium chloride, glycerin, D-mannitol and the like.

Examples of the buffering agent include buffer solutions such as phosphates, acetates, carbonates, citrates and the like.

Examples of the soothing agent include benzyl alcohol and the like.

Examples of the preservative include parahydroxybenzoic acid esters, chlorobutanol, benzyl alcohol, phenethyl alcohol, dehydroacetic acid, sorbic acid and the like.

Examples of the antioxidant include sulfites, ascorbic acid, α-tocopherol and the like.

Examples of the colorant include water-soluble food coal tar dyes (e.g., food dyes such as Food Red No. 2 and No. 3, Food Yellow No. 4 and No. 5, Food Blue No. 1 and No. 2, and the like), water-insoluble lake dyes (e.g., aluminum salts of the aforementioned water-soluble Food coal tar dyes), natural dyes (e.g., β-carotene, chlorophyll, ferric oxide red) and the like.

Examples of the sweetening agent include saccharin sodium, dipotassium glycyrrhizinate, aspartame, stevia and the like.

Examples of the adsorbing include porous starch, calcium silicate (trade name: Florite RE), magnesium alumino metasilicate (trade name: Neusilin) and light anhydrous silicic acid (trade name: Sylysia).

Examples of the wetting agent include propylene glycol monostearate, sorbitan monooleate, diethylene glycol monolaurate and polyoxyethylenelauryl ether.

During production of an oral preparation, coating may be applied as necessary for the purpose of masking of taste, enteric property or durability.

Examples of the coating base to be used for coating include sugar coating base, aqueous film coating base, enteric film coating base and sustained-release film coating base.

As the sugar coating base, sucrose is used. Moreover, one or more kinds selected from talc, precipitated calcium carbonate, gelatin, gum arabic, pullulan, carnauba wax and the like may be used in combination.

Examples of the aqueous film coating base include cellulose polymers such as hydroxypropyl cellulose, hydroxypropylmethyl cellulose, hydroxyethyl cellulose, methylhydroxyethyl cellulose etc.; synthetic polymers such as polyvinylacetal diethylaminoacetate, aminoalkyl methacrylate copolymer E [Eudragit E (trade name)], polyvinylpyrrolidone etc.; and polysaccharides such as pullulan etc.

Examples of the enteric film coating base include cellulose polymers such as hydroxypropylmethyl cellulose phthalate, hydroxypropylmethyl cellulose acetate succinate, carboxymethylethyl cellulose, cellulose acetate phthalate etc.; acrylic polymers such as methacrylic acid copolymer L [Eudragit L (trade name)], methacrylic acid copolymer LD [Eudragit L-30D55 (trade name)], methacrylic acid copolymer S [Eudragit S (trade name)] etc.; and naturally occurring substances such as shellac etc.

Examples of the sustained-release film coating base include cellulose polymers such as ethyl cellulose etc.; and acrylic polymers such as aminoalkyl methacrylate copolymer RS [Eudragit RS (trade name)], ethyl acrylate-methyl methacrylate copolymer suspension [Eudragit NE (trade name)] etc.

The above-mentioned coating bases may be used after mixing with two or more kinds thereof at appropriate ratios. For coating, for example, a light shielding agent such as titanium oxide, red ferric oxide and the like can be used.

### E. Administration

The therapeutically effective amount or dose of a composition or medicament containing a GIP receptor agonist peptide to be administered to a subject will depend on the age, sex and weight of the patient, and the current medical condition of the patient. The skilled artisan will be able to determine appropriate dosages depending on these and other factors to achieve the desired biological response.

The dosage of the GIP receptor agonist peptide of the present disclosure is appropriately determined according to the subject of administration, symptom, administration method and the like. For example, when the GIP receptor agonist peptide of the present disclosure is administered orally to a subject prior to engaging in an act that will likely cause emesis or after the onset of emesis in a human subject (body weight of approximately 60 kg), the daily dose of the compound of the present disclosure is about 0.01 to 100 mg, about 1.0 to 50 mg, or about 1.0 to 20 mg. When the compound of the present disclosure is administered parenterally to an obesity or diabetes patient or a gastroparesis (body weight 60 kg), the daily dose of the compound of the present disclosure is about 0.001 to 30 mg, about 0.01 to 20 mg, or about 0.1 to 10 mg. These amounts can be administered in about 1 to several portions a day. In some embodiments, a therapeutically effective amount of a GIP receptor agonist peptide to prevent and/or treat emesis in a subject in need thereof may range from about 0.01 to 0.5 mg/kg/day, 0.1 to 5 mg/kg/day, 5 to 10 mg/kg/day, 10 to 20 mg/kg/day, 20 to 50 mg/kg/day, 10 to 100 mg/kg/day, 10 to 120 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day or 900 to 1000 mg/kg/day.

The GIP receptor agonist peptide of the present disclosure can be administered, for example, once per week (QW), or every 4 days, every 5 days, every 6 days, every seven days, twice per week, every other week, every 3 weeks, every month, every 2 months, every 3 months, every 4 months, every 5 months or every 6 months. In some embodiments, the GIP receptor agonist peptide of the present disclosure can be administered to the subject 1-2 times per week, or 1-2 times per 10 days, for 1-5 weeks, 1-5 months, or 1-5 years.

The GIP receptor agonist peptide of the present disclosure can be used in combination with another drug that does not adversely influence the GIP receptor agonist peptide of the present disclosure, for the purpose of, for example, promoting the action (antiemetic action) of the GIP receptor agonist peptide of the present disclosure, reducing the dose of the GIP receptor agonist peptide of the present disclosure, and the like.

Examples of a drug that can be used in combination with the GIP receptor agonist peptide of the present disclosure (hereinafter sometimes to be abbreviated as a concomitant drug) include anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, therapeutic agents for osteoporosis, vitamins, antidementia drugs, erectile dysfunction drugs, therapeutic drugs for urinary frequency or urinary incontinence, therapeutic agents for dysuria, central D2 receptor antagonists, prokinetic agents, antihistamines, muscarine receptor antagonists, serotonin 5HT3 receptor antagonists, somatostatin analogues, corticosteroids, benzodiazepine anxiolytics, NK-1 receptor antagonists, hypercalcemia therapeutic drug and the like. Specific examples of the concomitant drug include those mentioned below.

Examples of the anti-obesity agent include monoamine uptake inhibitors (e.g., phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), neuropeptide Y antagonists (e.g., velneperit), cannabinoid receptor antagonists (e.g., rimonabant, taranabant), ghrelin antagonists, ghrelin receptor antagonists, ghrelinacylation enzyme inhibitors, opioid receptor antagonists (e.g., GSK-1521498), orexin receptor antagonists, melanocortin 4 receptor agonists, 11β-hydroxysteroid dehydrogenase inhibitors (e.g., AZD-4017), pancreatic lipase inhibitors (e.g., orlistat, cetilistat), β3 agonists (e.g., N-5984), diacylglycerol acyltransferase 1 (DGAT1) inhibitors, acetylCoA carboxylase (ACC) inhibitors, stearoyl-CoA desaturated enzyme inhibitors, microsomal triglyceride transfer protein inhibitors (e.g., R-256918), Na-glucose cotransporter inhibitors (e.g., JNJ-28431754, remogliflozin), NFκ inhibitory (e.g., HE-3286), PPAR agonists (e.g., GFT-505, DRF-11605), phosphotyrosine phosphatase inhibitors (e.g., sodium vanadate, Trodusquemin), GPR119 agonists (e.g., PSN-821, MBX-2982, APD597), glucokinase activators (e.g., AZD-1656), leptin, leptin derivatives (e.g., metreleptin), CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin preparations: FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), GLP-1 receptor agonist, GLP-1 receptor/GIP receptor coagonist, glucagon receptor/GLP-1 receptor/GIP receptor triagonist, and the like.

Here, as the therapeutic agent for diabetes, for example, insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation), insulin sensitizers (e.g., pioglitazone or a salt thereof (e.g., hydrochloride), rosiglitazone or a salt thereof (e.g., maleate), Metaglidasen, AMG-131, Balaglitazone, MBX-2044, Rivoglitazone, Aleglitazar, Chiglitazar, Lobeglitazone, PLX-204, PN-2034, GFT-505, THR-0921, compound described in WO007/013694, WO2007/018314, WO2008/093639 or WO2008/099794), α-glucosidase inhibitors (e.g., voglibose, acarbose, miglitol, emiglitate), biguanides (e.g., metformin, buformin or a salt thereof (e.g., hydrochloride, fumarate, succinate)), insulin secretagogues (e.g., sulfonylurea (e.g., tolbutamide, glibenclamide, gliclazide, chlorpropamide, tolazamide, acetohexamide, glyclopyramide, glimepiride, glipizide, glybuzole), repaglinide, nateglinide, mitiglinide or calcium salt hydrate thereof), dipeptidyl peptidase IV inhibitors (e.g., Alogliptin or a salt thereof (e.g., benzoate), Vildagliptin, Sitagliptin, Saxagliptin, BI1356, GRC8200, MP-513, PF-00734200, PHX1149, SK-0403, ALS2-0426, TA-6666, TS-021, KRP-104, Trelagliptin or a salt thereof (e.g., succinate)), β3 agonists (e.g., N-5984), GPR40 agonists (e.g., Fasiglifam or a hydrate thereof, compound described in WO2004/041266, WO2004/106276, WO2005/063729, WO2005/063725, WO2005/087710, WO2005/095338, WO2007/013689 or WO2008/001931), SGLT2 (sodium-glucose cotransporter 2) inhibitors (e.g., Dapagliflozin, AVE2268, TS-033, YM543, TA-7284, Remogliflozin, ASP1941), SGLT1 inhibitors, 11p-hydroxysteroid dehydrogenase inhibitors (e.g., BVT-3498, INCB-13739), adiponectin or agonist thereof, IKK inhibitors (e.g., AS-2868), leptin resistance improving drugs, somatostatin receptor agonists, glucokinase activators (e.g., Piragliatin, AZD1656, AZD6370, TTP-355, compound described in WO006/112549, WO007/028135, WO008/047821, WO008/050821, WO008/136428 or WO008/156757), GPR119 agonists (e.g., PSN821, MBX-2982, APD597), FGF21, FGF analogue, ACC2 inhibitors, GLP-1 receptor agonist, GLP-1 receptor/GIP receptor coagonist, glucagon receptor/GLP-1 receptor/GIP receptor triagonist, and the like can be mentioned.

As the therapeutic agent for diabetic complications may include, aldose reductase inhibitors (e.g., tolrestat, epalrestat, zopolrestat, fidarestat, CT-112, ranirestat (AS-3201), lidorestat), neurotrophic factor and increasing agents thereof (e.g., NGF, NT-3, BDNF, neurotrophic production/secretion promoting agent described in WO01/14372 (e.g., 4-(4-chlorophenyl)-2-(2-methyl-1-imidazolyl)-5-[3-(2-methylphenoxy)propyl]oxazole), compound described in WO2004/039365), PKC inhibitors (e.g., ruboxistaurin mesylate), AGE inhibitors (e.g., ALT946, N-phenacylthiazolium bromide (ALT766), EXO-226, Pyridorin, pyridoxamine), GABA receptor agonists (e.g., gabapentin, pregabalin), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), apoptosis signal regulating kinase-1 (ASK-1) inhibitors, GLP-1 receptor agonist, GLP-1 receptor/GIP receptor coagonist, glucagon receptor/GLP-1 receptor/GIP receptor triagonist, and the like can be mentioned.

As the therapeutic agent for hyperlipidemia, HMG-CoA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compound described in WO97/10224, for example, N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidin-4-acetic acid), fibrate compounds (e.g., bezafibrate, clofibrate, simfibrate, clinofibrate), anion exchange resin (e.g., colestyramine), probucol, nicotinic acid drugs (e.g., nicomol, niceritrol, niaspan), ethyl icosapentate, phytosterol (e.g., soysterol, gamma oryzanol (γ-oryzanol)), cholesterol absorption inhibitors (e.g., zechia), CETP inhibitors (e.g., dalcetrapib, anacetrapib), ω-3 fatty acid preparations (e.g., ω-3-fatty acid ethyl esters 90 (ω-3-acid ethyl esters 90)) and the like can be mentioned.

Examples of the antihypertensive agent include angiotensin converting enzyme inhibitors (e.g., captopril, enalapril, delapril, etc.), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil, etc.), calcium antagonists (e.g., manidipine, nifedipine, amlodipine, efonidipine, nicardipine, cilnidipine, etc.), p blockers (e.g., metoprolol, atenolol, propranolol, carvedilol, pindolol, etc.), clonidine and the like.

As the diuretic, for example, xanthine derivatives (e.g., theobromine sodium salicylate, theobromine calcium salicylate and the like), thiazide preparations (e.g., ethiazide, cyclopenthiazide, trichloromethiazide, hydrochlorothiazide, hydroflumethiazide, benzylhydrochlorothiazide, penfluthiazide, poly5thiazide, methyclothiazide and the like), antialdosterone preparations (e.g., spironolactone, triamterene and the like), carbonic anhydrase inhibitors (e.g., acetazolamide and the like), chlorobenzenesulfonamide agents (e.g., chlortalidone, mefruside, indapamide and the like), azosemide, isosorbide, ethacrynic acid, piretanide, bumetanide, furosemide and the like can be mentioned.

Examples of the chemotherapeutic include alkylating agents (e.g., cyclophosphamide, ifosfamide), antimetabolites (e.g., methotrexate, 5-fluorouracil), anticancer antibiotics (e.g., mitomycin, adriamycin), plant-derived anticancer agents (e.g., vincristine, vindesine, Taxol), cisplatin, carboplatin, etoposide and the like. Among others, a 5-fluorouracil derivative Furtulon or Neofurtulon or the like is an example. Also a composition comprising a GIP receptor agonist peptide of the disclosure can be administered before, after or during the administration of the following anti-cancer agents: cisplatin, carboplatin. Oxaliplatin, cyclophosphamide, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel and chlorambucil.

Examples of the immunotherapeutic include microbial or bacterial components (e.g., muramyl dipeptide derivative, Picibanil), polysaccharides having immunoenhancing activity (e.g., lentinan, sizofiran, Krestin), cytokines obtained by genetic engineering approaches (e.g., interferon, interleukin (IL)), colony-stimulating factors (e.g., granulocyte colony-stimulating factor, erythropoietin) and the like. Among others, interleukins such as IL-1, IL-2, IL-12 and the like are some examples.

Examples of the anti-inflammatory drug include nonsteroidal anti-inflammatory drugs such as aspirin, acetaminophen, indomethacin and the like.

As the antithrombotic agent, for example, heparin (e.g., heparin sodium, heparin calcium, enoxaparin sodium, dalteparin sodium), warfarin (e.g., warfarin potassium), anti-thrombin drugs (e.g., aragatroban, dabigatran), FXa inhibitors (e.g., rivaroxaban, apixaban, edoxaban, YM150, compound described in WO02/06234, WO2004/048363, WO2005/030740, WO2005/058823 or WO2005/113504), thrombolytic agents (e.g., urokinase, tisokinase, alteplase, nateplase, monteplase, pamiteplase), platelet aggregation inhibitors (e.g., ticlopidine hydrochloride, clopidogrel, prasugrel, E5555, SHC530348, cilostazol, ethyl icosapentate, beraprost sodium, sarpogrelate hydrochloride) and the like can be mentioned.

Examples of the therapeutic agent for osteoporosis include alfacalcidol, calcitriol, elcatonin, calcitonin salmon, estriol, ipriflavone, pamidronate disodium, alendronate sodium hydrate, incadronate disodium, risedronate disodium and the like.

Examples of the vitamin include vitamin B1, vitamin B12 and the like.

Examples of the antidementia drug include tacrine, donepezil, rivastigmine, galanthamine and the like.

Examples of the erectile dysfunction drug include apomorphine, sildenafil citrate and the like.

Examples of the therapeutic drug for urinary frequency or urinary incontinence include flavoxate hydrochloride, oxybutynin hydrochloride, propiverine hydrochloride and the like.

Examples of the therapeutic agent for dysuria include acetylcholine esterase inhibitors (e.g., distigmine) and the like.

Examples of the central D2 receptor antagonist include typical psychotropic drugs (prochlorperazine, haloperidol, chlorpromazine, and the like), serotonin dopamine antagonists (perospirone, risperidone, and the like), and multi-acting receptor targeted antipsychotic drugs (olanzapine and the like).

Examples of the prokinetic agent include peripheral D2 receptor antagonists (metoclopramide, domperidone, and the like) and 5HT4 receptor agonists (mosapride and the like).

Examples of the antihistamine include hydroxyzine, diphenhydramine, and chlorpheniramine.

Examples of the muscarinic receptor antagonist include central muscarinic receptor antagonists (scopolamine and the like) and peripheral muscarinic receptor antagonists (butylscopolamine and the like).

Examples of the serotonin 5HT3 receptor antagonist include granisetron, ondansetron, azasetron, indisetron, palonosetron, and ramosetron.

Examples of the somatostatin analogue include octreotide.

Examples of the corticosteroid include dexamethasone, betamethasone, and methylprednisolone.

Examples of the benzodiazepine anxiolytic include lorazepam and alprazolam, examples of the NK-1 receptor antagonist include aprepitant and fosaprepitant, and examples of the hypercalcemia therapeutic drug include bisphosphonate.

Moreover, a drug confirmed to have a cachexia-ameliorating action either in animal models or clinically, i.e., a cyclooxygenase inhibitor (e.g., indomethacin), a progesterone derivative (e.g., megestrol acetate), glucocorticoid (e.g., dexamethasone), a metoclopramide drug, a tetrahydrocannabinol drug, an agent for improving fat metabolism (e.g., eicosapentaenoic acid), growth hormone, IGF-1, or an antibody against a cachexia-inducing factor TNF-α, LIF, IL-6 or oncostatin M or the like can also be used in combination with the compound of the present disclosure.

Alternatively, a glycation inhibitor (e.g., ALT-711), a nerve regeneration-promoting drug (e.g., Y-128, VX853, prosaptide), an antidepressant (e.g., desipramine, amitriptyline, imipramine), an antiepileptic drug (e.g., lamotrigine, Trileptal, Keppra, Zonegran, Pregabalin, Harkoseride, carbamazepine), an antiarrhythmic drug (e.g., mexiletine), an acetylcholine receptor ligand (e.g., ABT-594), an endothelin receptor antagonist (e.g., ABT-627), a monoamine uptake inhibitor (e.g., tramadol), a narcotic analgesic (e.g., morphine), a GABA receptor agonist (e.g., gabapentin, MR preparation of gabapentin), an α2 receptor agonist (e.g., clonidine), a local analgesic (e.g., capsaicin), an antianxiety drug (e.g., benzothiazepine), a phosphodiesterase inhibitor (e.g., sildenafil), a dopamine receptor agonist (e.g., apomorphine), midazolam, ketoconazole or the like may be used in combination with the compound of the present disclosure.

The time of administration of the GIP receptor agonist peptide of the present disclosure and that of the concomitant drug are not limited, and they may be administered simultaneously or in a staggered manner to the administration subject.

Examples of such administration mode include the following:

(1) administration of a single preparation obtained by simultaneously processing the GIP receptor agonist peptide of the present disclosure and the concomitant drug, (2) simultaneous administration of two kinds of preparations of the GIP receptor agonist peptide of the present disclosure and the concomitant drug, which have been separately produced, by the same administration route, (3) administration of two kinds of preparations of the GIP receptor agonist peptide of the present disclosure and the concomitant drug, which have been separately produced, by the same administration route in a staggered manner, (4) simultaneous administration of two kinds of preparations of the GIP receptor agonist peptide of the present disclosure and the concomitant drug, which have been separately produced, by different administration routes, (5) administration of two kinds of preparations of the compound of the present disclosure and the concomitant drug, which have been separately produced, by different administration routes in a staggered manner (e.g., administration in the order of the GIP receptor agonist peptide of the present disclosure and the concomitant drug, or in the reverse order) and the like.

The dose of the concomitant drug can be appropriately determined based on the dose employed in clinical situations. The mixing ratio of the GIP receptor agonist peptide of the present disclosure and a concomitant drug can be appropriately determined depending on the administration subject, symptom, administration method, target disease, combination and the like. When the subject of administration is human, for example, a concomitant drug can be used in 0.01-100 parts by weight relative to 1 part by weight of the GIP receptor agonist peptide of the present disclosure.

By combining the GIP receptor agonist peptide of the present disclosure and concomitant drug: (1) the dose of the GIP receptor agonist peptide of the present disclosure or a concomitant drug can be reduced as compared to single administration of the GIP receptor agonist peptide of the present disclosure or a concomitant drug,

(2) the drug to be used in combination with the GIP receptor agonist peptide of the present disclosure can be selected depending on the condition of patients (mild, severe and the like),

(3) the period of treatment can be set longer by selecting a concomitant drug having different action and mechanism from those of the GIP receptor agonist peptide of the present disclosure,

(4) a sustained treatment effect can be designed by selecting a concomitant drug having different action and mechanism from those of the GIP receptor agonist peptide of the present disclosure, and

(5) a synergistic effect can be afforded by a combined use of the GIP receptor agonist peptide of the present disclosure and a concomitant drug, and the like, can be achieved.

### F. EXAMPLES

The abbreviations used in the present specification mean the following (Table 2). A hyphen in terms such as α-MePhe and the like as described herein may be omitted, and the event of omission also represents the same meaning.

In the amino acid sequences used in the present specification, the left terminal represents N terminal and the right terminal represents C terminal.

TABLE 2
Commonly used abbreviations in the present disclosure.

| | |
|---|---|
| Ac | acetyl |
| Aib | α-aminoisobutyric acid |
| Ambz (4) | 4-aminomethylbenzoyl |
| GABA | γ-aminobutyric acid |
| Iva | isovaline |
| Lys (Ac) | Nε-acetyllysine |
| α-MePhe | α-methylphenylalanine |
| MeTyr | N-Methyltyrosine |
| Hda | 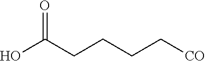 |
| Doda | 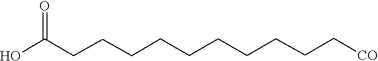 |
| Trda | 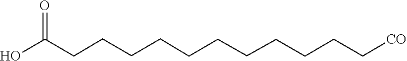 |
| Teda | 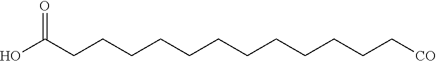 |
| Peda | 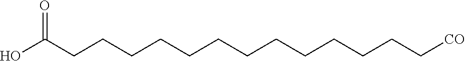 |
| Heda | 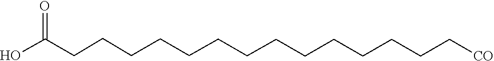 |
| Hepda | 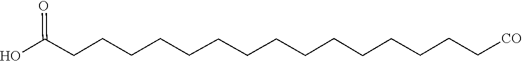 |
| Oda | 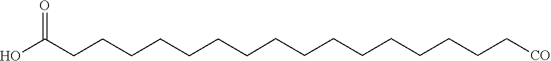 |
| Eda | 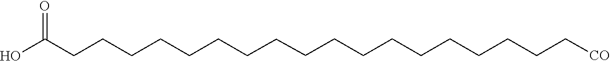 |
| Dda | 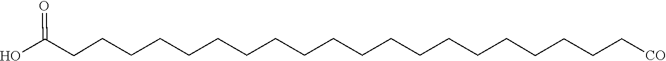 |
| Pal | 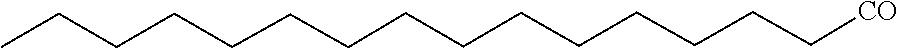 |
| PEG(2) | 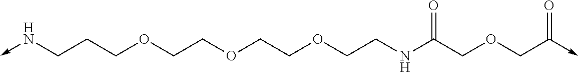 |
| PEG3 | 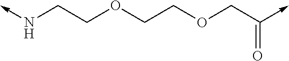 |
| (PEG3) 2 | 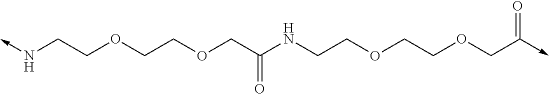 |
| (PEG3) 3 | 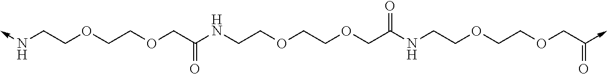 |
| (PEG3) 4 | 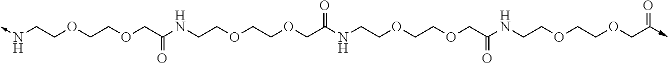 |
| (PEG3) 5 | 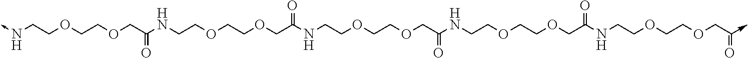 |
| PEG (4) | 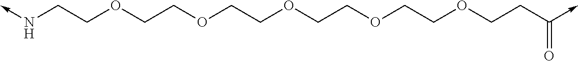 |
| γGlu | 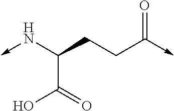 |
| (γGlu) 2 | 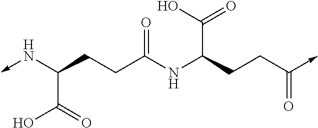 |
| (γGlu) 3 | 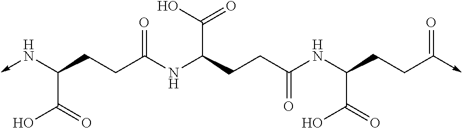 |
| (γGlu) 2- PEG3 | 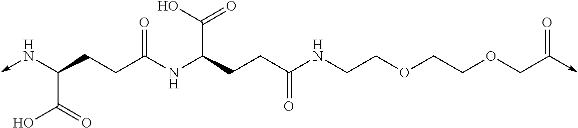 |
| AMBZ-PEG3 | 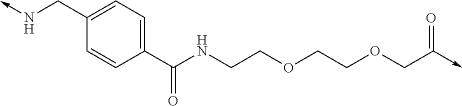 |
| GABA- (PEG3) 2 | 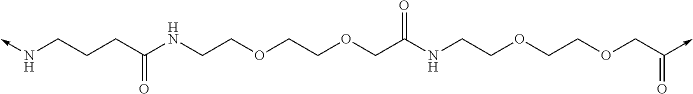 |
| GABA-GGG | 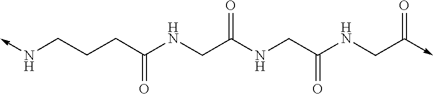 |
| GG | 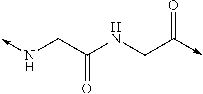 |
| GGG | 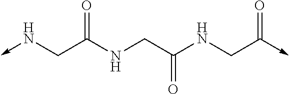 |
| GGGG | 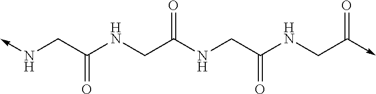 |
| GGGGG | 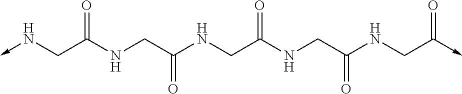 |
| GGGGGG | 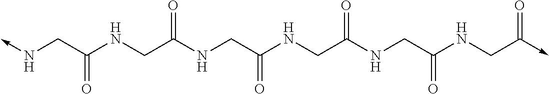 |
| G9 | 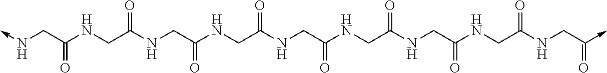 |
| NpipAc | 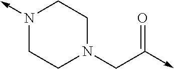 |
| NpipAc- PEG3 | 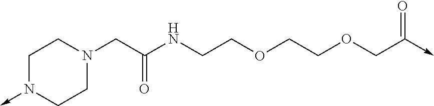 |
| Tra | 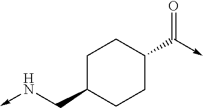 |
| Tra-GGG | 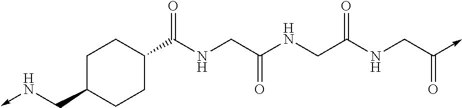 |
| Tra-PEG3 | 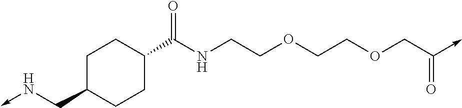 |
| γGlu-PEG3 | 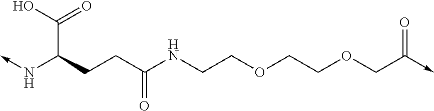 |
| γGlu- (PEG3) 2 | 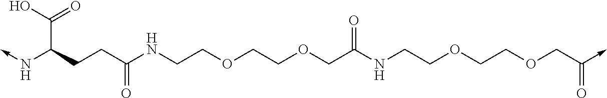 |
| γGlu-AMBZ- PEG3 | 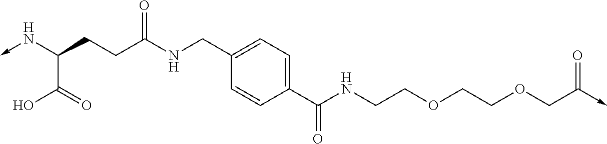 |
| γGlu-GGG | 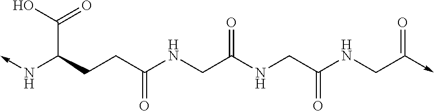 |
| εLys | 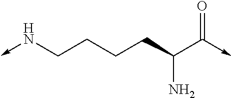 |
| εLys-GGG | 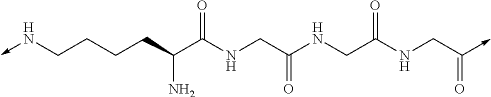 |
| εLys-PEG3 | 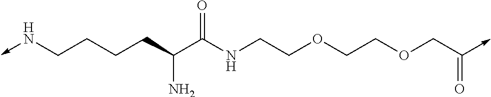 |
| gE | 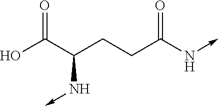 |
| OEG = AEEA = PEG3 | 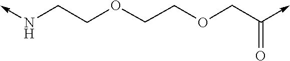 |
| GGGGG | 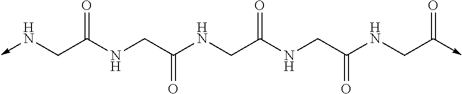 |
| GGEEE = G2E3 | 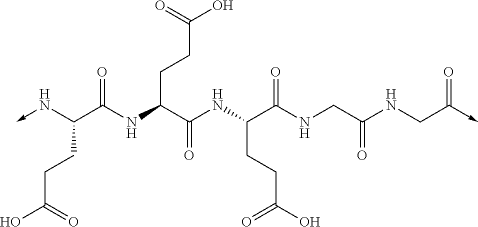 |
| G3gEgE | 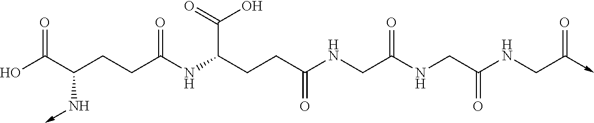 |
| 2OEGgEgE | 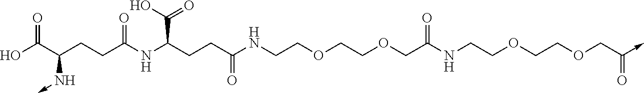 |
| OEGgEgE | 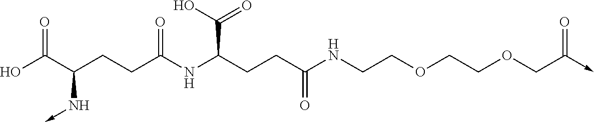 |
| GGPAPAP | 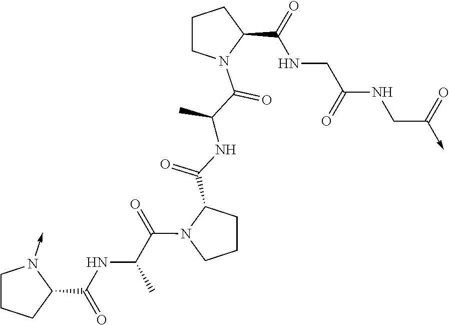 |
| 2OEGgE | 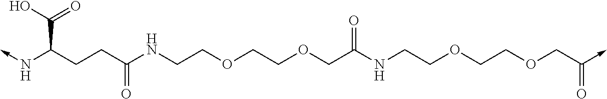 |
| 3OEGgEgE | 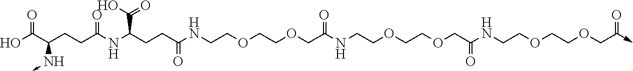 |
| G4gE | 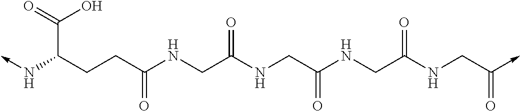 |
| G5gE | 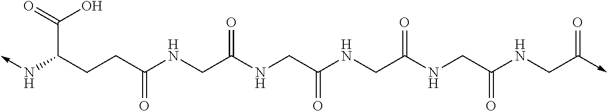 |
| 2OEGgEgEgE | 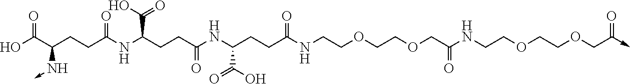 |
| 2OEG | 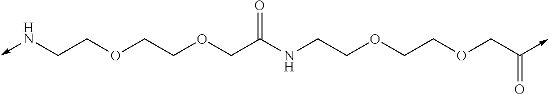 |
| G5gEgE | 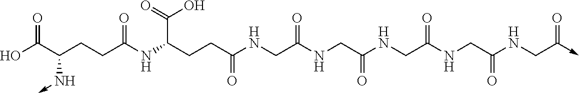 |
| gE = γGlu | 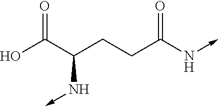 |
| C15DA = C₁₅ diacid | 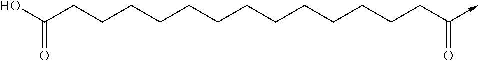 |
| C16DA = C₁₆ diacid | 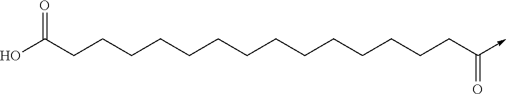 |
| C16 | 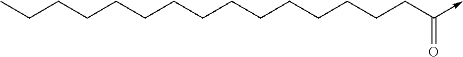 |
| C17DA = C₁₇ diacid | 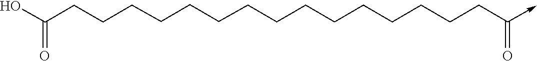 |
| C18DA = C₁₈ diacid | 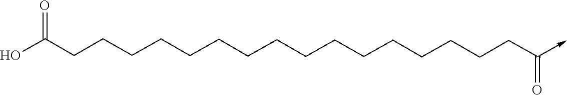 |
| C18 | 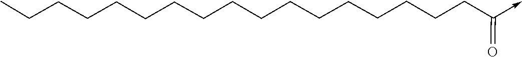 |
| C20DA = C₂₀ diacid | 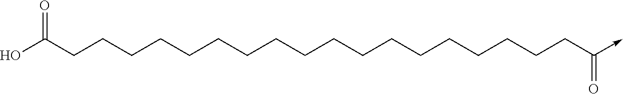 |
| Ac | 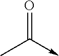 |
| Me-Tyr = MeTyr | 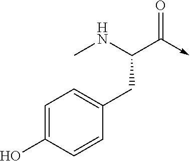 |
| Aib | 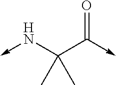 |

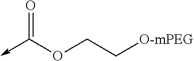

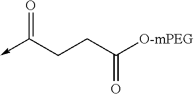

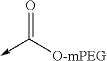

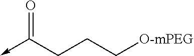

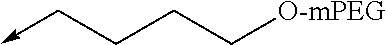

PEG linkers used for Cys. PEG = 5 − 30 kDa PEG

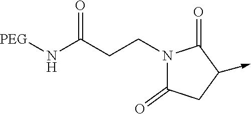

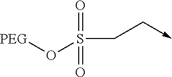

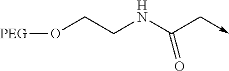

In the specification, where bases, amino acids, etc. are denoted by their codes, they are based on conventional codes in accordance with the IUPAC-IUB Commission on Biochemical Nomenclature or by the common codes in the art, examples of which are shown below. For amino acids that may have an optical isomer, L-form is presented unless otherwise indicated (e.g., “Ala” is L-form of Ala). In addition, “D-” means a D-form (e.g., “D-Ala” is D-form of Ala), and “DL-” means a racemate of a D-form and an L-form (e.g., “DL-Ala” is DL racemate of Ala).

The present disclosure is explained in detail in the following by referring to the following Reference Examples, Examples, Test Examples and Formulation Examples, which are mere embodiments and not to be construed as limitative. In addition, the present disclosure may be modified without departing from the scope of invention.

The term “room temperature” in the following Examples indicates the range of generally from about 10° C. to about 35° C. As for “%”, the yield is in mol/mol %, the solvent used for chromatography is in % by volume and other “%” is in % by weight.

NMP: methylpyrrolidone
THF: tetrahydrofuran

### DMF: N,N-dimethylformamide

WSC: 1-(3-dimethylaminopropyl)-3-ethylcarbodiimide hydrochloride

### DCC: N,N′-Dicyclohexylcarbodiimide

DIPCDI: N,N′-diisopropylcarbodiimide
HOBt: 1-hydroxybenzotriazole monohydrate
Oxyma: ethyl 2-cyano-2-(hydroxyimino)acetate

### Example 1. Synthesis Schemes

Exemplary methods for synthesizing GIP receptor agonist peptides are disclosed for example in Applicant's International PCT Application No. PCT/JP2018/013540, filed on Mar. 30, 2018, ranging from pages 162 to 213, the disclosure of which is specifically incorporated herein by reference in its entirety.

### Example 2. Synthesis of Selective GIP Receptor Agonist Peptides of the Present Disclosure. Compound No. 72; SEQ ID NO: 73

The peptide compound 72 was synthesized using standard Fmoc chemistry.

1. Resin preparation: 1-chloro-2-[chloro-phenyl-(p-tolyl)methyl]benzene (35 mmol, 1.00 eq) was added DIEA (27.2 g, 210 mmol, 36.6 mL, 6.0 eq) and FMOC-GLY-OH (10.4 g, 35 mmol, 1.00 eq) in DCM (600 mL). The mixture was agitated with N₂ for 2 h at 20° C., then added MeOH (70.0 mL) and agitated with N₂ for another 30 min. The resin was washed with DMF (900 mL times 5). Then 20% piperidine in DMF (600 mL) was added and the mixture was agitated with N₂ for 15 min (5 min+10 min) at 20° C. Then the resin was washed with DMF (900 mL times 5) and the mixture was filtered to get the resin.

2. Coupling: a solution of FMOC-ARG(PBF)-OH (68.1 g, 105 mmol, 3.00 eq), HBTU (37.8 g, 99.8 mmol, 2.85 eq) and DIEA (27.2 g, 210 mmol, 36.6 mL, 6.0 eq) in DMF (400 mL) was added to the resin and agitated with N₂ for 30 min at 20° C. The resin was then washed with DMF (900 ml, times 5).

3. Deprotection: 20% piperidine in DMF (600 mL) was added to the resin and the mixture was agitated with N₂ for 15 min (5 min+10 min) at 20° C. The resin was washed with DMF (900 mL times 5) and filtered to get the resin.

4. Repeat step 2 to 3 for next amino acid coupling.

| # | Materials | Coupling reagents |
|---|---|---|
| 1 | FMOC-GLY-OH (1.0 eq) | DIEA (6.0 eq) |
| 2 | FMOC-ARG(PBF)-OH (3.0 eq) | HBTU (2.85 eq) and DIEA (6.0 eq) |
| 3 | FMOC-GLN(TRT)-OH (3.0 eq) | HBTU (2.85 eq) and DIEA (6.0 eq) |
| 4 | FMOC-ALA-OH (3.0 eq) | HBTU (2.85 eq) and DIEA (6.0 eq) |
| 5 | FMOC-LEU-OH (3.0 eq) | HBTU (2.85 eq) and DIEA (6.0 eq) |
| 6 | FMOC-LEU-OH (3.0 eq) | HBTU (2.85 eq) and DIEA (6.0 eq) |
| 7 | FMOC-TRP(BOC)-OH (3.0 eq) | HBTU (2.85 eq) and DIEA (6.0 eq) |
| 8 | FMOC-ASN(TRT)-OH (3.0 eq) | HBTU (2.85 eq) and DIEA (6.0 eq) |
| 9 | FMOC-VAL-OH (3.0 eq) | HBTU (2.85 eq) and DIEA (6.0 eq) |
| 10 | FMOC-PHE-OH (3.0 eq) | HBTU (2.85 eq) and DIEA (6.0 eq) |
| 11 | FMOC-LYS(DDE)-OH (3.0 eq) | HBTU (2.85 eq) and DIEA (6.0 eq) |
| 12 | FMOC-AIB-OH (3.0 eq) | HBTU (2.85 eq) and DIEA (6.0 eq) |
| 13 | FMOC-GLN(TRT)-OH (4.0 eq) | HATU (3.8 eq) and DIEA (8.0 eq) |
| 14 | FMOC-ALA-OH (3.0 eq) | HBTU (2.85 eq) and DIEA (6.0 eq) |
| 15 | FMOC-AIB-OH (3.0 eq) | HBTU (2.85 eq) and DIEA (6.0 eq) |
| 16 | FMOC-ARG(PBF)-OH (4.0 eq) | HOAT (4.0 eq) and DIC (4.0 eq) |
| 17 | FMOC-ASP(OTBU)-OH (3.0 eq) | HBTU (2.85 eq) and DIEA (6.0 eq) |
| 18 | FMOC-LEU-OH (4.0 eq) | HBTU (3.8 eq) and DIEA (8.0 eq) |
| 19 | FMOC-AIB-OH (4.0 eq) | HBTU (3.8 eq) and DIEA (8.0 eq) |
| 20 | FMOC-ILE-OH (4.0 eq) | HATU (3.8 eq) and DIEA (8.0 eq) |
| 21 | FMOC-SER(TBU)-OH (4.0 eq) | HBTU (3.8 eq) and DIEA (8.0 eq) |
| 22 | FMOC-TYR(TBU)-OH (4.0 eq) | HBTU (3.8 eq) and DIEA (8.0 eq) |
| 23 | FMOC-ASP(OTBU)-OH (4.5 eq) | HBTU (3.8 eq) and DIEA (8.0 eq) |
| 24 | FMOC-SER(TBU)-OH (4.0 eq) | HBTU (3.8 eq) and DIEA (8.0 eq) |
| 25 | FMOC-ILE-OH (3.0 eq) | HOBT (3.0 eq) and DIC (8.0 eq) |
| 26 | FMOC-PHE-OH (5.0 eq) | HBTU (4.75 eq) and DIEA (10.0 eq) |
| 27 | FMOC-THR(TBU)-OH (5.0 eq) | HBTU (4.75 eq) and DIEA (10.0 eq) |
| 28 | FMOC-GLY-OH (5.0 eq) | HBTU (4.75 eq) and DIEA (10.0 eq) |
| 29 | FMOC-GLU(OTBU)-OH (6.0 eq) | HATU (5.7 eq) and DIEA (12.0 eq) |
| 30 | FMOC-AIB-OH (5.0 eq) | HBTU (4.75 eq) and DIEA (10.0 eq) |
| 30 | FMOC-N-ME-TYR(TBU)-OH (4.0 eq) | HATU (3.8 eq) and DIEA (8.0 eq) |
| 31 | BOC₂O(5.0 eq) | DIEA (10.0 eq) |

5. Add 3% H2N.NH₂/DMF and react on 700 mL for 30 min (10 min+20 min). Drain and wash with DMF (900 mL) for 5 times.

6. Repeat Step 2 and 3 for all other amino acids: 1-(9H-fluoren-9-yl)-3-oxo-2,7,10-trioxa-4-azadodecan-12-oic acid, 1-(9H-fluoren-9-yl)-3-oxo-2,7,10-trioxa-4-azadodecan-12-oic acid, FMOC-GLU-OTBU, FMOC-GLU-OTBU, 18-(tert-butoxy)-18-oxooctadecanoic acid.

### Peptide Cleavage and Purification:

1. After coupling, the resin was washed with DMF (900 mL) for 5 times. After last step, the resin was washed with MeOH (500 mL) for 4 times, and dried under vacuum. Then the peptide resin (270 g) was treated with the cleavage cocktail (2.5 L, 92.5% TFA/2.5% 3-Mercaptopropionic acid/2.5% TIS/2.5% H₂O) for 2.5 hours at 20° C. The cleavage cocktail was concentrated under reduced pressure to about 900 mL. Then the residue was precipitated with cold isopropyl ether (9.0 L), filtered and washed two times with isopropyl ether (500 mL). Dry the crude peptide under vacuum 2 hours to get 150 g. and LCMS (EW15791-26-P1A1, Rt=1.586 min) showed the desired MS was detected.

2. The crude peptide was purified by Prep-HPLC (A: 0.075% TFA in H2, B: ACN) and then was second purified by Prep-HPLC (A: 0.5% HOAc in H₂O, B: ACN) to give the peptide compound 72 (15.89 g, 3.46 mmol, 9.87% yield, 96.79% purity, HOAC) was obtained as a white solid, which was confirmed by LCMS (Rt=1.567 mi) and HPLC(Rt 10.095 min).

Purification Conditions:

First Purification condition

| | |
|---|---|
| Dissolution | Dissolve in 20% ACN in H₂O |
| condition | |
| Instrument | Gilson GX-281 |
| Mobile Phase | A: H₂O (0.075% TFA in H₂O) |
| | B: ACN |
| Gradient | 16-46-60 min. Retention time: 41 min |
| Column | luna, c18, 10 um, 100 A + Gemini, 5 um, c18, 110 A |
| Flow Rate | 20 mL/Min |
| Wavelength | 214/254 nm |
| Oven Tem. | 50° C. |

Second Purification condition

| | |
|---|---|
| Dissolution | Dissolve in 20% ACN in H₂O |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 35-65-30 min. Retention time: 55 min |
| Column | luna, c18, 10 um, 100 A + Gemini, 5 um, c18, 110 A |
| Flow Rate | 20 mL/Min |
| Wavelength | 214/254 nm |
| Oven Tem. | 30° C. |

### Example 3. Synthesis of Selective GIP Receptor Agonist Peptides of the Present Disclosure. Compound No. 293; SEQ ID NO: 294

The peptide compound 293 was synthesized using standard Fmoc chemistry.

1. Resin preparation: The Rink Amine MBHA resin (1.20 mmol, 1.00 eq, 6.00 g, Sub 0.20 mmol/g) in DMF (50 mL) was agitated with N₂ for 2 hrs at 20° C. Then 20% piperidine in DMF (80 mL) was added and the mixture was agitated with N₂ for 15 min at 20° C. Then the mixture was filtered to get the resin. The resin was washed with DMF (80 mL*5) and filtered to get the resin.

2. Coupling: a solution of FMOC-SER(TBU)-OH (3.00 eq) and HBTU (2.85 eq), DIEA (6.00 eq) in DMF (50 mL) was added to the resin and agitated with N₂ for 30 min at 20° C. The resin was then washed with DMF (80 mL*3).

3. Deprotection: 20% piperidine in DMF (80 mL) was added to the resin and the mixture was agitated with N₂ for 15 min at 20° C. The resin was washed with DMF (80 mL*5) and filtered to get the resin.

4. Repeat step 2 to 3 for the coupling of following amino acids: (1-38)

| # | Materials | Coupling reagents |
|---|---|---|
| 1 | FMOC-PRO-OH(3.00 eq) | HBTU (2.85 eq) and DIEA (6.00 eq) |
| 2 | FMOC-PRO-OH(3.00 eq) | HBTU (2.85 eq) and DIEA (6.00 eq) |
| 3 | FMOC-PRO-OH(3.00 eq) | HBTU (2.85 eq) and DIEA (6.00 eq) |
| 4 | FMOC-ALA-OH(3.00 eq) | HBTU (2.85 eq) and DIEA (6.00 eq) |
| 5 | FMOC-GLY-OH(3.00 eq) | HBTU (2.85 eq) and DIEA (6.00 eq) |
| 6 | FMOC-SER(TBU)-OH(3.00 eq) | HBTU (2.85 eq) and DIEA (6.00 eq) |
| 7 | FMOC-SER(TBU)-OH(3.00 eq) | HBTU (2.85 eq) and DIEA (6.00 eq) |
| 8 | FMOC-PRO-OH(3.00 eq) | HBTU (2.85 eq) and DIEA (6.00 eq) |
| 9 | FMOC-SER(TBU)-OH(3.00 eq) | HBTU (2.85 eq) and DIEA (6.00 eq) |
| 10 | FMOC-GLN(TRT)-OH (3.00 eq) | HBTU (2.85 eq) and DIEA (6.00 eq) |
| 11 | FMOC-ALA-OH (3.00 eq) | HBTU (2.85 eq) and DIEA (6.00 eq) |
| 12 | FMOC-LEU-OH (3.00 eq) | HBTU (2.85 eq) and DIEA (6.00 eq) |
| 13 | FMOC-LEU-OH (3.00 eq) | HBTU (2.85 eq) and DIEA (6.00 eq) |
| 14 | FMOC-TRP(BOC)-OH (3.00 eq) | HBTU (2.85 eq) and DIEA (6.00 eq) |
| 15 | FMOC-ASN(TRT)-OH (3.00 eq) | HBTU (2.85 eq) and DIEA (6.00 eq) |
| 16 | FMOC-VAL-OH (3.00 eq) | HBTU (2.85 eq) and DIEA (6.00 eq) |
| 17 | FMOC-PHE-OH (3.00 eq) | HBTU (2.85 eq) and DIEA (6.00 eq) |
| 18 | FMOC-ASN(TRT)-OH (3.00 eq) | HBTU (2.85 eq) and DIEA (6.00 eq) |
| 19 | FMOC-AIB-OH (3.00 eq) | HBTU (2.85 eq) and DIEA (6.00 eq) |
| 20 | FMOC-GLN(TRT)-OH (3.00 eq) | HATU (2.85 eq) and DIEA (6.00 eq) |
| 21 | FMOC-LYS(DDE)-OH (3.00 eq) | HBTU (2.85 eq) and DIEA (6.00 eq) |
| 22 | FMOC-AIB-OH (3.00 eq) | HBTU (2.85 eq) and DIEA (6.00 eq) |
| 23 | FMOC-ARG(PBF)-OH (3.00 eq) | HATU (2.85 eq) and DIEA (6.00 eq) |
| 24 | FMOC-ASP(OTBU)-OH (3.00 eq) | HBTU (2.85 eq) and DIEA (6.00 eq) |
| 25 | FMOC-LEU-OH (3.00 eq) | HBTU (2.85 eq) and DIEA (6.00 eq) |
| 26 | FMOC-ALA-OH (3.00 eq) | HBTU (2.85 eq) and DIEA (6.00 eq) |
| 27 | FMOC-ILE-OH (3.00 eq) | HBTU (2.85 eq) and DIEA (6.00 eq) |
| 28 | FMOC-SER(TBU)-OH (3.00 eq) | HBTU (2.85 eq) and DIEA (6.00 eq) |
| 29 | FMOC-TYR(TBU)-OH (3.00 eq) | HBTU (2.85 eq) and DIEA (6.00 eq) |
| 30 | FMOC-ASP(OTBU)-OH (3.00 eq) | HBTU (2.85 eq) and DIEA (6.00 eq) |
| 31 | FMOC-SER(TBU)-OH (3.00 eq) | HBTU (2.85 eq) and DIEA (6.00 eq) |
| 32 | FMOC-ILE-OH (3.00 eq) | HBTU (2.85 eq) and DIEA (6.00 eq) |
| 33 | FMOC-PHE-OH (3.00 eq) | HBTU (2.85 eq) and DIEA (6.00 eq) |
| 34 | FMOC-THR(TBU)-OH (3.00 eq) | HBTU (2.85 eq) and DIEA (6.00 eq) |
| 35 | FMOC-GLY-OH (3.00 eq) | HBTU (2.85 eq) and DIEA (6.00 eq) |
| 36 | FMOC-GLU(OTBU)-OH (3.00 eq) | HBTU (2.85 eq) and DIEA (6.00 eq) |
| 37 | FMOC-AIB-OH (3.00 eq) | HBTU (2.85 eq) and DIEA (6.00 eq) |
| 38 | FMOC-N-ME-TYR(TBU)-OH (3.00 eq) | HATU (2.85 eq) and DIEA (6.00 eq) |

5. To a solution of DIEA (10.00 eq) and Boc2O (10.00 eq) in DMF (50 mL) was added to the resin and agitated with N₂ for 1 hour at 20° C. Then the resin was washed with, DMF (80 mL*3).

6. Add 3% N₂H₄.H₂O/DMF and react on 20 min and then repeat it for one more time. Drain and wash with DMF (80 mL*5).

7. Repeat step 2 to 3 for the coupling of following amino acids: (1-4)

| # | Materials | Coupling reagents |
|---|---|---|
| 1 | FMOC-AEEA-OH (3.00 eq) | HBTU (2.85 eq) and DIEA (6.00 eq) |
| 2 | FMOC-AEEA-OH (3.00 eq) | HBTU (2.85 eq) and DIEA (6.00 eq) |
| 3 | FMOC-GLU-OTBU (3.00 eq) | HBTU (2.85 eq) and DIEA (6.00 eq) |
| 4 | FMOC-GLU-OTBU (3.00 eq) | HBTU (2.85 eq) and DIEA (6.00 eq) |

8. Coupling: a solution of 18-(tert-butoxy)-18-oxooctadecanoic acid (2.00 eq) and HOBt (2.00 eq), DIC (2.00 eq) in DMF (50 mL) was added to the resin and agitated with N₂ for 12 hrs at 20° C. The resin was then washed with DMF (80 mL*3).

9. The coupling reaction was monitored by ninhydrin color reaction.

Peptide Cleavage and Purification:

1. The resin was washed with MeOH (80 mL*3) and dried under vacuum to get 12 g peptide resin. Then 100 mL of cleavage buffer (90% TFA/3% 3-Mercaptopropionic acid/3% TIS/4% H2O) was added to the flask containing the side chain protected peptide resin at 20° C. and the mixture was stirred for 2 hrs. The peptide was precipitated with cold isopropyl ether (1000 mL) and centrifuged (3 mi at 3000 rpm). Wash the peptide precipitation with isopropyl ether for two more times. Dry the crude peptide over vacuum for 2 hrs.

2. The crude peptide was purified by Prep-HPLC (A: 0.075% TFA in H2, B: ACN) and then was second purified by Prep-HPLC (A: 0.5% HOAc in H2, B: ACN) to give the peptide compound 293 (423.7 mg, 95.73% purity, HOAC) was obtained as a white solid, which was confirmed by LCMS (Rt=1.605 mi) and HPLC.

Purification Conditions:

First Purification condition

| | |
|---|---|
| Dissolution | Dissolve in 20% TFA-10% ACN-H2O |
| condition | |
| Instrument | Gilson GX-281 |
| Mobile Phase | A: H₂O (0.075% TFA in H₂O) |
| | B: ACN |
| Gradient | 25-45-60 min. Retention time: 50 min |
| Column | luna, c18, 10 um, 100 A + Gemini, 5 um, c18, 110 A |
| Flow Rate | 20 mL/Min |
| Wavelength | 214/254 nm |
| Oven Tem. | 30° C. |

Second Purification condition

| | |
|---|---|
| Dissolution | Dissolve in 20% ACN in H₂O |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 30-45-60 min. Retention time: 61 min |
| Column | luna, c18, 10 um, 100 A + Gemini, 5 um, c18, 110 A |
| Flow Rate | 20 mL/Min |
| Wavelength | 214/254 nm |
| Oven Tem. | 30° C. |

### Example 4. Synthesis of Selective GIP Receptor Agonist Peptides of the Present Disclosure. Compound No. 45; SEQ ID NO: 46

The peptide compound 45 was synthesized using standard Fmoc chemistry.

1. Resin preparation: the 2-CTC Resin (800 mg, 0.400 mmol, 1.00 eq, Sub 0.50 mmol/g) was added Fmoc-Ser(tBu)-OH (153 mg, 0.400 mmol, 1.00 eq) and DIEA (207 mg, 1.60 mmol, 0.279 mL, 4.00 eq) in DCM (5.00 mL). The mixture was agitated with N₂ for 2 h at 25° C., then added MeOH (0.800 mL) agitated with N₂ for another 30 min. The resin was washed with DMF (30.0 mL*5). Then 20% piperidine in DMF (30.0 mL) was added and the mixture was agitated with N₂ for 15 min at 25° C. Then the mixture was filtered to get the resin. The resin was washed with DMF (30.0 mL*5) and filtered to get the resin.

2. Coupling: A solution of Fmoc-Pro-OH (4.5 mg 1.20 mmol, 3.00 eq), DIEA (310 mg, 2.40 mmol, 0.418 mL, 6.00 eq) and HBTU (432 mg, 1.14 mmol, 2.85 eq) in DMF (5.00 mL) was added to the resin and agitated with N₂ for 30 min at 25° C. The resin was then washed with DMF (30.0 mL*5).

3. Deprotection: 20% piperidine in DMF (30.0 mL) was added to the resin and the mixture was agitated with N₂ for 15 min at 25° C.

4. Repeat Step 2 and 3 for the coupling of following amino acids: (1-37):

| # | Materials | Coupling reagents |
|---|---|---|
| 1. | FMOC-PRO-OH (3.00 eq) | HBTU (2.85 eq) and DIEA (6.00 eq) |
| 2. | FMOC-PRO-OH (3.00 eq) | HBTU (2.85 eq) and DIEA (6.00 eq) |
| 3. | FMOC-ALA-OH (3.00 eq) | HBTU (2.85 eq) and DIEA (6.00 eq) |
| 4. | FMOC-GLY-OH (3.00 eq) | HBTU (2.85 eq) and DIEA (6.00 eq) |
| 5. | FMOC-SER(TBU)-OH (3.00 eq) | HBTU (2.85 eq) and DIEA (6.00 eq) |
| 6. | FMOC-SER(TBU)-OH (3.00 eq) | HBTU (2.85 eq) and DIEA (6.00 eq) |
| 7. | FMOC-PRO-OH (3.00 eq) | HBTU (2.85 eq) and DIEA (6.00 eq) |
| 8. | FMOC-SER(TBU)-OH (3.00 eq) | HBTU (2.85 eq) and DIEA (6.00 eq) |
| 9. | FMOC-GLN(TRT)-OH (3.00 eq) | HBTU (2.85 eq) and DIEA (6.00 eq) |
| 10. | FMOC-ALA-OH (3.00 eq) | HBTU (2.85 eq) and DIEA (6.00 eq) |
| 11. | FMOC-LEU-OH (3.00 eq) | HBTU (2.85 eq) and DIEA (6.00 eq) |
| 12. | FMOC-LEU-OH (3.00 eq) | HBTU (2.85 eq) and DIEA (6.00 eq) |
| 13. | FMOC-TRP(BOC)-OH (3.00 eq) | HBTU (2.85 eq) and DIEA (6.00 eq) |
| 14. | FMOC-ASN(TRT)-OH (3.00 eq) | HBTU (2.85 eq) and DIEA (6.00 eq) |
| 15. | FMOC-VAL-OH (3.00 eq) | HBTU (2.85 eq) and DIEA (6.00 eq) |
| 16. | FMOC-PHE-OH (3.00 eq) | HBTU (2.85 eq) and DIEA (6.00 eq) |
| 17. | FMOC-ASN(TRT)-OH (3.00 eq) | HBTU (2.85 eq) and DIEA (6.00 eq) |
| 18. | FMOC-AIB-OH (3.00 eq) | HATU (2.85 eq) and DIEA (6.00 eq) |
| 19. | FMOC-GLN(TRT)-OH (6.00 eq) | HOAT (6.00 eq) and DIC (6.00 eq) |
| 20. | FMOC-LYS(DDE)-OH (3.00 eq) | HBTU (2.85 eq) and DIEA (6.00 eq) |
| 21. | FMOC-AIB-OH (3.00 eq) | HATU (2.85 eq) and DIEA (6.00 eq) |
| 22. | FMOC-ARG(PBF)-OH (6.00 eq) | HATU (5.70 eq) and DIEA (12.0 eq) |
| 23. | FMOC-ASP(OTBU)-OH (3.00 eq) | HBTU (2.85 eq) and DIEA (6.00 eq) |
| 24. | FMOC-LEU-OH (3.00 eq) | HBTU (2.85 eq) and DIEA (6.00 eq) |
| 25. | FMOC-ALA-OH (3.00 eq) | HBTU (2.85 eq) and DIEA (6.00 eq) |
| 26. | FMOC-ILE-OH (3.00 eq) | HBTU (2.85 eq) and DIEA (6.00 eq) |
| 27. | FMOC-SER(TBU)-OH (3.00 eq) | HBTU (2.85 eq) and DIEA (6.00 eq) |
| 28. | FMOC-TYR(TBU)-OH (3.00 eq) | HBTU (2.85 eq) and DIEA (6.00 eq) |
| 29. | FMOC-ASP(OTBU)-OH (3.00 eq) | HBTU (2.85 eq) and DIEA (6.00 eq) |
| 30. | FMOC-SER(TBU)-OH (3.00 eq) | HBTU (2.85 eq) and DIEA (6.00 eq) |
| 31. | FMOC-ILE-OH (3.00 eq) | HBTU (2.85 eq) and DIEA (6.00 eq) |
| 32. | FMOC-PHE-OH (3.00 eq) | HBTU (2.85 eq) and DIEA (6.00 eq) |
| 33. | FMOC-THR(TBU)-OH (3.00 eq) | HBTU (2.85 eq) and DIEA (6.00 eq) |
| 34. | FMOC-GLY-OH (3.00 eq) | HBTU (2.85 eq) and DIEA (6.00 eq) |
| 35. | FMOC-GLU(OTBU)-OH (6.00 eq) | HOAT (6.00 eq) and DIC (6.00 eq) |
| 36. | FMOC-AIB-OH (3.00 eq) | HATU (2.85 eq) and DIEA (6.00 eq) |
| 37. | FMOC-N-ME-TYR(TBU)-OH (4.00 eq) | HATU (3.80 eq) and DIEA (8.00 eq) |

5. Coupling: Boc2O/DIPEA/DMF (10/5/85) 30.0 mL for 30 min, then the resin was washed with DMF (30.0 mL*5).

6. Deprotection: Dde was treated with Hydrazine hydrate/DMF (3/97) 30.0 mL for 30 min, then the resin was washed with DMF (30.0 mL*5).

7. Repeat Step 2 and 3 for the coupling of following amino acids: (1-3):

| | | |
|---|---|---|
| 1 | Fmoc-Gly-Gly-Gly-OH | HBTU (2.85 eq) and DIEA (6.00 eq) |
| | (3.00 eq) | |
| 2 | Fmoc-Gly-Gly-OH (3.00 eq) | HBTU (2.85 eq) and DIEA (6.00 eq) |
| 3 | 18-(tert-butoxy)-18- | HBTU (2.85 eq) and DIEA (6.00 eq) |
| | oxoctadecanoic acid (3.00 eq) | |

Peptide Cleavage and Purification:

1. The resin was washed with MeOH (30.0 mL*3) and dried under vacuum to get 3.20 g peptide resin. Then 32.0 mL of cleavage buffer (92.5% TFA/2.5% 3-Mercaptopropionic acid/2.5% TIS/2.5% H2O) was added to the flask containing the side chain protected peptide resin at 25° C. and the mixture was stirred for 2.5 h. The peptide was precipitated with cold isopropyl ether (200 mL) and centrifuged (3 min at 3000 rpm). Wash the peptide precipitation with tert-butyl methyl ether for two more times (200 mL). Dry the crude peptide over vacuum for 2 h to give the crude peptide (1.40 g).

2. The crude peptide was purified by prep-HPLC (TFA condition; A: 0.075% TFA in H2O, B: CH3CN) to give the peptide, then the peptide compound 45 was purified by prep-HPLC (HOAC condition; A: 0.5% HOAC in H2O, B: ACN) to give the final product peptide compound 45 (181 mg, 99.21% purity, HOAC) was obtained as a white solid.

Purification Conditions:

First Purification condition

| | |
|---|---|
| Dissolution | Dissolve in 30% ACN in H₂O |
| condition | |
| Instrument | Gilson GX-281 |
| Mobile Phase | A: H₂O (0.075% TFA in H₂O) |
| | B: ACN |
| Gradient | 26-46-60 min. Retention time: 40 min |
| Column | luna, c18, 10 um, 100 A + Gemini, 5 um, c18, 110 A |
| Flow Rate | 20 mL/Min |
| Wavelength | 214/254 nm |
| Oven Tem. | 50° C. |

Second Purification condition

| | |
|---|---|
| Dissolution | Dissolve in 30% ACN in H₂O |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 30-60-50 min. Retention time: 65 min |
| Column | luna, c18, 10 um, 100 A + Gemini, 5 um, c18, 110 A |
| Flow Rate | 20 mL/Min |
| Wavelength | 214/254 nm |
| Oven Tem. | 50° C. |

### Example 5. Synthesis of Selective GIP Receptor Agonist Peptides of the Present Disclosure. Compound No. 27; SEQ ID NO: 28

The peptide compound 27 was synthesized using standard Fmoc chemistry.

1. Resin preparation: The Rink Amine MBHA resin (1.20 mmol, 1.00 eq, 6.00 g, Sub 0.20 mmol/g) in DMF (50 mL) was agitated with N₂ for 2 hrs at 20° C. Then 20% piperidine in DMF (80 mL) was added and the mixture was agitated with N₂ for 15 min at 20° C. Then the mixture was filtered to get the resin. The resin was washed with DMF (80 mL*5) and filtered to get the resin.

2. Coupling: a solution of FMOC-SER(TBU)—OH (3.00 eq) and HBTU (2.85 eq), DIEA (6.00 eq) in DMF (50 mL) was added to the resin and agitated with N₂ for 30 min at 20° C. The resin was then washed with DMF (80 mL*3).

3. Deprotection: 20% piperidine in DMF (80 mL) was added to the resin and the mixture was agitated with N₂ for 15 min at 20° C. The resin was washed with DMF (80 mL*5) and filtered to get the resin.

4. Repeat step 2 to 3 for the coupling of following amino acids: (1-38)

| # | Materials | Coupling reagents |
|---|---|---|
| 1 | FMOC-PRO-OH(3.00 eq) | HBTU (2.85 eq) and DIEA (6.00 eq) |
| 2 | FMOC-PRO-OH(3.00 eq) | HBTU (2.85 eq) and DIEA (6.00 eq) |
| 3 | FMOC-PRO-OH(3.00 eq) | HBTU (2.85 eq) and DIEA (6.00 eq) |
| 4 | FMOC-ALA-OH(3.00 eq) | HBTU (2.85 eq) and DIEA (6.00 eq) |
| 5 | FMOC-GLY-OH(3.00 eq) | HBTU (2.85 eq) and DIEA (6.00 eq) |
| 6 | FMOC-SER(TBU)-OH(3.00 eq) | HBTU (2.85 eq) and DIEA (6.00 eq) |
| 7 | FMOC-SER(TBU)-OH(3.00 eq) | HBTU (2.85 eq) and DIEA (6.00 eq) |
| 8 | FMOC-PRO-OH(3.00 eq) | HBTU (2.85 eq) and DIEA (6.00 eq) |
| 9 | FMOC-LYS(DDE)-OH (3.00 eq) | HBTU (2.85 eq) and DIEA (6.00 eq) |
| 10 | FMOC-GLN(TRT)-OH (3.00 eq) | HBTU (2.85 eq) and DIEA (6.00 eq) |
| 11 | FMOC-ALA-OH (3.00 eq) | HBTU (2.85 eq) and DIEA (6.00 eq) |
| 12 | FMOC-LEU-OH (3.00 eq) | HBTU (2.85 eq) and DIEA (6.00 eq) |
| 13 | FMOC-LEU-OH (3.00 eq) | HBTU (2.85 eq) and DIEA (6.00 eq) |
| 14 | FMOC-TRP(BOC)-OH (3.00 eq) | HBTU (2.85 eq) and DIEA (6.00 eq) |
| 15 | FMOC-ASN(TRT)-OH (3.00 eq) | HBTU (2.85 eq) and DIEA (6.00 eq) |
| 16 | FMOC-VAL-OH (3.00 eq) | HBTU (2.85 eq) and DIEA (6.00 eq) |
| 17 | FMOC-PHE-OH (3.00 eq) | HBTU (2.85 eq) and DIEA (6.00 eq) |
| 18 | FMOC-ASN(TRT)-OH (3.00 eq) | HBTU (2.85 eq) and DIEA (6.00 eq) |
| 19 | FMOC-AIB-OH (3.00 eq) | HBTU (2.85 eq) and DIEA (6.00 eq) |
| 20 | FMOC-GLN(TRT)-OH (3.00 eq) | HATU (2.85 eq) and DIEA (6.00 eq) |
| 21 | FMOC-ALA-OH (3.00 eq) | HBTU (2.85 eq) and DIEA (6.00 eq) |
| 22 | FMOC-AIB-OH (3.00 eq) | HBTU (2.85 eq) and DIEA (6.00 eq) |
| 23 | FMOC-ARG(PBF)-OH (3.00 eq) | HATU (2.85 eq) and DIEA (6.00 eq) |
| 24 | FMOC-ASP(OTBU)-OH (3.00 eq) | HBTU (2.85 eq) and DIEA (6.00 eq) |
| 25 | FMOC-LEU-OH (3.00 eq) | HBTU (2.85 eq) and DIEA (6.00 eq) |
| 26 | FMOC-AIB-OH (3.00 eq) | HBTU (2.85 eq) and DIEA (6.00 eq) |
| 27 | FMOC-ILE-OH (3.00 eq) | HBTU (2.85 eq) and DIEA (6.00 eq) |
| 28 | FMOC-SER(TBU)-OH (3.00 eq) | HBTU (2.85 eq) and DIEA (6.00 eq) |
| 29 | FMOC-TYR(TBU)-OH (3.00 eq) | HBTU (2.85 eq) and DIEA (6.00 eq) |
| 30 | FMOC-ASP(OTBU)-OH (3.00 eq) | HBTU (2.85 eq) and DIEA (6.00 eq) |
| 31 | FMOC-SER(TBU)-OH (3.00 eq) | HBTU (2.85 eq) and DIEA (6.00 eq) |
| 32 | FMOC-ILE-OH (3.00 eq) | HBTU (2.85 eq) and DIEA (6.00 eq) |
| 33 | FMOC-PHE-OH (3.00 eq) | HBTU (2.85 eq) and DIEA (6.00 eq) |
| 34 | FMOC-THR(TBU)-OH (3.00 eq) | HBTU (2.85 eq) and DIEA (6.00 eq) |
| 35 | FMOC-GLY-OH (3.00 eq) | HBTU (2.85 eq) and DIEA (6.00 eq) |
| 36 | FMOC-GLU(OTBU)-OH (3.00 eq) | HBTU (2.85 eq) and DIEA (6.00 eq) |
| 37 | FMOC-AIB-OH (3.00 eq) | HBTU (2.85 eq) and DIEA (6.00 eq) |
| 38 | FMOC-N-ME-TYR(TBU)-OH (3.00 eq) | HATU (2.85 eq) and DIEA (6.00 eq) |

5. To a solution of DIEA (10.00 eq) and Boc2O (10.00 eq) in DMF (50 mL) was added to the resin and agitated with N₂ for 1 hour at 20° C. Then the resin was washed with DMF (80 mL*3).

6. Add 3% N₂H₄.H₂O/DMF and react on 20 min and then repeat it for one more time. Drain and wash with DMF (80 mL*5).

7. Repeat step 2 to 3 for the coupling of following amino acids: (1-4)

| # | Materials | Coupling reagents |
|---|---|---|
| 1 | FMOC-AEEA-OH (3.00 eq) | HBTU (2.85 eq) and DIEA (6.00 eq) |
| 2 | FMOC-AEEA-OH (3.00 eq) | HBTU (2.85 eq) and DIEA (6.00 eq) |
| 3 | FMOC-GLU-OTBU (3.00 eq) | HBTU (2.85 eq) and DIEA (6.00 eq) |
| 4 | FMOC-GLU-OTBU (3.00 eq) | HBTU (2.85 eq) and DIEA (6.00 eq) |

7. Coupling: a solution of 18-(tert-butoxy)-18-oxooctadecanoic acid (2.00 eq) and HOBt (2.00 eq), DIC (2.00 eq) in DMF (50 mL) was added to the resin and agitated with N₂ for 12 hrs at 20° C. The resin was then washed with DMF (80 mL*3).

8. The coupling reaction was monitored by ninhydrin color reaction.

Peptide Cleavage and Purification:

1. The resin was washed with MeOH (80 mL*3) and dried under vacuum to get 12 g peptide resin. Then 100 mL of cleavage buffer (90% TFA/3% 3-Mercaptopropionic acid/3% TIS/4% H2O) was added to the flask containing the side chain protected peptide resin at 20° C. and the mixture was stirred for 2 hrs. The peptide was precipitated with cold isopropyl ether (1000 mL) and centrifuged (3 min at 3000 rpm). Wash the peptide precipitation with isopropyl ether for two more times. Dry the crude peptide over vacuum for 2 hrs.

2. The crude peptide was purified by Prep-HPLC (A: 0.075% TFA in H2O, B: ACN) and then was second purified by Prep-HPLC (A: 0.5% HOAc in H2O, B: ACN) to give the peptide compound 27 (267.3 mg, 97.25% purity, HOAC) was obtained as a white solid, which was confirmed by LCMS (Rt=1.637 min) and HPLC.

Purification Conditions:

First Purification condition

| | |
|---|---|
| Dissolution | Dissolve in 10% ACN-H2O |
| condition | |
| Instrument | Gilson GX-281 |
| Mobile Phase | A: H₂O (0.075% TFA in H₂O) |
| | B: ACN |
| Gradient | 23-53-60 min. Retention time: 47 min |
| Column | luna, c18, 10 um, 100 A + Gemini, 5 um, c18, 110 A |
| Flow Rate | 20 mL/Min |
| Wavelength | 214/254 nm |
| Oven Tem. | 30° C. |

Second Purification condition

| | |
|---|---|
| Dissolution | Dissolve in 10% ACN-H2O |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 23-53-60 min. Retention time: 60 min |
| Column | luna, c18, 10 um, 100 A + Gemini, 5 um, c18, 110 A |
| Flow Rate | 20 mL/Min |
| Wavelength | 214/254 nm |
| Oven Tem. | 30° C. |

### Example 6. Synthesis of Selective GIP Receptor Agonist Peptides of the Present Disclosure. Compound No. 50; SEQ ID NO: 51

The peptide compound 5G was synthesized using standard Fmoc chemistry.

1. Resin preparation: The Rink Amine MBHA resin (0.6 mmol, 1.00 eq, 1.88 g, Sub 0.32 mmol/g) in DMF (2 G mL) was agitated with N₂ for 2 hrs at 20° C. Then 2 G % piperidine in DMF (2 G mL) was added and the mixture was agitated with N₂ for 15 min at 20° C. Then the mixture was filtered to get the resin. The resin was washed with DMF (2 G mL*5) and filtered to get the resin.

2. Coupling: a solution of FMOC-SER(TBU)-OH (3.00 eq) and HBTU (2.85 eq), DIEA (6.00 eq) in DMF (1 G mL) was added to the resin and agitated with N₂ for 4 G min at 20° C. The resin was then washed with DMF (20 mL*5).

3. Deprotection: 20% piperidine in DMF (20 mL) was added to the resin and the mixture was agitated with N₂ for 15 min at 20° C. The resin was washed with DMF (20 mL*5) and filtered to get the resin.

4. Repeat step 2 to 3 for the coupling of following amino acids: (1-38)

| # | Materials | Coupling reagents |
|---|---|---|
| 1 | FMOC-PRO-OH(3.00 eq) | HBTU (2.85 eq) and DIEA (6.00 eq) |
| 2 | FMOC-PRO-OH(3.00 eq) | HBTU (2.85 eq) and DIEA (6.00 eq) |
| 3 | FMOC-PRO-OH(3.00 eq) | HBTU (2.85 eq) and DIEA (6.00 eq) |
| 4 | FMOC-ALA-OH(3.0 eq) | HBTU (2.85 eq) and DIEA (6.00 eq) |
| 5 | FMOC-GLY-OH(3.00 eq) | HBTU (2.85 eq) and DIEA (6.00 eq) |
| 6 | FMOC-SER(TBU)-OH(3.00 eq) | HBTU (2.85 eq) and DIEA (6.00 eq) |
| 7 | FMOC-SER(TBU)-OH(3.00 eq) | HBTU (2.85 eq) and DIEA (6.00 eq) |
| 8 | FMOC-PRO-OH(3.00 eq) | HBTU (2.85 eq) and DIEA (6.00 eq) |
| 9 | FMOC-SER(TBU)-OH(3.00 eq) | HBTU (2.85 eq) and DIEA (6.00 eq) |
| 10 | FMOC-GLN(TRT)-OH (3.00 eq) | HBTU (2.85 eq) and DIEA (6.00 eq) |
| 11 | FMOC-ALA-OH (3.00 eq) | HBTU (2.85 eq) and DIEA (6.00 eq) |
| 12 | FMOC-LEU-OH (3.00 eq) | HBTU (2.85 eq) and DIEA (6.00 eq) |
| 13 | FMOC-LEU-OH (3.00 eq) | HBTU (2.85 eq) and DIEA (6.00 eq) |
| 14 | FMOC-TRP(BOC)-OH (3.00 eq) | HBTU (2.85 eq) and DIEA (6.00 eq) |
| 15 | FMOC-ASN(TRT)-OH (3.00 eq) | HBTU (2.85 eq) and DIEA (6.00 eq) |
| 16 | FMOC-VAL-OH (3.00 eq) | HBTU (2.85 eq) and DIEA (6.00 eq) |
| 17 | FMOC-PHE-OH (3.00 eq) | HBTU (2.85 eq) and DIEA (6.00 eq) |
| 18 | FMOC-ASP(OTBU)-OH (3.00 eq) | HBTU (2.85 eq) and DIEA (6.00 eq) |
| 19 | FMOC-AIB-OH (3.00 eq) | HBTU (2.85 eq) and DIEA (6.00 eq) |
| 20 | FMOC-GLN(TRT)-OH (3.00 eq) | HATU (2.85 eq) and DIEA (6.00 eq) |
| 21 | FMOC-ALA-OH (3.00 eq) | HBTU (2.85 eq) and DIEA (6.00 eq) |
| 22 | FMOC-LYS(DDE)-OH (3.00 eq) | HBTU (2.85 eq) and DIEA (6.00 eq) |
| 23 | FMOC-ARG(PBF)-OH (3.00 eq) | HATU (2.85 eq) and DIEA (6.00 eq) |
| 24 | FMOC-ASP(OTBU)-OH (3.00 eq) | HBTU (2.85 eq) and DIEA (6.00 eq) |
| 25 | FMOC-LEU-OH (3.00 eq) | HBTU (2.85 eq) and DIEA (6.00 eq) |
| 26 | FMOC-ALA-OH (3.00 eq) | HBTU (2.85 eq) and DIEA (6.00 eq) |
| 27 | FMOC-ILE-OH (3.00 eq) | HBTU (2.85 eq) and DIEA (6.00 eq) |
| 28 | FMOC-SER(TBU)-OH (3.00 eq) | HBTU (2.85 eq) and DIEA (6.00 eq) |
| 29 | FMOC-TYR(TBU)-OH (3.00 eq) | HBTU (2.85 eq) and DIEA (6.00 eq) |
| 30 | FMOC-ASP(OTBU)-OH (3.00 eq) | HBTU (2.85 eq) and DIEA (6.00 eq) |
| 31 | FMOC-SER(TBU)-OH (3.00 eq) | HBTU (2.85 eq) and DIEA (6.00 eq) |
| 32 | FMOC-ILE-OH (3.00 eq) | HBTU (2.85 eq) and DIEA (6.00 eq) |
| 33 | FMOC-PHE-OH (3.00 eq) | HBTU (2.85 eq) and DIEA (6.00 eq) |
| 34 | FMOC-THR(TBU)-OH (3.00 eq) | HBTU (2.85 eq) and DIEA (6.00 eq) |
| 35 | FMOC-GLY-OH (3.00 eq) | HBTU (2.85 eq) and DIEA (6.00 eq) |
| 36 | FMOC-GLU(OTBU)-OH (3.00 eq) | HBTU (2.85 eq) and DIEA (6.00 eq) |
| 37 | FMOC-AIB-OH (3.00 eq) | HBTU (2.85 eq) and DIEA (6.00 eq) |
| 38 | FMOC-N-ME-TYR(TBU)-OH (3.00 eq) | HATU (2.85 eq) and DIEA (6.00 eq) |

5. To a solution of DIEA (1 mL) and Boc2O (2 mL) in DMF (17 mL) was added to the resin and agitated with N₂ for 1 hour at 20° C. Then the resin was washed with DMF (30 mL*3).

6. Add 3% N₂H₄.H₂O/DMF and react on 20 min and then repeat it for one more time. Drain and wash with DMF (30 mL*5).

7. Repeat step 2 to 3 for the coupling of following amino acids: (1-2)

| # | Materials | Coupling reagents |
|---|---|---|
| 1 | FMOC-GLY-GLY-OH | HBTU (2.85 eq) and DIEA (6.00 eq) |
| | (3.00 eq) | |
| 2 | FMOC-GLY-GLY-GLY-OH | HBTU (2.85 eq) and DIEA (6.00 eq) |
| | (3.00 eq) | |

8. Coupling: a solution of 18-(tert-butoxy)-18-oxohexadecanoic acid (3.00 eq) and DIEA (6.00 eq), HBTU (2.85 eq) in DMF (15 mL) was added to the resin and agitated with N₂ for 1 hrs at 20° C. The resin was then washed with DMF (30 mL*3).

9. The coupling reaction was monitored by ninhydrin color reaction.

Peptide Cleavage and Purification:

1. After coupling, the resin was washed with DMF for 5 times. After last step, the resin was washed with MeOH for 3 times, and dried under vacuum. Then the peptide resin (4 g) was treated with the cleavage cocktail (40 mL, 92.5% TFA/2.5% 3-Mercaptopropionic acid/2.5% TIS/2.5% H2O) for 2.5 hours. The peptide was concentrated under reduced pressure and precipitated with cold isopropyl ether, filtered and washed two times with isopropyl ether to give 1.5 g residue.

2. The crude peptide was purified by Prep-HPLC (A: 0.075% TFA in H2O, B: ACN) and then was second purified by Prep-HPLC (A: 0.5% HOAc in H2O, B: ACN) to give the peptide compound 50 (88 mg, 98.60% purity, HOAC) was obtained as a white solid, which was confirmed by LCMS (Rt=1.630 min) and HPLC (Rt=13.029 min).

Purification Conditions:

First Purification condition

| | |
|---|---|
| Dissolution | Dissolve in 10% TFA in H₂O |
| condition | |
| Instrument | Gilson GX-281 |
| Mobile Phase | A: H₂O (0.075% TFA in H₂O) |
| | B: ACN |
| Gradient | 32-52-60 min. Retention time: 60 min |
| Column | Gemini, 10 um, C18, 100 A + luna, C18, 10 um, |
| | 110 A 50 mm*25 mm |
| Flow Rate | 20 mL/Min |
| Wavelength | 214/254 nm |
| Oven Tem. | 50° C. |

Second Purification condition

| | |
|---|---|
| Dissolution | Dissolve in 20% ACN in H₂O |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 30-60 -60 min. Retention time: 13 min |
| Column | Gemini, 10 um, C18, 100 A + luna, C18, 10 um, |
| | 110 A 50 mm*25 mm |
| Flow Rate | 20 mL/Min |
| Wavelength | 214/254 nm |
| Oven Tem. | 30° C. |

### Example 7. Synthesis of Selective GIP Receptor Agonist Peptides of the Present Disclosure. Compound No. 41; SEQ ID NO: 42

The peptide compound 41 was synthesized using standard Fmoc chemistry.

1. Resin preparation: the 2-CTC Resin (800 mg, 0.400 mmol, 1.00 eq, Sub 0.50 mmol/g) was added Fmoc-Gly-OH (119 mg, 0.400 mmol, 1.00 eq) and DIEA (207 mg, 1.60 mmol, 0.279 mL, 4.00 eq) in DCM (5.00 mL). The mixture was agitated with N₂ for 2 h at 25° C., then added MeOH (0.800 mL) agitated with N₂ for another 30 min. The resin was washed with DMF (30.0 mL*5). Then 20% piperidine in DMF (30.0 mL) was added and the mixture was agitated with N₂ for 15 min at 25° C. Then the mixture was filtered to get the resin. The resin was washed with DMF (30.0 mL*5) and filtered to get the resin.

2. Coupling: A solution of Fmoc-Lys(Boc)-OH (562 mg 1.20 mmol, 3.00 eq), DIEA (310 mg, 2.40 mmol, 0.418 mL, 6.00 eq) and HBTU (432 mg, 1.14 mmol, 2.85 eq) in DMF (5.00 mL) was added to the resin and agitated with N₂ for 30 min at 25° C. The resin was then washed with DMF (30.0 mL*5).

3. Deprotection: 20% piperidine in DMF (30.0 mL) was added to the resin and the mixture was agitated with N₂ for 15 min at 25° C.

4. Repeat Step 2 and 3 for the coupling of following amino acids: (1-29):

| # | Materials | Coupling reagents |
|---|---|---|
| 1. | FMOC-GLN(TRT)-OH (3.00 eq) | HBTU (2.85 eq) and DIEA (6.00 eq) |
| 2. | FMOC-ALA-OH (3.00 eq) | HBTU (2.85 eq) and DIEA (6.00 eq) |
| 3. | FMOC-LEU-OH (3.00 eq) | HBTU (2.85 eq) and DIEA (6.00 eq) |
| 4. | FMOC-LEU-OH (3.00 eq) | HBTU (2.85 eq) and DIEA (6.00 eq) |
| 5. | FMOC-TRP(BOC)-OH (3.00 eq) | HBTU (2.85 eq) and DIEA (6.00 eq) |
| 6. | FMOC-ASN(TRT)-OH (3.00 eq) | HBTU (2.85 eq) and DIEA (6.00 eq) |
| 7. | FMOC-VAL-OH (3.00 eq) | HBTU (2.85 eq) and DIEA (6.00 eq) |
| 8. | FMOC-PHE-OH (3.00 eq) | HBTU (2.85 eq) and DIEA (6.00 eq) |
| 9. | FMOC-LYS(DDE)-OH (3.00 eq) | HBTU (2.85 eq) and DIEA (6.00 eq) |
| 10. | FMOC-AIB-OH (3.00 eq) | HATU (2.85 eq) and DIEA (6.00 eq) |
| 11. | FMOC-GLN(TRT)-OH (6.00 eq) | HOAT (6.00 eq) and DIC (6.00 eq) |
| 12. | FMOC-ALA-OH (3.00 eq) | HBTU (2.85 eq) and DIEA (6.00 eq) |
| 13. | FMOC-AIB-OH (3.00 eq) | HATU (2.85 eq) and DIEA (6.00 eq) |
| 14. | FMOC-ARG(PBF)-OH (6.00 eq) | HATU (5.70 eq) and DIEA (12.00 eq) |
| 15. | FMOC-ASP(OTBU)-OH (3.00 eq) | HBTU (2.85 eq) and DIEA (6.00 eq) |
| 16. | FMOC-LEU-OH (3.00 eq) | HBTU (2.85 eq) and DIEA (6.00 eq) |
| 17. | FMOC-ALA-OH (3.00 eq) | HBTU (2.85 eq) and DIEA (6.00 eq) |
| 18. | FMOC-ILE-OH (3.00 eq) | HBTU (2.85 eq) and DIEA (6.00 eq) |
| 19. | FMOC-SER(TBU)-OH (3.00 eq) | HBTU (2.85 eq) and DIEA (6.00 eq) |
| 20. | FMOC-TYR(TBU)-OH (3.00 eq) | HBTU (2.85 eq) and DIEA (6.00 eq) |
| 21. | FMOC-ASP(OTBU)-OH (3.00 eq) | HBTU (2.85 eq) and DIEA (6.00 eq) |
| 22. | FMOC-SER(TBU)-OH (3.00 eq) | HBTU (2.85 eq) and DIEA (6.00 eq) |
| 23. | FMOC-ILE-OH (3.00 eq) | HBTU (2.85 eq) and DIEA (6.00 eq) |
| 24. | FMOC-PHE-OH (3.00 eq) | HBTU (2.85 eq) and DIEA (6.00 eq) |
| 25. | FMOC-THR(TBU)-OH (3.00 eq) | HBTU (2.85 eq) and DIEA (6.00 eq) |
| 26. | FMOC-GLY-OH (3.00 eq) | HBTU (2.85 eq) and DIEA (6.00 eq) |
| 27. | FMOC-GLU(OTBU)-OH (6.00 eq) | HOAT (6.00 eq) and DIC (6.00 eq) |
| 28. | FMOC-AIB-OH (3.00 eq) | HATU (2.85 eq) and DIEA (6.00 eq) |
| 29. | FMOC-N-ME-TYR(TBU)-OH (4.00 eq) | HATU (3.80 eq) and DIEA (8.00 eq) |

5. Coupling: Boc2O/DIPEA/DMF (10/5/85) 30.0 mL for 30 min, then the resin was washed with DMF (30.0 mL*5).

6. Deprotection: Dde was treated with Hydrazine hydrate/DMF (3/97) 30.0 mL for 30 min, then the resin was washed with DMF (30.0 mL*5).

7. Repeat Step 2 and 3 for the coupling of following amino acids: (1-5):

| | | |
|---|---|---|
| 1 | Fmoc-AEEA-OH (3.00 eq) | HBTU (2.85 eq) and DIEA (6.00 eq) |
| 2 | Fmoc-AEEA-OH (3.00 eq) | HBTU (2.85 eq) and DIEA (6.00 eq) |
| 3 | Fmoc-Glu-OtBu | HBTU (2.85 eq) and DIEA (6.00 eq) |
| 4 | Fmoc-Glu-OtBu | HBTU (2.85 eq) and DIEA (6.00 eq) |
| 5 | 18-(tert-butoxy)-18- | HBTU (2.85 eq) and DIEA (6.00 eq) |
| | oxoctadecanoic acid (3.00 eq) | |

Peptide Cleavage and Purification:

1. The resin was washed with MeOH (30.0 mL*3) and dried under vacuum to get 3.00 g peptide resin. Then 30.0 mL of cleavage buffer (92.5% TFA/2.5% 3-Mercaptopropionic acid/2.5% TIS/2.5% H2O) was added to the flask containing the side chain protected peptide resin at 25° C. and the mixture was stirred for 2.5 h. The peptide was precipitated with cold isopropyl ether (200 mL) and centrifuged (3 min at 3000 rpm). Wash the peptide precipitation with tert-butyl methyl ether for two more times (200 mL). Dry the crude peptide over vacuum for 2 h to give the crude peptide (1.20 g).

2. The crude peptide was purified by prep-HPLC (TFA condition; A: 0.075% TFA in H2O, B: CH3CN) to give the peptide, then the peptide was purified by prep-HPLC (HOAC condition; A: 0.5% HOAC in H2O, B: ACN) to give the final product peptide compound 41 (120 mg, 96.8% purity, HOAC) was obtained as a white solid.

Purification Conditions:

First Purification condition

| | |
|---|---|
| Dissolution | Dissolve in 20% ACN in H₂O |
| condition | |
| Instrument | Gilson GX-281 |
| Mobile Phase | A: H₂O (0.075% TFA in H₂O) or |
| | B: ACN |
| Gradient | 23-53-60 min. Retention time: 40 min |
| Column | luna, c18, 10 um, 100 A + Gemini, 5 um, c18, 110 A |
| Flow Rate | 20 mL/Min |
| Wavelength | 214/254 nm |
| Oven Tem. | 50° C. |

Second Purification condition

| | |
|---|---|
| Dissolution | Dissolve in 20% ACN in H₂O |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 26-46-50 min. Retention time: 70 min |
| Column | luna, c18, 10 um, 100 A + Gemini, 5 um, c18, 110 A |
| Flow Rate | 20 mL/Min |
| Wavelength | 214/254 nm |
| Oven Tem. | 50° C. |

### Example 8. Synthesis of Selective GIP Receptor Agonist Peptides of the Present Disclosure. Compound No. 294; SEQ ID NO: 295

The peptide compound 294 was synthesized using standard Fmoc chemistry.

1. Resin preparation: the 2-CTC Resin (1.20 g, 0.600 mmol, 1.00 eq, Sub 0.50 mmol/g) was added FMOC-GLY-OH (179 mg, 0.600 mmol, 1.00 eq) and DIEA (310 mg, 2.40 mmol, 0.40 mL, 4.00 eq) in DCM (10 mL). The mixture was agitated with N₂ for 2 h at 25° C., then added MeOH (1.20 mL), agitated with N₂ for another 30 m. The resin was washed with DMF (20 mL*5). Then 20% piperidine in DMF (20 mL) was added and the mixture was agitated with N₂ for 15 min at 25° C. Then the mixture was filtered to get the resin. The resin was washed with DMF (20 mL*5) and filtered to get the resin.

2. Coupling: A solution of FMOC-ARG(PBF)-OH (1.17 g 1.80 mmol, 3.00 eq), DIEA (465 mg, 3.60 mmol, 0.60 mL, 6.00 eq) and HBTU (646 mg, 1.71 mmol, 2.85 eq) in DMF (10 mL) was added to the resin and agitated with N₂ for 35 me at 25° C. The resin was then washed with DMF (20 mL*5).

3. Deprotection: 20% piperidine in DMF (20 mL) was added to the resin and the mixture was agitated with N₂ for 15 min at 25° C.

4. Repeat step 2 to 3 for the coupling of following amino acids: (1-29)

| # | Materials | Coupling reagents |
|---|---|---|
| 1 | FMOC-GLN(TRT)-OH (3.00 eq) | HBTU (2.85 eq) and DIEA (6.00 eq) |
| 2 | FMOC-ALA-OH (3.00 eq) | HBTU (2.85 eq) and DIEA (6.00 eq) |
| 3 | FMOC-LEU-OH (3.00 eq) | HBTU (2.85 eq) and DIEA (6.00 eq) |
| 4 | FMOC-LEU-OH (3.00 eq) | HBTU (2.85 eq) and DIEA (6.00 eq) |
| 5 | FMOC-TRP(BOC)-OH (3.00 eq) | HBTU (2.85 eq) and DIEA (6.00 eq) |
| 6 | FMOC-ASN(TRT)-OH (3.00 eq) | HBTU (2.85 eq) and DIEA (6.00 eq) |
| 7 | FMOC-VAL-OH (3.00 eq) | HBTU (2.85 eq) and DIEA (6.00 eq) |
| 8 | FMOC-PHE-OH (3.00 eq) | HBTU (2.85 eq) and DIEA (6.00 eq) |
| 9 | FMOC-ASP(OTBU)-OH (3.00 eq) | HBTU (2.85 eq) and DIEA (6.00 eq) |
| 10 | FMOC-AIB-OH (3.00 eq) | HBTU (2.85 eq) and DIEA (6.00 eq) |
| 11 | FMOC-GLN(TRT)-OH (3.00 eq) | HATU (2.85 eq) and DIEA (6.00 eq) |
| 12 | FMOC-ALA-OH (3.00 eq) | HBTU (2.85 eq) and DIEA (6.00 eq) |
| 13 | FMOC-AIB-OH (3.00 eq) | HBTU (2.85 eq) and DIEA (6.00 eq) |
| 14 | FMOC-ARG(PBF)-OH (3.00 eq) | HATU (2.85 eq) and DIEA (6.00 eq) |
| 15 | FMOC-ASP(OTBU)-OH (3.00 eq) | HBTU (2.85 eq) and DIEA (6.00 eq) |
| 16 | FMOC-LYS(DDE)-OH (3.00 eq) | HBTU (2.85 eq) and DIEA (6.00 eq) |
| 17 | FMOC-AIB-OH (3.00 eq) | HBTU (2.85 eq) and DIEA (6.00 eq) |
| 18 | FMOC-ILE-OH (3.00 eq) | HATU (2.85 eq) and DIEA (6.00 eq) |
| 19 | FMOC-SER(TBU)-OH (3.00 eq) | HBTU (2.85 eq) and DIEA (6.00 eq) |
| 20 | FMOC-TYR(TBU)-OH (3.00 eq) | HBTU (2.85 eq) and DIEA (6.00 eq) |
| 21 | FMOC-ASP(OTBU)-OH (3.00 eq) | HBTU (2.85 eq) and DIEA (6.00 eq) |
| 22 | FMOC-SER(TBU)-OH (3.00 eq) | HBTU (2.85 eq) and DIEA (6.00 eq) |
| 23 | FMOC-ILE-OH (3.00 eq) | HATU (2.85 eq) and DIEA (6.00 eq) |
| 24 | FMOC-PHE-OH (3.00 eq) | HATU (2.85 eq) and DIEA (6.00 eq) |
| 25 | FMOC-THR(TBU)-OH (3.00 eq) | HATU (2.85 eq) and DIEA (6.00 eq) |
| 26 | FMOC-GLY-OH (3.00 eq) | HATU (2.85 eq) and DIEA (6.00 eq) |
| 27 | FMOC-GLU(OTBU)-OH (4.00 eq) | HATU (3.80 eq) and DIEA (8.00 eq) |
| 28 | FMOC-AIB-OH (3.00 eq) | HATU (2.85 eq) and DIEA (6.00 eq) |
| 29 | FMOC-N-ME-TYR(TBU)-OH (3.00 eq) | HATU (2.85 eq) and DIEA (6.00 eq) |

5. To a solution of DIEA (2 mL) and Boc2O (4 mL) in DMF (34 mL) was added to the resin and agitated with N₂ for 1 hour at 20° C. Then the resin was washed with DMF (30 mL*3).

6. Add 3% N₂H₄.H₂O/DMF and react on 20 min and then repeat it for one more time. Drain and wash with DMF (30 mL*5).

7. Repeat step 2 to 3 for the coupling of following amino acids: (1-2)

| # | Materials | Coupling reagents |
|---|---|---|
| 1 | FMOC-GLY-GLY-OH | HBTU (2.85 eq) and DIEA (6.00 eq) |
| | (3.00 eq) | |
| 2 | FMOC-GLY-GLY-GLY-OH | HBTU (2.85 eq) and DIEA (6.00 eq) |
| | (3.00 eq) | |

8. Coupling: a solution of 18-(tert-butoxy)-18-oxohexadecanoic acid (3.00 eq) and DIEA (6.00 eq), HBTU (2.85 eq) in DMF (15 mL) was added to the resin and agitated with N₂ for 1 hrs at 20° C. The resin was then washed with DMF (30 mL*3).

9. The coupling reaction was monitored by ninhydrin color reaction.

Peptide Cleavage and Purification:

1. After coupling, the resin was washed with DMF for 5 times. After last step, the resin was washed with MeOH for 3 times, and dried under vacuum. Then the peptide resin (3.8 g) was treated with the cleavage cocktail (40 mL, 92.5% TFA/2.5% 3-Mercaptopropionic acid/2.5% TIS/2.5% H2O) for 2.5 hours. The peptide was concentrated under reduced pressure and precipitated with cold isopropyl ether, filtered and washed two times with isopropyl ether to give 1.4 g residue.

2. The crude peptide was purified by Prep-HPLC (A: 0.075% TFA in H2, B: ACN) and then was second purified by Prep-HPLC (A: 0.5% HOAc in H2, B: ACN) to give the compound 294 (287.2 mg, 98.74% purity, HOAC) was obtained as a white solid, which was confirmed by LCMS (Rt=1.605 mi) and HPLC (Rt=11.541 min).

Purification Conditions:

First Purification condition

| | |
|---|---|
| Dissolution | Dissolve in 10% TFA in H₂O |
| condition | |
| Instrument | Gilson GX-281 |
| Mobile Phase | A: H₂O (0.075% TFA in H₂O) |
| | B: ACN |
| Gradient | 23-53-60 min. Retention time: 39 min |
| Column | Gemini, 10 um, C18, 100 A + luna, C18, 10 um, |
| | 110 A 50 mm*25 mm |
| Flow Rate | 20 mL/Min |
| Wavelength | 214/254 nm |
| Oven Tem. | 50° C. |

Second Purification condition

| | |
|---|---|
| Dissolution | Dissolve in 20% ACN in H₂O |
| condition | |
| Instrument | Gilson GX-281 |
| Mobile Phase | A: H₂O (0.5% HOAc in H₂O) |
| | B: ACN |
| Gradient | 0.4M NH₄Ac 25 min, 0.5% HOAc 10 min, |
| | 30-60 -60 min. Retention time: 11.57 min |
| Column | Gemini, 10 um, C18, 100 A + luna, C18, 10 um, |
| | 110 A 50 mm*25 mm |
| Flow Rate | 20 mL/Min |
| Wavelength | 214/254 nm |
| Oven Tem. | 30° C. |

Table 3 below lists exemplary GIP receptor agonist peptides made according to methods described in Example 1-8.

TABLE 3
Exemplary GIP receptor agonist peptides made according to the methods provided in Examples 1-8.

| CPMD | | | N- | C- | | | | | | | | | | | | |
|---|---|---|---|---|---|---|---|---|---|---|---|---|---|---|---|---|
| No. | LINKER | LIPID | TER | TER | 1 | 2 | 3 | 4 | 5 | 6 | 7 | 8 | 9 | 10 | 11 | 12 |
| 1 | 2OEGgE | C18DA | Me | NH2 | Y | Aib | E | G | T | F | I | S | D | Y | S | I |
| 2 | 2OEGgE | C18DA | Me | NH2 | Y | Aib | E | G | T | F | I | S | D | Y | S | I |
| 3 | 2OEGgE | C18DA | Me | NH2 | Y | Aib | E | G | T | F | I | S | D | Y | S | I |
| 4 | 2OEGgE | C18DA | Me | NH2 | Y | Aib | E | G | T | F | I | S | D | Y | S | I |
| 5 | 2OEGgE | C18DA | Me | NH2 | Y | Aib | E | G | T | F | I | S | D | Y | S | I |
| 6 | 2OEGgE | C18DA | Me | NH2 | Y | Aib | E | G | T | F | I | S | D | Y | S | I |
| 7 | 2OEGgEgE | C17DA | Me | OH | Y | Aib | E | G | T | F | I | S | D | Y | S | I |
| 8 | 2OEGgEgE | C17DA | Me | NH2 | Y | Aib | E | G | T | F | I | S | D | Y | S | I |
| 9 | 2OEGgEgE | C17DA | Me | OH | Y | Aib | E | G | T | F | I | S | D | Y | S | I |
| 10 | 2OEGgE | C17DA | Me | OH | Y | Aib | E | G | T | F | I | S | D | Y | S | I |
| 11 | 2OEGgEgE | C17DA | Me | NH2 | Y | Aib | E | G | T | F | I | S | D | Y | S | I |
| 12 | gEgEgE | C17DA | Me | NH2 | Y | Aib | E | G | T | F | I | S | D | Y | S | I |
| 13 | 2OEGgEgE | C17DA | Me | NH2 | Y | Aib | E | G | T | F | I | S | D | Y | S | I |
| 14 | 2OEGgEgE | C17DA | Me | NH2 | Y | Aib | E | G | T | F | I | S | D | Y | S | I |
| 15 | 2OEGgEgE | C17DA | Me | NH2 | Y | Aib | E | G | T | F | I | S | D | Y | S | I |
| 16 | 2OEGgEgE | C17DA | Me | NH2 | Y | Aib | E | G | T | F | I | S | D | Y | S | I |
| 17 | 2OEGgEgE | C18DA | Me | NH2 | Y | Aib | E | G | T | F | I | S | D | Y | S | I |
| 18 | 2OEGgEgE | C18DA | Me | NH2 | Y | Aib | E | G | T | F | I | S | D | Y | S | I |
| 19 | 2OEGgEgE | C18DA | Me | NH2 | Y | Aib | E | G | T | F | I | S | D | Y | S | I |
| 20 | G5gE | C17DA | Me | OH | Y | Aib | E | G | T | F | I | S | D | Y | S | I |
| 21 | 2OEGgEgE | C18DA | Me | NH2 | Y | Aib | E | G | T | F | I | S | D | Y | S | I |
| 22 | G5gE | C17DA | Me | NH2 | Y | Aib | E | G | T | F | I | S | D | Y | S | I |
| 23 | 2OEGgE | C18DA | Me | NH2 | Y | Aib | E | G | T | F | I | S | D | Y | S | I |
| 24 | G5gE | C18DA | Me | NH2 | Y | Aib | E | G | T | F | I | S | D | Y | S | I |
| 25 | 2OEGgE | C17DA | Me | NH2 | Y | Aib | E | G | T | F | I | S | D | Y | S | I |
| 26 | 2OEGgEgE | C18DA | Me | NH2 | Y | Aib | E | G | T | F | I | S | D | Y | S | I |
| 27 | 2OEGgEgE | C18DA | Me | NH2 | Y | Aib | E | G | T | F | I | S | D | Y | S | I |
| 28 | 2OEGgEgE | C18DA | Me | NH2 | Y | Aib | E | G | T | F | I | S | D | Y | S | I |
| 29 | 2OEGgEgE | C17DA | Me | NH2 | Y | Aib | E | G | T | F | I | S | D | Y | S | I |
| 30 | 2OEGgEgE | C17DA | Me | NH2 | Y | Aib | E | G | T | F | I | S | D | Y | S | I |
| 31 | 2OEGgEgE | C17DA | Me | NH2 | Y | Aib | E | G | T | F | I | S | D | Y | S | I |
| 32 | 2OEGgEgE | C17DA | Me | NH2 | Y | Aib | E | G | T | F | I | S | D | Y | S | I |
| 33 | 2OEGgEgE | C17DA | Me | NH2 | Y | Aib | E | G | T | F | I | S | D | Y | S | I |
| 34 | G5gEgE | C18DA | Me | NH2 | Y | Aib | E | G | T | F | I | S | D | Y | S | I |
| 35 | 2OEGgEgE | C18DA | Me | NH2 | Y | Aib | E | G | T | F | I | S | D | Y | S | I |
| 36 | 2OEGgEgE | C17DA | Me | OH | Y | Aib | E | G | T | F | I | S | D | Y | S | I |
| 37 | 2OEGgEgE | C18DA | Me | OH | Y | Aib | E | G | T | F | I | S | D | Y | S | I |
| 38 | G5 | C18DA | Me | NH2 | Y | Aib | E | G | T | F | I | S | D | Y | S | I |
| 39 | 2OEGgEgE | C20DA | Me | OH | Y | Aib | E | G | T | F | I | S | D | Y | S | I |
| 40 | G5 | C20DA | Me | OH | Y | Aib | E | G | T | F | I | S | D | Y | S | I |
| 41 | 2OEGgEgE | C18DA | Me | OH | Y | Aib | E | G | T | F | I | S | D | Y | S | I |
| 42 | 2OEGgEgE | C18DA | Me | OH | Y | Aib | E | G | T | F | I | S | D | Y | S | I |
| 43 | G5 | C18DA | Me | OH | Y | Aib | E | G | T | F | I | S | D | Y | S | I |
| 44 | G5 | C18DA | Me | OH | Y | Aib | E | G | T | F | I | S | D | Y | S | I |
| 45 | G5 | C18DA | Me | OH | Y | Aib | E | G | T | F | I | S | D | Y | S | I |
| 46 | 2OEGgEgE | C18DA | Me | OH | Y | Aib | E | G | T | F | I | S | D | Y | S | I |
| 47 | G5 | C18DA | Me | OH | Y | Aib | E | G | T | F | I | S | D | Y | S | I |
| 48 | G5 | C18DA | Me | NH2 | Y | Aib | E | G | T | F | I | S | D | Y | S | I |
| 49 | 2OEGgEgE | C18DA | Me | OH | Y | Aib | E | G | T | F | I | S | D | Y | S | I |
| 50 | G5 | C18DA | Me | NH2 | Y | Aib | E | G | T | F | I | S | D | Y | S | I |
| 51 | G5 | C18DA | Me | OH | Y | Aib | E | G | T | F | I | S | D | Y | S | I |
| 52 | G5 | C180A | Me | OH | Y | Aib | E | G | T | F | I | S | D | Y | S | I |
| 53 | G5 | C18DA | Me | OH | Y | Aib | E | G | T | F | I | S | D | Y | S | I |
| 54 | G5 | C18DA | Me | OH | Y | Aib | E | G | T | F | I | S | D | Y | S | I |
| 55 | 2OEGgEgE | C18DA | Me | OH | Y | Aib | E | G | T | F | I | S | D | Y | S | I |
| 56 | 2OEGgEgE | C18DA | Me | OH | Y | Aib | E | G | T | F | I | S | D | Y | S | I |
| 57 | 2OEGgEgE | C18DA | Me | NH2 | Y | Aib | E | G | T | F | I | S | D | Y | S | I |
| 58 | 2OEGgEgE | C18DA | Me | NH2 | Y | Aib | E | G | T | F | I | S | D | Y | S | I |
| 59 | 2OEGgEgE | C18DA | Me | NH2 | Y | Aib | E | G | T | F | I | S | D | Y | S | I |
| 60 | G5 | C18DA | Me | OH | Y | Aib | E | G | T | F | I | S | D | Y | S | I |
| 61 | 2OEGgEgE | C18DA | Me | OH | Y | Aib | E | G | T | F | I | S | D | Y | S | I |
| 62 | 2OEGgEgE | C18DA | Me | OH | Y | Aib | E | G | T | F | I | S | D | Y | S | I |
| 63 | G5 | C18DA | Me | OH | Y | Aib | E | G | T | F | I | S | D | Y | S | I |
| 64 | G6 | C18DA | Me | OH | Y | Aib | E | G | T | F | I | S | D | Y | S | I |
| 65 | 2OEGgEgE | C20DA | Me | OH | Y | Aib | E | G | T | F | I | S | D | Y | S | I |
| 66 | 2OEGgEgE | C18DA | Me | OH | Y | Aib | E | G | T | F | I | S | D | Y | S | I |
| 67 | G5 | C20DA | Me | OH | Y | Aib | E | G | T | F | I | S | D | Y | S | I |
| 68 | 2OEGgEgE | C20DA | Me | NH2 | Y | Aib | E | G | T | F | I | S | D | Y | S | I |
| 69 | 2OEGgEgE | C20DA | Me | NH2 | Y | Aib | E | G | T | F | I | S | D | Y | S | I |
| 70 | G5 | C18DA | Me | OH | Y | Aib | E | G | T | F | I | S | D | Y | S | I |
| 71 | G5 | C18DA | Me | OH | Y | Aib | E | G | T | F | I | S | D | Y | S | I |
| 72 | 2OEGgEgE | C18DA | Me | OH | Y | Aib | E | G | T | F | I | S | D | Y | S | I |
| 73 | 2OEGgEgE | C18DA | Me | OH | Y | Aib | E | G | T | F | I | S | D | Y | S | I |
| 74 | 2OEGgEgE | C18DA | Me | OH | Y | Aib | E | G | T | F | I | S | D | Y | S | I |
| 75 | G5 | C18DA | Me | OH | Y | Aib | E | G | T | F | I | S | D | Y | S | I |
| 76 | G5 | C18DA | Me | OH | Y | Aib | E | G | T | F | I | S | D | Y | S | I |
| 77 | G5 | C18DA | Me | OH | Y | Aib | E | G | T | F | I | S | D | Y | S | I |
| 78 | G5 | C18DA | Me | OH | Y | Aib | E | G | T | F | I | S | D | Y | S | I |
| 79 | G4gE | C18DA | Me | OH | Y | Aib | E | G | T | F | I | S | D | Y | S | I |
| 80 | G4gE | C18DA | Me | OH | Y | Aib | E | G | T | F | I | S | D | Y | S | I |
| 81 | G5 | C18DA | Me | OH | Y | Aib | E | G | T | F | I | S | D | Y | S | I |
| 82 | G5 | C18DA | Me | OH | Y | Aib | E | G | T | F | I | S | D | Y | S | I |
| 83 | G5 | C18DA | Me | OH | Y | Aib | E | G | T | F | I | S | D | Y | S | I |
| 84 | G5 | C18DA | Me | OH | Y | Aib | E | G | T | F | I | S | D | Y | S | I |
| 85 | 2OEGgEgE | C18DA | Me | OH | Y | Aib | E | G | T | F | I | S | D | Y | S | I |
| 86 | 2OEGgEgE | C18DA | Me | OH | Y | Aib | E | G | T | F | I | S | D | Y | S | I |
| 87 | 2OEGgEgE | C18DA | Me | OH | Y | Aib | E | G | T | F | I | S | D | Y | S | I |
| 88 | G5 | C18DA | Me | OH | Y | Aib | E | G | T | F | I | S | D | Y | S | I |
| 89 | 2OEGgEgE | C18DA | Me | NH2 | Y | Aib | E | G | T | F | I | S | D | Y | S | I |
| 90 | 2OEGgEgE | C18DA | Me | NH2 | Y | Aib | E | G | T | F | I | S | D | Y | S | I |
| 91 | 2OEGgE | C18DA | Me | NH2 | Y | Aib | E | G | T | F | I | S | D | Y | S | I |
| 92 | OEGgEOEGgE | C18DA | Me | NH2 | Y | Aib | E | G | T | F | I | S | D | Y | S | I |
| 93 | OEGgEgE | C18DA | Me | NH2 | Y | Aib | E | G | T | F | I | S | D | Y | S | I |
| 94 | 2OEGgEgE | C17DA | Me | NH2 | Y | Aib | E | G | T | F | I | S | D | Y | S | I |
| 95 | G5 | C17DA | Me | OH | Y | Aib | E | G | T | F | I | S | D | Y | S | I |
| 96 | G5 | C18DA | Me | OH | Y | Aib | E | G | T | F | I | S | D | Y | S | I |
| 97 | 2OEGgEgE | C18DA | Me | NH2 | Y | Aib | E | G | T | F | I | S | D | Y | S | I |
| 98 | G5 | C17DA | Me | OH | Y | Aib | E | G | T | F | I | S | D | Y | S | I |
| 99 | 2OEGgEgE | C20DA | Me | NH2 | Y | Aib | E | G | T | F | I | S | D | Y | S | I |
| 100 | 2OEGgEgE | C18DA | Me | NH2 | Y | Aib | E | G | T | F | I | S | D | Y | S | I |
| 101 | 2OEGgEgE | C20DA | Me | NH2 | Y | Aib | E | G | T | F | I | S | D | Y | S | I |
| 102 | GGPAPAP | C18DA | Me | OH | Y | Aib | E | G | T | F | I | S | D | Y | S | I |
| 103 | GGPAPAP | C18DA | Me | OH | Y | Aib | E | G | T | F | I | S | D | Y | S | I |
| 104 | GGPAPAPgE | C18DA | Me | OH | Y | Aib | E | G | T | F | I | S | D | Y | S | I |
| 105 | GGPAPAP | C18DA | Me | OH | Y | Aib | E | G | T | F | I | S | D | Y | S | I |
| 106 | 2OEGgEgE | C18DA | Me | NH2 | Y | Aib | E | G | T | F | I | S | D | Y | S | I |
| 107 | 2OEGgEgE | C18DA | Me | NH2 | Y | Aib | E | G | T | F | I | S | D | Y | S | I |
| 108 | G5 | C18DA | Me | OH | Y | Aib | E | G | T | F | I | S | D | Y | S | I |
| 109 | 2OEGgEgE | C18DA | Me | NH2 | Y | Aib | E | G | T | F | I | S | D | Y | S | I |
| 110 | 2OEGgEgE | C18DA | Me | OH | Y | Aib | E | G | T | F | I | S | D | Y | S | I |
| 111 | G5 | C20DA | Me | OH | Y | Aib | E | G | T | F | I | S | D | Y | S | I |
| 112 | G5 | C20DA | Me | OH | Y | Aib | E | G | T | F | I | S | D | Y | S | I |
| 113 | G5 | C18DA | Me | OH | Y | Aib | E | G | T | F | I | S | D | Y | S | I |
| 114 | G5 | C18DA | Me | OH | Y | Aib | E | G | T | F | I | S | D | Y | S | I |
| 115 | G5 | C18DA | Me | OH | Y | Aib | E | G | T | F | I | S | D | Y | S | I |
| 116 | G5 | C18DA | Me | OH | Y | Aib | E | G | T | F | I | S | D | Y | S | I |
| 117 | GGPAPAPgE | C18DA | Me | OH | Y | Aib | E | G | T | F | I | S | D | Y | S | I |
| 118 | G5 | C18DA | Me | OH | Y | Aib | E | G | T | F | I | S | D | Y | S | I |
| 119 | 2OEGgEgE | C18DA | Me | OH | Y | Aib | E | G | T | F | I | S | D | Y | S | I |
| 120 | G5 | C18DA | Me | OH | Y | Aib | E | G | T | F | I | S | D | Y | S | I |
| 121 | G5 | C18DA | Me | OH | Y | Aib | E | G | T | F | I | S | D | Y | S | I |
| 122 | 2OEGgEgE | C18DA | Me | OH | Y | Aib | E | G | T | F | I | S | D | Y | S | I |
| 123 | 2OEGgEgE | C18DA | Me | OH | Y | Aib | E | G | T | F | I | S | D | Y | S | I |
| 124 | G5 | C18DA | Me | OH | Y | Aib | E | G | T | F | I | S | D | Y | S | I |
| 125 | 2OEGgEgE | C17DA | Me | OH | Y | Aib | E | G | T | F | I | S | D | Y | S | I |
| 126 | 2OEGgEgE | C17DA | Me | OH | Y | Aib | E | G | T | F | I | S | D | Y | S | I |
| 127 | 2OEGgEgE | C17DA | Me | OH | Y | Aib | E | G | T | F | I | S | D | Y | S | I |
| 128 | G5 | C18DA | H | OH | Y | Aib | E | G | T | F | I | S | D | Y | S | I |
| 129 | 2OEGgEgE | C17DA | H | OH | Y | Aib | E | G | T | F | I | S | D | Y | S | I |
| 130 | 2OEGgEgE | C17DA | H | OH | Y | Aib | E | G | T | F | I | S | D | Y | S | I |
| 131 | 2OEGgEgE | C17DA | H | OH | Y | Aib | E | G | T | F | I | S | D | Y | S | I |
| 132 | 2OEGgEgE | C17DA | H | OH | Y | Aib | E | G | T | F | I | S | D | Y | S | I |
| 133 | 2OEGgEgE | C17DA | H | OH | Y | Aib | E | G | T | F | I | S | D | Y | S | I |
| 134 | 2OEGgEgE | C17DA | H | OH | Y | Aib | E | G | T | F | I | S | D | Y | S | I |
| 135 | 2OEGgEgE | C17DA | H | OH | Y | Aib | E | G | T | F | I | S | D | Y | S | I |
| 136 | GGPAPAPgE | C18DA | Me | OH | Y | Aib | E | G | T | F | I | S | D | Y | S | I |
| 137 | G5 | C18DA | H | OH | Y | Aib | E | G | T | F | I | S | D | Y | S | I |
| 138 | G5 | C18DA | H | OH | Y | Aib | E | G | T | F | I | S | D | Y | S | I |
| 139 | G5 | C18DA | H | OH | Y | Aib | E | G | T | F | I | S | D | Y | S | I |
| 140 | 2OEGgEgE | C17DA | H | OH | Y | Aib | E | G | T | F | I | S | D | Y | S | I |
| 141 | 2OEGgEgE | C18DA | H | OH | Y | Aib | E | G | T | F | I | S | D | Y | S | I |
| 142 | 2OEGgEgE | C17DA | H | OH | Y | Aib | E | G | T | F | I | S | D | Y | S | I |
| 143 | 2OEGgEgE | C17DA | H | OH | Y | Aib | E | G | T | F | I | S | D | Y | S | I |
| 144 | G5 | C18DA | H | OH | Y | Aib | E | G | T | F | I | S | D | Y | S | I |
| 145 | G5 | C18DA | H | OH | Y | Aib | E | G | T | F | I | S | D | Y | S | I |
| 146 | G5 | C18DA | H | OH | Y | Aib | E | G | T | F | I | S | D | Y | S | I |
| 147 | G5 | C18DA | H | OH | Y | Aib | E | G | T | F | I | S | D | Y | S | I |
| 148 | G5 | C18DA | H | OH | Y | Aib | E | G | T | F | I | S | D | Y | S | I |
| 149 | G5 | C18DA | H | OH | Y | Aib | E | G | T | F | I | S | D | Y | S | I |
| 150 | G5 | C18DA | H | OH | Y | Aib | E | G | T | F | I | S | D | Y | S | I |
| 151 | G5 | C18DA | H | OH | Y | Aib | E | G | T | F | I | S | D | Y | S | I |
| 152 | G5 | C18DA | H | OH | Y | Aib | E | G | T | F | I | S | D | Y | S | I |
| 153 | 2OEGgEgE | C18DA | H | OH | Y | Aib | E | G | T | F | I | S | D | Y | S | I |
| 154 | 2OEGgEgE | C18DA | H | OH | Y | Aib | E | G | T | F | I | S | D | Y | S | I |
| 155 | 2OEGgEgE | C18DA | H | OH | Y | Aib | E | G | T | F | I | S | D | Y | S | I |
| 156 | 2OEGgEgE | C18DA | H | OH | Y | Aib | E | G | T | F | I | S | D | Y | S | I |
| 157 | 2OEGgEgE | C18DA | H | OH | Y | Aib | E | G | T | F | I | S | D | Y | S | I |
| 158 | 2OEGgEgE | C18DA | H | OH | Y | Aib | E | G | T | F | I | S | D | Y | S | I |
| 159 | 2OEGgEgE | C20DA | Me | OH | Y | Aib | E | G | T | F | I | S | D | Y | S | I |
| 160 | 2OEGgE | C18DA | H | OH | Y | Aib | E | G | T | F | I | S | D | Y | S | I |
| 161 | 2OEGgE | C18DA | H | OH | Y | Aib | E | G | T | F | I | S | D | Y | S | I |
| 162 | 2OEGgE | C18DA | H | OH | Y | Aib | E | G | T | F | I | S | D | Y | S | I |
| 163 | 2OEGgE | C18DA | H | OH | Y | Aib | E | G | T | F | I | S | D | Y | S | I |
| 164 | 2OEGgE | C18DA | H | OH | Y | Aib | E | G | T | F | I | S | D | Y | S | I |
| 165 | 2OEGgE | C18DA | H | OH | Y | Aib | E | G | T | F | I | S | D | Y | S | I |
| 166 | 2OEGgE | C18DA | H | OH | Y | Aib | E | G | T | F | I | S | D | Y | S | I |
| 167 | 2OEGgEgE | C18DA | H | OH | Y | Aib | E | G | T | F | I | S | D | Y | S | I |
| 168 | 2OEGgEgE | C18DA | H | OH | Y | Aib | E | G | T | F | I | S | D | Y | S | I |
| 169 | 2OEGgE | C18DA | H | OH | Y | Aib | E | G | T | F | I | S | D | Y | S | I |
| 170 | 2OEGgE | C18DA | H | OH | Y | Aib | E | G | T | F | I | S | D | Y | S | I |
| 171 | 2OEGgE | C18DA | H | OH | Y | Aib | E | G | T | F | I | S | D | Y | S | I |
| 172 | 2OEGgE | C18DA | H | OH | Y | Aib | E | G | T | F | I | S | D | Y | S | I |
| 173 | 2OEGgEgE | C18DA | Me | OH | Y | Aib | E | G | T | F | I | S | D | Y | S | I |
| 174 | 2OEGgEgE | C18DA | Me | OH | Y | Aib | E | G | T | F | I | S | D | Y | S | I |
| 175 | 2OEGgEgE | C18DA | Mo | OH | Y | Aib | E | G | T | F | I | S | D | Y | S | I |
| 176 | 2OEGgEgE | C18DA | Me | OH | Y | Aib | E | G | T | F | I | S | D | Y | S | I |
| 177 | 2OEGgEgE | C18DA | Me | OH | Y | Aib | E | G | T | F | I | S | D | Y | S | I |
| 178 | 2OEGgEgE | C18DA | Me | OH | Y | Aib | E | G | T | F | I | S | D | Y | S | I |
| 179 | G5 | C18DA | Me | OH | Y | Aib | E | G | T | F | I | S | D | Y | S | I |
| 180 | G5 | C18DA | Me | OH | Y | Aib | E | G | T | F | I | S | D | Y | S | I |
| 181 | 2OEGgE | C18DA | Me | NH2 | Y | Aib | E | G | T | F | I | S | D | Y | S | I |
| 182 | 2OEGgE | C18DA | Me | NH2 | Y | Aib | E | G | T | F | I | S | D | Y | S | I |
| 183 | 2OEGgE | C18DA | Me | NH2 | Y | Aib | E | G | T | F | I | S | D | Y | S | I |
| 184 | 2OEGgE | C18DA | Me | NH2 | Y | Aib | E | G | T | F | I | S | D | Y | S | I |
| 185 | 2OEGgE | C18DA | Me | NH2 | Y | Aib | E | G | T | F | I | S | D | Y | S | I |
| 186 | 2OEGgE | C18DA | Me | NH2 | Y | Aib | E | G | T | F | I | S | D | Y | S | I |
| 187 | 2OEGgE | C18DA | | OH | Y | Aib | E | G | T | F | I | S | D | Y | S | I |
| 188 | 2OEGgE | C18DA | | OH | Y | Aib | E | G | T | F | I | S | D | Y | S | I |
| 189 | 2OEGgE | C18DA | | OH | Y | Aib | E | G | T | F | I | S | D | Y | S | I |
| 190 | 2OEGgE | C18DA | Me | NH2 | Y | Aib | E | G | T | F | I | S | D | Y | S | I |
| 191 | 2OEGgE | C18DA | Me | | Y | Aib | E | G | T | F | I | S | D | Y | S | I |
| 192 | 2OEGgEgE | C17DA | Mo | OH | Y | Aib | E | G | T | F | I | S | D | Y | S | I |
| 193 | gEgEgE | C17DA | Me | OH | Y | Aib | E | G | T | F | I | S | D | Y | S | I |
| 194 | 2OEGgEgE | C18DA | Me | OH | Y | Aib | E | G | T | F | I | S | D | Y | S | I |
| 195 | G5 | C18DA | Me | OH | Y | Aib | E | G | T | F | I | S | D | Y | S | I |
| 196 | 2OEGgEgE | C18DA | Me | OH | Y | Aib | E | G | T | F | I | S | D | Y | S | I |
| 197 | G5 | C18DA | Me | OH | Y | Aib | E | G | T | F | I | S | D | Y | S | I |
| 198 | G5 | C18DA | Me | OH | Y | Aib | E | G | T | F | I | S | D | Y | S | I |
| 199 | G5 | C18DA | Me | OH | Y | Aib | E | G | T | F | I | S | D | Y | S | I |
| 200 | 2OEGgEgE | C17DA | Me | OH | Y | Aib | E | G | T | F | I | S | D | Y | S | I |
| 201 | 2OEGgEgE | C18DA | Me | OH | Y | Aib | E | G | T | F | I | S | D | Y | S | I |
| 202 | 2OEGgEgE | C18 | Me | OH | Y | Aib | E | G | T | F | I | S | D | Y | S | I |
| | | (stea) | | | | | | | | | | | | | | |
| 203 | 2OEGgEgE | C18 | Me | OH | Y | Aib | E | G | T | F | I | S | D | Y | S | I |
| | | (stea) | | | | | | | | | | | | | | |
| 204 | 2OEGgEgE | C18DA | Me | NH2 | Y | Aib | E | G | T | F | I | S | D | Y | S | I |
| 205 | 2OEGgEgE | C18DA | Me | OH | Y | G | E | G | T | F | I | S | D | Y | S | I |
| 206 | 2OEGgEgE | C18DA | H | OH | Y | Aib | E | G | T | F | I | S | D | Y | S | I |
| 207 | OEGgEgE | C18DA | Me | OH | Y | Aib | E | G | T | F | I | S | D | Y | S | I |
| 208 | 2OEGgE | C18DA | Me | OH | Y | Aib | E | G | T | F | I | S | D | Y | S | I |
| 209 | 2OEGgEgE | C18DA | Me | OH | Y | Aib | E | G | T | F | I | S | D | Y | S | I |
| 210 | G5gEgE | C18DA | Me | OH | Y | Aib | E | G | T | F | I | S | D | Y | S | I |
| 211 | 2OEGgEgE | C18DA | Me | OH | Y | Aib | E | G | T | F | I | S | D | Y | S | I |
| 212 | 2OEGgEgE | C18DA | Me | OH | Y | Aib | E | G | T | F | I | S | D | Y | S | I |
| 213 | 2OEGgEgE | C18DA | Me | OH | Y | Aib | E | G | T | F | I | S | D | Y | S | I |
| 214 | 2OEGgEgE | C18DA | Me | OH | Y | Aib | E | G | T | F | I | S | D | Y | S | I |
| 215 | 2OEGgEgE | C18DA | Me | OH | Y | Aib | E | G | T | F | I | S | D | Y | S | I |
| 216 | 2OEGgEgE | C18DA | Me | OH | Y | Aib | E | G | T | F | I | S | D | Y | S | I |
| 217 | 2OEGgEgE | C18DA | Me | OH | Y | dA | E | G | T | F | I | S | D | Y | S | I |
| 218 | 2OEGgEgE | C18DA | Me | OH | Y | A | E | G | T | F | I | S | D | Y | S | I |
| 219 | 2OEG | C18DA | Me | OH | Y | Aib | E | G | T | F | I | S | D | Y | S | I |
| 220 | 2OEGgEgE | C18DA | Me | OH | Y | Aib | E | G | T | F | I | S | D | Y | S | I |
| 221 | 2OEGgEgE | C18DA | Me | OH | Y | Aib | E | G | T | F | I | S | D | Y | S | I |
| 222 | 2OEGgEgE | C18DA | Me | OH | Y | Aib | E | G | T | F | I | S | D | Y | S | I |
| 223 | 2OEGgEgE | C18DA | Me | OH | Y | Aib | E | G | T | F | I | S | D | Y | S | I |
| 224 | 2OEGgEgE | C18DA | Me | OH | Y | Aib | E | G | T | F | I | S | D | Y | S | I |
| 225 | 2OEGgEgE | C18DA | Me | OH | Y | Aib | E | G | T | F | I | S | D | Y | S | I |
| 226 | 2OEGgEgE | C18DA | Me | OH | Y | Aib | E | G | T | F | I | S | D | Y | S | I |
| 227 | 2OEGgEgE | C18DA | Me | NH2 | Y | Aib | E | G | T | F | I | S | D | Y | S | I |
| 228 | 2OEGgEgE | C18DA | Me | NH2 | Y | Aib | E | G | T | F | I | S | D | Y | S | I |
| 229 | 2OEGgEgE | C18DA | Me | NH2 | Y | Aib | E | G | T | F | I | S | D | Y | S | I |
| 230 | 2OEGgE | C18DA | H | OH | Y | Aib | E | G | T | F | I | S | D | Y | S | I |
| 231 | 2OEGgE | C18DA | Me | OH | Y | Aib | E | G | T | F | I | S | D | Y | S | I |
| 232 | 2OEGgEgE | C18DA | Me | NH2 | Y | Aib | E | G | T | F | I | S | D | Y | S | I |
| 233 | 2OEGgEgE | C18DA | Me | NH2 | Y | Aib | E | G | T | F | I | S | D | Y | S | I |
| 234 | 2OEGgEgE | C18DA | Me | NH2 | Y | Aib | E | G | T | F | I | S | D | Y | S | I |
| 235 | 2OEGgEgE | C18DA | Me | NH2 | Y | Aib | E | G | T | F | I | S | D | Y | S | I |
| 236 | 2OEGgE | C18DA | Me | OH | Y | Aib | E | G | T | F | I | S | D | Y | S | I |
| 237 | 2OEGgE | C18DA | Me | OH | Y | Aib | E | G | T | F | I | S | D | Y | S | I |
| 238 | 2OEGgEgE | C18DA | Me | NH2 | Y | Aib | E | G | T | F | I | S | D | Y | S | I |
| 239 | 2OEGgEgE | C18DA | Me | OH | Y | Aib | E | G | T | F | I | S | D | Y | S | I |
| 240 | 2OEGgEgE | C18DA | Me | OH | Y | Aib | E | G | I | F | I | S | D | Y | S | I |
| 241 | 2OEGgEgE | C18DA | H | OH | Y | Aib | E | G | T | F | I | S | D | Y | S | I |
| 242 | 2OEGgEgE | C18DA | H | OH | Y | Aib | E | G | T | F | I | S | D | Y | S | I |
| 243 | 2OEGgE | C18DA | Me | | Y | DA | E | G | T | F | I | S | D | Y | S | I |
| 244 | 2OEGgE | C18DA | Ac | | Y | Aib | E | G | T | F | I | S | D | Y | S | I |
| 245 | 2OEGgE | C18DA | Me | | Y | Aib | E | G | T | F | I | S | D | Y | S | I |
| 246 | 2OEGgE | C18DA | Me | | Y | Aib | E | G | T | F | I | S | D | Y | S | I |
| 247 | 2OEGgE | C18DA | Me | | Y | Aib | E | G | T | F | I | S | D | Y | S | I |
| 248 | 2OEGgE | C18DA | Me | | Y | Aib | E | G | T | F | I | S | D | Y | S | I |
| 249 | 2OEGgE | C18DA | | | Y | Aib | E | G | T | F | I | S | D | Y | S | I |
| 250 | G5 | C18DA | Me | NH2 | Y | Aib | E | G | T | F | I | S | D | Y | S | I |
| 251 | G5 | C18DA | Me | | Y | Aib | E | G | T | F | I | S | D | Y | S | I |
| 252 | 2OEGgE | C18DA | Me | NH2 | Y | Aib | E | G | T | F | I | S | D | Y | S | I |
| 253 | 2OEGgE | C18DA | Me | NH2 | Y | Aib | E | G | T | F | I | S | D | Y | S | I |
| 254 | 2OEGgE | C18DA | Me | NH2 | Y | Aib | E | G | T | F | I | S | D | Y | S | I |
| 255 | 2OEGgE | C17DA | Me | | Y | Aib | E | G | T | F | I | S | D | Y | S | I |
| 256 | 2OEGgEgE | C17DA | Me | NH2 | Y | Aib | E | G | T | F | I | S | D | Y | S | I |
| 257 | 2OEGgEgE | C17DA | Me | NH2 | Y | Aib | E | G | T | F | I | S | D | Y | S | I |
| 258 | 2OEGgEgE | C17DA | Me | NH2 | Y | Aib | E | G | T | F | I | S | D | Y | S | I |
| 259 | G5 | C17DA | Me | OH | Y | Aib | E | G | T | F | I | S | D | Y | S | I |
| 260 | G5gE | C17DA | Me | NH2 | Y | Aib | E | G | T | F | I | S | D | Y | S | I |
| 261 | G5gE | C17DA | Me | NH2 | Y | Aib | E | G | T | F | I | S | D | Y | S | I |
| 262 | G5gE | C18DA | Me | NH2 | Y | Aib | E | G | T | F | I | S | D | Y | S | I |
| 263 | 2OEGgE | C17DA | Me | NH2 | Y | Aib | E | G | T | F | I | S | D | Y | S | I |
| 264 | G5gEgE | C17DA | Me | NH2 | Y | Aib | E | G | T | F | I | S | D | Y | S | I |
| 265 | G5gE | C17DA | Me | NH2 | Y | Aib | E | G | T | F | I | S | D | Y | S | I |
| 266 | 2OEGgE | C17DA | Me | NH2 | Y | Aib | E | G | T | F | I | S | D | Y | S | I |
| 267 | 2OEGgEgE | C18DA | Me | NH2 | Y | Aib | E | G | T | F | I | S | D | Y | S | I |
| 268 | 2OEGgE | C17DA | Me | NH2 | Y | Aib | E | G | T | F | I | S | D | Y | S | I |
| 269 | 2OEGgEgE | C18DA | Me | NH2 | Y | Aib | E | G | T | F | I | S | D | Y | S | I |
| 270 | 2OEGgEgE | C17DA | Me | NH2 | Y | Aib | E | G | T | F | I | S | D | Y | S | I |
| 271 | 2OEGgEgE | C17DA | Me | NH2 | Y | Aib | E | G | T | F | I | S | D | Y | S | I |
| 272 | 2OEGgEgE | C20DA | Me | NH2 | Y | Aib | E | G | T | F | I | S | D | Y | S | I |
| 273 | G5 | C18DA | Me | NH2 | Y | Aib | E | G | T | F | I | S | D | Y | S | I |
| 274 | G5 | C18DA | Me | NH2 | Y | Aib | E | G | T | F | I | S | D | Y | S | I |
| 275 | G5 | C18DA | Me | NH2 | Y | Aib | E | G | T | F | I | S | D | Y | S | I |
| 276 | G5 | C18DA | Me | NH2 | Y | Aib | E | G | T | F | I | S | D | Y | S | I |
| 277 | G6 | C18DA | Me | NH2 | Y | Aib | E | G | T | F | I | S | L | Y | S | I |
| 278 | G6 | C18DA | Me | NH2 | Y | Aib | Q | G | T | F | I | S | D | Y | S | I |
| 279 | G6 | C18DA | Me | NH2 | Y | Aib | E | G | T | F | I | S | D | Y | S | I |
| 280 | G6 | C18DA | Me | NH2 | Y | Aib | E | G | T | F | I | S | D | Y | S | I |
| 281 | G5 | C18DA | Me | NH2 | Y | Aib | E | G | T | F | I | S | D | Y | S | I |
| 282 | G5K | C18DA | Me | NH2 | Y | Aib | E | G | T | F | I | S | L | Y | S | I |
| 283 | G4KG | C18DA | Me | NH2 | Y | Aib | E | G | T | F | I | S | L | Y | S | I |
| 284 | G5 | C18DA | Me | OH | Y | Aib | E | G | T | F | I | S | D | Y | S | I |
| 285 | G5 | C18DA | Me | OH | Y | Aib | E | G | T | F | I | S | D | Y | S | I |
| 286 | 2OEGgEgE | C18DA | Me | OH | Y | Aib | E | G | T | F | I | S | D | Y | S | I |
| 287 | 2OEGgEgE | C18DA | Me | OH | Y | Aib | E | G | T | F | I | S | D | Y | S | I |
| 288 | G5 | C18DA | Me | OH | Y | Aib | E | G | T | F | I | S | D | Y | S | I |
| 289 | GGPAPAP | C18DA | Me | OH | Y | Aib | E | G | T | F | I | S | D | Y | S | I |
| 290 | G5 | C18DA | Me | OH | Y | Aib | E | G | T | F | I | S | D | Y | S | I |
| 291 | G5 | C18DA | H | OH | Y | Aib | E | G | T | F | I | S | D | Y | S | I |
| 292 | G5 | C18DA | Me | OH | Y | Aib | E | G | T | F | I | S | D | Y | S | I |
| 293 | 2OEGgEgE | C18DA | Me | NH2 | Y | Aib | E | G | T | F | I | S | D | Y | S | I |
| 294 | GGGGG | C18DA | Me | OH | Y | Aib | E | G | T | F | I | S | D | Y | S | I |

| CPMD | | | | | | | | | | | | | | | | | |
|---|---|---|---|---|---|---|---|---|---|---|---|---|---|---|---|---|---|
| No. | 13 | 14 | 15 | 16 | 17 | 18 | 19 | 20 | 21 | 22 | 23 | 24 | 25 | 26 | 27 | 28 | 29 |
| 1 | A | L | D | R | Aib | H | Q | Aib | N | F | V | N | W | L | L | A | Q |
| 2 | A | L | D | R | I | H | Q | Aib | N | F | V | N | W | L | L | A | Q |
| 3 | A | Km | D | R | I | H | Q | Q | N | F | V | N | W | L | L | A | Q |
| 4 | A | L | D | R | Km | H | Q | Q | N | F | V | N | W | L | L | A | Q |
| 5 | A | L | D | R | I | H | Q | Q | N | F | V | N | W | L | L | A | Q |
| 6 | A | Km | D | R | Aib | A | Q | Aib | N | F | V | N | W | L | L | A | Q |
| 7 | A | Km | D | R | Aib | A | Q | Aib | N | F | V | N | W | L | L | A | Q |
| 8 | A | L | D | R | Aib | A | Q | Aib | Km | F | V | N | W | L | L | A | Q |
| 9 | A | L | D | R | Aib | A | Q | Aib | Km | F | V | N | W | L | L | A | Q |
| 10 | A | Km | D | R | Aib | A | Q | Aib | N | F | V | N | W | L | L | A | Q |
| 11 | A | L | D | R | Aib | Km | Q | Aib | N | F | V | N | W | L | L | A | Q |
| 12 | A | Km | D | R | Aib | A | Q | Aib | N | F | V | N | W | L | L | A | Q |
| 13 | Aib | L | D | R | Aib | A | Q | Aib | Km | F | V | N | W | L | L | A | Q |
| 14 | Aib | Km | D | R | Aib | A | Q | Aib | N | F | V | N | W | L | L | A | Q |
| 15 | Aib | L | D | R | Km | A | Q | Aib | N | F | V | N | W | L | L | A | Q |
| 16 | Aib | L | D | R | Aib | A | Q | Aib | N | F | V | N | W | L | L | A | Q |
| 17 | A | L | D | R | Aib | Km | Q | Aib | N | F | V | N | W | L | L | A | Q |
| 18 | A | L | D | R | Aib | H | Q | Aib | Km | F | V | N | W | L | L | A | Q |
| 19 | A | L | D | R | Aib | H | Q | Aib | N | F | V | N | W | L | L | A | Km |
| 20 | A | L | D | R | Km | H | Q | Aib | N | F | V | N | W | L | L | A | Q |
| 21 | A | L | D | R | Aib | A | Q | Aib | Km | F | V | N | W | L | L | A | Q |
| 22 | A | Km | D | R | Aib | A | Q | Aib | D | F | V | N | W | L | L | A | Q |
| 23 | A | L | D | R | Km | A | Q | Aib | D | F | V | N | W | L | L | A | Q |
| 24 | A | Km | D | R | Aib | A | Q | Aib | D | F | V | N | W | L | L | A | Q |
| 25 | A | L | D | R | Aib | Km | Q | Aib | D | F | V | N | W | L | L | A | Q |
| 26 | Aib | L | D | R | Km | A | Q | Aib | N | F | V | N | W | L | L | A | Q |
| 27 | Aib | L | D | R | Aib | A | Q | Aib | N | F | V | N | W | L | L | A | Q |
| 28 | Aib | Km | D | R | Aib | A | Q | Aib | N | F | V | N | W | L | L | A | Q |
| 29 | Aib | Km | D | R | Aib | A | Q | Aib | N | F | V | N | W | L | L | A | Q |
| 30 | A | L | D | R | Aib | A | Q | Aib | Km | F | V | N | W | L | L | A | Q |
| 31 | A | L | D | R | Aib | Km | Q | Aib | N | F | V | N | W | L | L | A | Q |
| 32 | Aib | Km | D | R | Aib | A | Q | Aib | N | F | V | N | W | L | L | A | Q |
| 33 | Aib | L | D | R | Aib | A | Q | Aib | N | F | V | N | W | L | L | A | Q |
| 34 | A | Km | D | R | Aib | A | Q | Aib | N | F | V | N | W | L | L | A | Q |
| 35 | A | L | D | R | Aib | A | Q | Aib | Km | F | V | N | W | L | L | A | Q |
| 36 | A | L | D | R | Aib | A | Q | Aib | Km | F | V | N | W | L | L | A | Q |
| 37 | A | L | D | R | Aib | A | Q | Aib | Km | F | V | N | W | L | L | A | Q |
| 38 | A | Km | D | R | Aib | A | Q | Aib | D | F | V | N | W | L | L | A | Q |
| 39 | A | L | D | R | Aib | A | Q | Aib | Km | F | V | N | W | L | L | A | Q |
| 40 | A | Km | D | R | Aib | A | Q | Aib | N | F | V | N | W | L | L | A | Q |
| 41 | A | L | D | R | Aib | A | Q | Aib | Km | F | V | N | W | L | L | A | Q |
| 42 | A | L | D | R | Aib | A | Q | Aib | Km | F | V | N | W | L | L | A | Q |
| 43 | A | Km | D | R | Aib | A | Q | Aib | D | F | V | N | W | L | L | A | Q |
| 44 | A | Km | D | R | Aib | A | Q | Aib | N | F | V | N | W | L | L | A | Q |
| 45 | A | Km | D | R | Aib | A | Q | Aib | N | F | V | N | W | L | L | A | Q |
| 46 | A | L | D | R | Aib | Km | Q | Aib | N | F | V | N | W | L | L | A | Q |
| 47 | Aib | Km | D | R | Aib | A | Q | Aib | D | F | V | N | W | L | L | A | Q |
| 48 | Aib | Km | D | R | Aib | A | Q | Aib | D | F | V | N | W | L | L | A | Q |
| 49 | A | L | D | R | Aib | Km | Q | Aib | D | F | V | N | W | L | L | A | Q |
| 50 | A | L | D | R | Km | A | Q | Aib | D | F | V | N | W | L | L | A | Q |
| 51 | A | L | D | R | Km | A | Q | Aib | D | F | V | N | W | L | L | A | Q |
| 52 | A | L | D | R | Km | A | Q | Aib | D | F | V | N | W | L | L | A | Q |
| 53 | Aib | Km | D | R | Aib | A | Q | Aib | D | F | V | N | W | L | L | A | Q |
| 54 | Aib | Km | D | R | Aib | A | Q | Aib | D | F | V | N | W | L | L | A | Q |
| 55 | A | L | D | R | Aib | A | Q | Aib | N | F | V | N | W | L | L | A | Km |
| 56 | A | L | D | R | Aib | A | Q | Aib | D | F | V | N | W | L | L | A | Km |
| 57 | A | L | D | R | Aib | A | Q | Aib | D | F | V | N | W | L | L | A | Km |
| 58 | A | L | D | R | Aib | A | Q | Aib | D | F | V | N | W | L | L | A | Km |
| 59 | A | L | D | R | Aib | A | Q | Aib | N | F | V | N | W | L | L | A | Km |
| 60 | A | Km | D | S | Aib | A | Q | Aib | D | F | V | N | W | L | L | A | Q |
| 61 | A | L | D | R | Aib | A | Q | Aib | D | F | V | N | W | L | L | A | Q |
| 62 | Aib | L | D | R | Aib | A | Q | Aib | D | F | V | N | W | L | L | A | Km |
| 63 | A | Km | D | R | Aib | A | Q | Aib | D | F | V | N | W | L | L | A | Q |
| 64 | Aib | L | D | S | Km | A | Q | Aib | D | F | V | N | W | L | L | A | Q |
| 65 | Aib | L | D | R | Aib | A | Q | Aib | Km | F | V | N | W | L | L | A | Q |
| 66 | Aib | L | D | R | Aib | A | Q | Aib | Km | F | V | N | W | L | L | A | Q |
| 67 | Aib | Km | D | R | Aib | A | Q | Aib | D | F | V | N | W | L | L | A | Q |
| 68 | A | L | D | R | Aib | Km | Q | Aib | N | F | V | N | W | L | L | A | Q |
| 69 | Aib | L | D | R | Aib | A | Q | Aib | N | F | V | N | W | L | L | A | Q |
| 70 | A | Km | D | R | Aib | A | Q | Aib | D | F | V | N | W | L | L | A | Q |
| 71 | A | Km | D | R | Aib | A | Q | Aib | D | F | V | N | W | L | L | A | Q |
| 72 | Aib | L | D | R | Aib | A | Q | Aib | Km | F | V | N | W | L | L | A | Q |
| 73 | Aib | L | D | R | Aib | Km | Q | Aib | D | F | V | N | W | L | L | A | Q |
| 74 | Aib | L | D | R | Aib | Km | Q | Aib | N | F | V | N | W | L | L | A | Q |
| 75 | A | Km | D | S | Aib | A | Q | Aib | D | F | V | N | W | L | L | A | Q |
| 76 | A | Km | D | K | Aib | A | Q | Aib | D | F | V | N | W | L | L | A | Q |
| 77 | Aib | L | D | S | Km | A | Q | Aib | D | F | V | N | W | L | L | A | Q |
| 78 | A | L | D | S | Km | A | Q | Aib | D | F | V | N | W | L | L | A | Q |
| 79 | A | Km | D | S | Aib | A | Q | Aib | D | F | V | N | W | L | L | A | Q |
| 80 | A | Km | D | R | Aib | A | Q | Aib | D | F | V | N | W | L | L | A | Q |
| 81 | A | Km | D | R | Aib | A | Q | Nb | D | F | V | N | W | L | L | A | Q |
| 82 | A | Km | D | R | Aib | A | Q | Aib | D | F | V | N | W | L | L | A | Q |
| 83 | Aib | Km | D | R | Aib | A | Q | Aib | D | F | V | N | W | L | L | A | Q |
| 84 | A | Km | D | R | Aib | A | Q | Aib | D | F | V | N | W | L | L | A | Q |
| 85 | Aib | L | D | R | Aib | A | Q | Aib | Km | F | V | N | W | L | L | A | Q |
| 86 | A | L | D | R | Aib | A | Q | Aib | Km | F | V | N | W | L | L | A | Q |
| 87 | Aib | L | D | R | Aib | Km | Q | Aib | D | F | V | N | W | L | L | A | Q |
| 88 | Aib | L | D | R | Km | A | Q | Aib | D | F | V | N | W | L | L | A | Q |
| 89 | Aib | L | D | R | Aib | A | Q | Aib | Km | F | V | N | W | L | L | A | Q |
| 90 | Aib | L | D | R | Aib | A | Q | Aib | Km | F | V | N | W | L | L | A | Q |
| 91 | Aib | L | D | R | Aib | A | Q | Aib | Km | F | V | N | W | L | L | A | Q |
| 92 | Aib | L | D | R | Aib | A | Q | Aib | Km | F | V | N | W | L | L | A | Q |
| 93 | Aib | L | D | R | Aib | A | Q | Aib | Km | F | V | N | W | L | L | A | Q |
| 94 | Aib | L | D | R | Aib | A | Q | Aib | Km | F | V | N | W | L | L | A | Q |
| 95 | Aib | Km | D | R | Aib | A | Q | Aib | N | F | V | N | W | L | L | A | Q |
| 96 | Aib | Km | D | R | Aib | A | Q | Aib | N | F | V | N | W | L | L | A | Q |
| 97 | A | L | D | R | Aib | A | Q | Aib | Km | F | V | N | W | L | L | A | Q |
| 98 | Aib | Km | D | R | Aib | A | Q | Aib | D | F | V | N | W | L | L | A | Q |
| 99 | A | L | D | R | Aib | Km | Q | Aib | N | F | V | N | W | L | L | A | Q |
| 100 | Aib | L | D | R | Aib | Km | Q | Aib | N | F | V | N | W | L | L | A | Q |
| 101 | Aib | L | D | R | Aib | A | Q | Aib | Km | F | V | N | W | L | L | A | Q |
| 102 | A | L | D | R | Aib | Km | Q | Aib | D | F | V | N | W | L | L | A | Q |
| 103 | A | L | D | R | Aib | A | Q | Aib | Km | F | V | N | W | L | L | A | Q |
| 104 | A | L | D | R | Aib | A | Q | Aib | Km | F | V | N | W | L | L | A | Q |
| 105 | A | Km | D | R | Aib | A | Q | Aib | D | F | V | N | W | L | L | A | Q |
| 106 | Aib | L | D | R | Aib | A | A | Aib | Km | F | V | N | W | L | L | A | Q |
| 107 | Aib | L | D | R | Aib | A | A | Aib | Km | F | V | N | W | L | L | A | Q |
| 108 | Aib | Km | D | R | Aib | A | Q | Aib | D | F | V | E | W | L | L | A | Q |
| 109 | Aib | L | D | R | Aib | Km | A | Aib | N | F | V | N | W | L | L | A | Q |
| 110 | Aib | L | D | R | Aib | A | Q | Aib | Km | F | V | E | W | L | L | A | Q |
| 111 | A | Km | D | R | Aib | A | Q | Aib | D | F | V | N | W | L | L | A | Q |
| 112 | A | Km | D | R | Aib | A | Q | Aib | N | F | V | N | W | L | L | A | Q |
| 113 | A | L | D | R | Km | A | Q | Aib | D | F | V | N | W | L | L | A | Q |
| 114 | A | Km | D | R | Aib | A | Q | Aib | N | F | V | N | W | L | L | A | Q |
| 115 | A | L | D | R | Km | A | Q | Aib | D | F | V | E | W | L | L | A | Q |
| 116 | A | Km | D | R | Aib | A | Q | Aib | D | F | V | N | W | L | L | A | Q |
| 117 | A | L | D | R | Aib | A | Q | Aib | Km | F | V | N | W | L | L | A | Q |
| 118 | A | Km | D | R | Aib | A | Q | Aib | D | F | V | E | W | L | L | A | Q |
| 119 | A | L | D | R | Aib | A | Q | Aib | Km | F | V | E | W | L | L | A | Q |
| 120 | A | Km | D | R | Aib | A | Q | R | D | F | V | E | W | L | L | A | Q |
| 121 | A | Km | D | R | Aib | A | Q | K1 | D | F | V | E1 | W | L | L | A | Q |
| 122 | K1 | L | D | R | E1 | A | Q | Aib | Km | F | V | N | W | L | L | A | Q |
| 123 | A | K1 | D | R | A | E1 | Q | Aib | Km | F | V | N | W | L | L | A | Q |
| 124 | A | Km | D | R | Aib | A | Q | K1 | D | F | V | E1 | W | L | L | A | Q |
| 125 | A | L | D | R | Aib | A | Q | Aib | Km | F | V | N | W | L | L | A | Q |
| 126 | Aib | L | D | R | Aib | A | Q | Aib | Km | F | V | N | W | L | L | A | Q |
| 127 | Aib | L | D | R | Aib | A | Q | Aib | Km | F | V | N | W | L | L | A | Q |
| 128 | A | Km | D | R | Aib | A | Q | Aib | D | F | V | N | W | L | L | A | Q |
| 129 | Aib | L | D | K | I | A | Q | Q | Km | F | V | E | W | L | L | A | Q |
| 130 | A | L | D | K | Aib | A | Q | Q | Km | F | V | E | W | L | L | A | Q |
| 131 | A | L | D | K | Aib | A | Q | A | Km | F | V | E | W | L | L | A | Q |
| 132 | A | L | D | K | I | A | Q | Aib | Km | F | V | E | W | L | L | A | Q |
| 133 | A | L | D | K | A | A | Q | Aib | Km | F | V | E | W | L | L | A | Q |
| 134 | A | L | D | K | I | A | Q | Q | Km | F | V | E | W | L | L | A | Q |
| 135 | A | L | D | K | I | A | Q | R | Km | F | V | E | W | L | L | A | Q |
| 136 | A | L | D | R | Aib | Km | Q | Aib | D | F | V | N | W | L | L | A | Q |
| 137 | Aib | Km | D | R | Aib | A | Q | Aib | D | F | V | N | W | L | L | A | Q |
| 138 | A | Km | D | R | Aib | A | Q | Aib | N | F | V | N | W | L | L | A | Q |
| 139 | Aib | Km | D | R | Aib | A | Q | Aib | D | F | V | E | W | L | L | A | Q |
| 140 | A | L | D | R | Aib | A | Q | Aib | Km | F | V | N | W | L | L | A | Q |
| 141 | Aib | L | D | R | Aib | A | Q | Aib | Km | F | V | N | W | L | L | A | Q |
| 142 | A | L | D | K | I | A | Q | Q | Km | F | V | E | W | L | L | A | Q |
| 143 | A | L | D | K | I | A | Q | R | Km | F | V | E | W | L | L | A | Q |
| 144 | A | Km | D | R | Aib | A | Q | Aib | D | F | V | E | W | L | L | A | Q |
| 145 | Aib | Km | D | R | A | A | Q | Aib | D | F | V | E | W | L | L | A | Q |
| 146 | Aib | Km | D | R | Aib | A | Q | A | D | F | V | E | W | L | L | A | Q |
| 147 | Aib | Km | D | R | Aib | A | Q | R | D | F | V | E | W | L | L | A | Q |
| 148 | Aib | Km | D | R | A | A | Q | A | D | F | V | E | W | L | L | A | Q |
| 149 | A | Km | D | R | Aib | A | Q | A | D | F | V | E | W | L | L | A | Q |
| 150 | Aib | Km | D | S | Aib | A | Q | Aib | D | F | V | N | W | L | L | A | Q |
| 151 | Aib | Km | D | S | A | A | Q | Aib | D | F | V | N | W | L | L | A | Q |
| 152 | Aib | Km | D | S | Aib | A | Q | A | D | F | V | N | W | L | L | A | Q |
| 153 | A | L | D | K | I | A | Q | Aib | Km | F | V | E | W | L | L | A | Q |
| 154 | A | L | D | K | A | A | Q | Aib | Km | F | V | E | W | L | L | A | Q |
| 155 | A | L | D | K | Aib | A | Q | A | Km | F | V | E | W | L | L | A | Q |
| 156 | A | L | D | K | Aib | A | Q | R | Km | F | V | E | W | L | L | A | Q |
| 157 | A | Aib | D | K | A | A | Q | Q | Km | F | V | E | W | L | L | A | Q |
| 158 | A | Aib | D | K | A | A | Q | A | Km | F | V | E | W | L | L | A | Q |
| 159 | Aib | L | D | R | Aib | A | Q | Aib | Km | F | V | N | W | L | L | A | Q |
| 160 | A | L | D | K | I | Km | Q | Aib | D | F | V | E | W | L | L | A | Q |
| 161 | A | L | D | K | A | Km | Q | Aib | D | F | V | E | W | L | L | A | Q |
| 162 | A | L | D | K | Aib | Km | Q | A | D | F | V | E | W | L | L | A | Q |
| 163 | A | L | D | K | Aib | Km | Q | A | N | F | V | E | W | L | L | A | Q |
| 164 | A | Aib | D | K | A | Km | Q | Q | D | F | V | E | W | L | L | A | Q |
| 165 | A | Aib | D | K | A | Km | Q | A | D | F | V | E | W | L | L | A | Q |
| 166 | A | Aib | D | K | I | Km | Q | A | D | F | V | E | W | L | L | A | Q |
| 167 | Aib | L | D | K | A | A | Q | Q | Km | F | V | E | W | L | L | A | Q |
| 168 | Aib | L | D | K | A | A | Q | A | Km | F | V | E | W | L | L | A | Q |
| 169 | Aib | L | D | K | A | Km | Q | Q | D | F | V | E | W | L | L | A | Q |
| 170 | Aib | L | D | K | A | Km | Q | Q | D | F | V | N | W | L | L | A | Q |
| 171 | Aib | L | D | K | A | Km | Q | A | D | F | V | N | W | L | L | A | Q |
| 172 | Aib | L | D | K | I | Km | Q | A | D | F | V | N | W | L | L | A | Q |
| 173 | A | L | D | K | I | A | Q | Aib | Km | F | V | E | W | L | L | A | Q |
| 174 | A | L | D | K | A | A | Q | Aib | Km | F | V | E | W | L | L | A | Q |
| 175 | A | L | D | K | I | A | Q | Aib | Km | F | V | N | W | L | L | A | Q |
| 176 | A | L | D | K | A | A | Q | Aib | Km | F | V | N | W | L | L | A | Q |
| 177 | A | L | D | R | I | A | Q | Q | Km | F | V | N | W | L | L | A | Q |
| 178 | A | L | D | R | A | A | Q | Q | Km | F | V | N | W | L | L | A | Q |
| 179 | A | Km | D | R | I | A | Q | R | N | F | V | E | W | L | L | A | Q |
| 180 | A | Km | D | R | I | A | Q | R | N | F | V | E | W | L | L | A | Q |
| 181 | A | L | D | R | Aib | H | Q | Q | E* | F | V | K* | W | L | L | A | Q |
| 182 | A | L | D | R | Aib | H | Q | E* | N | F | K* | N | W | L | L | A | Q |
| 183 | A | L | D | R | E* | H | Q | K* | N | F | V | N | W | L | L | A | Q |
| 184 | A | E* | D | R | K* | H | Q | Aib | N | F | V | N | W | L | L | A | Q |
| 185 | A | L | D | R | Aib | H | Q | Aib | N | F | V | N | E* | L | L | K* | Q |
| 186 | A | L | D | R | Aib | H | Q | Aib | N | F | V | N | W | Km | L | A | Q |
| 187 | A | Km | D | K | I | H | Q | Q | D | F | V | N | W | L | L | A | Q |
| 188 | A | M | D | K | Km | H | Q | Q | D | F | V | N | W | L | L | A | Q |
| 189 | A | M | D | K | I | H | Q | Q | D | F | V | N | W | L | L | A | Q |
| 190 | A | L | D | R | I | H | Q | Q | N | F | V | N | W | L | L | A | Q |
| 191 | A | L | D | R | Aib | H | Q | Q | N | F | V | N | W | L | L | A | Q |
| 192 | A | Km | D | R | Aib | A | Q | Aib | N | F | V | N | W | L | L | A | Q |
| 193 | A | Km | D | R | Aib | A | Q | Aib | N | F | V | N | W | L | L | A | Q |
| 194 | A | L | D | R | Aib | A | Q | Aib | Km | F | V | N | W | L | L | A | Q |
| 195 | A | Km | D | R | Aib | A | Q | Aib | N | F | V | N | W | L | L | A | Q |
| 196 | A | L | D | R | I | A | Q | Q | Km | F | V | N | W | L | L | A | Q |
| 197 | A | Km | D | R | I | A | Q | Q | N | F | V | N | W | L | L | A | Q |
| 198 | A | Km | D | R | I | A | Q | Q | D | F | V | N | W | L | L | A | Q |
| 199 | A | Km | D | R | I | A | Q | Q | D | F | V | N | W | L | L | A | Q |
| 200 | A | L | D | R | Aib | A | Q | Aib | Km | F | V | N | W | L | L | A | Q |
| 201 | Aib | L | D | R | Aib | H | Q | Aib | Km | F | V | N | W | L | L | A | Q |
| 202 | Aib | L | D | R | Aib | A | Q | Aib | Km | F | V | N | W | L | L | A | Q |
| 203 | A | L | D | R | Aib | A | Q | Aib | Km | F | V | N | W | L | L | A | Q |
| 204 | Aib | L | D | R | Aib | A | Q | Aib | Km | F | V | N | W | L | L | A | Q |
| 205 | Aib | L | D | R | Aib | A | Q | Aib | Km | F | V | N | W | L | L | A | Q |
| 206 | A | L | D | R | A | A | Q | Aib | Km | F | V | E | W | L | L | A | Q |
| 207 | Aib | L | D | R | Aib | A | Q | Aib | Km | F | V | N | W | L | L | A | Q |
| 208 | Aib | L | D | R | Aib | A | Q | Aib | Km | F | V | N | W | L | L | A | Q |
| 209 | Aib | L | D | R | Aib | A | Q | Aib | Km | F | V | N | W | L | L | A | Q |
| 210 | Aib | L | D | R | Aib | A | Q | Aib | Km | F | V | N | W | L | L | A | Q |
| 211 | Aib | L | D | K | Aib | A | Q | Aib | Km | F | V | N | W | L | L | A | Q |
| 212 | Aib | L | D | R | Aib | A | A | Aib | Km | F | V | N | W | L | L | A | Q |
| 213 | A | L | D | R | Aib | A | Q | Aib | Km | F | V | N | W | L | L | A | Q |
| 214 | Aib | L | D | R | A | A | Q | Aib | Km | F | V | N | W | L | L | A | Q |
| 215 | Aib | L | D | R | Aib | A | Q | A | Km | F | V | N | W | L | L | A | Q |
| 216 | A | L | D | R | A | A | Q | A | Km | F | V | N | W | L | L | A | Q |
| 217 | Aib | L | D | R | Aib | A | Q | Aib | Km | F | V | N | W | L | L | A | Q |
| 218 | Aib | L | D | R | Aib | A | Q | Aib | Km | F | V | N | W | L | L | A | Q |
| 219 | Aib | L | D | R | Aib | A | Q | Aib | Km | F | V | N | W | L | L | A | Q |
| 220 | Aib | L | D | K | Aib | A | Q | Aib | Km | F | V | N | W | L | L | A | Q |
| 221 | Aib | L | D | R | Aib | A | Q | Km | D | F | V | N | W | L | L | A | Q |
| 222 | Aib | L | D | R | Aib | A | Q | Aib | Km | F | V | E | W | L | L | A | Q |
| 223 | Aib | L | D | R | Km | A | Q | Aib | D | F | V | N | W | L | L | A | Q |
| 224 | Aib | Km | D | R | Aib | A | Q | Aib | D | F | V | N | W | L | L | A | Q |
| 225 | Aib | L | D | R | Aib | A | Q | Aib | D | F | V | N | W | L | L | A | Q |
| 226 | A | L | D | K | I | A | Q | Aib | Km | F | V | E | W | L | L | A | Q |
| 227 | A | L | D | R | Aib | Km | Q | A | N | F | V | N | W | L | L | A | Q |
| 228 | A | L | D | R | I | Km | Q | A | N | F | V | N | W | L | L | A | Q |
| 229 | A | L | D | R | I | Km | Q | Aib | N | F | V | N | W | L | L | A | Q |
| 230 | A | L | D | K | I | Km | Q | Q | D | F | V | N | W | L | L | A | Q |
| 231 | A | L | D | K | I | Km | Q | Q | D | F | V | N | W | L | L | A | Q |
| 232 | A | L | D | R | I | Km | Q | A | D | F | V | N | W | L | L | A | Q |
| 233 | A | L | D | R | I | Km | Q | Q | D | F | V | N | W | L | L | A | Q |
| 234 | A | L | D | R | I | Km | Q | Q | N | F | V | N | W | L | L | A | Q |
| 235 | A | L | D | R | I | Km | Q | Q | N | F | V | N | W | L | L | A | Q |
| 236 | A | L | D | R | I | Km | Q | Q | D | F | V | N | W | L | L | A | Q |
| 237 | A | L | D | K | I | A | Q | Km | D | F | V | N | W | L | L | A | Q |
| 238 | A | L | D | R | I | Km | Q | Q | N | F | V | E | W | L | L | A | Q |
| 239 | A | L | D | R | I | A | Q | Q | Km | F | V | E | W | L | L | A | Q |
| 240 | A | L | D | R | I | A | Q | R | Km | F | V | E | W | L | L | A | Q |
| 241 | A | L | D | K | A | A | Q | R | Km | F | V | E | W | L | L | A | Q |
| 242 | A | L | D | K | I | A | Q | R | Km | F | V | E | W | L | L | A | Q |
| 243 | A | L | D | R | I | H | Q | Q | N | F | V | N | W | L | L | A | Q |
| 244 | A | L | D | R | I | H | Q | Q | N | F | V | N | W | L | L | A | Q |
| 245 | A | Km | D | R | I | H | Q | Q | N | F | V | N | W | L | L | A | Q |
| 246 | A | L | D | R | Km | H | Q | Q | N | F | V | N | W | L | L | A | Q |
| 247 | A | L | D | R | I | H | Q | Km | N | F | V | N | W | L | L | A | Q |
| 248 | A | L | D | R | I | H | Q | Aib | N | F | V | N | W | L | L | A | Q |
| 249 | A | L | D | R | I | H | Q | Q | N | F | V | N | W | L | L | A | Q |
| 250 | A | Km | D | R | Aib | A | Q | Aib | N | F | V | N | W | L | L | A | Q |
| 251 | A | Km | D | R | Aib | A | Q | Aib | N | F | V | N | W | L | L | A | Q |
| 252 | A | Km | D | R | I | A | Q | Aib | N | F | V | N | W | L | L | A | Q |
| 253 | A | Km | D | R | Aib | A | Q | Q | N | F | V | N | W | L | L | A | Q |
| 254 | A | Km | D | R | Aib | A | Q | Aib | N | F | V | N | W | L | L | A | Q |
| 255 | A | Km | D | R | Aib | A | Q | Aib | N | F | V | N | W | L | L | A | Q |
| 256 | A | L | D | R | Aib | Km | Q | Aib | N | F | V | N | W | L | L | A | Q |
| 257 | A | L | D | R | Aib | H | Q | Aib | Km | F | V | N | W | L | L | A | Q |
| 258 | A | L | D | R | Aib | H | Q | Aib | N | F | V | N | W | L | L | A | Km |
| 259 | A | L | D | R | Km | H | Q | Aib | N | F | V | N | W | L | L | A | Q |
| 260 | A | Km | D | R | Aib | A | Q | Aib | N | F | V | N | W | L | L | A | Q |
| 261 | Aib | Km | D | R | Aib | A | Q | Aib | N | F | V | N | W | L | L | A | Q |
| 262 | Aib | Km | D | R | Aib | A | Q | Aib | N | F | V | N | W | L | L | A | Q |
| 263 | Aib | Km | D | R | Aib | A | Q | Aib | N | F | V | N | W | L | L | A | Q |
| 264 | Aib | Km | D | R | Aib | A | Q | Aib | N | F | V | N | W | L | L | A | Q |
| 265 | Aib | Km | D | R | Aib | A | Q | Aib | D | F | V | N | W | L | L | A | Q |
| 266 | A | L | D | R | Km | A | Q | Aib | N | F | V | N | W | L | L | A | Q |
| 267 | Aib | L | D | R | Aib | A | Q | Aib | Km | F | V | N | W | L | L | A | Q |
| 268 | A | L | D | R | Aib | Km | Q | Aib | N | F | V | N | W | L | L | A | Q |
| 269 | A | L | D | R | Aib | A | Q | Aib | Km | F | V | N | W | L | L | A | Q |
| 270 | Aib | L | D | R | Km | A | Q | Aib | N | F | V | N | W | L | L | A | Q |
| 271 | Aib | L | D | R | Km | A | Q | Aib | N | F | V | N | W | L | L | A | Q |
| 272 | A | L | D | R | Aib | A | Q | Aib | Km | F | V | N | W | L | L | A | Q |
| 273 | A | Km | D | R | Aib | A | Q | Aib | N | F | V | N | W | L | L | A | Q |
| 274 | A | Km | D | R | Aib | A | Q | Aib | D | F | V | N | W | L | L | A | Q |
| 275 | Aib | Km | D | R | Aib | A | Q | Aib | N | F | V | N | W | L | L | A | Q |
| 276 | Aib | Km | D | R | Aib | A | Q | Aib | D | F | V | N | W | L | L | A | Q |
| 277 | A | L | D | R | Km | H | Q | Aib | N | F | V | N | W | L | L | A | Q |
| 278 | A | L | D | R | Km | H | Q | Aib | N | F | V | N | W | L | L | A | Q |
| 279 | A | L | S | R | Km | H | Q | Aib | N | F | V | N | W | L | L | A | Q |
| 280 | A | L | K | R | Km | H | Q | Aib | N | F | V | N | W | L | L | A | Q |
| 281 | A | Km | D | K | Aib | H | Q | Aib | N | F | V | N | W | L | L | A | Q |
| 282 | A | L | D | R | Km | H | Q | Aib | N | F | V | N | W | L | L | A | Q |
| 283 | A | L | D | R | Km | H | Q | Aib | N | F | V | N | W | L | L | A | Q |
| 284 | Aib | L | D | R | Km | A | Q | Aib | D | F | V | N | W | L | L | A | Q |
| 285 | A | Km | D | R | I | A | Q | Q | D | F | V | N | W | L | L | A | Q |
| 286 | A | L | D | R | I | A | Q | Q | Km | F | V | N | W | L | L | A | Q |
| 287 | A | L | D | R | I | Km | Q | Q | D | F | V | N | W | L | L | A | Q |
| 288 | A | L | D | R | Km | A | Q | Q | D | F | V | N | W | L | L | A | Q |
| 289 | A | Km | D | R | Aib | A | Q | Aib | D | F | V | N | W | L | L | A | Q |
| 290 | A | L | D | R | Km | A | Q | Aib | N | F | V | N | W | L | L | A | Q |
| 291 | A | Km | D | R | A | A | Q | Aib | D | F | V | E | W | L | L | A | Q |
| 292 | A | Km | D | R | I | A | Q | Q | N | F | V | N | W | L | L | A | Q |
| 293 | A | L | D | R | Aib | Km | Q | Aib | N | F | V | N | W | L | L | A | Q |
| 294 | Aib | Km | D | R | Aib | A | Q | Aib | D | F | V | N | W | L | L | A | Q |

| | | | | | | | | | | | | | | SEQ |
|---|---|---|---|---|---|---|---|---|---|---|---|---|---|---|
| CPMD | | | | | | | | | | | | | | ID |
| No. | 30 | 31 | 32 | 33 | 34 | 35 | 36 | 37 | 38 | 39 | 40 | 41 | 42 | No. |
| 1 | Km | P | S | S | G | A | P | P | P | S | NH2 | | | 2 |
| 2 | Km | P | S | S | G | A | P | P | P | S | NH2 | | | 3 |
| 3 | R | P | S | S | G | A | P | P | P | S | NH2 | | | 4 |
| 4 | R | P | S | S | G | A | P | P | P | S | NH2 | | | 5 |
| 5 | Km | | | | | | | | | | | | | 6 |
| 6 | K | | | | | | | | | | | | | 7 |
| 7 | R | OH | | | | | | | | | | | | 8 |
| 8 | R | NH2 | | | | | | | | | | | | 9 |
| 9 | R | OH | | | | | | | | | | | | 10 |
| 10 | S | OH | | | | | | | | | | | | 11 |
| 11 | S | P | S | S | G | A | P | P | P | S | NH2 | | | 12 |
| 12 | S | P | S | S | G | A | P | P | P | S | NH2 | | | 13 |
| 13 | R | P | S | S | G | A | P | P | P | S | NH2 | | | 14 |
| 14 | R | P | S | S | G | A | P | P | P | S | NH2 | | | 15 |
| 15 | R | P | S | S | G | A | P | P | P | S | NH2 | | | 16 |
| 16 | Km | P | S | S | G | A | P | P | P | S | NH2 | | | 17 |
| 17 | R | NH2 | | | | | | | | | | | | 18 |
| 18 | R | NH2 | | | | | | | | | | | | 19 |
| 19 | R | NH2 | | | | | | | | | | | | 20 |
| 20 | R | P | S | S | G | A | P | P | P | S | OH | | | 21 |
| 21 | R | G | G | G | G | S | NH2 | | | | | | | 22 |
| 22 | R | P | S | S | G | A | P | P | P | S | NH2 | | | 23 |
| 23 | R | P | S | S | G | A | P | P | P | S | NH2 | | | 24 |
| 24 | R | P | S | S | G | A | P | P | P | S | NH2 | | | 25 |
| 25 | R | P | S | S | G | A | P | P | P | S | NH2 | | | 26 |
| 26 | R | P | S | S | G | A | P | P | P | S | NH2 | | | 27 |
| 27 | Km | P | S | S | G | A | P | P | P | S | NH2 | | | 28 |
| 28 | R | P | S | S | G | A | P | P | P | S | NH2 | | | 29 |
| 29 | S | P | S | S | G | A | P | P | P | S | NH2 | | | 30 |
| 30 | R | G | G | G | G | S | NH2 | | | | | | | 31 |
| 31 | S | G | G | G | G | S | NH2 | | | | | | | 32 |
| 32 | R | G | G | G | G | S | NH2 | | | | | | | 33 |
| 33 | Km | G | G | G | G | S | NH2 | | | | | | | 34 |
| 34 | R | P | S | S | G | A | P | P | P | S | NH2 | | | 35 |
| 35 | R | P | S | S | G | A | P | P | P | S | NHZ | | | 36 |
| 36 | R | G | OH | | | | | | | | | | | 37 |
| 37 | R | G | OH | | | | | | | | | | | 38 |
| 38 | S | P | S | S | G | A | P | P | P | S | NH2 | | | 39 |
| 39 | K | G | OH | | | | | | | | | | | 40 |
| 40 | S | P | S | S | G | A | P | P | P | S | OH | | | 41 |
| 41 | K | G | OH | | | | | | | | | | | 42 |
| 42 | S | G | OH | | | | | | | | | | | 43 |
| 43 | R | P | S | S | G | A | P | P | P | S | OH | | | 44 |
| 44 | R | P | S | S | G | A | P | P | P | S | OH | | | 45 |
| 45 | S | P | S | S | G | A | P | P | P | S | OH | | | 46 |
| 46 | S | G | OH | | | | | | | | | | | 47 |
| 47 | S | P | S | S | G | A | P | P | P | S | OH | | | 48 |
| 48 | S | P | S | S | G | A | P | P | P | S | NH2 | | | 49 |
| 49 | R | G | OH | | | | | | | | | | | 50 |
| 50 | S | P | S | S | G | A | P | P | P | S | NH2 | | | 51 |
| 51 | R | P | S | S | G | A | P | P | P | S | OH | | | 52 |
| 52 | S | P | S | S | G | A | P | P | P | S | OH | | | 53 |
| 53 | S | G | OH | | | | | | | | | | | 54 |
| 54 | R | P | S | S | G | A | P | P | P | S | OH | | | 55 |
| 55 | R | G | OH | | | | | | | | | | | 56 |
| 56 | R | G | OH | | | | | | | | | | | 57 |
| 57 | R | G | G | G | G | S | NH2 | | | | | | | 58 |
| 58 | R | P | S | S | G | A | P | P | P | S | NH2 | | | 59 |
| 59 | S | P | S | S | G | A | P | P | P | S | NH2 | | | 60 |
| 60 | S | G | OH | | | | | | | | | | | 61 |
| 61 | Km | G | OH | | | | | | | | | | | 62 |
| 62 | R | G | OH | | | | | | | | | | | 63 |
| 63 | R | G | OH | | | | | | | | | | | 64 |
| 64 | R | G | OH | | | | | | | | | | | 65 |
| 65 | K | G | OH | | | | | | | | | | | 66 |
| 66 | K | G | OH | | | | | | | | | | | 67 |
| 67 | R | G | OH | | | | | | | | | | | 68 |
| 68 | S | P | S | S | G | A | P | P | P | S | NH2 | | | 69 |
| 69 | Km | A | S | S | G | A | P | P | P | S | NH2 | | | 70 |
| 70 | R | P | S | S | G | OH | | | | | | | | 71 |
| 71 | S | P | S | S | G | OH | | | | | | | | 72 |
| 72 | R | G | OH | | | | | | | | | | | 73 |
| 73 | R | G | OH | | | | | | | | | | | 74 |
| 74 | R | G | OH | | | | | | | | | | | 75 |
| 75 | K | G | OH | | | | | | | | | | | 76 |
| 76 | K | G | OH | | | | | | | | | | | 77 |
| 77 | K | G | OH | | | | | | | | | | | 78 |
| 78 | K | G | OH | | | | | | | | | | | 79 |
| 79 | K | G | OH | | | | | | | | | | | 80 |
| 80 | K | G | OH | | | | | | | | | | | 81 |
| 81 | K(Ac) | G | OH | | | | | | | | | | | 82 |
| 82 | Q | G | OH | | | | | | | | | | | 83 |
| 83 | S | P | G | OH | | | | | | | | | | 84 |
| 84 | S | P | G | OH | | | | | | | | | | 85 |
| 85 | S | P | G | OH | | | | | | | | | | 86 |
| 86 | S | P | G | OH | | | | | | | | | | 87 |
| 87 | S | P | G | OH | | | | | | | | | | 88 |
| 88 | S | P | G | OH | | | | | | | | | | 89 |
| 89 | R | P | S | S | G | A | P | P | P | S | NH2 | | | 90 |
| 90 | S | P | S | S | G | A | P | P | P | S | NH2 | | | 91 |
| 91 | S | P | S | S | G | A | P | P | P | S | NH2 | | | 92 |
| 92 | S | P | S | S | G | A | P | P | P | S | NH2 | | | 93 |
| 93 | S | P | S | S | G | A | P | P | P | S | NH2 | | | 94 |
| 94 | S | P | S | S | G | A | P | P | P | S | NH2 | | | 95 |
| 95 | S | P | S | S | G | A | P | P | P | S | OH | | | 96 |
| 96 | S | P | S | S | G | A | P | P | P | S | OH | | | 97 |
| 97 | S | P | S | S | G | A | P | P | P | S | NH2 | | | 98 |
| 98 | R | G | OH | | | | | | | | | | | 99 |
| 99 | S | P | S | S | G | A | P | P | P | S | NH2 | | | 100 |
| 100 | S | P | S | S | G | A | P | P | P | S | NH2 | | | 101 |
| 101 | R | P | S | S | G | A | P | P | P | S | NH2 | | | 102 |
| 102 | S | P | S | S | G | A | P | P | P | S | OH | | | 103 |
| 103 | S | P | S | S | G | A | P | P | P | S | OH | | | 104 |
| 104 | S | P | S | S | G | A | P | P | P | S | OH | | | 105 |
| 105 | S | P | S | S | G | A | P | P | P | S | OH | | | 106 |
| 106 | R | P | S | S | G | A | P | P | P | S | NH2 | | | 107 |
| 107 | S | P | S | S | G | A | P | P | P | S | NH2 | | | 108 |
| 108 | R | G | OH | | | | | | | | | | | 109 |
| 109 | S | P | S | S | G | A | P | P | P | S | NH2 | | | 110 |
| 110 | K | G | OH | | | | | | | | | | | 111 |
| 111 | S | P | G | OH | | | | | | | | | | 112 |
| 112 | S | P | S | S | G | A | P | P | P | S | OH | | | 113 |
| 113 | S | P | G | OH | | | | | | | | | | 114 |
| 114 | S | P | G | OH | | | | | | | | | | 115 |
| 115 | R | G | OH | | | | | | | | | | | 116 |
| 116 | S | P | S | S | G | OH | | | | | | | | 117 |
| 117 | S | P | G | OH | | | | | | | | | | 118 |
| 118 | S | P | G | OH | | | | | | | | | | 119 |
| 119 | S | P | G | OH | | | | | | | | | | 120 |
| 120 | S | P | G | OH | | | | | | | | | | 121 |
| 121 | R | G | OH | | | | | | | | | | | 122 |
| 122 | R | G | OH | | | | | | | | | | | 123 |
| 123 | R | G | OH | | | | | | | | | | | 124 |
| 124 | S | P | G | OH | | | | | | | | | | 125 |
| 125 | S | P | G | OH | | | | | | | | | | 126 |
| 126 | S | P | G | OH | | | | | | | | | | 127 |
| 127 | R | G | OH | | | | | | | | | | | 128 |
| 128 | S | P | G | OH | | | | | | | | | | 129 |
| 129 | R | G | OH | | | | | | | | | | | 130 |
| 130 | R | G | OH | | | | | | | | | | | 131 |
| 131 | R | G | OH | | | | | | | | | | | 132 |
| 132 | R | G | OH | | | | | | | | | | | 133 |
| 133 | R | G | OH | | | | | | | | | | | 134 |
| 134 | R | P | S | S | G | A | P | P | P | S | OH | | | 135 |
| 135 | R | P | S | S | G | A | P | P | P | S | OH | | | 136 |
| 136 | S | P | G | OH | | | | | | | | | | 137 |
| 137 | R | G | OH | | | | | | | | | | | 138 |
| 138 | S | P | S | S | G | A | P | P | P | S | OH | | | 139 |
| 139 | R | G | OH | | | | | | | | | | | 140 |
| 140 | R | G | OH | | | | | | | | | | | 141 |
| 141 | R | G | OH | | | | | | | | | | | 142 |
| 142 | R | G | OH | | | | | | | | | | | 143 |
| 143 | R | G | OH | | | | | | | | | | | 144 |
| 144 | R | G | OH | | | | | | | | | | | 145 |
| 145 | R | G | OH | | | | | | | | | | | 146 |
| 146 | R | G | OH | | | | | | | | | | | 147 |
| 147 | R | G | OH | | | | | | | | | | | 148 |
| 148 | R | G | OH | | | | | | | | | | | 149 |
| 149 | R | G | OH | | | | | | | | | | | 150 |
| 150 | R | G | OH | | | | | | | | | | | 151 |
| 151 | R | G | OH | | | | | | | | | | | 152 |
| 152 | R | G | OH | | | | | | | | | | | 153 |
| 153 | R | G | OH | | | | | | | | | | | 154 |
| 154 | R | G | OH | | | | | | | | | | | 155 |
| 155 | R | G | OH | | | | | | | | | | | 156 |
| 156 | R | G | OH | | | | | | | | | | | 157 |
| 157 | R | G | OH | | | | | | | | | | | 158 |
| 158 | R | G | OH | | | | | | | | | | | 159 |
| 159 | R | G | OH | | | | | | | | | | | 160 |
| 160 | R | G | OH | | | | | | | | | | | 161 |
| 161 | R | G | OH | | | | | | | | | | | 162 |
| 162 | R | G | OH | | | | | | | | | | | 163 |
| 163 | R | G | OH | | | | | | | | | | | 164 |
| 164 | R | G | OH | | | | | | | | | | | 165 |
| 165 | R | G | OH | | | | | | | | | | | 166 |
| 166 | R | G | OH | | | | | | | | | | | 167 |
| 167 | R | G | OH | | | | | | | | | | | 168 |
| 168 | R | G | OH | | | | | | | | | | | 169 |
| 169 | R | G | OH | | | | | | | | | | | 170 |
| 170 | R | G | OH | | | | | | | | | | | 171 |
| 171 | R | G | OH | | | | | | | | | | | 172 |
| 172 | R | G | OH | | | | | | | | | | | 173 |
| 173 | R | G | OH | | | | | | | | | | | 174 |
| 174 | R | G | OH | | | | | | | | | | | 175 |
| 175 | R | G | OH | | | | | | | | | | | 176 |
| 176 | R | G | OH | | | | | | | | | | | 177 |
| 177 | S | P | S | S | G | A | P | P | P | S | OH | | | 178 |
| 178 | S | P | G | OH | | | | | | | | | | 179 |
| 179 | S | p | S | S | G | A | P | P | P | S | OH | | | 180 |
| 180 | S | P | G | OH | | | | | | | | | | 181 |
| 181 | Km | P | S | S | G | A | P | P | P | S | | | | 182 |
| 182 | Km | P | S | S | G | A | P | P | P | S | | | | 183 |
| 183 | Km | P | S | S | G | A | P | P | P | S | | | | 184 |
| 184 | Km | P | S | S | G | A | P | P | P | S | | | | 185 |
| 185 | Km | P | S | S | G | A | P | P | P | S | | | | 186 |
| 186 | R | | | | | | | | | | | | | 187 |
| 187 | K | G | K | K | N | D | W | K | H | N | I | T | Q | 188 |
| 188 | K | G | K | K | N | D | W | K | H | N | I | T | Q | 189 |
| 189 | Km | G | K | K | N | D | W | K | H | N | I | T | Q | 190 |
| 190 | Km | P | S | S | G | A | P | P | P | S | | | | 191 |
| 191 | Km | | | | | | | | | | | | | 192 |
| 192 | S | | | | | | | | | | | | | 193 |
| 193 | K | | | | | | | | | | | | | 194 |
| 194 | R | G | OH | | | | | | | | | | | 195 |
| 195 | S | P | S | S | G | A | P | P | P | S | OH | | | 196 |
| 196 | S | P | G | OH | | | | | | | | | | 197 |
| 197 | S | P | G | OH | | | | | | | | | | 198 |
| 198 | S | P | S | S | G | A | P | P | P | S | OH | | | 199 |
| 199 | S | P | G | OH | | | | | | | | | | 200 |
| 200 | K | G | OH | | | | | | | | | | | 201 |
| 201 | R | G | OH | | | | | | | | | | | 202 |
| 202 | R | G | OH | | | | | | | | | | | 203 |
| 203 | K | G | OH | | | | | | | | | | | 204 |
| 204 | R | G | NH2 | | | | | | | | | | | 205 |
| 205 | R | G | OH | | | | | | | | | | | 206 |
| 206 | R | G | OH | | | | | | | | | | | 207 |
| 207 | R | G | OH | | | | | | | | | | | 208 |
| 208 | R | G | OH | | | | | | | | | | | 209 |
| 209 | R | G | OH | | | | | | | | | | | 210 |
| 210 | R | G | OH | | | | | | | | | | | 211 |
| 211 | R | G | OH | | | | | | | | | | | 212 |
| 212 | R | G | OH | | | | | | | | | | | 213 |
| 213 | R | G | OH | | | | | | | | | | | 214 |
| 214 | R | G | OH | | | | | | | | | | | 215 |
| 215 | R | G | OH | | | | | | | | | | | 216 |
| 216 | R | G | OH | | | | | | | | | | | 217 |
| 217 | R | G | OH | | | | | | | | | | | 218 |
| 218 | R | G | OH | | | | | | | | | | | 219 |
| 219 | R | G | OH | | | | | | | | | | | 220 |
| 220 | R | G | OH | | | | | | | | | | | 221 |
| 221 | R | G | OH | | | | | | | | | | | 222 |
| 222 | R | G | OH | | | | | | | | | | | 223 |
| 223 | R | G | OH | | | | | | | | | | | 224 |
| 224 | R | G | OH | | | | | | | | | | | 225 |
| 225 | Km | G | OH | | | | | | | | | | | 226 |
| 226 | R | G | OH | | | | | | | | | | | 227 |
| 227 | S | P | S | S | G | A | P | P | P | S | | | | 228 |
| 228 | S | P | S | S | G | A | P | P | P | S | | | | 229 |
| 229 | S | P | S | S | G | A | P | P | P | S | | | | 230 |
| 230 | R | G | OH | | | | | | | | | | | 231 |
| 231 | R | G | OH | | | | | | | | | | | 232 |
| 232 | R | P | S | S | G | A | P | P | P | S | | | | 233 |
| 233 | R | P | S | S | G | A | P | P | P | S | | | | 234 |
| 234 | R | P | S | S | G | A | P | P | P | S | | | | 235 |
| 235 | S | P | S | S | G | A | P | P | P | S | | | | 236 |
| 236 | R | G | OH | | | | | | | | | | | 237 |
| 237 | R | G | OH | | | | | | | | | | | 238 |
| 238 | R | P | S | S | G | A | P | P | P | S | | | | 239 |
| 239 | S | P | S | S | G | A | P | P | P | S | OH | | | 240 |
| 240 | S | P | S | S | G | A | P | P | P | S | OH | | | 241 |
| 241 | R | G | OH | | | | | | | | | | | 242 |
| 242 | R | G | OH | | | | | | | | | | | 243 |
| 243 | Km | | | | | | | | | | | | | 244 |
| 244 | Km | | | | | | | | | | | | | 245 |
| 245 | K | | | | | | | | | | | | | 246 |
| 246 | K | | | | | | | | | | | | | 247 |
| 247 | K | | | | | | | | | | | | | 248 |
| 248 | Km | | | | | | | | | | | | | 249 |
| 249 | Km | | | | | | | | | | | | | 250 |
| 250 | R | | | | | | | | | | | | | 251 |
| 251 | R | | | | | | | | | | | | | 252 |
| 252 | R | | | | | | | | | | | | | 253 |
| 253 | R | | | | | | | | | | | | | 254 |
| 254 | R | | | | | | | | | | | | | 255 |
| 255 | K | | | | | | | | | | | | | 256 |
| 256 | R | | | | | | | | | | | | | 257 |
| 257 | R | | | | | | | | | | | | | 258 |
| 258 | R | | | | | | | | | | | | | 259 |
| 259 | R | P | S | S | G | A | P | P | P | S | | | | 260 |
| 260 | R | P | S | S | G | A | P | P | P | S | | | | 261 |
| 261 | R | P | S | S | G | A | P | P | P | S | | | | 262 |
| 262 | R | P | S | S | G | A | P | P | P | S | | | | 263 |
| 263 | R | P | S | S | G | A | P | P | P | S | | | | 264 |
| 264 | R | | | | | | | | | | | | | 265 |
| 265 | R | | | | | | | | | | | | | 266 |
| 266 | R | P | S | S | G | A | P | P | P | S | | | | 267 |
| 267 | R | | | | | | | | | | | | | 268 |
| 268 | S | P | S | S | G | A | P | P | P | S | | | | 269 |
| 269 | R | | | | | | | | | | | | | 270 |
| 270 | S | P | S | S | G | A | P | P | P | S | | | | 271 |
| 271 | R | G | G | G | G | S | | | | | | | | 272 |
| 272 | R | | | | | | | | | | | | | 273 |
| 273 | S | P | S | S | G | A | P | P | P | S | | | | 274 |
| 274 | R | P | S | S | G | A | P | P | P | S | | | | 275 |
| 275 | S | P | S | S | G | A | P | P | P | S | | | | 276 |
| 276 | R | P | S | S | G | A | P | P | P | S | | | | 277 |
| 277 | R | P | S | S | G | A | P | P | P | S | R | | | 278 |
| 278 | R | P | S | S | G | A | P | P | P | S | R | | | 279 |
| 279 | R | P | S | S | G | A | P | P | P | S | R | | | 280 |
| 280 | R | P | S | S | G | A | P | P | P | S | R | | | 281 |
| 281 | K | G | K | K | N | N | W | K | H | N | | | | 282 |
| 282 | R | P | S | S | G | A | P | P | P | S | R | | | 283 |
| 283 | R | P | S | S | G | A | P | P | P | S | R | | | 284 |
| 284 | R | G | OH | | | | | | | | | | | 285 |
| 285 | R | P | S | S | G | OH | | | | | | | | 286 |
| 286 | R | G | OH | | | | | | | | | | | 287 |
| 287 | R | P | S | S | G | OH | | | | | | | | 288 |
| 288 | R | P | S | S | G | OH | | | | | | | | 289 |
| 289 | K | G | OH | | | | | | | | | | | 290 |
| 290 | S | P | G | OH | | | | | | | | | | 291 |
| 291 | R | G | OH | | | | | | | | | | | 292 |
| 292 | S | P | S | S | G | A | P | P | P | S | OH | | | 293 |
| 293 | S | P | S | S | G | A | P | P | P | S | NH2 | | | 294 |
| 294 | R | G | OH | | | | | | | | | | | 295 |

N-TER means N-terminus;
C-TER means C-terminus;
C17DA means C₁₇ diacid;
C18DA means C₁₈ diacid;
C20DA means C₂₀ diacid

Biological Examples

Methods for performing GIP and GLP receptor binding assays, assays for inhibition of emesis, vomiting and nausea, caused by various stimuli, including from drug or chemotherapy induced emesis are specifically described in Applicant's International PCT Application No. PCT/JP2018/013540, filed on Mar. 30, 2018, ranging from pages 213 to 255, and are specifically incorporated herein by reference in their entirety.

### Example 9. Evaluation of Peptide Agonist Activity on Human GIPR and Human GLP1R by Measuring Intracellular cAMP Accumulation

GIPR Assay

HEK-293T cells overexpressing full-length human GIPR with a sequence identical to GenBank accession number NM_000164 with an N-terminal FLAG tag are purchased from Multispan, Inc (Hayward, Calif.). Cells are cultured per the manufacturer's protocol in DMEM with 10% fetal bovine serum and 1 μg/mL puromycin and stored in frozen aliquots to be used as assay ready cells. On the day of the assay, cells are removed from frozen storage, washed two times in 1× Kreb's Ringer Buffer (Zenbio, Research Triangle Park, N.C.), and re-suspended to a concentration of 4×10⁵ cells/mL in 1× Kreb's Ringer Buffer. 50 nL of test compound in 100% DMSO spanning a final concentration range of 3×10⁻¹⁰-5.08×10⁻¹⁵ M are acoustically dispensed in low volume, white, 384-well polypropylene plates (Corning, Tewksbury, Mass.), followed by the addition of 4×10³ cells per well in total volume of 10 μL. Cells are incubated with test compound for 1 hr at room temperature in the dark, and cAMP accumulation is measured using the Cisbio HiRange cAMP assay kit (Bedford, Mass.) per the manufacturer's protocol. Anti-cAMP antibody and d2-cAMP tracer reagents diluted in lysis/detection buffer are incubated in the dark for 1 hr, and results are measured on an Envision plate reader (Perkin Elmer, Waltham, Mass.). Data is normalized using 1 nM GIP as 100% activity, and DMSO alone as 0% activity.

HEK-293T cells overexpressing full-length human GLP-1R with a sequence identical to GenBank accession number NM_002062 with an N-terminal FLAG tag may be purchased from Multispan, Inc (Hayward, Calif.). Cells are cultured per the manufacturer's protocol in DMEM with 10% fetal bovine serum and 1 μg/mL puromycin and stored in frozen aliquots to be used as assay ready cells. On the day of the assay, cells are removed from frozen storage, washed two times in 1× Kreb's Ringer Buffer (Zenbio, Research Triangle Park, N.C.), and re-suspended to a concentration of 4×10⁵ cells/mL in 1× Kreb's Ringer Buffer. 50 nL of test compound in 100% DMSO spanning a final concentration range of 1×10⁻⁶-1.69×10⁻¹¹ M are acoustically dispensed in low volume, white, 384-well polypropylene plates (Corning, Tewksbury, Mass.), followed by the addition of 4×10³ cells per well in total volume of 10 μL. Cells are incubated with test compound for 1 hr at room temperature in the dark, and cAMP accumulation is measured using the Cisbio HiRange cAMP assay kit (Bedford, Mass.) per the manufacturer's protocol. Anti-cAMP antibody and d2-cAMP tracer reagents diluted in lysis/detection buffer are incubated in the dark for 1 hr, and results are measured on an Envision plate reader (Perkin Elmer, Waltham, Mass.). Data is normalized using 1 nm GLP-1 as 100% activity, and DMSO alone as 0% activity.

TABLE 4
GIP receptor selective activation of various GIP
receptor agonist peptides of the disclosure

| | | | Human GLP1R |
|---|---|---|---|
| Compound | Sequence | Human GIPR cAMP | cAMP EC50, |
| No. | ID No. | EC50, HEK293T (nM) | HEK293T (nM) |
| 1 | 2 | 0.0004 | 10.4713 |
| 2 | 3 | 0.0003 | 524.8075 |
| 3 | 4 | 0.0006 | 10.4713 |
| 4 | 5 | 0.001 | 38.9045 |
| 5 | 6 | 0.0007 | 123.0269 |
| 6 | 7 | 0.0004 | 83.1764 |
| 7 | 8 | 0.0004 | 588.8437 |
| 8 | 9 | 0.0003 | 41.4477 |
| 9 | 10 | 0.0004 | 7.9433 |
| 10 | 11 | 0.0002 | 37.1535 |
| 11 | 12 | 0.0003 | 1000 |
| 12 | 13 | 0.0002 | 954.9926 |
| 13 | 14 | 0.0004 | 48.9779 |
| 14 | 15 | 0.0003 | 171.7908 |
| 15 | 16 | 0.0003 | 35.8922 |
| 16 | 17 | 0.0004 | 35.0752 |
| 21 | 22 | 0.0004 | 34.6737 |
| 22 | 23 | 0.0005 | 524.8075 |
| 23 | 24 | 0.0003 | 50.1187 |
| 24 | 25 | 0.0004 | 190.5461 |
| 25 | 26 | 0.0019 | 144.544 |
| 26 | 27 | 0.0003 | 38.9045 |
| 27 | 28 | 0.0004 | 87.0964 |
| 28 | 29 | 0.0003 | 20.893 |
| 29 | 30 | 0.0004 | 416.8694 |
| 30 | 31 | 0.0006 | 104.7129 |
| 31 | 32 | 0.0003 | 169.8244 |
| 32 | 33 | 0.0003 | 1000 |
| 33 | 34 | 0.0008 | 436.5158 |
| 34 | 35 | 0.0004 | 1000 |
| 35 | 36 | 0.0007 | 89.1251 |
| 36 | 37 | 0.0007 | 87.0964 |
| 37 | 38 | 0.0005 | 44.6684 |
| 38 | 39 | 0.0006 | 45.7088 |
| 39 | 40 | 0.0017 | 13.1826 |
| 40 | 41 | 0.0007 | 5.6 |
| 41 | 42 | 0.0011 | 13.8038 |
| 42 | 43 | 0.0008 | 5.4954 |
| 43 | 44 | 0.001 | 61.6595 |
| 44 | 45 | 0.0017 | 10.7152 |
| 45 | 46 | 0.0006 | 8.2224 |
| 46 | 47 | 0.0003 | 104.7129 |
| 47 | 48 | 0.0008 | 147.9108 |
| 48 | 49 | 0.0007 | 87.0964 |
| 49 | 50 | 0.0006 | 107.1519 |
| 50 | 51 | 0.0007 | 20.4174 |
| 51 | 52 | 0.0006 | 14.7911 |
| 52 | 53 | 0.0005 | 16.9824 |
| 53 | 54 | 0.0005 | 47.863 |
| 54 | 55 | 0.0006 | 120.2264 |
| 55 | 56 | 0.0009 | 741.3102 |
| 56 | 57 | 0.0006 | >1000 |
| 57 | 58 | 0.0006 | >1000 |
| 58 | 59 | 0.0004 | >1000 |
| 59 | 60 | 0.0005 | >1000 |
| 60 | 61 | 0.0003 | 100 |
| 61 | 62 | 0.0007 | >1000 |
| 62 | 63 | 0.0004 | >1000.0000 |
| 63 | 64 | 0.0002 | 75.8578 |
| 64 | 65 | 0.0002 | 109.6478 |
| 65 | 66 | 0.001 | 40.738 |
| 66 | 67 | 0.0008 | 32.3594 |
| 67 | 68 | 0.0013 | 33.8844 |
| 68 | 69 | 0.0007 | >1000.0000 |
| 69 | 70 | 0.0008 | 102.3293 |
| 70 | 71 | 0.0002 | 43.6516 |
| 71 | 72 | 0.0003 | 34.6737 |
| 72 | 73 | 0.0005 | 43.1519 |
| 73 | 74 | 0.0003 | 363.0781 |
| 74 | 75 | 0.0005 | 467.7351 |
| 75 | 76 | 0.0005 | 549.5409 |
| 76 | 77 | 0.0006 | 28.8403 |
| 77 | 78 | 0.0003 | 16.9824 |
| 78 | 79 | 0.0003 | 26.9153 |
| 80 | 81 | 0.0003 | 114.8154 |
| 81 | 82 | 0.0002 | 33.8844 |
| 82 | 83 | 0.0003 | 17.378 |
| 83 | 84 | 0.0007 | 66.0693 |
| 84 | 85 | 0.0002 | 58.8844 |
| 85 | 86 | 0.0004 | 41.6869 |
| 86 | 87 | 0.0003 | 58.8844 |
| 87 | 88 | 0.0004 | >1000.0000 |
| 88 | 89 | 0.0004 | 13.4896 |
| 89 | 90 | 0.0004 | 42.658 |
| 90 | 91 | 0.0005 | 72.4436 |
| 91 | 92 | 0.0005 | 30.903 |
| 92 | 93 | 0.0006 | 36.3078 |
| 93 | 94 | 0.0006 | 29.5121 |
| 94 | 95 | 0.0006 | 63.0957 |
| 95 | 96 | 0.0004 | 17.378 |
| 96 | 97 | 0.0003 | 9.5499 |
| 97 | 98 | 0.0006 | 52.4807 |
| 98 | 99 | 0.0004 | 112.2018 |
| 99 | 100 | 0.0005 | 478.6301 |
| 100 | 101 | 0.0006 | >1000.0000 |
| 101 | 102 | 0.0009 | 39.8107 |
| 102 | 103 | 0.0005 | 117.4898 |
| 103 | 104 | 0.0017 | 29.5121 |
| 104 | 105 | 0.0005 | 66.0693 |
| 105 | 106 | 0.0005 | 562.3413 |
| 106 | 107 | 0.0004 | 12.0226 |
| 107 | 108 | 0.0004 | 45.7088 |
| 108 | 109 | 0.0002 | 41.6869 |
| 109 | 110 | 0.0004 | 204.1738 |
| 110 | 111 | 0.0003 | 100 |
| 111 | 112 | 0.0001 | 14.1254 |
| 112 | 113 | 0.0003 | 21.3796 |
| 113 | 114 | 0.0003 | 13.4896 |
| 114 | 115 | 0.0001 | 11.749 |
| 115 | 116 | 0.0003 | 40.738 |
| 116 | 117 | 0.001 | 109.6478 |
| 117 | 118 | 0.002 | 44.6684 |
| 118 | 119 | 0.0007 | 91.2011 |
| 119 | 120 | 0.0009 | 50.1187 |
| 120 | 121 | 0.0003 | 15.8489 |
| 121 | 122 | 0.0005 | 89.1251 |
| 122 | 123 | 0.0008 | 3.5481 |
| 123 | 124 | 0.0005 | 74.131 |
| 124 | 125 | 0.0003 | 123.0269 |
| 125 | 126 | 0.0005 | 19.9526 |
| 126 | 127 | 0.0004 | 30.903 |
| 127 | 128 | 0.0007 | 63.0957 |
| 128 | 129 | 0.0009 | 7.2444 |
| 129 | 130 | 0.0004 | 114.8154 |
| 130 | 131 | 0.0003 | 95.4993 |
| 131 | 132 | 0.0004 | 151.3561 |
| 132 | 133 | 0.0009 | 66.0693 |
| 133 | 134 | 0.0012 | 58.8844 |
| 134 | 135 | 0.0006 | 169.8244 |
| 135 | 136 | 0.0007 | 8.3176 |
| 136 | 137 | 0.0007 | 63.0957 |
| 137 | 138 | 0.0013 | 3.4674 |
| 138 | 139 | 0.0008 | 2.0417 |
| 139 | 140 | 0.0015 | 8.9125 |
| 140 | 141 | 0.0007 | 3.9811 |
| 141 | 142 | 0.0005 | 5.2481 |
| 142 | 143 | 0.0004 | 158.4893 |
| 143 | 144 | 0.0004 | 4.8978 |
| 144 | 145 | 0.0009 | 15.1356 |
| 145 | 146 | 0.0018 | 9.3325 |
| 146 | 147 | 0.0008 | 7.0795 |
| 147 | 148 | 0.0008 | 1.6596 |
| 148 | 149 | 0.0004 | 8.7096 |
| 149 | 150 | 0.0007 | 15.8489 |
| 150 | 151 | 0.0004 | 13.4896 |
| 151 | 152 | 0.0003 | 5.0119 |
| 152 | 153 | 0.0006 | 33.1131 |
| 153 | 154 | 0.0003 | 6.7608 |
| 154 | 155 | 0.0003 | 8.9125 |
| 155 | 156 | 0.0008 | 85.1138 |
| 156 | 157 | 0.0013 | 10.2329 |
| 157 | 158 | 0.0003 | 436.5158 |
| 158 | 159 | 0.0005 | 338.8442 |
| 159 | 160 | 0.0009 | 11.749 |
| 160 | 161 | 0.0003 | 537.0318 |
| 161 | 162 | 0.0001 | 85.1138 |
| 162 | 163 | 0.0004 | 588.8437 |
| 163 | 164 | 0.0002 | 218.7762 |
| 164 | 165 | 0.0002 | >1000 |
| 165 | 166 | 0.0001 | >1000 |
| 166 | 167 | 0.0005 | 74.131 |
| 167 | 168 | 0.0009 | 38.0189 |
| 168 | 169 | 0.0009 | 16.5959 |
| 169 | 170 | 0.0008 | 177.8279 |
| 170 | 171 | 0.0005 | 61.6595 |
| 171 | 172 | 0.0006 | 83.1764 |
| 172 | 173 | 0.0011 | 93.3254 |
| 173 | 174 | 0.0003 | 104.7129 |
| 174 | 175 | 0.0004 | 47.863 |
| 175 | 176 | 0.0007 | 58.8844 |
| 176 | 177 | 0.0003 | 40.738 |
| 177 | 178 | 0.0003 | 100 |
| 178 | 179 | 0.0006 | 186.2087 |
| 179 | 180 | 0.0008 | 2.884 |
| 180 | 181 | 0.0009 | 5.7544 |
| 181 | 182 | 0.0004 | |
| 182 | 183 | 0.0525 | 213.7962 |
| 183 | 184 | 0.0007 | 162.181 |
| 184 | 185 | 0.0005 | |
| 185 | 186 | 0.001 | 1000 |
| 186 | 187 | 0.0003 | 1000 |
| 187 | 188 | 0.0032 | 562.3413 |
| 188 | 189 | 0.0019 | 131.8257 |
| 189 | 190 | 0.0011 | 218.7762 |
| 190 | 191 | 0.0005 | 97.7237 |
| 191 | 192 | 0.0032 | 97.7237 |
| 192 | 193 | 0.0002 | 177.8279 |
| 200 | 201 | 0.0006 | 77.6247 |
| 201 | 202 | 0.0009 | 61.6595 |
| 202 | 203 | 0.0042 | 2.5119 |
| 203 | 204 | 0.0013 | 4.1687 |
| 204 | 205 | 0.0003 | 6.6069 |
| 205 | 206 | 0.0008 | 33.1131 |
| 206 | 207 | 0.0004 | 26.3027 |
| 207 | 208 | 0.0004 | 36.3078 |
| 208 | 209 | 0.0003 | 9.3325 |
| 209 | 210 | 0.0004 | 20.893 |
| 210 | 211 | 0.0003 | 10.7152 |
| 211 | 212 | 0.0005 | 23.4423 |
| 212 | 213 | 0.0003 | 42.658 |
| 213 | 214 | 0.0011 | 40.738 |
| 214 | 215 | 0.0014 | 28.8403 |
| 215 | 216 | 0.0005 | 61.6595 |
| 216 | 217 | 0.0005 | 79.4328 |
| 217 | 218 | 0.0006 | 134.8963 |
| 218 | 219 | 0.0005 | 38.0189 |
| 219 | 220 | 0.0008 | 17.378 |
| 220 | 221 | 0.0005 | 0.3802 |
| 293 | 294 | 0.0004 | 128.825 |
| 294 | 295 | 0.0009 | 38.9045 |

Table 4 provides the selective binding activity of the GIPR agonist peptides of the present disclosure. As can be seen, the peptide compounds provided here have a Human GLP1R cAMP EC₅₀/Human GIPR cAMP EC₅₀ ratios ranging from about 800 to about 10,000,000, thus indicating incredibly selective GIPR agonist binding activity. Most of the GIPR agonist peptide compounds display Human GLPIR cAMP EC₅₀/Human GIPR cAMP EC₅₀ ratios of greater than 1,000, or greater than 5,000, or greater than 10,000, or greater than 50,000, or greater than 100,000, or greater than 500,000.

### Example 10. Oral Glucose Tolerance Test

An oral glucose tolerance test (OGTT) was carried out using C57BL/6J mice with a 50% dextrose solution dosed at 5 ml/kg. Testing concentrations of 0.1 nmol/kg, 0.3 nmmol/kg, 3 nmol/kg or 10 nmol/kg were selected depending on the peptide. Each peptide or a vehicle (control group) was subcutaneously administered 24 hours (unless shown otherwise) before glucose loading and the blood glucose was measured at 0, 15, 30, and 60 minutes after glucose dosing. The action of the compound was calculated by the calculation formula below and expressed as the % decrease in glucose as measured over 60 min using AUC.

% inhibition=(1−(AUC cpd−AUC naïve/AUC veh−AUC naïve))×100. Results are shown in Table 5. As shown in Table 5, it is verified that the compounds of the present invention suppress increase in blood glucose level caused by oral glucose loading.

TABLE 5
Oral glucose tolerance test

| | | Oral Glucose Tolerance Test |
|---|---|---|
| | | Percent decrease in blood |
| Compound | Sequence | glucose AUC (%) over 60 mins |
| No. | ID No. | at different compound doses (nmol/kg) |
| 8 | 9 | 54% (3 nmol/kg) |
| 11 | 12 | 28% (3 nmol/kg) |
| 12 | 13 | 30% (3 nmol/kg) |
| 13 | 14 | 55% (3 nmol/kg) |
| 14 | 15 | 25% (3 nmol/kg) |
| 15 | 16 | 34% (3 nmol/kg) |
| 16 | 17 | 40% (3 nmol/kg) |
| 18 | 19 | 8% (0.1 nmol/kg) |
| 20 | 21 | 30% (3 nmol/kg) |
| 21 | 22 | 32% (3 nmol/kg) |
| 23 | 24 | 46% (3 nmol/kg) |
| 25 | 26 | 9% (3 nmol/kg) |
| 26 | 27 | 44% (3 nmol/kg); 72 hrs |
| | | pretreatment |
| 27 | 28 | 45% (3 nmol/kg) |
| 30 | 31 | 27% (3 nmol/kg) |
| 31 | 32 | 43% (3 nmol/kg) |
| 33 | 34 | 72% (3 nmol/kg) |
| 35 | 36 | 47%, (3 nmol/kg) |
| 36 | 37 | 28% (3 nmol/kg) |
| 37 | 38 | 54% (3 nmol/kg) |
| 38 | 39 | 97% (3 nmol/kg) |
| 39 | 40 | 24% (3 nmol/kg) |
| 40 | 41 | 102% (3 nmol/kg) |
| 41 | 42 | 45% (3 nmol/kg), 15% (10 nmol/kg) |
| 42 | 43 | 62% (3 nmol/kg) |
| 43 | 44 | 97% (3 nmol/kg) |
| 44 | 45 | 119% (3 nmol/kg) |
| 45 | 46 | 95%, 43%, 68% (3 nmol/kg) |
| 47 | 48 | 48% (3 nmol/kg) |
| 48 | 49 | 87% (3 nmol/kg) |
| 49 | 50 | 36% (3 nmol/kg) |
| 50 | 51 | 89, 69% (3 nmol/kg) |
| 51 | 52 | 50% (3 nmol/kg) |
| 52 | 53 | 65% (3 nmol/kg) |
| 53 | 54 | 59% (3 nmol/kg) |
| 54 | 55 | 43% (3 nmol/kg) |
| 55 | 56 | 13% (3 nmol/kg) |
| 56 | 57 | 47% (3 nmol/kg) |
| 57 | 58 | 48% (3 nmol/kg) |
| 58 | 59 | 34% (3 nmol/kg) |
| 61 | 62 | 44% (3 nmol/kg) |
| 62 | 63 | 16% (3 nmol/kg) |
| 63 | 64 | 41% (3 nmol/kg) |
| 64 | 65 | 53% (3 nmol/kg) |
| 66 | 67 | 67% (3 nmol/kg) |
| 70 | 71 | 56% (3 nmol/kg) |
| 71 | 72 | 68% (3 nmol/kg) |
| 72 | 73 | 73% (0.1 nmol/kg), 66%(0.3 nmol/kg), |
| | | 38% (3 nmol/kg) |
| 73 | 74 | 49% (3 nmol/kg) |
| 74 | 75 | 18% (3 nmol/kg) |
| 80 | 81 | 22% (3 nmol/kg) |
| 81 | 82 | 66% (3 nmol/kg) |
| 82 | 83 | 72% (3 nmol/kg) |
| 83 | 84 | 68% (3 nmol/kg) |
| 84 | 85 | 72% (3 nmol/kg) |
| 85 | 86 | 47% (3 nmol/kg) |
| 86 | 87 | 29% (3 nmol/kg) |
| 87 | 88 | 50% (3 nmol/kg) |
| 88 | 89 | 64% (3 nmol/kg) |
| 89 | 90 | 32% (3 nmol/kg) |
| 90 | 91 | 41% (3 nmol/kg) |
| 91 | 92 | 44% (3 nmol/kg) |
| 93 | 94 | 24% (3 nmol/kg) |
| 94 | 95 | 40% (3 nmol/kg) |
| 96 | 97 | 80% (3 nmol/kg) |
| 99 | 100 | 35% (3 nmol/kg) |
| 100 | 101 | 17% (3 nmol/kg) |
| 104 | 105 | 78% (3 nmol/kg) |
| 105 | 106 | 82% (3 nmol/kg) |
| 106 | 107 | 40% (3 nmol/kg) |
| 107 | 108 | 28% (3 nmol/kg) |
| 108 | 109 | 65% (3 nmol/kg) |
| 110 | 111 | 57% (3 nmol/kg) |
| 111 | 112 | 44% (3 nmol/kg) |
| 114 | 115 | 50% (3 nmol/kg) |
| 115 | 116 | 40% (3 nmol/kg) |
| 117 | 118 | 30% (3 nmol/kg) |
| 119 | 120 | 12% (3 nmol/kg) |
| 122 | 123 | 19% (3 nmol/kg) |
| 123 | 124 | 10% (3 nmol/kg) |
| 124 | 125 | 06% (3 nmol/kg) |
| 128 | 129 | 57% (3 nmol/kg) |
| 136 | 137 | 19% (3 nmol/kg) |
| 137 | 138 | 23% (3 nmol/kg) |
| 138 | 139 | 80% (3 nmol/kg) |
| 139 | 140 | 93% (3 nmol/kg) |
| 141 | 142 | 16% (3 nmol/kg) |
| 144 | 145 | 35% (3 nmol/kg) |
| 145 | 146 | 52% (3 nmol/kg) |
| 146 | 147 | 52% (3 nmol/kg) |
| 148 | 149 | 51% (3 nmol/kg) |
| 149 | 150 | 13% (3 nmol/kg) |
| 153 | 154 | 58% (3 nmol/kg) |
| 154 | 155 | 51% (3 nmol/kg) |
| 155 | 156 | 6% (3 nmol/kg) |
| 156 | 157 | 13% (3 nmol/kg) |
| 159 | 160 | 54% (3 nmol/kg) |
| 161 | 162 | 4% (3 nmol/kg) |
| 167 | 168 | 2% (3 nmol/kg) |
| 174 | 175 | 41% (3 nmol/kg) |
| 176 | 177 | 53% (3 nmol/kg) |
| 177 | 178 | 47% (3 nmol/kg) |
| 179 | 180 | 58% (3 nmol/kg) |

As shown in Table 5, the GIPR agonist peptide compounds of the present disclosure suppress an increase in blood glucose level caused by oral glucose loading.

### Example 11: PYY-1119-Induced Vomiting in Dogs

Effects of single subcutaneous administration of the GIPR agonist compounds of the present disclosure on Neuropeptide Y2 receptor (Y2R) agonist compound PYY-1119 (4-imidazolecarbonyl-Ser-D-Hyp-Iva-Pya(4)-Cha-Leu(Me)-Asn-Lys-Aib-Thr-Arg-Gln-Arg-Cha-NH₂) (10 μg/kg [about 5 nmol/kg], s.c.) induced emesis were evaluated in dogs. The GIPR agonist peptide compounds of the present disclosure or vehicle (0.09% [w/v] Tween 80/10% DMSO/PBS) were administered subcutaneously (sc) at 3-10 nmol/kg to female beagle dogs (10 months old), followed by sc injections with Y2R agonist ((4-imidazolecarbonyl-Ser-D-Hyp-Iva-Pya(4)-Cha-Leu(Me)-Asn-Lys-Aib-Thr-Arg-Gln-Arg-Cha-NH₂), 10 μg/kg), 10 μg/kg) at 1 hour or specified hours in the table postdose. Emetic episodes were counted for 2 hours after administration (by blinded analysis).

Table 6 shows that the compounds suppressed the PYY-1119-induced emetic symptoms. In the below table, results are shown as percent inhibition (%) at the dose of peptide compound (nmol/kg) shown, at the hour(s) postdose of PYY-1119, calculated as (1−(number of emetic episodes with peptide compound/number of emetic episodes with vehicle))×100.

TABLE 6
Suppression of PYY-1119-induced vomiting in dogs.

| | | Inhibition (%) of PYY-1119-induced |
|---|---|---|
| | | emetic event at 1 hour or 8 hours(upper) |
| Compound | Sequence | and 72 hours(lower) post-dose at different |
| No. | ID No. | compound doses |
| 11 | 12 | 79% 1 h (10 nmol/kg) |
| | | 100% (10 nmol/kg) |
| 13 | 14 | 71% (3 nmol/kg) |
| | | 100% (3 nmol/kg) |
| 14 | 15 | 87% (10 nmol/kg) |
| | | 84% (10 nmol/kg) |
| 15 | 16 | 82% (10 nmol/kg) |
| | | 100% (10 nmol/kg) |
| 16 | 17 | 90% 1 h (10 nmol/kg) |
| | | 100% (10 nmol/kg) |
| 21 | 22 | 71% (3 nmol/kg) |
| | | 88% (3 nmol/kg) |
| 27 | 28 | 93% (3 nmol/kg) |
| | | 90% (3 nmol/kg) |
| 41 | 42 | 88% (3 nmol/kg) |
| | | 78% (3 nmol/kg) |
| 45 | 46 | 70% (3 nmol/kg) |
| | | 90% (3 nmol/kg) |
| 50 | 51 | 96% (3 nmol/kg) |
| | | 97% (3 nmol/kg) |
| 72 | 73 | 100% (3 nmol/kg) |
| | | 97% (3 nmol/kg) |
| 108 | 109 | 93% (3 nmol/kg) |
| | | 96% (3 nmol/kg) |
| 110 | 111 | 76% (3 nmol/kg) |
| | | 98% (3 nmol/kg) |
| 141 | 142 | 93% (3 nmol/kg) |
| | | 89% (3 nmol/kg) |
| 153 | 154 | 93% (3 nmol/kg) |
| | | 89% (3 nmol/kg) |
| 154 | 155 | 93% (3 nmol/kg) |
| | | 98% (3 nmol/kg) |
| 156 | 157 | 58% (3 nmol/kg) |
| | | 55% (3 nmol/kg) |
| 161 | 162 | 78% (3 nmol/kg) |
| | | 86% (3 nmol/kg) |
| 163 | 164 | 59% (3 nmol/kg) |
| | | 76% (3 nmol/kg) |
| 174 | 175 | 75% (3 nmol/kg) |
| | | 87% (3 nmol/kg) |
| 176 | 177 | 90% (3 nmol/kg) |
| | | 90% (3 nmol/kg) |
| 177 | 178 | 84% (3 nmol/kg) |
| | | 83% (3 nmol/kg) |
| 179 | 180 | 25% (3 nmol/kg) |
| | | 45% (3 nmol/kg) |

As shown in Table 6, it is verified that the peptide compounds of the present invention inhibited PYY-1119 induced emesis, including symptoms of vomiting in dogs.

### Example 12: Vomiting Suppression Test in Ferrets

1. Effect of subcutaneously administered GIP receptor agonist peptide in morphine-induced acute emetic model.

To evaluate the antiemetic effect, the GIP receptor agonist peptides other than natural human GIP are subcutaneously administered into male ferrets 24 hrs before morphine administration. Up to 60 minutes after morphine administration, the condition of the ferrets is monitored to record the frequencies and time points of abdominal contraction motions, vomiting behaviors, licking with the tongue, and fidgety behavior occurring. GIP receptor agonist peptide compounds of the present disclosure dosed at 0.3-10 nmol/kg attenuated the morphine (0.6 mg/kg, s.c.)-induced emesis in the ferrets.

GIP receptor agonist peptides are dissolved with a vehicle (0.09 w/v % tween 80/10% DMSO/saline), respectively, to prepare test solutions. 0.5 mg/kg of the test solutions and the vehicle are subcutaneously administered to ferrets (4 in each group), respectively. At the time of each of 4 or 24 hours after administration, 0.6 mg/kg of morphine is subcutaneously administered. Up to 60 minutes after morphine administration, the condition of the ferrets is monitored to record the number of animals that did not vomit, the number of emetic episodes, the latency period in minutes to observe the emetic episodes, the duration of the observed emesis if any.

TABLE 7
Percent inhibition of emetic symptoms induced by morphine in ferrets
treated with GIPr agonist peptides of the present disclosure.

| | | % inhibition of emetic |
|---|---|---|
| | | events at doses (nmol/kg) |
| Compound | Sequence | and timepoints shown in |
| No. | ID No. | ferret morphine model |
| 72 | 73 | 24 hrs: 95% (1 nmol/kg); |
| | | 100% (3 nmol/kg); 100% |
| | | (10 nmol/kg) |
| 84 | 85 | 24 hrs: 65% (1 nmol/kg); |
| | | 86% (3 nmol/kg); 100% |
| | | (10 nmol/kg) |
| 50 | 51 | 4 hrs: 27% (0.3 nmol/kg); |
| | | 69% (1 nmol/kg); 95% |
| | | (3 nmol/kg) |

Results from the above example, clearly illustrate that peptide compounds 72, 84, and 50 are effective in completely inhibiting the frequency of emesis, including frequency of vomiting in ferrets dosed with morphine.

### Example 13: Apomorphine (ApoM) Testing Observation Protocol of Apomorphine-Induced Emesis Symptoms in Dogs

Dogs are transferred to an observation cage (700 mm W×700 mm D×700 mm H [W×D×H], without food) on 1 day before each apomorphine challenge. The dogs are weighed by using an electronic balance then test peptide compounds and vehicle will be administrated via the subcutaneous route. Apomorphine is challenged at 8 hr after the administration and emetic events will be monitored for 1 h by video recording. The second apomorphine challenge will be 72 hr after the administration and emetic events will be recorded by the same protocol. Emesis symptoms are continuously recorded using a video camera and stored on a blue ray disc. Observation of symptoms include retching (a rhythmic contraction of the abdomen) and vomiting (vomiting behavior, including the elimination of vomitus or similar behavior). Besides, the combination of retching and vomiting is defined as emesis, and the number of episodes, latency (time elapsed from morphine administration until the onset of the first emesis symptom), duration (time elapsed between the onset of the first and final episodes of emesis), and frequency (number of animals showing emesis/number of experimental animals) of each of these symptoms is calculated. The latency in cases where emesis symptoms are not noted is taken as the maximum value (1 h for apomorphine challenge) at the end of observation. When the duration of the emesis symptoms is less than 1 min, the duration is recorded, for convenience, as 1 min. Table 8 shows the results of the ApoM testing:

| | | % inhibition of emetic events at doses |
|---|---|---|
| Compound | Sequence | (nmol/kg) shown in dog apomorphine model |
| No. | ID No. | after 8 hrs(upper) and 72 hours(lower) |
| 13 | 14 | 47% (10 nmol/kg) |
| | | 48% (10 nmol/kg) |
| 15 | 16 | 47% (10 nmol/kg) |
| | | 48% (10 nmol/kg) |
| 16 | 17 | 63% (10 nmol/kg) |
| | | 68% (10 nmol/kg) |
| 36 | 37 | 64% (30 nmol/kg) |
| | | 72% (30 nmol/kg) |
| 41 | 42 | 55% (60 nmol/kg) |
| | | 69% (60 nmol/kg) |
| 45 | 46 | 70% (30 nmol/kg) |
| | | 72% (30 nmol/kg) |
| 50 | 51 | 82% (30 nmol/kg) |
| | | 86% (30 nmol/kg) |
| 85 | 86 | 69% (10 nmol/kg) |
| | | 78% (10 nmol/kg) |
| 108 | 109 | 50% (10 nmol/kg) |
| | | 54% (10 nmol/kg) |
| 128 | 129 | 56% (10 nmol/kg) |
| | | 60% (10 nmol/kg) |

Results from the above example, clearly illustrate that Compounds 36, 45, 50, and 85, are effective in inhibiting the frequency of emetic events (>70% inhibition) in ferrets dosed with apomorphine.

### Example 14: Serum Half-Life and Percentage Remaining at 48 Hours

Serum Half-Life Analysis

Human plasma (mixed gender: sodium heparin is used as anti-coagulant; pre-adjusted to pH 7.4—NB alternative species may be used) is spiked with each test peptide (500 nM) and incubated·cn=3) at 37° C. for 48 hours in a 5% CO₂ environment. Aliquots are taken at 0. 1, 2, 4, 7, 24 and 46 hours and pH adjusted to pH 3 with 20% formic acid prior to analysis. Appropriate positive control compounds will be incubated in parallel, in addition to a no plasma control, sampled at 0 and 8 hours. All samples will be treated with ice-cold acetonitrile/methanol (4:1 (v/v)) containing internal standard prior to centrifugation at 2000 g and 4° C. for 10 minutes and subjected to LC-MS/MS analysis.

Sample Analysis

The samples are analyzed by LC-MS/MS using a 6500 (or equivalent) triple quadrupole mass spectrometer (AB Sciex) coupled to an appropriate Liquid Chromatography (LC) system. Protein binding and stability values are determined via peak area ratios using multiple reaction monitoring (MRM) parameters following compound optimisation. Multiple reaction monitoring (MRM) is a highly sensitive method of targeted mass spectrometry (MS) that can be used to selectively detect and quantify peptides based on the screening of specified precursor peptide-to-fragment ion transitions.

Table 9 shows the results of the Serum half-life of the compounds and the percentage remaining at 48 hours:

| Compound | Sequence | Serum t_{1/2} | % Remaining |
|---|---|---|---|
| No. | ID No. | (hours) | at 48 hr |
| 1 | 2 | >48 | 100 |
| 2 | 3 | 24.13 | 27 |
| 3 | 4 | 15.94 | 13 |
| 4 | 5 | 17.14 | 15 |
| 15 | 16 | >48 | 131.1 |

Table 9 provides two sets of data points related to the pharmacokinetic activity of the GIPR agonist peptides of the present disclosure. Optimum values for the use of the GIPR agonist peptides of the present disclosure range between a serum T1/2 (half life) of >48 hours for once weekly dosing. As can be seen from Table 9, when the T1/2 in serum approaches 48 hours and greater, the amount remaining after 48 hours exceeds 90%, which indicates that the peptide is available to exert its pharmacological activity for the duration of 5-7 days.

### Example 15: Human and Dog PPB

Human Plasma Protein Binding (PPB)

Stock Solutions

Stock solutions: (1000 μM) of the peptides are prepared in DMSO.

Plasma Protein Binding (PPB) Analysis

Human plasma (mixed gender; containing K2-EDTA as anti-coagulant; pre-adjusted to pH 7.4—NB alternative species may be used) is spiked individually with each test peptide (1000 nmol/L), sampled for analysis and then incubated (n=4) at 37° C. in a water bath for 30 minutes. Following the incubation period, the plasma is sampled for analysis, then transferred to ultracentrifugation tubes and centrifuged (n=3) at −450,000 g and 4° C. for 3 hours, after which the supernatant is sampled for analysis. An additional aliquot of the supernatant is taken at the end of the centrifugation period to determine the total protein concentration. An aliquot of the incubated plasma will be stored at 4° C. for 3 hours and then sampled for analysis. At the point of sampling, all samples are matrix-matched, treated with ice-cold acetonitrile/methanol (4:1 (vfv)) containing internal standard, centrifuged al 2000 g and 4° C. for 10 minutes and stored prior to LC-MS/MS analysis. An appropriate positive control compound control will be incubated and centrifuged in parallel; control plasma is also centrifuged to generate samples for matrix-matching. Fraction unbound in plasma (Fuₚₗₐₛₘₐ) values is determined by comparison of the analyte response in plasma to the analyte response in the supernatant, determined via peak area response ratios.

Plasma Stability Analysis

Human plasma (mixed gender: sodium heparin is used as anti-coagulant; pre-adjusted to pH 7.4—NB alternative species may be used) is spiked with each test peptide (500 nM) and incubated·cn=3) at 37° C. for 48 hours in a 5% Co₂ environment. Aliquots are taken at 0. 1, 2, 4, 7, 24 and 46 hours and pH adjusted to pH 3 with 20% formic acid prior to analysis. Appropriate positive control compounds will be incubated in parallel, in addition to a no plasma control, sampled at 0 and 8 hours. All samples will be treated with ice-cold acetonitrile/methanol (4:1 (v/v)) containing internal standard prior to centrifugation at 2000 g and 4° C. for 10 minutes and subjected to LC-MS/MS analysis.

Sample Analysis

The samples are analyzed by LC-MS/MS using a 6500 (or equivalent) triple quadrupole mass spectrometer (AB Sciex) coupled to an appropriate Liquid Chromatography (LC) system. Protein binding and stability values are determined via peak area ratios using multiple reaction monitoring (MRM) parameters following compound optimisation. Multiple reaction monitoring (MRM) is a highly sensitive method of targeted mass spectrometry (MS) that can be used to selectively detect and quantify peptides based on the screening of specified precursor peptide-to-fragment ion transitions.

Dog PPB values presented below are obtained essentially as described for Human PPB samples, with the difference being that dog serum is used instead of human serum. Table 10 is provided with the values of (Fu,plasma) as fraction unbound expressed as a percentage compared to the percent bound. i.e. if the value is 0.0123, then the fraction unbound is (0.0123/100)%, which is 1.23% of the peptide is unbound and 98.77% is bound in plasma. Methods and analysis for performing the dog PPB protocol and results are the same as noted herein as for the Human PPB protocol and analysis.

Table 10 shows the results of the dog PBB and Human PBB:

| Compound | SEQ ID | Human PPB | |
|---|---|---|---|
| No. | No. | (FUₚₗₐₛₘₐ) | Dog PPB (FUₚₗₐₛₘₐ) |
| 8 | 9 | 0.0009 | 0.0016 |
| 11 | 12 | 0.0038 | ≤0.0024 |
| 13 | 14 | <0.002 | <0.002 |
| 15 | 16 | 0.0017 | 0.0021 |
| 16 | 17 | <0.0012 | ≤0.00116 |
| 19 | 20 | 0.0011 | 0.0005 |
| 27 | 28 | 0.0013 | <0.002 |
| 36 | 37 | <0.002 | 0.0034 |
| 37 | 38 | 0.0010 | 0.0012 |
| 39 | 40 | 0.0112 | 0.0015 |
| 40 | 41 | <0.002 | 0.0004 |
| 41 | 42 | <0.002 | 0.0007 |
| 44 | 45 | 0.0007 | 0.0004 |
| 50 | 51 | <0.002 | 0.0006 |
| 72 | 73 | 0.0036 | 0.0062 |
| 84 | 85 | 0.0009 | 0.0003 |
| 293 | 294 | <0.002 | <0.002 |
| 294 | 295 | <0.002 | 0.0111 |

As can be seen in Table 10, the GIPR agonist peptides of the present disclosure provide a percent of unbound or active drug in human serum for antiemetic activity, which ranges from about 0.1% to about 0.4%. The efficacy of the GIPR agonist peptide will be related to the exposure to the amount of unbound drug in plasma, i.e. the proportion free peptide to penetrate into surrounding tissues. The bound peptide in plasma can also serve as a reservoir for free peptide removed by various elimination processes thus prolonging the duration of action. These GIPR agonist peptides also demonstrate that due to the high proportion of the drug being bound in human for example, (99.9% to 98.9%), the duration of action can be extended for longer periods of time. GIPR agonist peptides of the present disclosure provide an optimum range of unbound to plasma protein for once weekly dosing to human subjects between less than 0.5% unbound. It is believed that GIPR agonist peptides of the present disclosure having a free fraction of about 0.1% to about 0.5% translates to a peptide having a desirable pK profile, demonstrating suitable absorption and desired elimination to prevent excessive accumulation. Several compounds in Table 10 demonstrate optimum free unbound peptide, in human plasma, for example, compounds 8, 11, 15, 16, 27, 41, 50, 72, 84, 293 and 294 suitable for once per week (QW) dosing.

### Example 16: Solubility of the Compounds

Solubility Measurement pH 7.4

Solubility of the GIPR Agonist Compounds

3 mg of peptides are weighted out in a small glass vial. 100 uL of 200 mM Phosphate buffer pH 7.4 are added and the vial is sonicated/votexed as necessary for a maximum of 1 min. A visual inspection is performed, If the sample is fully dissolved, the solubility is recorded as 30 mg/mL. If insoluble material is observed in the tube the addition of 100 uL of buffer and mixing is repeated until complete dissolution. If the peptide is not soluble in 500 uL of buffer, it is labeled as solubility <6 mg/mL. The solubility can be confirmed by RP-HPLC after filtration on 0.2 μm filter on an Agilent 1200 system with a Kinetex column form Phenomenex® (2.6 μm EVO C18 100 Å, LC Column 50×3.0 mm) kept at 40° C., the eluent A is 0.05% TFA in Water, B is 0.035% TFA in Acetonitrile at a 0.6 ml/min flow rate. The gradient was from 20 to 70 over 5 min, the column is then washed for Imin at 90% B. UV monitoring at 215 nm was used to monitor peptide concentration.

Table 11 shows the results of the solubility of the compounds in phosphate buffer at pH 7.4:

| Compound | Sequence | pH 7.4 Phosphate Buffer - |
|---|---|---|
| No. | ID No. | Solubility (mg/mL) |
| 1 | 2 | 30 |
| 2 | 3 | 30 |
| 3 | 4 | 30 |
| 4 | 5 | 30 |
| 5 | 6 | 27 |
| 6 | 7 | 15 |
| 7 | 8 | 30 |
| 8 | 9 | 15 |
| 9 | 10 | 30 |
| 10 | 11 | 30 |
| 11 | 12 | 30 |
| 12 | 13 | 30 |
| 13 | 14 | 30 |
| 14 | 15 | 30 |
| 15 | 16 | 30 |
| 16 | 17 | 30 |
| 17 | 18 | 30 |
| 18 | 19 | 30 |
| 19 | 20 | 30 |
| 20 | 21 | 15 |
| 21 | 22 | 30 |
| 22 | 23 | 30 |
| 23 | 24 | 30 |
| 24 | 25 | 30 |
| 25 | 26 | 30 |
| 26 | 27 | 30 |
| 27 | 28 | 30 |
| 28 | 29 | 30 |
| 29 | 30 | 30 |
| 30 | 31 | 30 |
| 31 | 32 | 30 |
| 32 | 33 | 30 |
| 33 | 34 | 30 |
| 34 | 35 | 30 |
| 35 | 36 | 30 |
| 36 | 37 | 60 |
| 37 | 38 | 30 |
| 38 | 39 | 30 |
| 39 | 40 | 30 |
| 40 | 41 | 30 |
| 41 | 42 | 30 |
| 42 | 43 | 30 |
| 43 | 44 | 30 |
| 44 | 45 | 30 |
| 45 | 46 | 60 |
| 46 | 47 | 30.00 |
| 47 | 48 | 30 |
| 48 | 49 | 30 |
| 49 | 50 | 30.00 |
| 50 | 51 | 60 |
| 51 | 52 | 30 |
| 52 | 53 | 30 |
| 53 | 54 | 30 |
| 54 | 55 | 30 |
| 55 | 56 | 30 |
| 56 | 57 | 30 |
| 57 | 58 | 30 |
| 58 | 59 | 30 |
| 59 | 60 | 30 |
| 60 | 61 | 30 |
| 61 | 62 | 30 |
| 62 | 63 | 30 |
| 63 | 64 | 15 |
| 64 | 65 | 30 |
| 65 | 66 | 30 |
| 66 | 67 | 30 |
| 67 | 68 | 30 |
| 68 | 69 | 30 |
| 69 | 70 | 30 |
| 70 | 71 | 30 |
| 71 | 72 | 30 |
| 72 | 73 | 60 |
| 73 | 74 | 30 |
| 74 | 75 | 30 |
| 75 | 76 | 30 |
| 76 | 77 | 30 |
| 77 | 78 | 30 |
| 78 | 79 | 30 |
| 79 | 80 | 30 |
| 80 | 81 | 30 |
| 81 | 82 | 30 |
| 82 | 83 | 30 |
| 83 | 84 | 30 |
| 84 | 85 | 60 |
| 85 | 86 | 30 |
| 86 | 87 | 30 |
| 87 | 88 | 30 |
| 88 | 89 | 30 |
| 89 | 90 | 60 |
| 90 | 91 | 60 |
| 91 | 92 | 60 |
| 92 | 93 | 60 |
| 93 | 94 | 60 |
| 94 | 95 | 60 |
| 95 | 96 | 60 |
| 96 | 97 | 60 |
| 97 | 98 | 60 |
| 98 | 99 | 30 |
| 99 | 100 | 60 |
| 100 | 101 | 60 |
| 101 | 102 | 60 |
| 102 | 103 | 60 |
| 103 | 104 | 60 |
| 104 | 105 | 60 |
| 105 | 106 | 60 |
| 106 | 107 | 30 |
| 107 | 108 | 60 |
| 108 | 109 | 60 |
| 109 | 110 | 60 |
| 110 | 111 | 60 |
| 111 | 112 | 30 |
| 112 | 113 | 30 |
| 113 | 114 | 30 |
| 114 | 115 | 30 |
| 115 | 116 | 30 |
| 116 | 117 | 30 |
| 117 | 118 | 30 |
| 118 | 119 | 30 |
| 119 | 120 | 30 |
| 120 | 121 | 30 |
| 121 | 122 | 15 |
| 122 | 123 | 30 |
| 123 | 124 | 30 |
| 124 | 125 | 30 |
| 125 | 126 | 30 |
| 126 | 127 | 30 |
| 127 | 128 | 30 |
| 128 | 129 | 30 |
| 129 | 130 | 30 |
| 130 | 131 | 30 |
| 131 | 132 | 30 |
| 132 | 133 | 30 |
| 133 | 134 | 30 |
| 134 | 135 | 30 |
| 135 | 136 | 30 |
| 136 | 137 | 30 |
| 137 | 138 | 30 |
| 138 | 139 | 30 |
| 139 | 140 | 30 |
| 140 | 141 | 30 |
| 141 | 142 | 30 |
| 142 | 143 | 30 |
| 143 | 144 | 30 |
| 144 | 145 | 30 |
| 145 | 146 | 30 |
| 146 | 147 | 30 |
| 147 | 148 | 15 |
| 148 | 149 | 30 |
| 149 | 150 | 30 |
| 150 | 151 | 30 |
| 151 | 152 | 30 |
| 152 | 153 | 30 |
| 153 | 154 | 30 |
| 154 | 155 | 30 |
| 155 | 156 | 30 |
| 156 | 157 | 30 |
| 157 | 158 | 30 |
| 158 | 159 | 30 |
| 159 | 160 | 30 |
| 160 | 161 | 30 |
| 161 | 162 | 30 |
| 162 | 163 | 30 |
| 163 | 164 | 30 |
| 164 | 165 | 30 |
| 165 | 166 | 30 |
| 166 | 167 | 30 |
| 167 | 168 | 30 |
| 168 | 169 | 30 |
| 169 | 170 | 30 |
| 170 | 171 | 30 |
| 171 | 172 | 30 |
| 172 | 173 | 30 |
| 173 | 174 | 30 |
| 174 | 175 | 30 |
| 175 | 176 | 30 |
| 176 | 177 | 30 |
| 177 | 178 | 30 |
| 178 | 179 | 30 |
| 179 | 180 | 30 |
| 180 | 181 | 15 |
| 243 | 244 | 7 |
| 244 | 245 | 9.4 |
| 245 | 246 | 7.5 |
| 246 | 247 | 0.7 |
| 247 | 248 | 9.9 |
| 248 | 249 | 6.8 |
| 249 | 250 | 6.8 |
| 250 | 251 | 0.7 |
| 251 | 252 | 7 |
| 252 | 253 | 7.3 |
| 253 | 254 | 7.4 |
| 254 | 255 | 7.2 |
| 255 | 256 | 10 |
| 256 | 257 | 6 |
| 257 | 258 | 7.5 |
| 258 | 259 | 6 |
| 259 | 260 | <6 |
| 260 | 261 | <6.00 |
| 261 | 262 | <6.00 |
| 262 | 263 | <6.00 |
| 263 | 264 | 7.5 |
| 264 | 265 | <6.00 |
| 265 | 266 | 7.5 |
| 266 | 267 | <6.00 |
| 267 | 268 | <6.00 |
| 268 | 269 | 10 |
| 269 | 270 | 7.5 |
| 270 | 271 | 7.5 |
| 271 | 272 | 6 |
| 272 | 273 | 7.5 |
| 273 | 274 | 6 |
| 274 | 275 | 10 |
| 275 | 276 | <15 |
| 276 | 277 | <15 |
| 277 | 278 | <15 |
| 278 | 279 | <15 |
| 279 | 280 | <15 |
| 280 | 281 | <15 |
| 281 | 282 | <15 |
| 282 | 283 | <15 |
| 283 | 284 | <15 |
| 284 | 285 | <15 |
| 285 | 286 | <6 |
| 286 | 287 | <6 |
| 287 | 288 | <6 |
| 288 | 289 | <6 |
| 289 | 290 | 10 |
| 290 | 291 | 7.5 |
| 291 | 292 | <6 |
| 292 | 293 | <6 |
| 293 | 294 | 30 |
| 294 | 295 | 30 |

As shown in Table 11, several of the tested GIPR agonist peptides demonstrate high solubility in physiological buffer (Phosphate buffer at pH 7.4) of 15 mg/mL and above. Compounds 1-180, 293, and 294 exhibit a solubility in phosphate buffer at pH 7.4 of 15 mg/mL or greater, which are the preferred compounds for dosing in volumes that facilitate once per week or QW dosing. Compounds and 243-292 have a solubility of less than 15 mg/mL, for example less than 15 mg/mL, or from 10 mg/mL to 15 mg/mL are less preferred, and peptide compounds having less than 10 mg/mL solubility as described in Example 16 are excluded from the GIPR agonist peptides that are suitable for QW dosing. In some embodiments, GIPR agonist peptide compounds of the present disclosure having less than 15 mg/mL solubility as described in Example 16 are excluded from the GIPR agonist peptides that are suitable for QW dosing.

### Example 17: Summary of Pharmacokinetic (PK) and Pharmacodynamic (PD) Studies of Selective GIP Receptor Agonist Peptides

Pharmacokinetic (PK) were conducted in dog in order to determine the half-life after IV and SC dosing. The peptide was dissolved in 10% DMSO/0.09% Polysorbate/PBS pH 7.4 to a concentration of 3 nmol/mL and the animal were dose with a volume of 1 mL/kg SC or IV. Blood sample were collected at 0, 0.033, 0.083, 0.25, 0.5, 1, 2, 4, 6, 8, 12, 24, 48, 72, 96, 120, 168, 336 hours for IV dosing and 0.25, 0.5, 1, 2, 4, 6, 8, 12, 24, 48, 72, 96, 120, 168, 336 for SC dosing, EDTA-K2 was used as anticoagulant. The plasma concentration of the peptide was measured using LCMS. Allometric scaling of lipidated peptide pharmacokinetics including T1/2 and MRT is known in the art for rodent to dog and mini pig and to humans. In one illustrative embodiment, a lipidated peptide was shown to have MRT=145 hrs following s.c. dosing in dog and is dosed QW in humans. See for example, Discovery and Development of Liraglutide and Semaglutide. Knudsen, L. B.; Lau, J. Frontiers in Endocrinology, 2019, vol 10, Article 155.

Table 12 shows PK and PD data of selective compounds:

| Compound | Dog PK IV & SC 3 nmol/kg | |
|---|---|---|
| No. | IV T½ (SC MRT 0-last) in hours | |
| 45 | 62 | (125) |
| 16 | 38 h | (59) |
| 15 | 57 h | (64) |
| 84 | 62 | (82) |
| 50 | 74 | (103) |
| 41 | 115 | (119) |
| 72 | 92 | (125) |
| 27 | 77 | (109) |
| 293 | 68 | (83) |
| 294 | 59 | (97) |

As shown above in Table 12, peptide compounds 45, 15, 84, 50, 41, 72, 27, 293, and 294 all demonstrate exemplary pharmacokinetic activity providing the optimal exposure for once per week dosing (QW). As shown in Table 12, the IV T1/2 life (data provided for dogs) ranging from IV T12 life ranging from 57 to 115 hours when dosed at 3 nmol/kg. In comparison, compound no. 16 demonstrates an unacceptably short mean residence time (MRT) of 59 hours for once per week dosing. GIPR agonist compounds of the present disclosure are therapeutically effective for once-per week QW dosing when the MRT as shown in Table 12 are greater than 60 hours, or greater than 70 hours, or greater than 80 hours, when tested under the conditions shown in Example 17.

Formulation Example 1

| | | |
|---|---|---|
| (1) | Compound 10 | 10.0 mg |
| (2) | Lactose | 70.0 mg |
| (3) | Cornstarch | 50.0 mg |
| (4) | Soluble starch | 7.0 mg |
| (5) | Magnesium stearate | 3.0 mg |

Compound 10 (10.0 mg) and magnesium stearate (3.0 mg) are granulated with an aqueous soluble starch solution (0.07 mL) (7.0 mg as soluble starch), dried and mixed with lactose (70.0 mg) and cornstarch (50.0 mg). The mixture is compressed to give a tablet.

Formulation Example 2

| | | | |
|---|---|---|---|
| (1) | Compound 5 | 5.0 | mg |
| (2) | Sodium chloride | 20.0 | mg |
| (3) | Distilled water to total amount | 2 | mL |

Compound 5 (5.0 mg) and sodium chloride (20.0 mg) are dissolved in distilled water, and water is added to a total amount of 2.0 ml. The solution is filtered, and filled in a 2 ml ampoule under aseptic conditions. The ampoule is sterilized and tightly sealed to give a solution for injection.

### INDUSTRIAL APPLICABILITY

The GIP receptor agonist peptides of the present disclosure have superior GIP receptor selective agonist activity, and are useful as a drug for the prophylaxis or treatment of emesis and conditions caused by associated with GIP receptor activity, for example, emesis and diseases associated with vomiting or nausea and the like. In one embodiment, the selective GIP receptor agonist peptides are useful as a drug or medicament, or for use in the prophylaxis or treatment of emesis and conditions caused by associated with GIP receptor activity, for example cyclic vomiting syndrome, and nausea and/or vomiting associated with administration of a chemotherapeutic or anti-cancer agent as illustrated herein.

All the publications, patents, and the patent applications cited herein are incorporated herein by reference in their entireties.

### [Free Text for Sequence Listing]

SEQ ID NO: 1: Natural human GIP (1-42 peptide)
SEQ ID NO: 2 to 295 Synthetic peptides (Formulas (I)-(V)

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the claims.

1. A GIP receptor agonist peptide represented by formula (I):
P¹-Tyr-A2-Glu-Gly-Thr-Phe-Ile-Ser-A9-Tyr-Ser-Ile-A13-A14-Asp-A16-A17-A18-Gln-A20-A21-Phe-Val-A24-Trp-Leu-Leu-Ala-Gln-A30-A31-A32-A33-A34-A35-A36-A37-A38-A39-A40-A41-A42-P², or a pharmaceutically acceptable salt thereof;
wherein
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3},
—SO₂—NR^{A2}R^{A3},
—C(═NR^{A1})—NR^{A2}R^{A3}, or
is absent,
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, D-Ala, Ala, Gly, or Pro;
A9: represents Asp or Leu;
A13: represents Aib, or Ala;
A14: represents Leu, Aib, Ile, or Nle;
A16: represents Arg, Ser, or Lys;
A17: represents Aib, Ala, or Ile;
A18: represents Ala, His, or Lys;
A19: represents Gln, or Ala;
A20: represents Aib, Gln, or Ala;
A21: represents Asp, Asn, or Lys;
A24: represents Asn, Gln, or Glu;
A30: represents Arg, Ser, Gln, or Lys;
A31: represents Gly, Pro, or a deletion;
A32: represents Ser, Lys, Pro, Gly, or a deletion;
A33: represents Ser, Lys, Gly, or a deletion;
A34: represents Gly, Asn, or a deletion;
A35: represents Ala, Asp, Ser, Asn, or a deletion;
A36: represents Pro, Trp, or a deletion;
A37: represents Pro, Lys, or a deletion;
A38: represents Pro, His, or a deletion;
A39: represents Ser, Asn, or a deletion;
A40: represents Ile, or a deletion;
A41: represents Thr, or a deletion; and
A42: represents Gln, or a deletion.

2. The GIP receptor agonist peptide according to claim 1 or the pharmaceutically acceptable salt thereof, wherein A31 is Gly, A32-A42 are deletion; or A32 is Gly, A 33-A42 are deletion.

3. The GIP receptor agonist peptide according to claim 1 or the pharmaceutically acceptable salt thereof, wherein A31 is Pro and A32 is Gly, and A33-A42 are deletion.

4. The GIP receptor agonist peptide according to any one of claims 1-3 or the pharmaceutically acceptable salt thereof, wherein P² is OH.

5. A GIP receptor agonist peptide represented by formula (II):
P¹-Tyr-A2-Glu-Gly-Thr-Phe-Ile-Ser-Asp-Tyr-Ser-Ile-A13-A14-Asp-A16-A17-A18-A19-A20-A21-Phe-Val-A24-Trp-Leu-Leu-Ala-A29-A30-A31-A32-A33-A34-A35-A36-A37-A38-A39-A40-A41-A42-P², or a pharmaceutically acceptable salt thereof, wherein:
P¹ represents a group represented by formula
—R^{A1},
—CO—R^{A1},
—CO—OR^{A1},
—CO—COR^{A1},
—SO—R^{A1},
—SO₂—R^{A1},
—SO₂—OR^{A1},
—CO—NR^{A2}R^{A3}
—SO₂—NR^{A2}R^{A3}, or
—C(═NR^{A1})—NR^{A2}R^{A3}
wherein R^{A1}, R^{A2}, and R^{A3} each independently represent a hydrogen atom, an optionally substituted hydrocarbon group, or an optionally substituted heterocyclic group;
P² represents —NH₂ or —OH;
A2: represents Aib, D-Ala, or Gly;
A13: represents Aib, or Ala;
A14: represents Leu, Aib, Ile, Nle, or Lys(R);
A16: represents Arg, Ser, or Lys;
A17: represents Aib, Ala, Ile, or Lys(R);
A18: represents Ala, His, or Lys(R);
A19: represents Gln or Ala;
A20: represents Aib, Gln, Arg, or Ala;
A21: represents Asp, Asn, or Lys(R);
A24: represents Asn, Gln, or Glu;
A29: represents Gln, or Lys(R)
A30: represents Arg, Lys, Ser, Gln, or Lys(R);
A31: represents Gly, Pro, or a deletion;
A32: represents Ser, Lys, Pro, Gly, or a deletion;
A33: represents Ser, Lys, Gly, or a deletion;
A34: represents Gly, Asn, or a deletion;
A35: represents Ala, Asp, Ser, Asn, or a deletion;
A36: represents Pro, Trp, or a deletion;
A37: represents Pro, Lys, or a deletion;
A38: represents Pro, His, or a deletion;
A39: represents Ser, Asn, or a deletion;
A40: represents Ile, or a deletion;
A41: represents Thr, or a deletion;
A42: represents Gln, or a deletion.
wherein in the residue Lys(R), the (R) portion represents X-L-, wherein L represents a linker, and is selected from the following group consisting of 2OEGgEgE, OEGgEgE, 2OEGgE, 3OEGgEgE, G5gEgE, 2OEGgEgEgE, 2OEG and G5gEgE; and X represents a lipid.

6. A GIP receptor agonist peptide represented by formula (IV):
P¹-Tyr-Aib-Glu-Gly-Thr-Phe-Ile-Ser-Asp-Tyr-Ser-Ile-A13-A14-Asp-A16-A17-A18-A19-A20-A21-Phe-Val-A24-Trp-Leu-Leu-Ala-A29-A30-A31-A32-A33-A34-A35-A36-A37-A38-A39-P², or a pharmaceutically acceptable salt thereof, wherein:
P¹ represents H or C₁₋₆ alkyl;
P² represents —NH₂ or —OH;
A13: represents Aib, Ala, or Lys;
A14: represents Leu, Aib, Lys, or Lys(R);
A16: represents Arg, Ser, or Lys;
A17: represents Aib, Ala, Ile, Glu, Lys, or Lys(R);
A18: represents Ala, His, Glu, Lys, or Lys(R);
A19: represents Gln or Ala;
A20: represents Aib, Ala, Gln, Arg, or Lys;
A21: represents Asp, Asn, Lys, or Lys(R);
A24: represents Asn or Glu;
A29: represents Gln, Lys, or Lys(R);
A30: represents Arg, Ser, Gln, Lys, Lys(Ac), or Lys(R);
A31: represents Gly, Pro, or a deletion;
A32: represents Ser, Gly, or a deletion;
A33: represents Ser, Gly, or a deletion;
A34: represents Gly or a deletion;
A35: represents Ala, Ser, or a deletion;
A36: represents Pro or a deletion;
A37: represents Pro or a deletion;
A38: represents Pro or a deletion; and
A39: represents Ser or a deletion;
wherein in the residue Lys(R), the (R) portion represents X-L-, wherein L represents a linker and is selected from the group consisting of 2OEGgE, 2OEGgEgE, G4gE, GGGGG, G5gE, G5gEgE, G6, gEgEgE, OEGgEgE, OEGgEOEGgE, GGPAPAP, and GGPAPAPgE; and X represents C₁₇-C₂₂ monoacid or C₁₇-C₂₂ diacid.

7. The GIP receptor agonist peptide according to claim 6 or the pharmaceutically acceptable salt thereof, wherein:
A17: represents Aib, Ala, Ile, Glu, or Lys(R);
A18: represents Ala, His, Glu, or Lys(R);
A21: represents Asp, Asn, or Lys(R); and
A29: represents Gln or Lys(R).

8. The GIP receptor agonist peptide according to claim 6 or the pharmaceutically acceptable salt thereof, wherein:
A13: represents Aib or Ala;
A14: represents Leu, Lys, or Lys(R);
A16: represents Arg;
A17: represents Aib, Lys, or Lys(R);
A18: represents Ala, Lys, or Lys(R);
A20: represents Aib;
A29: represents Gln;
A30: represents Arg, Ser, or Lys;
A31: represents Gly or Pro;
A33: represents Ser or a deletion; and
A35: represents Ala or a deletion;
wherein L is selected from the group consisting of 2OEGgE, 2OEGgEgE, OEGgEgE, OEGgEOEGgE, G5, GGPAPAP, and GGPAPAPgE.

9. The GIP receptor agonist peptide according to claim 8 or the pharmaceutically acceptable salt thereof, wherein:
A14: represents Leu or Lys(R);
A17: represents Aib or Lys(R);
A18: represents Ala or Lys(R); and
A21: represents Asp, Asn, or Lys(R).

10. The GIPR agonist peptide of any one of claims 5-9 or the pharmaceutically acceptable salt thereof, wherein the lipid X is C₁₇-C₂₀ monoacid or C₁₇-C₂₀ diacid.

11. The GIPR agonist peptide of claim 10 or the pharmaceutically acceptable salt thereof, wherein the lipid X is a C₁₈ diacid

12. The GIPR agonist peptide of any one of claims 5-11 or the pharmaceutically acceptable salt thereof, wherein the linker L is 2OEGgEgE or GGGGG.

13. The GIPR agonist peptide of any one of claims 5-12 or the pharmaceutically acceptable salt thereof, wherein (R) is 2OEGgEgE-C₁₈ diacid or GGGGG-C₁₈ diacid.

14. The GIPR agonist peptide of any one of claims 5-13 or the pharmaceutically acceptable salt thereof, wherein the peptide has a Lys(R) amino acid residue at amino acid position A14 and (R) is GGGGG-C₁₈ diacid.

15. The GIPR agonist peptide of any one of claims 5-13 or the pharmaceutically acceptable salt thereof, wherein the peptide has a Lys(R) amino acid residue at amino acid position A18 or A21 and (R) is 2OEGgEgE-C18 diacid

16. The GIPR agonist peptide of claim 5 or 6, represented by formula (V):
Me-Tyr-Aib-Glu-Gly-Thr-Phe-Ile-Ser-Asp-Tyr-Ser-Ile-A13-A14-Asp-Arg-A17-Ala-Gln-Aib-A21-Phe-Val-Asn-Trp-Leu-Leu-Ala-Gln-A30-A31-A32-A33-A34-A35-A36-A37-A38-A39-P², or a pharmaceutically acceptable salt thereof, wherein
P² represents —NH₂ or —OH;
A13: represents Aib or Ala;
A14: represents Leu, Lys, or Lys(R);
A17: represents Aib, Lys, or Lys(R);
A21: represents Asp, Asn, Lys, or Lys(R);
A30: represents Arg, Ser, Lys, or Lys(R);
A31: represents Gly or Pro;
A32: represents Ser, Gly, or a deletion;
A33: represents Ser or a deletion;
A34: represents Gly or a deletion;
A35: represents Ala or a deletion;
A36: represents Pro or a deletion;
A37: represents Pro or a deletion;
A38: represents Pro or a deletion; and
A39: represents Ser or a deletion,
wherein L is 2OEGgEgE or GGGGG; and X represents C₁₈ diacid.

17. The GIPR agonist peptide of claim 16 or the pharmaceutically acceptable salt thereof, wherein:
A14: represents Leu or Lys(R);
A17: represents Aib or Lys(R);
A21: represents Asp, Asn, or Lys(R); and
A30: represents Arg, Ser, Lys, or Lys(R).

18. The GIPR agonist peptide of claim 16 or 17 or the pharmaceutically acceptable salt thereof, represented by the formula:
Me-Y-Aib-E-G-T-F-I-S-D-Y-S-I-A-Km-D-R-Aib-A-Q-Aib-D-F-V-N-W-L-L-A-Q-S-P-G-OH;
Me-Y-Aib-E-G-T-F-I-S-D-Y-S-I-A-Km-D-R-Aib-A-Q-Aib-N-F-V-N-W-L-L-A-Q-S-P-S-S-G-A-P-P-P-S-OH;
Me-Y-Aib-E-G-T-F-I-S-D-Y-S-I-A-L-D-R-Km-A-Q-Aib-D-F-V-N-W-L-L-A-Q-S-P-S-S-G-A-P-P-P-S-NH₂;
Me-Y-Aib-E-G-T-F-I-S-D-Y-S-I-A-L-D-R-Aib-A-Q-Aib-Km-F-V-N-W-L-L-A-Q-K-G-OH;
Me-Y-Aib-E-G-T-F-I-S-D-Y-S-I-Aib-L-D-R-Aib-A-Q-Aib-Km-F-V-N-W-L-L-A-Q-R-G-OH;
Me-Y-Aib-E-G-T-F-I-S-D-Y-S-I-Aib-L-D-R-Aib-A-Q-Aib-N-F-V-N-W-L-L-A-Q-Km-P-S-S-G-A-P-P-P-S-NH₂.

19. The GIPR agonist peptide of claim 18 or the pharmaceutically acceptable salt thereof, represented by the formula:
Me-Y-Aib-E-G-T-F-I-S-D-Y-S-I-A-Km-D-R-Aib-A-Q-Aib-D-F-V-N-W-L-L-A-Q-S-P-G-OH; wherein Km is Lys-GGGGG-Cis diacid.

20. The GIPR agonist peptide of claim 18 or the pharmaceutically acceptable salt thereof, represented by the formula:
Me-Y-Aib-E-G-T-F-I-S-D-Y-S-I-A-Km-D-R-Aib-A-Q-Aib-N-F-V-N-W-L-L-A-Q-S-P-S-S-G-A-P-P-P-S-OH; wherein Km is Lys-GGGGG-C₁₈ diacid.

21. The GIPR agonist peptide of claim 18 or the pharmaceutically acceptable salt thereof, represented by the formula:
Me-Y-Aib-E-G-T-F-I-S-D-Y-S-I-A-L-D-R-Km-A-Q-Aib-D-F-V-N-W-L-L-A-Q-S-P-S-S-G-A-P-P-P-S-NH₂; wherein Km is Lys-GGGGG-Cis diacid.

22. The GIPR agonist peptide of claim 18 or the pharmaceutically acceptable salt thereof, represented by the formula:
Me-Y-Aib-E-G-T-F-I-S-D-Y-S-I-A-L-D-R-Aib-A-Q-Aib-Km-F-V-N-W-L-L-A-Q-K-G-OH; wherein Km is Lys-2OEGgEgE-C₁₈ diacid.

23. The GIPR agonist peptide of claim 18 or the pharmaceutically acceptable salt thereof, represented by the formula:
Me-Y-Aib-E-G-T-F-I-S-D-Y-S-I-Aib-L-D-R-Aib-A-Q-Aib-Km-F-V-N-W-L-L-A-Q-R-G-OH; wherein Km is Lys-2OEGgEgE-C₁₈ diacid.

24. The GIPR agonist peptide of claim 18 or the pharmaceutically acceptable salt thereof, represented by the formula:
Me-Y-Aib-E-G-T-F-I-S-D-Y-S-I-Aib-L-D-R-Aib-A-Q-Aib-N-F-V-N-W-L-L-A-Q-Km-P-S-S-G-A-P-P-P-S-NH₂; wherein Km is Lys-2OEGgEgE-C₁₈ diacid.

25. The GIP receptor agonist peptide according to any one of claims 1 to 24 or the pharmaceutically acceptable salt thereof, wherein the GIP receptor agonist peptide has a selectivity ratio, expressed as a ratio of (GLP1R EC50/GIPR EC50) of greater than 10, or greater than 100, or greater than 1,000, or greater than 100,000.

26. The GIP receptor agonist peptide according to any one of claims 1 to 24 or the pharmaceutically acceptable salt thereof, wherein the GIP receptor agonist peptide has a human IV T1/2 life of elimination of greater than 50 hours.

27. The GIP receptor agonist peptide according to any one of claims 7 and 9-15 or the pharmaceutically acceptable salt thereof, wherein the GIP receptor agonist peptide has a solubility of 15 mg/mL or greater.

28. A medicament comprising the GIP receptor agonist peptide according to any one of claims 1-27, or a pharmaceutically acceptable salt thereof.

29. A pharmaceutical composition comprising the GIP receptor agonist peptide according to any one of claims 1-27, or a pharmaceutically acceptable salt thereof.

30. The GIP receptor agonist peptide according to any one of claims 1-27 or the pharmaceutically acceptable salt thereof, or the medicament according to claim 28, or the pharmaceutical composition according to claim 29, which is administered to treat emesis as a monotherapy.

31. The GIP receptor agonist peptide according to any one of claims 1-27 or the pharmaceutically acceptable salt thereof, or the medicament according to claim 28, or the pharmaceutical composition according to claim 29, which is administered Q1W, or once per four to seven days, or once per four to five days, or once every four days, or once every five days, or once every six days, or once every seven days, or once every eight days, or once every nine days, or once every ten days.

32. The medicament according to claim 28, which is an activator of a GIP receptor.

33. The medicament according to claim 32, which is a suppressant for vomiting or nausea.

34. Use of the GIP receptor agonist peptide of any one of claims 1-27, or a salt thereof, or the medicament according to claim 28, or the pharmaceutical composition according to claim 29, for the manufacture of a suppressant for vomiting or nausea.

35. The GIP receptor agonist peptide of any one of claims 1-27, or a salt thereof, or the medicament according to claim 28, or the pharmaceutical composition according to claim 29, for use in suppressing vomiting or nausea.

36. A method for preventing or treating emesis in a subject, comprising administering an effective amount of the peptide of any one of claims 1-27, or a salt thereof, or the medicament according to claim 28, or the pharmaceutical composition according to claim 29, to the subject.

37. The method according to claim 36, wherein the emesis is nausea and/or vomiting.

38. The medicament according to claim 33, the use according to claim 34, the peptide, medicament, or pharmaceutical composition according to claim 35, or the method according to claim 37, where the emesis, vomiting or the nausea is caused by one or more conditions or causes selected from the following (1) to (10):
(1) Diseases accompanied by vomiting or nausea such as gastroparesis, gastrointestinal hypomotility, peritonitis, abdominal tumor, constipation, gastrointestinal obstruction, chronic intestinal pseudo-obstruction, functional dyspepsia, chemotherapy-induced nausea and vomiting (CINV), chronic unexplained nausea and/or vomiting, Cyclic vomiting syndrome (CVS), nausea and/or vomiting associated with gastroparesis, acute pancreatitis, chronic pancreatitis, hepatitis, hyperkalemia, cerebral edema, intracranial lesion, metabolic disorder, gastritis caused by an infection, postoperative disease, myocardial infarction, migraine, intracranial hypertension, and intracranial hypotension (e.g., altitude sickness);
(2) Vomiting and/or nausea induced by chemotherapeutic drugs such as (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome;
(3) Vomiting or nausea caused by radiation sickness or radiation therapy for the chest, the abdomen, or the like used to treat cancers;
(4) Vomiting or nausea caused by a poisonous substance or a toxin;
(5) Vomiting and nausea caused by pregnancy including hyperemesis gravidarium; and
(6) Vomiting and nausea caused by a vestibular disorder such as motion sickness or dizziness
(7) Opioid withdrawal;
(8) Pregnancy including hyperemesis gravidarium;
(9) A vestibular disorder such as motion sickness or dizziness; or
(10) A physical injury causing local, systemic, acute or chronic pain.

39. The method according to claim 36, wherein the emesis is a result of chemotherapy-induced nausea and vomiting (CINV), chronic unexplained nausea and/or vomiting, Cyclic vomiting syndrome (CVS), and nausea and/or vomiting associated with gastroparesis.

40. The method of claim 36, wherein the subject is a non-type 2 diabetes mellitus subject.

41. The method according to claim 36, wherein the emesis is delayed emesis or anticipatory emesis.

42. The method according to any one of claims 36-41, wherein emesis is treated in the subject without inducing anxiety or sedation in the subject.

43. The method according to any one of claims 36-42, wherein emesis is treated in the subject without inducing suppression of glucagon secretion when plasma glucose levels are above fasting levels.

44. The method according to any one of claims 36-43, wherein emesis is treated in the subject without substantially activating the GLP-1 receptor.

45. The method according to claim 43 or 44, wherein emesis is treated in the subject without concomitant, subsequent, or prior administration of a GLP-1 receptor agonist.

46. The method according to any one of claims 36-45, wherein emesis is treated in a subject not taking a medicament to control a metabolic syndrome disorder.

47. The method according to any one of claims 36-45, wherein emesis is treated in a subject taking a medicament to control a metabolic syndrome disorder.

48. The method according to claim 47, wherein the metabolic syndrome disorder is type 2 diabetes mellitus or obesity.

49. The method according to any one of claims 36-48, wherein the emesis is caused by or causes cyclic vomiting syndrome, or nausea or vomiting associated with chemotherapy.

50. The method according to claim 38 or 49, wherein where the chemotherapy or chemotherapeutic agent comprises: (i) alkylating agents (e.g., cyclophosphamide, carmustine, lomustine, chlorambucil, streptozocin, dacarbazine, ifosfamide, temozolomide, busulfan, bendamustine, and melphalan), cytotoxic antibiotics (e.g., dactinomycin, doxorubicin, mitomycin-C, bleomycin, epirubicin, actinomycin D, amrubicin, idarubicin, daunorubicin, and pirarubicin), antimetabolic agents (e.g., cytarabine, methotrexate, 5-fluorouracil, enocitabine, and clofarabine), vinca alkaloids (e.g., etoposide, vinblastine, and vincristine), other chemotherapeutic agents such as cisplatin, procarbazine, hydroxyurea, azacytidine, irinotecan, interferon α, interleukin-2, oxaliplatin, carboplatin, nedaplatin, and miriplatin; (ii) opioid analgesics (e.g., morphine); (iii) dopamine receptor D1D2 agonists (e.g., apomorphine); (iv) cannabis and cannabinoid products including cannabis hyperemesis syndrome

51. The method according to claim 36, wherein the subject has type 2 diabetes mellitus.

52. The method according to any one of claims 36-51, wherein the GIP receptor agonist peptide or medicament is administered subcutaneously, intravenously, intramuscularly, intraperitonealy, orally or via inhalation.

53. The method according to any one of claims 36-52, wherein the effective amount of the GIP receptor agonist peptide administered to the subject is about 0.01 to 0.5 mg/kg/day, 0.1 to 5 mg/kg/day, 5 to 10 mg/kg/day, 10 to 20 mg/kg/day, 20 to 50 mg/kg/day, 10 to 100 mg/kg/day, 10 to 120 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day or 900 to 1000 mg/kg/day.

54. The method according to any one of claims 36-53, wherein the subject is human.

55. The method according to any one of claims 36-54, wherein the GIP receptor agonist peptide or medicament is administered to the subject before, during, or after the subject develops the disease-state.

56. The method according to any one of claims 36-55, wherein the GIP receptor agonist peptide or medicament is administered to the subject once per week, or once per 5-7 days, or four to six times per month.

57. The method according to any one of claims 36-56, wherein the GIP receptor agonist peptide or medicament is administered to the subject for 1-5 weeks, 1-5 months, or 1-5 years.
